SEQUENCE LISTING (323 sequences)

SEQ ID NO: 1 - protein, 374 aa, Capsicum annuum
MCKRIKEETMNPRLPDEIILNILSYLPLSCLFKFRCVSKFWNTMFLHPHLTFHHYRNSHEFNLDDVTDSVKEREYALKERENEDRDEVMFATITTSSQGLRLQRTISIYEDEDDSPITGSKLRYRVECSILRQRLDIPNPQYPSLCIALDFVSHSQAVKLVSLHEDGYEILALGVGSDQTTYSWRDVNIPNFSHEKRDGIQIFFRMGVAYCIWHVQRDHGLIDVEIDVLDMVNETYIAHTTIPRGFLTNAYVMDCDGRLSFVEQVKNELHALVLNVNRKLRWEKWIFKLNFLKPDNKELMTFIAFADSSFLFFVKEDKISFCAYDIITRKIIMTHVFSSCFNSPEEVMDVIKMMMFHRNGGSSTLKNLYFEAFF

SEQ ID NO: 2 - protein, 298 aa, Capsicum annuum
MAANDSNTLGGPTNFSTAILIRVDQSGQGDFKKIQDAIDVVPSNNSELYFIWIKPGTYREKVVVPADKPFITLSGTKASNTIITSSEGGDTTESPTLTILASDFVARYLIIQNLFGRSGKAVALKVKGDRAAFYGCRIQSYQDTLLDDAGRHYYSNCYIEGAVDFIYGNGASLYERCHIHSVAAGAITAQGRNSPLENTGFTFLGCKITGTGGNTSLGRPWGSYSRVVFAYTYMASIIQPEGWNDWGDSTKQSTAYYGEYKCYGPGANRSNRVGWSRSLSNEDAAPFLTKNXSAGEVG

SEQ ID NO: 3 - protein, 310 aa, Capsicum annuum
MFLSVFLYRGVCRIVIKMNDLMTKSFLSYVDLKNQAMMDLEAGPDIEMGQLDPTDERNLSKFFEEVAAVKSDMEEINNLLVNLQDLNQEAKSAPSPKILQGLRDQINSDSVTVLRKAKIIKTRLELLDKSNLDNRGLSAAYKEGSPVERTRISVTSGLRIKLKDMMNDFQCLRESIVAEHKEGLRKQYTNANGEELSEEVIEKMMQERVFDGIVGKEVVENRERHEAVNEIQKSLVELHQVFLDMAVMVETQGDQMNNIEQNVVNAGGYMKDGTKELDRAKRMKRRRTWACWIGVVVLFFLLLCIIAILF

SEQ ID NO: 4 - protein, 550 aa, Capsicum annuum
MTADFSFRKNSGPFNGRWFTVFCSILILSVSGGTYLFGLYSQYVKSSLGYDQTTLNLLSFFKDVGANVGIIAGLINEVCPPWVVLLIGSVMNFSGYIYLWLAVNGDELVKPRVWQMCLCLMIGANSQTFTNTGALVTCVKNFPESRGIVIGLLKGFVGLSGAIITQLFHAIYGNDGKSLVLLIAWLPPFVCCSLLKYVRVMKGVNRQENEVGIFYQLLYISLGLAGFLLMITILQNRVGFSLVEYRLSSFIVLLLLFAPVVVVIREELKLGKNRKQVFDEFVPLKGIEQGKLASRSQGKEVLWFKNVFKPPERGEDYTILQALLSIDMLILFITTIFGAGGTLTAIDNIGQIGKALGYPNTSIATFTSLVSIWGYLGRVVSGFVSEIFLTKYKVPRPLMLTFVLLLSCFGHILIAMGVPSVLDVALAILGFCFGAMAPLIFAIISELFGLKHYATLLNFGGAASPIGAYVFNVRLVGYFYDKEALKQMAALGLFRKPGDHLTCIGIECYRSSFLIIAAASFVGCIVSYILVLRTRKFYKGDIYKKFREQA

SEQ ID NO: 5 - protein, 193 aa, Capsicum annuum
MEGVGGDGASAAAALNQQRLELSKLFQYYLDKTTPHATYRWIGTFVFALLYALRVYSVQGFYVVTYGLGIYVLNLLIGFLSPLVDPELEPSDGPMLPTKGSDEFKPFIRRLPEFKFWYAITKAFCVAFVMTFFSLFDVPVFWPILLCYWIVLFVLTMRRQITHMIKYKYVPFNLGKQVNSLVIYVVVYISTSI

SEQ ID NO: 6 - protein, 657 aa, Capsicum annuum
MSDRVAQSSSDCDGRTGKSVHSVQSVWMAHWTRTSYNSAAETHNLAPNALANKENDQDSKPLKSVVKLETMSKLSKSVKRLRESETQTFEVINESLRTSSRTIAKETLGNWSLPLHNPRENVKTPFQDPLDCVKTQSYDVGCSTLASRPSLDNPSHLASHLVPYGDPGQYITKEGERTQKPVINRSFLAAKEELPRLETLEHEHGRSITPYLRQRNTFLLDRPSTSRNLPPEFGGEEFQKIPGLSFIRLLKDEPSPSQVPDQQKLPHPLHDIEIMRTRNTMDPVEGMTGYCPGISQTTHSMLITKGTDANLLEGNNVIANSRMWSEINAKASLSNRGSPSKSFGRYKGGMKLQIQSCSTGSERKKSIEGSKPSQFVLKNESSAETDTMDMDVLQEKNQLCGTSSSIVKKVNKMNRNSPPQLALDFPRKETGRKQFKLDINLELPAPTDNMEASSSRTESLDLGSILARAEQPSSSRTDLCAEGLPGQDPSNRWVKRLKVGAPVSLAFGTRSSRLVGEKSPEKSHEFPSKITEPAITSSELASCKLHGKELMTPDNTPGLAMNSNPSSMSVIKKDLDSLTSHCWIQRLLHERTTTSAKRPQPVVVCEPQTSKLELDDFQKKQLPSLGAMALMGKAMNGFQPCEFRRKGPLVVWNTRSF

SEQ ID NO: 7 - protein, 408 aa, Capsicum annuum
MDTCCVAVKSTAHLGRVSKGGFNNGEKEFFGEQIRGSLKNNLKFNQSSKSLKLEKKENKIKPGVAFSVITTENEQETLFLEETAPRFERRRANPKDVASVILGGGEGAKLFPLTSRAATPAVPVGGCYKLIDIPMSNCINSGINKVFVLTQYNSAPLNRHIARTYFGNGVSFGDGFVEVLAATQTPGETGKRWFQGTADAVRQFIWVFEDAKNKNIENILILSGDHLYRMDYMELVQNHIDRNSDITLSCALAGDSRASDFGLVKIDSRGRVVQFAEKPKGFDLKAMQVDTTLLGLSPQDAKKSPYIASMGVYVFKTDVLLKLLKWRYPTANDFGSEIIPAAIDEQNVQAYIFRDYWEDIGTIKSFYEANLELTSEFPKFQFYDPKTPFYTSPRFLPPTKIDNCKASG

SEQ ID NO: 8 - protein, 241 aa, Capsicum annuum
MTMLFRFMEFCIFFVIISRFSTQLPLSFKLSTEYYFKGIGVTLISPLFVFVLGNAIVIILYLKSGHSSPKDDSTNNVKIDLYDEYKQKCSMNKEVYCEQGKKQSILVEEAYCEQSKKQSKQSIPAEKAYCEQSKKQRKQSIVAERRVEKAIRRSHSGNSLCLSHDEKKHPRQKMIRSATVGCLKVINTDNVKPERTTTTTTTTSYPEDGMSSDEFRKTVEAFIARQQRFLREQEFSDVVSS

SEQ ID NO: 9 - protein, 238 aa, Capsicum annuum
MEYSLKNQNIKLEKTKALLSYKRRQRMTMLFRFMEFCIFLVIMSRFSTQFPFGFKLSTDYFKGVGVTLISPLFAFVLGNAIVIILFLKSGQSSAKVDSTNNVKIDLYDEYKQKCWVNKEVYCEQIEKQSIHVEKAYCEQSKKQSILRQKQLAEKKLDRSHSDSFTSLSHDEKPRRELVRSATVGCLKVIHTDNIKSMEDEMSNEEFRTTVEAFIARQQRFLREQELLSPVVSVKKKKC

SEQ ID NO: 10 - protein, 256 aa, Capsicum annuum
MESLNIHNIKVEKANAKLRYKKRQRVTTLFRFIEFCIFFVIISRFSTQLPLGIKLSTDYFKGVGVTLISPRFVFVLGNVIVIILFLKSGQSSDTTNNVKIDLYDEYKQKCSTNKEVCCEQIEKQSILVEKAYFEQIKKQRKQSIHVEKAYCEQSKKQRKQSTHVERQVTKKIPRSHSDNSLYLSSDEKKHPRKSLMRSATVRYTENVKPTTTMTTTSYPEDEMSNEEFRKTVENFIARQQRFLREEEFEAVVSYKS

SEQ ID NO: 11 - protein, 268 aa, Capsicum annuum
MRAALDTDVIVRCTQSRCGGTRLKANLCKFDDNHIVHCTTGFPIMVDLIVKRSSGGSTCTNYIYDHRAVQNFWIPYDCLLLDDDASNIIAKIIFLMNVPISLELNKQSISYSDNKTTTTTLTNKDTLISKILNFARTMKDFPSNELWYMSLVITKVTYVPHVDFIKMYDMVGEYYNLNTWKRTQEVLDSRIAAFELLQKKIGMKKMRFQASEFEDSNLLDTCSICQDEFLHGRIVSCIKDCSHVYHDICILEWLLRNRTCPYCRSKLA

SEQ ID NO: 12 - protein, 178 aa, Capsicum annuum
MIGRFELQTSTIQWINSQGLIRMMSRFGIAQKGSVKTSYQNKRIRPSSQKNRGIRYLHAYGGLYWKKGTQDVSQTARQLISEDKDELLAAVPLLICVTPNQGWSFLLHTWENELAILEFIHLLVETMDRHFGNVCELDIMFHLEKAHFMLEEMVMNGCIVETSKANILTPIQLMDKAS

SEQ ID NO: 13 - protein, 88 aa, Capsicum annuum
MGIRFILMVNKQGQTRLAQYYEYLTLEERRALEGEIVRKCLARNEQQCSFVEHRNYKIVYRRYASLFFLVGVDNDEFERLVESLSSQV

SEQ ID NO: 14 - protein, 527 aa, Capsicum annuum
MASETNTGLSNGNGNAHSTPQRTYQVVVAATRNMGIGKEGKLPWRLPSDLKFFKGITGVTSDPTKKNAVVMGRKTWESIPIEHRPLPGRLNVVLTRSGSFDIATAENVVICGSLGSALQLLAASPYCLSIENVFVIGGGEIFRASLNAHGCDAIHITEIETDIECDTFTPAIDTSVFQPWYSSFPVVENKIRYSFTTYVRVKNSGVETVNQANNEIPENGLGSSSIEVKSFSFLPKMIFEKHEEHMYLSLVEEIISNGILKDDRTGTGTLSKFGCQMRFNLRRSFPLLTTKKVFWRGVVEELLWFISGSTSAKVLQEKGIRIWDGNASRDYLDSIGLRDREEGDLGPVYGFQWRHFGARYIDMHTDYSGQGFDQLADVINKVKNNPDDRRIILSAWNPSDLKLMALPPCHMFAQFYVANGELSCQMYQRSADMGLGVPFNIASYALLTCMIAHVSDLVPGDFVHVIGDAHVYRNHVRPLQDQLQKLPRPFPVLKINPQKKDIDSFVAADFNLIGYDPHQKIEMKMAI

SEQ ID NO: 15 - protein, 477 aa, Capsicum annuum
MAATHLVSHSPSLPKSLKYPSNPIPKFSFFKPLNPISRNLSICAAVSTSEPQTHKFQHCFKKSEDGFLYCEGVKVEDVMETVERRPFYLYSKPQITRNVEAYMAALEGLNSIIGYAIKANNNLKILEHLRALGCGAVLVSGNELKLALTAGFDPTRCIFNGNGKLLEDLVLAAQAGVFVNIDSEFDLDNIVAAARLAGKQVNVLLRINPDVDPQVHPYVATGNKSSKFGIRNEKLQWFLDSVKAHPQELKLVGAHCHLGSTITKVDIFRDAAVLMVNYIDEIRSQGFEIDYLNIGGGLGIDYYHSGAVLPSPRDLINTVRELVLSRNLNLIIEPGRSLIANTCCLVNRVTGVKTNGTKNFIVIDGSMAELIRPSLYDAYQHIELVSPASTEAAISKFDVVGPVCESADFLGKDRELPAPSRGTGLVVHDAGAYCMSMASTYNLKMRPPEYWVEEDGSVSKIRHGETFEDHLRFFDGL

SEQ ID NO: 16 - protein, 350 aa, Capsicum annuum
MSDYKSGPLERPVRPSLERFDIDAEDERKTRLGSIKKAAINASTKFRNSLTKRGRRNSRVMSVVVLEDEHDAEELKAVDALRQALILEELLPAKHDDYHLMLRFLKARKFDIEKTKQMWSDMLQWRREFGADTITEDFDFKEREEVLKYYPQGHHGVDKEGRPVYIERLGQVDSVKLLQATTMERYLKYHVQEFERTIHEKMPACSLAAKRHIDTSTTILDVQGVGLKNFNKSARDLIQRLQAIDGNNYPESLCRMYIINAGSGFRLLWNSVKSFLDPKTTQKIHVLGNKYQSKLLEIIDASELPEFLGGTCTCADKGGCMLSDKGPWNDPEIMRVTFNFVVLHSRGLIP

SEQ ID NO: 17 - protein, 251 aa, Capsicum annuum
MVRDGDHKCTHKFVISMDEKTIPEDDYANAKVPSMLQNVNKSTSFKKETNSSKVQRDNVAHGQLSPVREEFDIPTVDKAVDATWSKDVVKAGNFAKSKDVFPVHDVCKTSEGFGNPIFSGVMTLVMGIVTLVRLTKNMPRKLTDATLLAGSLYSADMIKEQTHGHVISTKDYISMMKRMEELEDKVITLNQKPASMPPEKEDMLNAAINRVGTLEQELLATKKALDEALVRQQELLSALEKKKKKKKFLVF

SEQ ID NO: 18 - protein, 289 aa, Capsicum annuum
MIGEARRAKQLVKVLNSEGKVVYAKPRYPTVKGSSDTSTAESGSEAEDIASPKAVRSYSHLRLTPVREEAKAIGMTGYTTKFPGYDEYVPMVDKAVDSTWKKEPSLQRLSTPKVMLPPANSEKSVEGFRARVLGLLMTFFMTVMILFRSVTCRVTKKLQDASTLEDPVVQDFTFDASPKEEFRPPSPTPAFTEAQLLSVVLKRLGELEDKVNTLQEKPSEMPYEKAELLNAAVCRVDALEAELIATKKALHEALMRQEELLAYIDSQEVAKFQVSFCYCMVVEHHQIST

SEQ ID NO: 19 - protein, 381 aa, Capsicum annuum
MSGPLDRFARPCFEGSSTHDERRERKSDFENSEDERRTRIGSLKKKALNASTKFKHSLKKKSSRRKSDGRVSSVSIEDIRDAEELQAVDQFRQALLLDELLPERHDDYYMMLRFLKARKFDIEKAKHMWADMLQWRKDFGADTITQAIFFIIFYHILVQTFRLKFFLYLIIHQDFEFPEMEEVVKYYPQGYHGIDKEGRPVYIERLGKVDPNKLMQVTTMDRYVKYHVREFEKSFLIKFPACTIAAKRQIDCSTTILDVQGVGLKNFSKSARELILRLQKIDGDNYPETLHQMFIINAGPGFRLLWNTVKSFLDPKTASKIHVLGNKYQNKLLEIVDASELPDFLGGACTCADRGGCMRSDKGPWQNPDILKVLSRPSQPL

SEQ ID NO: 20 - protein, 74 aa, Capsicum annuum
MVLLYLPCSKIKFEELSKDDYFSKSNEFANTWLKDKKKLFFSDLLSESACDLFFDFVKNGTRESLTSNTPKERQ

SEQ ID NO: 21 - protein, 96 aa, Capsicum annuum
MQGWQAVAFRISSDKAYFSGCGFYGAQDTLCDNAGRHYFKECYIEGSIHFIFGNDRSMYKPGAGYRYARCPGKGPTTRVYRTQPYLAFLPDVVSKT

SEQ ID NO: 22 - protein, 192 aa, Capsicum annuum
MSNPTFKKTIDVGENKNNHPNIVTGGRRLTSVEMNEKRAKGLCFFYDEKYTMGNVCKAKKQLYLIDVVEEEERVKEVELIQEQELQDATHEFMTISLQAYTGVTGYQTLRVTGYNEKRPLQVLIDTGNTHNFIDQEVAKKLGCKAEPITEQSVCVVDGRKVQTAYVCKNLQWLLQGTTFCHASNPIGADWHP

SEQ ID NO: 23 - protein, 170 aa, Capsicum annuum
MDKRGGKGRQKIEMKLIESKEARTVAFSKRKKGLGEKAKEYSNLTGADVGFILFTQAGKPHSYGSPSIEKVIDKFHELKQNDSQQNHTNMSKTNAFEAFEDLRKEVQAFKEKDLDSEMPTNKHMLEHKLKHFEAYKSRLDKIKKEIKGDVPAEQFKFISEVALDLEEMSS

SEQ ID NO: 24 - protein, 216 aa, Capsicum annuum
MSTVNYLEALKLEEKLASDRKALKITDTLLSLQHPPTYTVGKRETVHNLLIPDSELKAIGAELHFTQRGGDITFHGPHQAILYPIVSLRYIGLGARKYVEKLELTMIELASMXYIGLGARKRCETGVWVDDRKIGAIGVRISSGITSHGLAFNMDPDLNYFKHIVPCGIADKDVTSLKKEADVELPAEEVIQEKLIACFVKIFGYSNVILKDKSKL

SEQ ID NO: 25 - protein, 132 aa, Capsicum annuum
MDEIPFNQRVKVASVHLEGDAIAWHRAFIKSRNSVVEPTWTEYILSLNERFRDNFDDPMEALKNLKQTSGVREYQAKFDRLLTGVNLSIENEISCYLGGLKLELNKVVRIQNPKTLMQAYKISRLQEKVFEA

SEQ ID NO: 26 - protein, 89 aa, Capsicum annuum
LIESKEACTVAFSKRKKGLDEKAKEYSNLTGADVGFILFTQAGKPHSYGSPSIEKVIDKFHELKQNDSQQNHTNMSKTNAFEAFEDLRK

SEQ ID NO: 27 - protein, 170 aa, Capsicum annuum
MDKRGGKGRQKIEMNLIESKEARTVAFSKRKNGLGKKAKDYSNLTGADVGFILFTQAGKPHSYGSPSIEKIIDKFHELKQNDSQQNHTDMSKTNVFEAFEDLRKEVQAFKEKDLGSELPTDKHILENKLKQFEAYKLRLDKIKNEIKGDVQAEQFKFISEVALDLEEMSS

SEQ ID NO: 28 - protein, 151 aa, Capsicum annuum
MVMMKEPTPRRDKENYNNINKNSYSSMRCPHCAGPLSKEMETSEWTVPPLIRDSFSMIGSAVGGTTSAFYGFNHVMPIVRRWVKGPMWLHFLVGAPPVIVFSSACAGLAGGAVPACAQLASSSYHAAISSSTLPPTASQDENMRKSTSSTL

SEQ ID NO: 29 - protein, 523 aa, Capsicum annuum
MFVRLVRTSCAAVRPLSTEAATTVKSTRSGGTSTTTSLEGGTSSATSSRGRDTLGKRLLSLVYAKRSAVITIRKWKEEGHPVRKYELNRIVRELRRHKRYKHALEICEWMRVQDDIQLLSGDYAVHLDLIAKIRGMNSAEKFFEDLPDKLKVQTTCTALLHTYVQHKNIVKAEALMEKMSECGFLKYPLPYNHMLSLYISQGQLDKVPGLIQELKKNTSPDIVTYNLELAVFASQNNVEAAEKTFLELKKAKLDPDWITFSTLTNIYTKNSLPDKAKSTLREMEKRISKKARSAYSSLISLHTNLESKDEVFRIWKEMKSIFGKLSDVECICIISSLLKLDEFGEAMNLYTEWEAVTVTKDTRIANLILAAYINKNKMEKAIDFQERMVQKGISPSCTTWELLTRGYLKQKEIDKVLEFFKKTVTSVSKWDPDKKMVQEMFHVVEEQGNIQVAEQLLVTLRHAKYVNTEIYNALLRTYVNAGKMPMIVAERMKKDNVKMDDETQKLIGLTSKMTVTEVPNGVA

SEQ ID NO: 30 - protein, 221 aa, Capsicum annuum
MATLLSSHQDITYPTHTFFSTVPFYDFKIEATVTKKASEVDKWIFQTIQIHRRRLHKLLIGLDIEWRAPHYPTVQLPSTALLQLCVGRRCLIFQLLHADCIPHSLQFFLGNPNFTFLGVGVHGDALKLNKDYGLYVANTVDLNKLALVVKEVEEYGRIGLKRMAYEVLGKVMKKPFDVTMSEWDADELCVEQVEYAAIDAFVSFELGISLFSELFSRGGGV

SEQ ID NO: 31 - protein, 470 aa, Capsicum annuum
MSSRQDSDVDDDFSDLYKEYTGPVRSNTTKPQDKTVTEKRPHVGSDEEEEDARDPNAVPTDFTSREAKVWEAKSKATERNWKKRKEEEMICKICGESGHFTQGCPSTLGASRKSQDFFERVPARENHVKALFTEKVINQIEKDVGCKIKLEEKFIIVSGKDRLILRKGVDAVHKIKEEADKKGPSSSQVSRSRSPERRSPVRSRSPVRSRMARSDSQRSNHSPQSASQLHHRYGRQEKVVDDRGHDDFHKIARGNSQAYGNDGARGRSSLSKSPARPAYMSNSYSSYDGHGQGRDIYRSDGWDASRRGSDMKPNRDFDHPVPQSLESVELEYQKDAIDLGRIRDKEEDEENHKHREAIREIRGNYMKKLAVLRVEHAKQWEEFLQLDALRRQQLAEQRMSASGFGGYNQQSYQEYENSAANPHYSGPNIPMEPRGRYPNPMDNYHPSRPHDSYDFQRQRRDDFGKAYNRY

SEQ ID NO: 32 - protein, 355 aa, Capsicum annuum
MAFRLSQLPLEERVRTWLLEGPPIHRFDALKHLAPDNPVDELLRVLQMYAQLVQGLWVPKSSLVYGTNSGVGVLARNFVLYEFTKSIFIKKSAFGRWPEFLKAATPALKSLAVERPDMNDWKLKEHPDKNFENLYGDVVKEQQATWECLGKQINDVLPGRGNRSTMKNPLSPNANIPALPSGDKPTSTSVLRTSMSEEIREALPRALQSVFKTYKVCSFQQIRQRLRELGVFKSYPLKGTPRRADLFDPPPEDIQAVINQIAVNIHGVYVLKSSPDNPQYDALRKVVIDLFMAEGPSAKLKKPSVDEAAKSQLKRDITDVEFLKVSSSFLVMRIKEYLLLALLSELINVVISINH

SEQ ID NO: 33 - protein, 100 aa, Capsicum annuum
MWLEKWSPNFKPEEDSPIVPIWVLLPGLPYHCHTWNYVKQTASPIGTPLSMDLATDNKTIPSMAKVRVEVDLTKPKINAMWIGSEDTNNPNEEFSSTEDE

SEQ ID NO: 34 - protein, 288 aa, Capsicum annuum
MSLSMEKHKCKLCSKKFLSGKALGGHVRSHLIPLPLPPKSTPPLNQDSGSRSESTLSLCSSENQQHLLEHMDFYYGLRENPKKSFRMIDPEFYESETESERNPSNRKRFKGSPNKEKAADFVSLSLYSDEKDIAMCLMMLSRDVWKDSKLNQCEICHRIFKTSQALGSHRTIHKIKNCNNYEHEARKNSSKRKLASVKNAQKLHECPFCGKKFQSGQALGGHKRSHFVTMSTSTTGSSSSTTKFGDYSLMEYSNSADFPNGYIDLNMPAPMEDNDFSQVEFQQEQEFA

SEQ ID NO: 35 - protein, 202 aa, Capsicum annuum
MKPKRRTEIVSTTGNSHIPDEILFDILTRIPSVKSLLRFRCVSKSFCSIISQSSFTEAHHQKGSSNHLVASFPGRFTRQTLIGNLENEKKAHKNNFQLGFSLFDYLNEPYFLKLLYLVSLNGLICLWNNSNDVAVCNPFTRQHVFLPKVIMTQTVVFNCCFLGLDPTTKKHKVLMAQQFSNSTPHWNIFTLGVDKSWREIHC

SEQ ID NO: 36 - protein, 370 aa, Capsicum annuum
MTHNKHQIVYAASQVHPKNWYPIPLIFCRPWGKLPENVPCHPKLADFANCMKKKGRGMSIFISIIDGDYHERAEDAKAACKQLSTYIDYKNCEGVAEIVVAPNMSEGFRGIVQTMGLGNLKPNIIVMRYPEIWRRENLTEIPATFVGIINDCVVANKAVVIVKGLDEWPNEYQRQYGTIDLYWIVRDGGLMLLLSQLLLTKDSFEGCKIQVFCIAEEDSDAEGLKADVKKFLYDLRMQAEVIVISMKSWEGQGEQQESIEAFSAAQHRIASYLAEMKERAERDKTPLMADGKPVVVNEQQVEKFLYTTLKLNSTILKYSRMAAVVFVSLPPPPANHPAFFYMEYMDLLVENVPRLLIVRGYRRDVVTLFT

SEQ ID NO: 37 - protein, 406 aa, Capsicum annuum
MSENNGQKSEPSSVILYCRVCKRGFNSAGALGGHMRSHGAAGENNNRNYGEQVSEPKYTINNFRRDKSESQKHSYNLRTNSNRLVAGNNRSTSDDRVDVEKSEYYYGRDGKEKQHSSREEEDLANCLVMLSSKSCDLSDDNNNSESRNKAKEVEKGMFQCKACKKVFNSHQALGGHRASHKKVKGCYAAKLDVDDDDTTNSNNDDNDIDEDLMISTGDLILHQESNSSQSQSPSSSSFSRKRSRVHQCSICQRVFSSGQALGGHKRCHWLTSSIPETTFIPSFQEIQYHNQEQVLYNKPIFTNHHQPLDLNFPAQLGNPAEVGLKLHSNPFEQESPKSSLQLWTDQQVDINQNENQNHKCKYSLSREEDIKSKEAKLSNLKDVNLDGGSSWLQIGIGPTPDILPTL

SEQ ID NO: 38 - protein, 529 aa, Capsicum annuum
MSINTRRCLFLLEKCRNMKQLKQAHGQVITCGLGENSFALSRLLAFCSHPNLGSPAYGWKILEQTQEPTICIFNTMCKAFLLKGELNRIIGIYKNMLKIGMYPDNYTLPYVLKACARMKSCHLGELVHGQILKLGFLIDIFVGNTLIGFYTAFDNVEAARFVFHEIPCSCVVSWTVLISGYAKRGDVYEAKLIFDECLVKDRGVWGSMISGYVQNNCFKEGLKLFRQMQMTGIDPDEAILVSVLSACAHLGSLDIGIWIHRYVEKLRMPVSVKLGTAFIDMYAKCGCLNTAEKLFDEMPQRDLICWNAMLSGFAVNGNGLKALKLFNEMQIVGIRPDDITFISMFTACSYGGIANEGLNLLNLMCNVYHIEPKGEHYGCIIDILGRAGLLEEARNIVQNMPGSSDPSEEAIAWRALLSASANHALVDLTEAEAAAERIAQLERHSGAYILLSNIYVAAGKHDSARRIRKNMLSQYIDKVPGCSSLEINGAVHEFIAGEKTHPQLVQIHELLGTINHHLDTSIHILHGSD

SEQ ID NO: 39 - protein, 357 aa, Capsicum annuum
MGSSSRKREIIEDFAIIGGLVGVQFMYAGNSVVSSYLMLLGFKPSSLIILSSLATFLILCPFCVMFERSQWPRQMSIKLLIQLFLISFGGVTLFQSLFMEGIKFTSPAMATAMPNLAPGFIFLIAWGFGLEKVELRCKYSRAKFAGTLMCVTGAILMSLMQKSTNAQRDLPSSPPHDKDNFFDTEKIKGSLYLMAAIIVLSSNIVLQAATLGDFPAPISLCAITSLIGMVLTGMVQLVQQGSVEIGLPLIRIPDIIGYSLLAGIVSGACVSFNNWAMKKRGPVLVSVFSPVGTLLTVVLSAVTLKDRFTIGSLGGMFLMFTGLYFVLWAKKSEGFLNNNMTNSSSESEFDVEKPLLN

SEQ ID NO: 40 - protein, 570 aa, Capsicum annuum
MHAKLHQKLIDFELFKKYAIQLHTKKHQNFFSSKSISSITKPHQENDFNSRILNSGSKPSAYCLVNLIRVCTNLGWFSRGEQLRCCILKSGYVNNVFVGSALVNFYVKFQLLGNAQQLFDEMTERNVVTWNTLISGYARSGQFRNALNLFIQLERSEQSSDSYSISAALSACGALGLVQLGKLMHSKIVKFGVECSIVVSNSLIDMYGKCGCAEESNRVFKRMMEKDTISWNSVIAANARNRALDQAVDFLCQMPEPDIVSYNEVISGVAQFGIIEDAVDMLSRMPSPNSSSWNSIITGYVNRGRAKESLEFFRNMHLSNVLMDQFTFSSLLSGIANIAAITWGRLMHCCTVKYGVDETLVVGSALIDMYSKCGHMKEADILFGWLPVKNLITWNTIISGHAHNGNSDEVIRLFEQLKSVKDLQPDGITFVNVLAACWHNKMSFQAANEYFELMVNDYGICPMPEHCTSMIKLLGQEGDVSKAEKMIYELGFEKCELVWKALLGASVTCGDIKMAEVTAAKVTELEGDDEFVYVLMSNMYALHRKWIDVGGMRGWMKEKRVRKEAGRSWI

SEQ ID NO: 41 - protein, 94 aa, Capsicum annuum
MTSISTIRSAIKLNSKSASSTTSLPSFSASTRSASSPLRRLSFSRIPSELGGLQSMLPLHNAVATARMTSCLSSTSRSCRALSQGTLCCTSPDL

SEQ ID NO: 42 - protein, 375 aa, Capsicum annuum
MSPFSNWSSLWPLLLLLXXXXXXXXXXLGSLPLFPARTLKNDPLTELASDIQNNTWHAFVKLLDAGKGSEVTGMSELKKYFQKFGYLSIPDQNITDFFDDNLESAVLSYQRNLGLTVTGKLDEETMKEIILPRCGMSDSAHDHDHDHSSSSLLHTTKNYAYFYGRPRWMKSSPMTLSYAFSPNAMIDYIDASEIRAVFQRAFSRWSFAIPVNFTETDDYYTADIKIGFYNGDHGDGEPFDGVLGVLAHAFSPENGRFHLDGAEIWAVDFDQERSKVAVDLESVVTHEIGHVLGLAHSSVKDAVMYPSLSPRTKKRDLKLDDVEGVQALYGSNPNFKYTSSLEHDTSSSNNIGIGKRTTSKWTLFFGMVTLMFMMI

SEQ ID NO: 43 - protein, 516 aa, Capsicum annuum
MHGCETSFCIVCPGIFKHNICFYDIFQDESHPAAIDIWKSRYSSFYYGCSERGPHFSSAVREGPSNGYLLIATSGGLNQQRTGITDAVVVARILNATLVVPELDHHSFWKDDSDFSDIFDVGWFISYLARDVTIVKRVPEKVMTSMEKPPYTMRVPRKSEPEYYLEQVLPILLRRWVLQLTKFDYRLSSELDEELQKLRCRVNYHALRFTKPIRNLGQKLVLRMRKMEKRFIAVHLRFEPDMLAFSGCYYGGGDKERYELGEIRKRWDTLPELSPDEERTRGKCPLTPHEVGLMLRALGFKNDTYLYVASGEIYGGEKTLQPLRALFPNFYTKEMLAGEELQPFLPYSSRLAAIDYIVCDESDVFVTNNNGNMAKILAGRRRYMGHKRTIRPNAKRLSALFMARDKMDWATFSRKVKSCQRGFMGEPEEVKPGRDFHEFPSACICKKPFNFSDVTSKENEYQISRRNPTVMESKDRYLSNSSNLEQQSSQKFREARLKDGHGSLIEDFDPDNPLSD

SEQ ID NO: 44 - protein, 2274 aa, Capsicum annuum
MDGDSVSTLTSLMDYNDNDYQSHLSGEDSSKVSSVLHPYALPKFDFDDSLQGHLRFDSLVENEVFLGIPTQEDNHWIEDFSRGSSGIEFSSSATDSCSIPRRNNVWSEATSTESVEMLLKSVGQEEMVRGDTIMEESDAGNELGCLIQPAASSLRLDDKRDDVKDSSSAAPADESVEFTGSFSRCERTKIEDVHIVCAPERQGMGPIADGCSDIAGERCSEVNTEEKLQTEIKSVDENLGEAKTLQSESLPDNSNRQPSVPVIQSAINECLTDSLPASTEILASQHNSTNCHSGNTSGLPSEHHKPEEKQISVSKESSMGDEKSRGRAVESETCTSNASPPSLASLKLEVVKEIPTETRMIKSEEPCVQDSECGLSTEGCKEDISSVGSAERVFSKDLKDNLRVVGNSILCEVKEASVSSICLDTRDTDNQEGSSKGRIERVSAMQMSDGLTASPEKEVNNLDGHSPLNLVTSEACAVSEISELSKENNGNGIHSLEGSSNIQEASVSTELVETPVPENLETGNDADRVSQGYACAGDHVPLPVPAGSRDICGEGFSRVVDVDATNADVSGDKEQEKVLPVENEMERSCVRDHGVRSSSMEGESEQISDQGHRSKFESSTLNNQALDVGFDDRNLILGGDSVSVPLLSGSDAIATEIVDHDEKLKPVSVMGGSGHFAGKGEMQVVLGAEAEVSTVKESSEGAGQVGPLSNDGKDTGDCCMEIKPMVVEPNVLAQDNPDTASHVEQAAIAEANIECCKHVEECATNSGSIIKEDAVFGAGAESAPLEKNKEMKRETVKLVEVGASEHYGLLGLLLNSCLCELKHDISSLFLLSLCVLWSFEIPIYSGTADGYDTDWSVSIFYSRSREKMLEISFRSFEGSSDVIGGRKEDSVAVQSCTALSLSEKKTTKSRRRAVVKNVAPLVDTTEIGGKAQPTSITSGENASNKADRSFTFDVSPLVGNAKGEADKSITSTQACLPTELKAGDRLHLTSASKQTDTKIMQEISHASPLVPDKGTPSGGAKSDRKARRGSGKSGKENPRKGSQLKEINSSKQSDRGNKSSVQCSPSVAVQKMQFEAGTVERNITKSSGVVSFPTSNLPDLNTSSPASVLFHQPFTDQQQVQLRAQIFVYGTLIQGTAPDEACMVSAFGTSDGGRSLWEPAWRACVERVHGQRSRGGNNETPSHPRSGSPRTPDQANKQAVHQNKVTTSAAGRASGKATNSPVISPMVSLSSPLWNMPTPSRDGLSSARGAVVDYKALSSMHPYQTPPARTFVGHTASWLPQAPFPGPWVASPQNSAFDISAQLPALPVTESVKLTPVKESSLSISAGAKHAPPGSVAHAGDSGILSGASLHENKQASVLPAQYSADQKSRKRKKASSTEDRAQKTKLGTSSESVTAPVVCTQLSNKAPSSDEFGQFSSVAVVPLVAHGQTGAASVPIIGGHFSTSVVIAPPSSSAPKNTSEIPITSGPSSAGISKRELDLGKKTKTSNNLSKVEEAKLQAEDAATNAAAAVSHCQDVWSQLDNNKNSDLPSDIEVKLTSAAVAVAAATSVAKAAAAAAKLASNAALQAKLMADEAMMAYDANNPSQTNTAAFPNIVNNLGSATPASVLKGQDVGNGSSSIIFAAREASRRRIEAASAASRHAENLDAIVKAAELAAEAVSHAGKVVTLADPLPLTQLVEAGPDSYWKVSQTLSGQVVKANKVNEDESSIPIVEKAPGIFSKRSEGPSVEEMHPTIPDFQPTTVSGNIVEDDMRNEEVIQTPVTGVEKDVRGAKGHSMPEGSKTIGLVAESSHDLVEACGDVESSRMQEGSLVEVFKDSDDGKRAWYSAKVLTLKNGKALVCYTDHQSDEGLEQLKDWVPLDVGSDELPRIRPAHPVTALQGEKKRRRAAVKEYTWYVGDRVDAWIDYRWREGVIAEKNKRDETTFSVNFPAYGDTAVVRAWHLRPTLVWKDGEWVELSRSRHDFLSQSDTPKEKRMKLGNHAGEDTGNDGLSKKMDPLVPVTNEPATLLPLSVNEKTFNIGSSKDDNKPNTLRTMRSGLQKEGSKVFGVPKPGKKRKFMEVSKHYDSDKGSKSNVPPGSAKFTKYLMPQATGTGGWKINSRTDLKEKQAIEARRKLPKPGKPPSSARNLKDNSITSTGDASGADHTVGEAIVYDKHEAKQPNVVNFVSNAEEGAEDPVKFRSEALPTNIPKKASTSLNRGEGMKKKIPISNLKSSKVELKDKMIPETNEPRRSNRRIQPTSRLLEGLQSSLIISKLPSVSHDKSSRSHSRGASR

SEQ ID NO: 45 - protein, 601 aa, Capsicum annuum
MNGLVKDENQRRVGKKQNGSSWKCAYGYSQHRRCKSASDKNVALSKGGDRQSVQKELNQPPVLPHSTRSCKASPLHELSKTIGKDAAPEHRASLEKDIEQLQMRLQQEKSMRMVLERAMGRASSTLSPGHRHFPAQTKELIAEIELLEVEVANREQHVLSLYRSVFEECISRPSSEQSSGMTSPAHSKVESRKHPSIISSAFCSSKKFPLRTFQALAAINDLGKRNLSHSKSRHASFYNSKANVHIQKSCSEHAKEQGQVMSMEKSPLARTLKDHLYQCPSKLSEEMVRCMAAIYCWLRITESTSTEQKRSPLSSRSSTNVIIPQHDIKEERDWFCKSTIEISWIATDKNNVSRASYAISNYRVLVEQLERVNLSEMETNAKMAFWINLYNSVVMHAYLAYGIPQNSLRRLALFHKAAYNVGGQVLSANAIEQSIFGLRTPRIGRWLETILSTALWKRSGEERQLISSKFALQEFQPLVCFALCSGAVSDPMLKVYTASNIQEELEAAKKEFLQANIVVKKSKRVLLPKVLERYAKEASIPSDDLLHWVMESVEKKLHDSIQKCVDRRTNKKASQLIDWLPYSSRFQYVISKDFTEKPWWA

SEQ ID NO: 46 - protein, 284 aa, Capsicum annuum
MTGTTEENQPKVQESEPQAPSIPTSSSSDEKEKQPEMDQQKWGTHIMGPPAVPTSHPDNQKAAALWRAADQKEEFHPQPYVVYTPVDHRPTNNPLESVVNMFNSWSNRAETIARNIWHNLRTGPSVTEAAWGKLNLTAKALTEGGFEPLYKQIFSTDPNEQLKKTFACYLSTTTGPVAGTLYLSSTKVAFCSDRPLSFKAPSGQEAWSYYKVAIPLTNIGTMNPIVMRENPPERYIQIVTIDGHDFWFMGFVNFEKATHHLLDALSNFKAQPPHVGEVPQPASN

SEQ ID NO: 47 - protein, 98 aa, Capsicum annuum
MYTSRQKIHKDKDAEPTEFEESVAQRCIELFEQAFFDLENTNQELKNELKDLYINSASYVKVVVIHVPYRLRKAWSWRRNSVARYLLNYFYNFVCEAR

SEQ ID NO: 48 - protein, 231 aa, Capsicum annuum
MIGKRSIPVIEKFTGASFPGSRTMDGATSPRSPLDFKIQSPRGLKSFDFGGVGLAIVAALEKSGGKNGETPANKAVYNRNSNRSLPIPVISSSKNPARYTAEFEDSLEEYTIVTCRAPGNKSYTKVYDDKKEFQGRKCNRSSVFNISPANIGGFPAYPDSDFLSSCHLCQKKLHGKDIYMYRGEKAFCSTECRYRQIVMDDHKEKCSSRSADVASSPYGNGQIFATEILAI

SEQ ID NO: 49 - protein, 170 aa, Capsicum annuum
MVEIAFGYYNDSVTATLLKAYLAEFIATLVFVFAGVGSAISDNKMTSDAALDPAAVTIGLAVGGHITILTGFFYWIAQLLGSTIACLLLKFVTHGLAIPTHGVAAGLSGFQGVVMEIITTFALVYTVYATAADPKKGSANILAAGPFSGGSMNPARTFGPHKTGSIGLGL

SEQ ID NO: 50 - protein, 401 aa, Capsicum annuum
MEESYLLGWKKNNDSDSWLSSVEEDEEMKVDLPSVHQPQTPMEPMDFLSRSWSLSASELSKALAQKQKHYSFERKFSVINESVSPPPQLPETKILNSHNGRKTGGIGRWFQHKDSNNSSVKKKEKVRLENAHMHSVLSIAGLAAALAXXXXXXXXXXXXAAAAENSNGSSSKMSMALASATELLASHCLEMAESAGADHERVASVVRSAVDIHSASDLVTLTAAAATALRGEAALKSRLPKEAKRNASISPCDRSMVETQSFAAAFPGDMDEHEPPFAGELLHLTRKGLQRWKRVSVYINKKSQVVIKLKSKHVGGAFSKNNKCVVYEVCDESEPWLFNKEIENLDLYLGVKTAQGLLEFKCRNKSHKQKWVDGIQKLLDKTSLFEDTEGSMTMLKINKSF

SEQ ID NO: 51 - protein, 331 aa, Capsicum annuum
MNFLRRLIASPSSRSQVSEFAKRSFKTSTNHTGSIVESASTSLVHGIHQFHCPDVVGILARLSDCIASKEGNILNADIFVPDNNDVFYSRSEFLFDPAKWPREQMDADFSKIAKNFNAMKSIVRVPDIDPKYKIAVLASKQDHCLVDLLHGWQDGRLPIDITSVISNHERGPNTHVIRFLERHGIPYYYLPMTKENKREGEILEIVKDTDFLVLARYMQVLSGDFLKSYNKDIINIHHGLLPSFKGSNPSKQAFDAGVKLIGATTHFVSEELDSGPIIEQLVERVSHRDNLRTFIQKSEDVEKRCLAKAIKSYCELRIFPHEANKTVVFRS

SEQ ID NO: 52 - protein, 234 aa, Capsicum annuum
MFLQRVISTTLPSSTRLFSSIGSHDLSKAISELNQELESVFGEPPPSGLSGSIDNQSMTTRNSKLNTVEMDGKESFAGLTHIGSKGEAQMVDVSRKDISKRVAIARGKVILGQKVFDLVSANQMGKGDVVSVARLAGICGAKQTSNLIPLCHNINLTHVRVDLALNPRDFSVEIEGEAASDGKTGVEMEALTAVTVAGLTVYDMCKAASKNIQITDIRLEHKTGGKSGDWSREK

SEQ ID NO: 53 - protein, 49 aa, Capsicum annuum
MRVVEVEAPSPMRYMMGAAVMMIGVVLPLAYMMFRNKRVPSSPSYPKQT

SEQ ID NO: 54 - protein, 527 aa, Capsicum annuum
MEEQEGSLLHLTMEKGPNSGQNLSYKPGSKIQIGRVIRGNTISIKDEGISSKHLSIHFESGLWVINDLGSSNGTFLNKIAIDPSRPTKLNNGDVIKIGEETSIKVGIEGMRVDLGENRRRNTRRKGLGVIDEDKELGFGNGGIGNVGVGLKRTTRSSKRGKNEAGNVDEVESFSVFEGTTRRTRGSRKVESVKNGVDNVNEAENVDVEREGKRCPRRTGRSKEVESVIHAVASANAAENNDVVERDGKRCPRKTRGSKKADVVDNAMEESESLAVVEEEQQKKPSPRRTRGSRKVGSVKDGTDGVQENESLAVVEEERERKPSPRRTRGSKRAQNIKWTDSVEEPKNSAAIDVDKEKKVCSRRTRGSRKEEDVENRHENAGEIRELKKLGKGKGSTAKMVHSEEDELMEKLERDPKDCEEAIQSNVNEGVERNMLEGVRVEFGRDGCEGSVSNVGFNISEDVQMEEEVNLEEMTLGGWFDYLEVNLPKQIIDATEKMILGMTQKTEKYQEFMLQQKNAKDCDGIPKG

SEQ ID NO: 55 - protein, 376 aa, Capsicum annuum
MVATILRYGIIGVGMMGREHLMNLYHLKSEGVAVVCIADPHIPSQIYAQKLAESFDWPLQVFAGHKQLLESGLCDVVVVSTPNMTHYEILMDIINHPKPHHVLVEKPLCTNVSDCKKVINAAKARSDMLVQVGLEYRYMPPVAKLIDIVKEGALGKVKMVSIREHRFPFLVKVDNWNRFNCNTGGTLVEKCCHFFDLMRLFIGANPVRVMASGAMDLNHRDEVYDGRVPDIIDNAYVIVEFDNGCRGMLDLCMFAEGGKNEQEISVVGDIGKGEALVPESIVRWGTRAEGRNSVQTLLAEDKRIKYDGLHHGSSYLEHLQFLSAVRARGEQAPAVGLHDGLVSVAIGVAAQLSIQKGRSVAIEEVMHEQLLQSTKS

SEQ ID NO: 56 - protein, 191 aa, Capsicum annuum
MELLLKVYIYKEGERPIFHEPHLTGIYSSEGWFMKLMEQNRYFVTKDPEKAHLFYLPYSSRQLQMAVYVPKSHNLRPLSIFLRDYVNMLAAKYPFWNRTHGTDHFLVACHDWGPYILKDHKELIRNTMKALCNADISKGIFKAGKDVSLPETTIRNVHKPLRNVGGKRVSQRPILTFFAGNMHGPVRPKLL

SEQ ID NO: 57 - protein, 100 aa, Capsicum annuum
MSYAEHMKSSKYCLCPMGFEVNSPRIVESIYYECVPVIIADNLALPFDEVLDWSAFSLTVAEKDIPKLKEILLSIPLKRYQVMQHNVKKLQAFPLEINAY

SEQ ID NO: 58 - protein, 512 aa, Capsicum annuum
MLISKSMFPLPNVCMVLEKISFSILLPAFYATLADSFLADLDELSDNEADVIDEENLDAEQMEEDGDLADIEALNYDDLDNVSKLQKSRRYIDIMQKVEDALERESDVTDKGVVLEDDPEYQLIVDCNALSVDIENEIIIIHNFIRDKYRLKFPELESLVHHPVDYARVVKKIGNEMDLTLVDLEGLLPSAIIMVVSVTASTTSGKPLPEDVLQKTVEACDRALALDSAKKKVLDFVESRMGYIAPNLSAVVGSAVAAKLMGTAGGLSSLAKMPACNVQLLGAKRKNLAGFSTATSQFHIGYIEQTELFQSTPPSLRMRACRLVAAKSTLAARVDSINGDPSGKTGRSLREEIRKKIEKWQEPPPAKQPKPLPVPDSEPKKKRGGRRLRKMKERYAITDMRKLANRMQFGIPEESSLGDGLGEGYGMLGQAGSGKLRVSVGQSKLAAKVAKKFKEKSYGSSGATSGLTSSLAFTPVQGIELSNPQALANQLGSGTQSTYFSDTGTFSKIKRT

SEQ ID NO: 59 - protein, 99 aa, Capsicum annuum
MHRGTKQLLNMIFLFDIDIMHRGTSKVISVATTNVRDLRNTLTFLTDNDDARVIGKLIAKISKEADVYAIAYEPGKNERIEVRLGIILDTIQKSGIIFV

SEQ ID NO: 60 - protein, 555 aa, Capsicum annuum
MCVNAIIISLTRSDSCDVDRSVSLTNEMRKFLRRIIVVLLFRVSWKKLFVIGTILTVFRIIFQIYSLPNPLTGWIISPPLVVSSYQRANDGKNSRELSVTSDNQLKKSKHLPAVVSVNSTARNSREVSVTSDNKLKMNKHLAGFVSLNSTARTNRSMQVLQRRERLLRRRRSKMNAHLKIIFPPPPVVPDHVQRSIAALKPNEALAYAKSEIEKAPLITDDPDLYAPLFRNISVFKRSYELMELLLKVYIYKEGERPIFHEPHLTGIYSSEGWFMKLMEQNRYFVTRDPEKAHLFYLPYSSRQLQMAVYVPNSHNLRPLSIFLRDYVNMLAAKYPFWNRTHGTDHFLVACHDWGPYILKDHKELIRNTIKALCNADISEGIFKAGKDVSLPETTIRNVRKPLRNVGGKRVSQRPILAFFAGNMHGPVRPKLLKYWKDRDEDMKIYGPLPTRVSRIMSYAEHMKSSKYCLCPMGFEVNSPRIVESIYYECVPVIIADNLALPFDEVLDWSAFSLTVAEKDIPKLKEILLSIPLKRYQVMQHNVKKLQAFPLEINAY

SEQ ID NO: 61 - protein, 790 aa, Capsicum annuum
MLSRAYTTDTQSKSSDIAATILAAASPPQILAACDAVESFLHKHTADQTRWFFSITFPTLICKIFGFNESSGSSSAAVKSSSPSGWIDIAALANDSQLADRIFSLLSPTGVLLSSIAAADGLSLVKYVFPVERLPEWVRYMLQNERDSRVLSDLCPLFKNRLKEDSVKGSSFQVQLNVFEYYMFWFVYYPVCRGNSEGPQTVRVRRSKRFRLENWAYSIPGLSSTKRGLEQKNEGNLYMRLLYSYLRAYVPVGDVKSHQPYRSSLLHYSFAYDTPFVEKAEFLVNTLIHFWLVDNDFSPLPMNLCKSFGVSLPFRSLFGETPPTSGLGEVVNVFVKYLNLSSSAPSDGADQVDYTESPRWKVGGTFNATQSRNVVPVVDSGNSWNSWIQRPLYRFILRTFLYCPVESSIKNASQVFTLWVSYLEPWTICMEEFAELDANLAKCNKSTLKDVTPSTPHGYTSSWQAFVLANYLYYSSLVMHFIGFAHKFLHTEPEVIVKMVSKVITILTSSSELVNLIKNVDTVFHLKPTGSSKGVLNALHRYVPAIREQLQDWEDGLSETDADGSFLHENWNKDLRLFSDGEDGGQKLLQLFVLRAESELQSIGGENLSQNLQCLDRLKSELCQLFGGPILKPMNTTETLQCGHLRDEIFKPRSFANRAMVDIKYKGDWMKRPISDDEIGWLAKVLVKLSGWLNESLGLNPVESSQDSPSWSYLDLSTDARSEYGPMEMMKVVFCSFISWLLMLRGAGVTFMREHGVRVNLRVLASKKVVVALLIVAAFSLLRRTFLRVG

SEQ ID NO: 62 - protein, 146 aa, Capsicum annuum
MPQKVEDALERESDVMDKGVVLEDDPEYQLIVDCNALSVDIENEIVIIHNFIRDKYRLKFPKLESLVHHPVDYAQVVKKIRNEMDLTLVELEGLLPSAIIMVVSVTASTTSGKPLPEDVLQKTVEACDRALALDSAKKKVLDFVES

SEQ ID NO: 63 - protein, 220 aa, Capsicum annuum
MEQEHQSPISLNNLNNNKTTSSSALTKRPITLISSKEETNHKKSKKLDDTKHPLYHGVRKRSWGKWVSEIREPRKKSRIWLGTFATPEMAARAHDVAAIAIKGHLAMLNFPELAHQFPKPASKCAKDIQSAAAKAAALDISPRNCMEQESKGELMEVVPCSPDEERETNEDPFLDLPDLLMDLTQEFDEFCYSNYFEFWPEEDLYLWDYSHGTVLEVPRP

SEQ ID NO: 64 - protein, 94 aa, Capsicum annuum
MVEEDSKKLDKQEEEDHEELVLKSSKNKKDEMSSTTTSTMRRKNNKCATGAGVGASKKDRHNKINTAHDPRHRRMRLTRNSSQILQFARLSRVR

SEQ ID NO: 65 - protein, 229 aa, Capsicum annuum
MEQHQSPTTFNNNKQTSSSSLTKRPITLTSSKQETNHKKNKKIDGSKHPLYHGVRRRSWGKWVSEIREPRKKSRIWLGTFATPEMAARAHDVAAIAIKGHVALLNFPELAQQFPKPASKCAKDVQTAAAKAAALDISPRNSMEVPCSPYGETESTESSLGSSNDDPFLDLPDLLMDLTQKFDEVCHYNSSWDLAGAGSGSNDSCEFWPEEDLYLLDYSHETALEVPQLQ

SEQ ID NO: 66 - protein, 560 aa, Capsicum annuum
MFIPAIKGQGTDEQQKKWLPLAQKMQLIGCYAQTELGHGSNVQGLETTATFDPQTDEFVIHSPTLTSSKWWPGGLGKVSTHAVVYARLITDGKDYGINGFIVQLRSLEDHKPLPGVTVGDIGMKFGNGAYNSMDNGVLRFDHVRIPRDQMLMRVSQVTKEGKYVQSDVPRQLLYGTMVYVRQTIVADASLAMSRAVCIATRYSAVRRQFGSQNGGQETQVIDYKTQQNRLFPLLASAYAFRFVGEWLKWLYTDVTQRLQANDFSTLPEAHACTAGLKSLTTSATADGIEECRKLCGGHGYLCSSGLPELFAVYVPACTYEGDNVVLQLQVARFLMKTVSQLGTGTKPVGTTSYMGRIEYLMQCRSDVKQAMDWMKPSAIVEAYEARAARMSVACAKRLSKFDNQEEGFAELAADLVEAAVAHCQLIVVSKFVEKLQQDIPGKGVKQQLEVLCGIYSLYLLHKHQGDFLGTGYITSKQASLANDQLRALYSKLRPNAISLVDAFNYTDHYLGSILGRYDGNVYPKLYEAAWKDPLNDSDVADGFHEYVRPLLKQQLRTARL

SEQ ID NO: 67 - protein, 115 aa, Capsicum annuum
MEGVDYLADERKKAEFDVDEMKIIWAGSRHHFQLSDRISKLVANDPGFSKEGRTMLPRKELFKSTLRKAAYGWKRIIELRLTEEEATVLRRYVDEPAFTDLHWVTSCFDFFFCLC

SEQ ID NO: 68 - protein, 499 aa, Capsicum annuum
METSTLIRPNNNNNIKSKNKKQTSPLLTFLFLFTLILLFFYFKYFISPSLLPISPKKSLPILPLPIQCNPINRLQGKFLWYAPHSGFSNQLGEFKNAILMAKILNRTVIVPPVLDHHAVVLGSCPKFRVMEASELRLSVWNHSIQLLRDCRYVSMADIVDLSPLVSDSTVRFVDFRAFVSTWCGINLDVICSKDQNIPSPLSESLWQCGSLLSGYYGSFSGCLSALEEDCRTTVWTYQKDGEDGALDSFQPDDQLKKKKKISFIRRRKDVYKTLGPGSAAESATVLAFGSLFTAPYKGSESHIDIHEAPNDSIIQTLIEKIEFLPFVPEIINAGKKFALQTIKGPFLCAQLRLLDGQFKNHHKATFLSLKQKIESLKQTGQKQIHIFVMTDLPMANWTGNYLGSLAESDVKLFVIREDDDLVQETAKDVMVAGHGLEVGSVSQISAGIKKRRHPQSLTDFLLYIEEVVCSCASLGFVGTAGSTIAESIELMRKHDICSG

SEQ ID NO: 69 - protein, 258 aa, Capsicum annuum
MAAVDFLVAVDSFPKPDDKIRSTSFQVQGGGNAGNALTCAARLGLSPRIISKVGDDSQGKAILEEFEADGVDTSFMVVSKGGHSTFSYVVVDSQTAFTSKHDVERSSFVESVVNGSAVICLIFGFWISNSIFYITYTSLKRVSVSTILSEDENCLCKNQKKLKTRTCIYTPGYPPMIPEDLSKSNLSSALDDVRLVYFDGVLQEAELLSALVVAREAHQRNIPIVVDAESKRERVDDLLNLATYVVCSARFPQASLFH

SEQ ID NO: 70 - protein, 174 aa, Capsicum annuum
MYEEENDTTTCSLTILGFYFTTLLNFLQLKYQGNNKPDPFSTHPKTMRVSVTCLLLYCLAYGVKQRFSSHPIYRKFSDLVRNVMVFFTSLSLASIASVLFPDSFSHVVFSLCFLLFGGEMLHWIFKKMMQHFEEGEEWHFWRRRDGEPAVWNYVRRRMNMVGYPMEERFISLTV

SEQ ID NO: 71 - protein, 171 aa, Capsicum annuum
MTTNSHSVSSSTTQSANSSPVTLSSPLYLLPYDSSGMILVTTTFNGTEYGSWCKGMLLGLSCKNKLEMVNDTVPKPPSTDPFFQAWVRLHGYPNSGSFGVQSKYKKTAAFAHVSDSQSPAPFDSTSNIQVQGNSGMPRITKEQFDQLLNLFHQSKVFAASQEIFAGSANFA

SEQ ID NO: 72 - protein, 136 aa, Capsicum annuum
MLIAFIFPAMVDLLQLMYQASNKSPFELHPIKMYLAITSFLIYCFSYDAQLRLLNISTSHTPNYPKIIATVFGPLTLATYSSLLFPSLCPIIYLISISYSAIQLQVNCKLEELLYRLYRKFMSYSADRPHSSILPI

SEQ ID NO: 73 - protein, 170 aa, Capsicum annuum
MDKRGGKGRQKIEMKLIESKEARTVAFSKRKNGLDKKAKEYSNLTGEDVGFILFTQAGKPHSYGSPSIEKVIDKFHELKQNDSQQNHTDMSKTNVFEAFEDLRKEVQAFKEKYLDSKMSTNKHMLENRLKQFEAYKSWLDKIKKEIKGDVPGEQFKFISEVALDLEEMSS

SEQ ID NO: 74 - protein, 349 aa, Capsicum annuum
MTRRCSHCSTNGHNSRTCPNRGVKLFGVRLTDGLIRKSASMGNLTHFGGCGGGGGGSSTGLNGVVHDSPGDTPDHPAVGGAAADGYGSEDFVAGSSSSRERKKGVPWTEEEHRMFLLGLQKLGKGDWRGIARNYVITRTPTQVASHAQKYFIRQSNMSRRKRRSSLFDIVADEVSKFSLSGDTPMVSRDFLAEDPQAEMQSNNPLPPTPVVDEECESMGSAASANSIDGEHSLPIPESSQYPHPLVYPAYVAPFFPMPYPCWPGYTAEPAKAETHEVLKPTAVHSKSPINVDELVGMSKLSLGESIGNAKPPSLSLKLVEGSSRQSAFHANPSSGSSGMNSSHSPIHAV

SEQ ID NO: 75 - protein, 135 aa, Capsicum annuum
MEGALQLPVIDLSSTDETLFTDSLRKACIENGCFYLINHDLEDDLIQRVFDGSREFFALSVGEKMKVLRKNHRGYTPFYDEKLDLVDNPKGDPKESIYFGAPEDISPCGNLDQWPPEEIFPCWRSTMEEYYRVIL

SEQ ID NO: 76 - protein, 548 aa, Capsicum annuum
MFDRQWTVPDEEWRHIIDDFAATFGVTRHSLLESFTFFLLDDEGVPALKEACQLLPEISSPTIHPKVAQVLLERGNPDAALMVLRWSGQDGTQLISLREAVTAVRVRVECGLLTEAFTYQRLICAKIKGKKLRNEQFQSASAEVEDQCRSWGLWVETLVTEICCLCIRRNLVDRMIELPWSADEEKHLHKCLLDFAAKDPSSTIGSLLVVFYLQRHRYVEAYQIDQKLQSMEENFISQNSVSEEVLARIRSINHWRTCLVDKGIELLPDTLQQQVRTGKLPEVVVTCNDTVSISERSNAEAQEPVLTSLLANPPTDSTLIQQVDIVKPSVPDAPFVLGGSLNLPSFKVGHYSSPSSPAHFFNDAGVLNSESILGKKLKFDEILTPTSRRPPASVMKITRNLSREPSISRLRNSQTYKVSPEKFQNGFSKESYNFHQTAGNNVISLSGNRGILKDSLEDSYTSYPGNHLLSDAADRSQMLPLNDSMDITWSHEERGPSTVHPETNGGPRWRSDDTSEDEDHLSRDGFAEVVSPARTLRGVRRSRRIARR

SEQ ID NO: 77 - protein, 414 aa, Capsicum annuum
MLNDGQPSSFCGTTHSWISLQGALKHLASIDPLELCDEAKVEHCRATRDLSSCGRHVQSVLNSCGHASLCEECSQRCDVCPICRIPLPKDASRLRLRLYYECIEAGLISKKCDDRLQEKEDRDKRLVADIERLYALFDVALENNLVSLICHYVTDVCMDENAVSSDPIIAFLLDEVVVKDWCKRTFNNILTEIQAIYNLTMSALQEKVSLFLKFSVKLGGISNVIDVLESSFKGSLSAKLQDLHHLQECILKTKQHMEIMIWCIRHEFLESVKSRHKNFASWRALALERKSAAINRAWPDSVNHSDEYNTSTLFIEDALSNIEAAEQGDVDDHEEELALYLQKDGGSLYSRSKIEGMAAWYPFESLRAAADILFLRGSSDLVVAKHAIVSFRFCGFHLSLSLSNRSLSNRSLSH

SEQ ID NO: 78 - protein, 65 aa, Capsicum annuum
MVTAANVVNVPGINTLGVSMARIDYAPGGINPPHVHLRTTEIIYVLHGQLLVRFINTVNVLFYGN

SEQ ID NO: 79 - protein, 164 aa, Capsicum annuum
MERGSDEEDGETSRKKLRLSKDQSVILEESFKEHNTLNPKQKLALAKRLGLRPRQVEVWFQNRRARTKLKQTEVDCEFLKRCCENLTEENRRLQKEVQELRALKLSPQFYMQMTPPTTLTMCPSCERVGAPPSSSSGPTSTPMGQAQPRPMPFNLWANALHPRS

SEQ ID NO: 80 - protein, 528 aa, Capsicum annuum
MGDNTAPNTSKKQKKESEVAPNHAKHSKFYNHFLFKASLIVIFLVVLPLFTSEAPDFINQTLQTRSWEVLQLIFVGIAVSYGLFSRKSDETDKEHNTKFDNAQSYVSGLLHVSSVFDDEADIHDENKVQTWNNQYHRGNNPVVVVAKENHSLNEHRAINSRIGEKPLLLPIRSLKSRVLDDKMEGHDDEFSSKSRKEGSVVSSPKKLVDNMEENVVRPSPIPWRSRSAKNVEMKDENNHCYNSKSSSKEDTNEFKKIEPFSLRSQSFRSPKTNNYSNSSSPRTLSPSQSRKLSPSLSFSSESQSKIVNEDILRKKIFRKAAPPPPPPPPPPPPHFFYKNSPLLKSSSSVTSNGAVSSEKELRRSIRSVPMESTRGEKFQTPRKAYSGTELRPFAGSTRAREYGAEFASPEDEETFVKDKGTTDKMMHESYNFATKSRFMEFPNEDKKEYVEKFLVETDQEDSETESEDYDYFEENANKTEVGTDQTDEGPDVDKKADEFIAKFREQIRLQRIESIRRSAGQPTRNLVR

SEQ ID NO: 81 - protein, 185 aa, Capsicum annuum
MKRLTILLYLLSCSAGSIVQDLWSLSGPDEKNGITSYGLVENDSDLVHGLLEVEGALVGSSRTEKDCSQFDNDRVTLLLRPEPRNPLDMMQKGSWSILDQRFLYETEFEEGEGEGALDPQEDLFNHIVWAPRIWSPWGFLFDCIERPNELGFPYWSRSFRDKRIIYDEEDELQENNLGFLQSGTM

SEQ ID NO: 82 - protein, 50 aa, Capsicum annuum
MFVSSIFWTLAPWKNMLLLKHGRIEILDQNTMYGWYELPKQEFLNNKQPV

SEQ ID NO: 83 - protein, 303 aa, Capsicum annuum
MIWHVQNENFILDSTRIFMKAFHLLLFDGSFIFPECILIFGLILLLMIDSTSDQKDIPWLYFISSTSLVMSITALLFRWREEPMISFSGNFQTNNFNEIFQFLILLCSTLCIPLSVEYIECTEIAITEFLLFVLTATLGGMFLCSANDLITIFVAPECFSLCSYLLSGYTKKDVRSNEATMKYLLMGGASSSILVHGFSWLYGSSGGEIELQEIVNGLINTQMYNSPGISIALIFITVGIGFKLSPAPSHQWTPDVYERSMVRSRNSYLSIYLEMFGFFKTPWACRREILSLFGPRQNFYLFK

SEQ ID NO: 84 - protein, 155 aa, Capsicum annuum
MSRRGTAEKKTAKSDPIYRNRLVNMLVNRILKHGKKSLAYQIIYRAVKKIQQKTETNPLSVLRQAIRGVTPDITVKARRVGGSTHQVPIEIGSTQGKALAIRWLLAASRKRPGRNMAFKLSSELVDAAKGSGDAIRKKEETHRMAEANRAFAHFR

SEQ ID NO: 85 - protein, 83 aa, Capsicum annuum
MDSSMCSSAPDPEMWIIQGTLAWRTPPIRVRSNVDPTFDSLVGYERSGGDHHDSSLLKNPYIPYQCMDSYLSSTGLGSTSMGK

SEQ ID NO: 86 - protein, 55 aa, Capsicum annuum
MFQFARLSLACPWIQQQFERFPYSRISGSMLIFNSPKHFVDYYALPRLWMPRYPP

SEQ ID NO: 87 - protein, 477 aa, Capsicum annuum
MFLPFTYANRGNKPILSLKMVYLFSTASSSSASGEFLKHLLNNKNGYGMERALNAVTCKLDSRCVDEVLEKCAVSDPKMGLRFFIWAGIQSKYRHSSYMYGRAYKLLEVDRKPQVIRDVIEGYRVQKCVTSGKMFKVLLNLCREGKDAVLGLWVLREMKESNCRPDTIAYNVVIRLLCEKGEMDEAMGLMSEMEVSDLYPDVITYVMMIKGLSEVGRLEEACGLTKAMRGHGCMPNTVAYSALLHGICRFGNLERALELLREMEKDGGQCKPNVVTYTTVVQNFVEKCQSIEALSILDQMKDFGCKPNRVLICTLIQGLCKDGHVEEARKVINRVTESGIPYDSCCSLLVLSLFRIGKLNEAEIFFRKMLAGGLKPDGLTSSTIIRWLCQRSRILDGCHLIDAIEQSGSVSSIDSDIYSILMAGLCEANHLAEAAKLANLMVVKGIQLKGPFVKNVIECLKRCGKEDLASNIDNIKS

SEQ ID NO: 88 - protein, 395 aa, Capsicum annuum
MYIEEEKGGEVESGEEEVVVGGGVVEKVEKLDTGGEVLCSSDSSGKNSVVVLDVRRVMVGVGARALFYPTLLYNVVRNKIQMEFRWWDWIDEFVLLGAVPFQSDVKRLKELGVSGVVTLNEPYETLVPTSLYEAHGICHLVLPTRDYLFAPSLNNICQAVEFIHENASNGQSTYVHCKAGRGRSTTIVLCYLVKYKQMTPNDAYNYVKSIRPRVLLASAQRQVSHRFASCFDPFSLRCMNLMRLMVDTPVDLVLFFWLLLLILFFHFNNQAVQEFYHLMVKKTYSCYPLTSVIPRSSRFLARRNLLAFDDGAVVVITEADLDGYDSNLDSRVAGSEIWADLNLIYRVRVASGAALARLSCMWLRCHADQKIPNQKLNAENKQLESFTVDIHVFSS

SEQ ID NO: 89 - protein, 307 aa, Capsicum annuum
MAYFCSENLILFFISIFCSICFSSSTPFIVLHGLGQSCNDAGSTFYTSQLSLLSRSNGYCLEIGNGAYDSYTMPLENQVQIACEKVKAMKELQQGYNLVGLSQGNMVARGLIEFCDEAPPVKNFISIGGPNAGVASTTCAGGPWCAGTGGVSGVGIYSDYVQTHYAPSGYIKLPNDIAGYLKGCRYLPKLNNEIPGANNSIYKQRFTSLQNLVLIMFANDGVITPTESSWFGFYQDGTYSKVLPPQQTNIYIEDLFGLQTLDKAAKIKFIKVPGYHLGMDIQELQQYVVPYLINGAPKNKVATQVVH

SEQ ID NO: 90 - protein, 145 aa, Capsicum annuum
MDSIVEALNSAYQDFVAAAAIVLESKESSNGQKTAATDAALENFKQRWELFRVACDQAEEFVEAVKQRIGSECLVDEATGSVSAKPGQSASSGLQPISAVRLEQMSKAVRCLVIELQRGSGASSASAHPNPSAPFDARFSEDVAQ

SEQ ID NO: 91 - protein, 802 aa, Capsicum annuum
MARWDQILSLPVQSPPTLEFSSAELVWSKVEGWRDNIDRVALIPFARVDDFVRGESANKECPTRFHVEARRRRPAETTYKPKVDGILEYILYWCSFGPDDHRKGGIVRPSRNTYVPKKKPSGRPNTKRGCTCHFIVKRLIAEPSVALIIYNQDKHVDKKGLPCHGPQDKKSAGTRAMYAPYISEDLRLRIMSLLYVGVSVETIMQRHNESVEKQGGPCNRDDLLTHRYVRRQERSIRRSTYELDTDDAVSVSMWVENHQNQVFFYENFSDSDPFILGIQTEWQLQQLIRFGNHGLLVSDSKFGTNKLKYPIHSLVVFNSDNKAIPVAWIITPRCASGDTLRWMRALYNRVHMKDPKWKLAGFIVDDPLADILAIREVFQCSVLICFWRVRRAWHKSLIKKCSELETCAAIAKRLGQAVHRICKGDGTADLFEEFMEDFVDVADFLDYFKAIWYPRLGLWTSALRTLPLASQETCSSMEYYHNQLKLRLLNEKDKSVYQRADWLVDKLGTTVHSYFWLDEYSGKDDFARYWKDEWMSGLTAWQKSLQIPDSDVLVEGEYSKVVDQEDRHQVHVIWNPASEYALCDCNWAKMGNLCEHILKSIKYLRDKGSITPSVSMFQYMQALVDMLHCPPHDSLIRDHALSLAVWVQKQLNAQLGPGSGQSKRQALQLTASTRGVELVNASNRTHTLTDAVDDLAELQHSSSATGNLSSRQVDRVATENGTLTDMSHAVGHPSVEMQTSPLSISACASQLFSLDGITSANVFSDNGGVMIGEELDITKNLPCTDTSFANLDGFQDGGLGQS

SEQ ID NO: 92 - protein, 216 aa, Capsicum annuum
MDALIFISMFIIFLHVKAQANTPSCSPISCANSSTFNIQYPFTLHQIQPQHCAGLPDFVLQCKQNMTILNFPSYGDLVIKSISYDLQKLELIDPKNCVHGVFLNLNLSNTPFSYYYVLKEYQYLNCSAEISSFLQVPCLSGIGHHVYVVETSFVVTDFCNHVKTVGIPFSYSPFLSDNTFGLGLTWHLANSQDSLTAKFTAGLFEGLNEIVDDKGM

SEQ ID NO: 93 - protein, 140 aa, Capsicum annuum
MPERRVTGTVVFLKVHGQEYGYGAHDYPSSGVFEVEPRSCPGFIFRRSVLLGSTDMSRSEVRSFMEHISEKYHGDSYHLIAKNCNHFADEVCSRLTGKPIPGWVNRLARVGSFCNCILPESIQVTRIRHLPDHQGSSGWF

SEQ ID NO: 94 - protein, 60 aa, Capsicum annuum
MKKVMEALRGECVVIIGVYVMGSVGKTTLVEEISQRVKQERLFDEVVMVTITQKPDLKRI

SEQ ID NO: 95 - protein, 234 aa, Capsicum annuum
MPKHVAVIMDGNRRWAKNRGLPVALGYEAGIRSVRKLVELCGNWGINALTLFAFSSENWSRPKVEVDILMGLFEKMLKSELETLASAGIRLSIIGDSSKLPKSLQDLIEKAVKTTEENSRLHLLVAVNYSGQYDVVQACQTIAQKVKDGIIEPEDISSFLVEQELQTNCTDFPCPDLLIRTSGELRLSNFLLWQLAYTELYFSNAQWPDFGEAEFSETLCSFQQRQRRYGGQDS

SEQ ID NO: 96 - protein, 159 aa, Capsicum annuum
MIDSAFLDFHRAGIRISIIGDTSMLHKSLQELIDKAVKTTKENSQLHVVIAVNYSGQYDVVQACQTIAQKVKDGIIEPEDISSFLVEQELQTNCTDFPCPDLLIRTSGELRLSNFLLWQLAYAELFFSHSHWPDFGEAEFLEALCSFQQRQRRYGSQGS

SEQ ID NO: 97 - protein, 770 aa, Capsicum annuum
MEAATTTTVSVAEHNHIMWRMRLHSAIRTVVAYAIIGCSTLYGPPWFKKLAAFPAFSYVTATLVTSDSTLGDTLSGCWHAIIATVQTMPLSMLSLWIATAPNGSNRLFPVVSALALALSSLLVAVVECTDIRCKKIAFGQLVLVFADGVIRGVHTSAVVHPLRVAYSTVLGMVASLLALLLPYPRLAYFEVRKLHQLYAENSKERVEIYSRAITSQDNLIAVERLSKAKFLAQTAAKLLQSAKLLKGGLMWETPWIRFFKSCSEIPGENGIQNMELSIRGMEISLTSCPSFSTGLVKEELKDTLIHMSEQIRRKSDQVTHQETNGSDFANKSIFLHDDTMSPTQTSLPAFFFLFCAKMLLDNSSTSYNEDTQRLCHKSWMKIRPRKETLMFALKCSVSLGLAMWIGLLFDKQNGFWAGLTVASTLAQGKLATFTLANAQAQGIAFGSVYGVLGCSVFQKATNLRFLALLPWIIFSSFLKHSRISGHAGGISALLGAVMILGRKYSDPANEFAIIRLTETFIGLSCFIAVQFLLNPKRAASLARNQLYCTLDILKDCMNQIAQKDQRELRGLIEKQRKLKSHILNLQKLCLNAELEPDFWFLPFNATCYKKLQGSLSKIADLFYYVVYNINNMSHDFQSHAVDCKELHDSIYDELEYLKETTISLISTFLDKPSLMKLFPDDDDQEVKILHDLEEGKLSKKNDEKTERGLCFFLERSKEVIDGILSSQDKQELKGKTIISLYALEFCITSMLKVIKDIKMTMKELSQWESS

SEQ ID NO: 98 - protein, 677 aa, Capsicum annuum
MSIGGDTATNSAAVSPRVSTTXYHRRPVTDSDTEKSFDHLPMGFDNNNNNNTNIINIVGVNLLDHHNHSHGTITRCLLPCKCMPECMVIRVEEWLACLGSRKYMRRMMSGLLMILVVISVFLKFSILMNGNVVEDIIGKENELFVVQIIKNDWVNAQKVVSETVSSVSSSSSGAVQKRQMKELPVPEIWMKPSNDSYFKCIARRRNRTRTGSSTSGYVLVHANGGLNQMRTGICDMVAITKILNATLVIPSLDHKSFWTDPSDFKDIFDWRRFIDVLKDDIKIVESLPQKYAAVTPIQKAPVSWSKQAITFCLLKLKFVAFLQGSYYRREVLPLLKKRKVIQFTHTDSRLANNGLASSIQRLRCRANYEALRYTNEIEELGKKLVDRLGDNGEPYIALHLRYEKDMLAFTGCSNNLTAQEAEELRIMRYQVKHWKEKEIDANEKRLQGGCPMSPREAALFLKAMGYPSATRIYIVAGEIYGNNSMDAFRAEYPNVFSHSILTTKEELNAFKHYQNRLAALDYLVALESDVFVYTYDGNMAKAVQGHRKFEGFRKTISPDRSLLSLHFYTYSSLNYLGFEQTNNFLLVLIFHFRLNVVRLIDLLDKGTISWEKFASEVKELHSNKLGAPNPREPGESPRLEENFYANPFPGCICDRSLMEVSNQQLKKRPSVKAAAQR

SEQ ID NO: 99 - protein, 446 aa, Capsicum annuum
MREILHIQGGQCGNQIGAKFWEVVCAEHGIDSTGRYGGDSDLQLERINVYYNEATCGRFVPRAVLMDLEPGTMDSIRSGPYGQIFRPDNFVFGQSGAGNNWAKGHYTEGAELIDAVLDVVRKEAENCDCLQGFQVCHSLGGGTGSGMGTLLISKIREEYPDRMMLTFSVFPSPKVSDTVVEPYNATLSVHQLVENADECMVLDNEALYDICFRTLKLTTPSFGDLNHLISATMSGVTCCLRFPGQLNSDLRKLAVNLIPFPRLHFFMVGFAPLTSRGSQQYRALTVPELTQQMWDAKNMMCAADPRHGRYLTASAMFRGKMSTKEVDEQMLNVQNKNSSYFVEWIPNNVKSTVCDIPPTGLKMASTFIGNSTSIQEMFRRVSEQFTAMFRRKAFLHWYTGEGMDEMEFTEAESNMNDLVSEYQQYQDATADEEGEYYEDEEEAEED

SEQ ID NO: 100 - protein, 185 aa, Capsicum annuum
MASFMKHSSMILFFACTLFLQGALGEITCENLPTNVCSFSISSSAKRCLLENSANEVGKMEYQCKTSEVMVGNIREYIETDECVNACGVDRNSVGISSDALLEPLFTAKLCSRPCYQNCPNIVDLYFNLAAGEGVYLPDLCNKQRTSPHRAMIELLSSGAAADAPAPFASQDIVVGAPDIAPASF

SEQ ID NO: 101 - protein, 467 aa, Capsicum annuum
MCAENGGENGKNALPSFTEFSLDVLMNATNGFCADNIVSEHGEKAPNVVYKGLLENGQWVAVKRFNRSAWPDSRQFLDEAKIVGNLRSERLANLLGCCCEGEQRLLVAEFMPNETLAKHLFHWDSQPMKWAMRLRVALYLAQAMEYCCSKGRSLYHDLNAYRVLFDQDGNPRLSCFGLMKNSRDGKSYSTNLAFTPPEYMRTGRVTSESVVYSFGTMLLDLLSGKHIPPSHALDLIRVKNLSMLMDSCLEGDFSNDDGTELVRLATRCLQYEARERPNAKSLVNSLITIQKETEVPSHVLLGIRHGTATPLQPLLLTTTGEACLRRDLTALHEILVQTGYKDDEGIANELSFQMWTNQMQETLNSKQQGDAAFRAKDFTTAIECYTQFNDGGTMVSPTIYTRRCLCYLMSDMAQEALGDAMQAQVISSEWPTAFYLQSVALFSLGMENDAQEALKAATKLEAKRNKN

SEQ ID NO: 102 - protein, 309 aa, Capsicum annuum
MSLYIGREALKLWKRICTETTIEINLLAENWKYILGGVIFQYIHGLGARGVHYLHRPGPTLQDVGFFLLPELGSDKAYISETVFTTIFLSFVLWTFHPFIFKTKKIYTVLIWCRVLAFLVACQFLRMVTFYSTQLPGPNYHCREGSKLATLPRPNSVFEVLLLNFPRGILYGCGDLIFSSHTIFSLVFVRVYHKYGTRRFIKKCAWLATIAQSFLIVASRKHYTVDVVVAWYTVNLVVFFIDKKLPELPDRTSAASLLPVSKDSKTKEEDHKLLNGNGDPEDWRPRTQINGMIMEVGNAVHVEVAMNGV

SEQ ID NO: 103 - protein, 149 aa, Capsicum annuum
MWSSNSNLVNQMNPNTLQLPMAWTNDPSHVHTKPHLWHLSFYWGRNTYFLFPNWPGNNRTMYGLGIIFVFVLAILVEFFSNLKLVKPGSNRGAAVFFQAGVQAIRAGFAYMVMLAVMSYNGGVFIAAILGHAVGYVVFGSPIFKKDMDR

SEQ ID NO: 104 - protein, 103 aa, Capsicum annuum
MPMGAGFFKKVRAYSETWDLGLVMNEGKLFEFSPPAAYVVVGLIKLAKPRFPLPFTYVVENSHRHSISDLTESSKLCFSCSRPVMPPFICLTPFSSECLMLII

SEQ ID NO: 105 - protein, 167 aa, Capsicum annuum
MATYVVVVLSTRMPSWSKSPFYTSKDAGSKETCTKDVFFSALFSPKAKEENVSLFFNSSFGFLRIAVTGEKPIFFSLRMREKVRGKNTFSLCEIRKWRTHSIRWVHRIKRKAALSWQSFRWQETLGLIGASERNESKPKIDQGSLPAKPIGEGLKDGTCKVDRAPVV

SEQ ID NO: 106 - protein, 144 aa, Capsicum annuum
MAYFGFTRSFGNNRRRCDVHALISRGCNTCQFGPHISPIYHVRIVARCSPRIHVRRTSYQSLTIRTSIWFYSIYRTEVMFFFALFRASSHSYLVPMVEIGGICPPKGIAVLDPWEIPFLNTLIPLSSGAAVTWAHHAILAGKEK

SEQ ID NO: 107 - protein, 72 aa, Capsicum annuum
MVVPRGMAAPILLKWFVSRDVPTGALSFNGTIITIPIPSFPLLVYLHSRSAKPERARRRKGQTLWPNRNEQR

SEQ ID NO: 108 - protein, 155 aa, Capsicum annuum
MDKIVSYPYFYVKDPVGWVAFAIFFSIWIFYAPNVLGHPDNYIPANSMPTPPHNVPEWYFLLIHAILHSIPDKSGGVAAIAPVFVCLLALPFFKSIYVRSSSFRPIHQGIFWFILADCLLLGWIECQPVEAPFVTIGQISSLVFFLFFAIMPFLG

SEQ ID NO: 109 - protein, 88 aa, Capsicum annuum
MSLLDSPQFLMEIEFSEFSPELGDHFEIFKHIRGFNVTIVTSANTQDDTLPPWSGFLQKMRGKLSKMSEKRNIRDQKYRLLVARLNMN

SEQ ID NO: 110 - protein, 75 aa, Capsicum annuum
MDQLMFPLYFHYEDVSRQDPLLKPNQANILEVPGSCKIIVVPKTTPSIKNGKLAMDISCGQKLIQTQRASTAKLF

SEQ ID NO: 111 - protein, 301 aa, Capsicum annuum
MQDSLVVTSKSSSNIIFSPTLEPVGAHNNSDISSDGGTPPILLRFSVDYSDSSRSSCELQISNVSDFYISDMIISYLAVKGETIYDDSLTDRFLSDYKCEEPISLLIANIFTNVDEECLLFPFLEDTAIASYSQECRTSGETVVQSDDSNLYMEIHHHILFEQSDAFTYIESNNQAECFDLYSFIRNLSNMLERPNILPKESQRYKSITLVLDLDETLVHSKLEHCNESDFIILIFFSMKEHVVYVNQRFHLRTFLERFVEMFKVVIFTASQSIYAKQLLDILDPDGNIISRCTYRESCIL

SEQ ID NO: 112 - protein, 389 aa, Capsicum annuum
MGEESPECEFWLPPQFLNDDDMFMGFKGNSKEEENERKRYFGFGSDFVNEFSYGFNMFAPKSDLSSPVDSLVGSSETESDEEEFFTELTRQMAHATLENHKVQNLSTSPQSTLCGVLGSKQGSNSETPNSPELQSRKGTVDLLYAAVGEVARMKKMEEEVGICHHKNGIWAPPRQTNPAFVDSKMSKPNVGSFYSNQPPPPPLSYQQFKMAQFQLLKQQQIMKQRHHGVLGQEKDGFWEYHLSQNNQQLTHGRDRNVADNRPLNKMAMSVWPTLQQAHHHQQKQQPTYGSGVRGVGLGNVGPKRECAGTGVFIPRRAGTQTETKKKQGGSTVLLPDRVVQALNLNLQSQVQPRFNNGGVLKYQNNNVAHLRKQPTAMSQEHQLPQEWTY

SEQ ID NO: 113 - protein, 221 aa, Capsicum annuum
MSGVKLLGINGSPASRRVEWALKIKGVEYEFIEEDLQNKSQLLLESNPVYKKIPVLLHNGKPIAESLVIIEYIDEVFEGPCILPKDPYDRAIARFWAKFLDEKCLPAVWKALWSQGEEQEEDKKEAYEVLKVLDNELKDKKFFGGDGIGFVDIAANFVGFWIGIVEEATGVVLVTSEKFPNFCAWRNKYLNCDQVKENMPSREMLLGYFKSRVQAVANGPK

SEQ ID NO: 114 - protein, 517 aa, Capsicum annuum
MTSRGLLMSFCSFYIVVILHLFAHTNAAAVPAKRFVAPFNRTSFPSDFIFGAASAAYQIEGGARLGGRGPSIWDTFTHKHPEKIFDRSTGDVAVDFYHRYKEDIRLLKEVGMKAFRLSISWSRILPYGKVSKGVNPEGIKFYNNVINEIISNGLTPFVTLFHWDTPQGLETEYGGFMSSKIVKDYADYADICFKEFGDRVKHWITLNEPLSYSMNGYAKGTFAPGRCSSYVGNCTKGNSATEPYIVAHNLLLAHATGVKVYRQKYQKTQKGKIGVTLVTHWFVPKIKTPLGLKAPQKALDFFLGWFLDPITYGDYPASMRANVGRRLPKFTPEQKKLVKGSMDFLGMNYYTTQYASPMLSVPRVNLSYTTDGHVDMTTQKDGKPIGTPTALNWLFIYPRGIYDLMIYLKQKYKNPPIYVTENGMADANNSTLPLKQALKDDMRIRYYEGHLWFLSKAIKAGANVKGHFAWSFLDDYEWDAGFTVRFGITFVDYKNGLKRYHKKSAYWYKKFLLNNGK

SEQ ID NO: 115 - protein, 592 aa, Capsicum annuum
MDKIKTTEESVEIDEQKWVNDSSVDHKGRVPLRGSTGVWKASLFIIVIEFSERLSYFGLATSLIIYLTKVIHQDLKTAAKSVNYWSGVTTLMPLLGGFLADAFLGRFSTVLVSSIVYLLGLLLLTMSRVVPSLKPCDTDLCHEPRKVHEEIFFLAIYLISIGTGGHKPSLESFGADQFDDDHPEERKKKMSFFNWWNFGLCCGLLLGVTLIVYVQDRVSWAMADLILTLVMASSIVIFVAGRPFYRYRKATGSPLTPMLQVFVAAIRKRNLLFPANPSHLYEIPKSETTQSRLLCHTDKLKFLDKAAILDNTQDSEVQQQNPWKLATVTKVEELKLVINMIPIWLTTIPFGICVAQAATFFIKQGVTLDRKIVHNFEIPPASIFALAAIGMIVSVSVYDKILMPILRRATGNERGISILQRIGIGMIFSVSTMIVAALVERKRLNLVHKNPLEGSSSMSVFWLAPQFLIIGIGDGFTLVGLQEYFYDQVPDSMRSLGIAFYLSVIGAANFVSSLLITIVDHITKNSSGKSWFGKDLNSSRLDYFYWLLAIITAVNLCVYVIVAMNYSYKNVHGKATVSVADCNDADDRNAMA

SEQ ID NO: 116 - protein, 121 aa, Capsicum annuum
MAHSLILLTTNVSMTFSSTNLDNQSPPVPPDEDECQMAIDSAPKALFSEIVKDKPPSLNNFFPLSFEADEAFTHSDLSTNNIQISSENRVRLYAPWKYSVIVKTMGRSFNHQFLKAKVLDL

SEQ ID NO: 117 - protein, 597 aa, Capsicum annuum
MDKINVEMEQIKKTESVDIDEQKMVYDSSVDHKGRVPLRSSTGVWKASLFIIVIEFSERLSYFGIATSLIIYLTKVIHQDLKTAAKSVNYWTGVTTLMPLLGGFLADAYLGRFSTVLLSSTIYFLGLLLLTMSRVIPGLQPCDSNFCDERRKVHEPIFFLAIYLISIGTGGHKPSLESFGADQFDDENPKERRKKMSFFNWWNFGLCCGLLLGVTLIVYVQEHVSWAMADVILTVVMASSIAIFYAGRPFYRYRKTTGSPFIPMLNVLVAAIRKRNLQLPSNPSHLHDVPKSETTQRRLLGHTKRLKFLDKAAILDVTQDSIEQQQNPWKLATVTKVEELKLIINMIPIWLTTLPFGICVAQAATFFIKQGVTMNRKIIHNFEIPPASIYALTAVGMIIAVTIYDKILVPFLRRTTGNERGISILQRIGIGMIFSISTMVVAALVERKRLNVVHKNPLEGSASMSVFWLAPQFLILGIGDGFSLVGLQEYFYDQVPDSMRSLGIALYLSVIGAANFISSLLITIVDHITEESGKSWFGKDLNSSRLDYFYWLLAIITAVNLCVYVFVARSYTYKNVDSRTTMTVADCDDGDNRETMV

SEQ ID NO: 118 - protein, 351 aa, Capsicum annuum
MDHEMEALVRNQTWNLVPKPKDVQPVSCKWVYKIKRKADGNIDRYKARLVSHGFSQKYGEDYKETFSPVAKMTLVRVVLAMAASHGWKLWQLDIKNALLYGELDKDIYMEQPLGYVSNLHPNYVCNLKKELYELKQASRAWYGKIAQYLIFCGYVALHLDPSLFVKKQEDLLVIVLLYVDDMIITGNNDDEVARLQEELAIRFEIKKLGELHHFLGLEVTNTSKGIFVTQEGYAKKLVDRFGMKHSKKCSTTLEISTRLRREEGSLLADPKPFRALVGSLLYLNITRSDIVFSVRYVRRFMQSPRKPHLEATKRILKYINSTLDMGLFFQKKNDLILVGYTYADFGSDMDV

SEQ ID NO: 119 - protein, 719 aa, Capsicum annuum
MXGGFAIGDYVVERQIGVGSFSTVWLACHCDDGTEVAIKEISTARLNDKLKENLKSEIVILQRITHPNIIRLHDMIEEPGKIYIILEYCRGGDLSMYIQRRQGRIAEATAKHFMQQLASGLKILRENNLIHRDLKPQNLLLSATDDNSTLKIADFGFARSLPSRVLAETLCGSPLYMAPEIMQLQKYDAKADLWSVGAILFQLVTGKTPFTGNNQIQLLQNIVKSTKLQFPLDAKNLSPQCIDLCQKLLRCNPVERLTFEEFFNHPFLAQKQPDEFWNQRSQRVIDGFSLTEERNPVRSIDETIQEDGLPLPDDDSSSPDFAEISQRHDAKDYRKDVASFSNKTGPPLNCSNITHKPDVIGSSPGQHRPSEGKLKESSSVDHRLCPRVADSFELIEREYVLVSGSLMDSFSAAGASSISNTPFRSSRSLQASGNIDPKLGEPVLIVGPATNSIGPVLRLESPSSSPGASQGSTDIVDPPEKPTAGITRIESLQHSASAITELVNEKIAAGKHLEAFSIELVILAIWKQALHICHMQAASAIEGSPNQEITKLTEILRDIRVSPKIKEDTCNTLGLENVLSHIQRAFLNEVGKGEEIAKHIEPGNAEVPDAMEMIFQSALELGRKGAVDEYMGRTKTAVVLYSKAVRLLAFLLVEAPSLIVNPPFVLTNTDCCRLGNYIDVLKKRQSVSMSQIMTLLKCEDRPDPREDVKNRSEEKSVNM

SEQ ID NO: 120 - protein, 178 aa, Capsicum annuum
CLTEHGTARGAASHKLLMPEKTLTKDWKHLFTANRIASKGAIERFITAQGAFSVKPMVLYHMDKYFVIRFASKEERDGILCFEPHYLLKRPVIMKPWSSEFNFKEEILSTIPLWAKLPNLPLNCWNAVALSKIGRSLRQLLYADECTTHKIRISFARILVEVDVSLELPKSIKVQDPK

SEQ ID NO: 121 - protein, 298 aa, Capsicum annuum
MADAIKYFELNTGAKIPSVGLGTFQSPPGVVGQAAETAIKVGYRHIDCAQVYGNEKEIGEVLKKLFEDGVVKRDDLFITSKLWCTDHAPEDVPVAFNKTLDDLQLDYIDLYLIHWPVRMKNAVDSFKPENLIPADIPSTWQAMEKLYDSGKARAIGVSNFSTKKLGDLLEIARVPPAVNQVECHPTCQRTELKELCKSRGVHLSGYSPLGSPGFVLLKGDEVLKQPVVISVAEKLGKTPAQVCLRWGIQMGHSVLPKSTHESRIKENFDIFDWSIPDDLFTKFSEIEQVSCLNALLYV

SEQ ID NO: 122 - protein, 125 aa, Capsicum annuum
MEGIGKIACFVLLCMVVVAPHAEALNCGQVTSAMAPCVPYLMGRGPLGGCCGGVKGLMGAAKTPADRKTACTCLKSAANSMKGIDAGKAAGLPAACGVNIPYKISPSTDCSKYVNSPSLFCALTI

SEQ ID NO: 123 - protein, 141 aa, Capsicum annuum
MVNSHVDEIPADLDDSMVKLFVDDMHLLNAPWKYFVIVKTFGSKFTHQYLNTKLDALWRLAEPLCSIDLGPDFYIVKLQNPESQVKVWQGGPWFIAGSFVSIRKWDPNFVPSNSMIDYTAIWIHLPQLLCHHWTSEAKLSL

SEQ ID NO: 124 - protein, 202 aa, Capsicum annuum
MLFVCCHKTLLDPVSLSVSLRHPIPVVTYSLSRLSEIISPIKTIRLIRDRESDASMIKKLKEGDLVICPEGTTCREPFLLRFSSLFADLTDEIVPVVVENKMSTFHPTTARGWKGMDLFYFFMDPSPIYEVTFLNKLSPELTGKLGKPSHEVANYIQRAIATTLSYECTNFTRKDKYRALAGNDGTIEEKGRRFKAETFMGC

SEQ ID NO: 125 - protein, 511 aa, Capsicum annuum
MGITTFPTIEKCTLEGREKDTVVADLDGTLLRSRTSFAYFALVAFDVCGVLRLMFLLLVSPLIGFLYYFISESAGIQGIIFATFAGMKVSDTESTARVVLPKYYSEDLHPESLRVFSSCGKRCVLTANPRIMVEPFLKDYLGADIVIGTEIETYKGRATGFVRSPGVLIGQKKADALRKTFGESQPEIGLGDRDTDIPFMTLCKEGYFVPSKPKVKALTADKLPKPIIFHDGRLVQKPTPLMALLIILWIPFGFLLACMRISIGLLLPISLHYYVCWLSGIRFIIKGVPPPPIKKSKDKTGILFVCCHRTLLDPFFLSVALGRHIPTVSYSLSRISEIISPIKTIRLTRDRASDASIIKKLLKEGDLAICPEGTTCREPFLLRFSSLFAELTDEIVPVALESKTSMFHPTTARGWKGMDSFYFFMDPSPIYKVTFLNKLSSELICKFGKPSHEVANYIQRAIATTLSYECTNFTRKDKYRALAGNDGTVAEKGRRIKAKKLASYSYDKLVR

SEQ ID NO: 126 - protein, 472 aa, Capsicum annuum
MVGFAKMGWLSSVNNNKSSKIADDVNKMGILAFETAKIMSRLLCLYKSLSDSEISKLKKEMKSRGVAYLNSKDDGFLYSLACAERLEDLDKAAAAVARLGHKCSDFGLNRFDLVYTDLKLGIIDFGKLEYGSKEIEKRVDKMEKLINATSGLYSALENLTELEISDRKMKQWKERKASGQLQKVNLDMLNQKLEQQRKQVRQFREISLWNQTFDKSVGHMARIVCIIYARICLVFGPYIPVLPSLSLRNMRASQQKEIIKVQPENCLIEPIREQVISRSGPIPTTSKPTLVRFYSRKSIFFLCDDEGFGSEKLAKNNRVFHAAGPLTLGGSGLALRYANVISLVEKYSNPSETVDLNSRENLYQMLPGNLKKTVRSKLSKNLKCMDEDESLAEGWRDALKQIMEWLAPMAHNTINWQLERNLEKTKFDIKPSVLLLQTLHYSDKEKTEAAIADILVALSCIYKCENRQWSEP

SEQ ID NO: 127 - protein, 84 aa, Capsicum annuum
MDNEKCQCSNFRQYKHDENSPKTWPELIGVLGDEAAKLIKAEMPNLQIIQIIHPPWALTQDLCSDRVRILVDGFNRVSMPPSIG

SEQ ID NO: 128 - protein, 455 aa, Capsicum annuum
MIVIMIVEEEEVAAMTTKWVWRRRSGGDDLSLNNSGAGTSVPSTAFFGKTLKKVNTKGASSPKVSNRSFRITFTAQQPEIDEKKQTEQDRWKGLVSDTSDDQQDITRGKGMVDPLFQAPVGTGTHHAVLSSYEYISQGLRQYNLDNTLEGFYIAPAFMDKLVVHITKNFLKLPNIKVPLILGIWGGKGQGKSFQCELVFRKMGINPIMMSAGELESGNAGEPAKLIRQRYREAAEIIRKGNMCVLFINDLDAGAGRMGGTTQYTVNNQMVNATLMNIADNPTNVQLPGMYNKQENARVPIIVTGNDFSTLYAPLIRDGRMEKFYWAPTREDRIGVCKGIFRTDSVPDEHVVRLVDAFPGQSIDFFGALRARVYDDEVRKWIERTGLEQVGERLLNSVEGPPVFEQPKMTIEKLLEYGKLLVQEQENVKRVQLADKYLKEAALGDANADAINTGNF

SEQ ID NO: 129 - protein, 687 aa, Capsicum annuum
MFNFFTFFCLIVSLSILASCELDEFIYTRFNEPNNSLTLSGVAKIGQNGYIQLTNDTSRLMGHAFYSSPFHFKNSSDSTAFSFSTCFALAIVPEYPKLGGHGLAFTISPSKDFSTALPSQYLGLLNASDIGNYSNHIFAVEFDTVRDFEFGDINDNHVGVNINSLASNKSAAAAYFNDDQSVKQDLNLKSGKVILAWVEYDSVTRLVNVTLSPTSSKPKVPLLSYHLDLSLIFKENMYVGFAASTGLLASSHYVLGWSFKLNGESKFLDLDSLPSLPGPKKKHTGLIIVISVISVVLALSLILVAVYLVWMFKNADVIEPWELEIGPHRYSYQELKKATRGFKESELLGRGGFGKVYKGVLRNSKAQIAVKRISHESKQGLREFVSEIASIGRLRHRNLVQLFGWCRRRGDLLLVYEFMPNGSLDNFLFDQPKMVLTWEQRFKIIKGVASGLLYLHEGYEQVVVHRDIKASNVLLDGELNGKLGDFGLARLYEHGSNPSTTKVVGTLGYLAPELPRTGRATTKTDVFAFGALLLEVVCGRRPIEHKAGPEEFVLMDLVWDKWREGKIIDVVDYRMKGEFNKSEVLMVLKLGLMCSNNKPLARPSIRQVVRYLEGEIGMPEALTAHDSDDFEGFGFDESEHWNSLGSTNIFKSSSFTCLSNGDENSSTYVSFSTSPLPLLYSGEIPRY

SEQ ID NO: 130 - protein, 245 aa, Capsicum annuum
MLSSLYLIHILLCIFLLLSSSDGTQVILVNNCKESIWPGILGGAGQTTLKDGGFHLSSGEQIVLDVPDKWSGRIWARQNCHFDENGKGKCDTGNCGGQLQCRGLGGEPPATIVEMTLGSSTSPLHFYDVSLVDGFNLPVSMKPVGGGAGCGVAQCEVDLNICCPSALEVKSGDRVVGCKSACLAIQSPKYCCTGQYANPKTCKPTVFANLFKAICPKAYSYAFDDASSLYTCRAPRYLVTFCPPK

SEQ ID NO: 131 - protein, 580 aa, Capsicum annuum
MGSMRLDEEFGGDRVLHIETDDEENEASHDNEEVDDDNDSGSDDLMTSHSPQPPFHGQHGPLDSPTWPQSYRRSMNMFTGVTPPSLSFLKGSGVLSSAYVRSQSPTPQHSLCKPLISSQSLEKEEVVPTLNPSKLSIVSSAPELPPSQQCSYIQSVLNAINALCGIGILSVPYALKEGGWCSLFLLLLFGVITFYTGTLLKKCLESSHGIETYPDIGQAAFGLLGRIFIAVKQDLCVVTNAGFPLNADSLVRGTICKCLILTLCFIRVLLYVAYNLRRSSSLQSSCIEYLIMMSDNLGAVFPNSHMEFAGIHLDSYQMCAIISTLVILPTVWLRNLSLLSIVSAGGVIALIIVVICLLWVGVINDVGFHPSGTALNIAKLPVTIGLYSFCYGSHSVFPNIYSSMKEPSRFPSVLLISFSIAFFSYLGVAVCGFLMFGENTNPQFTLNLPAKLITSKVAAWTVVVAPLTKYAITITPVAFGIEEFLPSPQLRTYGVSILIRTLLVFSTLVISLAVPYFGSVMSLIGSSLVMLVSLILPCACYIKLSKDRITRFQFAACTFVMVVGVISAVVGTYSALTSMA

SEQ ID NO: 132 - protein, 580 aa, Capsicum annuum
MAPPITFPSLVPMAMLIIAFLALCLIPQPAQAITRHYEFNITMQNVTRLCHTKSIVTVNGKFPGPRIVTREGDRLLIKVINHVPNNITLHWHGVRQLRNGWADGPAYITQCPIQTGQTYVYNYTIVGQRGTLWWHAHISWLRSTVYGPLIILPKENVSYPFVKPYKEVPIILGEWFNADPEAIISQALQTGGGPNVSDAYTINGLPGPLYNCSAKDTFKLKVKPGKTYLLRMINGALNDELFFSIANHTLTVVEADALYIKPFDTETLLLTPGQTTNVLLKTKSEYPDATFLMSARPYVTGQGTFDNSTVAGILEYESPTLHSTKSVKKIPLFKPVLPPLNDTTFAANFSKRFRSLANSQFPANVPQNVDKRLFFTVGLGTNPCDKNQTCQGPNGTKFAASINNVSFVQPTIALLQSHFSGQSKGVYSPFFPVNPLNWFNYTGNPPNNTMVNNGTKVLVLPFNTSVELIMQDTSILGAESHPLHLHGFNFFVVGQGFGNFDPNKDPAKFNLIDPVERNTIGVPSGGWVAIRFLADNPGVWFMHCHLEVHTSWGLKMAWLVLDGKLPNQKLIPPPSDLPQC

SEQ ID NO: 133 - protein, 156 aa, Capsicum annuum
MNNCVGHANYKIFFIFVMYAVVACIYSLVLLAGSITVDSSKDDLQSEGSYRVVYIISGLTLVPLSLALGFFLGWHFYLILQNKTTIEYQEGVRAMCVAEKGGYVYSHPYDIGAYENLISVLGPNVLCWPFPSTKHIDSGLRFRTSFDSIPGTPASK

SEQ ID NO: 134 - protein, 463 aa, Capsicum annuum
MSSSENGVHILIFPFPAQGHMLPLLDFTHQLLLHGFKITILVTPKNVPILDPLLSTHPHVNTLVFPFPGHPSLPAGVENVKDVGNSGNAPIIGGLSKLYSPIVEWFKAQSNRPVAIVYDFFLGWTHDLAQEVGVPGIVFYITGAGLVSILDDLWKNFEAYKGLGLVEFNGLPRSPRFMREHLPSVFLKFKEDDPTWEIVRNGFIANGRSFMSIFNTFEALENEFLGFLKKEMGHDRVYSVGPINLVGGPGRIGKSNMDERVFTWLDDCPDGSVLYVAFGSQKLLTKAQMEALTTGLEKSGVRFILVTKQLSAQQEDQGFGSVPKGFDQRVSGRGLVIKGWAPQVEILGHQAVGGFLSHCGWNSVMEGIVAGVLILGWPMEADQFINAWWLVDNMKTSVRVCEGSNSVPDPIELGLRISEAMSNNLFKERAQKMRDEAFEAIKIGGSSKRDLDWIVRELTQLKG

SEQ ID NO: 135 - protein, 436 aa, Capsicum annuum
MGVLVISCYCSSLLGYEGLEVVNPEGNNEDAGAEAQRGRWKQEDRDNYWKMMQKYIGADITSLVTLPVIICEPMTNLQKMAELMEYSYLLELADECEDPHMRLVYSATWFISVYYALQRTWKPFNPILGETYEFVNHAGITFIAEQVCHHPPIGAAHAENEHFKYDITSKVKSKFLGNSVEVYPLGRSRLTLKKSGVVLDLVPPPTKVLNLIFGRTWIDSPGEMILTNLTTGDKVLLYFQPCGWFGAGRYEVDGYVYNSEEEPKILMTGKWNESISYQPCDMEGEPLPDSTLKEVWKAAEAPKNDKFQYTYFAHKINSFDTAPKKLLSSDSRLRPDRYALEKGDMSKASSEKSRLEERQRADRRTREAKGDEFKPKWFNLSNEICPTPWGDLEVYEYNGKYHEHRTAIDSSGHVEEVDPKTIEFNPWQYDDSEVSA

SEQ ID NO: 136 - protein, 258 aa, Capsicum annuum
MSITWMLICIASLVSFLTSVDAKVRGVYTVGPWQDAHATFYGGSDASGTMGGACGYGNLYSTGYGVNNAALSTALFNSGLSCGACFEIKCTDPKWCTPGNPSILVTGTNFCPPNYALPNDNGGWCNPPRPHFDLAMPMFLKIAEYRAGIVPVTYRRVPCKKQGGIRFTINGFNYFNMIIVTNVAGAGDIQQVQVKGTNTKWIFMSRNWGQNWQSNVVLVGQALSFKVKASDHRYVTSYNVAPSNWQFGQTYEGKNFRV

SEQ ID NO: 137 - protein, 153 aa, Capsicum annuum
MEGHLTAKSDVYSFGVVLLEILSGKKAIDKNRPTGEHSLVDWAKPYLTNKRRVFRVLDARLEGQYSLTHAMKVAILALQCISMDPKSRPTMDEVVKALEQLQESKDVAKNDKKVRQVNQHSRSSFALKKSCKSSAEGTPAKAKYPRPSLSLLS

SEQ ID NO: 138 - protein, 298 aa, Capsicum annuum
MYILILLLALKWILLGLGSRNASGNGTDTSNSNSKRSSASIPPTPRSEGEILQSSNLRSYTFSELRAATRNFRPDSVVGEGGFGSVFKGWVDEHTLAASKPGTGIVIAVKKLNQEGWQGHREWLAEINYLGQLQHANLVKLIGYCLEEDHRLLVYEFMPKGSMENHLFRSEFCTCFIFFTRSFYQPLSWSLRMKVALGAARGLAFLHNAETKVIYRDFKTSNILLDSDYNAKLSDFGLARDGPVGDQSHVSTRVMGTYGYAAPEYLSTGLSPCYPISFSSSPPDGIICFLLHGSVYKR

SEQ ID NO: 139 - protein, 375 aa, Capsicum annuum
RLDSLGVPAENLEQLQPGLVAYVKNNKSQMGELVSALLPTNEEAMEVITEQQIESPKSTVSSSDNVKDLFQESMDWIQWLMFDGEPSRALEQLADTGQRGVCGAVWGNNDIAYRCRTCEHDPTCAICVPCFQNGNHKDHDYSIIYTGGGCCDCGDVTAWKREGFCSRHKGAEQIQPLPEEFANSMGPVLDLLLSCWRKKLLFPESISGKNARGNDHAAELKQVIDELTSAVVEMLLKFCKHSESLLSFISRRVSSSAGLLDILVRAEKFMISEENVKKIHELLLKLLGEPQFKYEFAKVFLSYYPTVVNEAVKKGNDRVFNNYPLLSTFSVQIFTVPTLTLRLVKEMNLLPMLLGCLEDIFVSCAGEDGKLQVML

SEQ ID NO: 140 - protein, 831 aa, Capsicum annuum
MSTIIAGWEVLECWEGILTQRFPEERVDCRVISRAMEEFSDFVDDKCLIDLPLTWENFTWARADLYETTLRVVEDIRFVMSHSLVPRYVTHDRRDILRTWMKLLAFVQGMNPQKRETGIHVEEENENMHLAFVLGHCVANIHCLLVGGAFSMSNTEDADDSFFNTHTENFEDQDSQRHAKVGRLSQESSVCSVAGRSPLEHSSKVPELKYDSFPVSSSVLCLTFECLRAIENWLIADNTLGPLLHILCPKKSSSPVNKFSALKRTLSKIRRGREIFKPQSPPSNDIRLSTSAEGYNEKYSCPSLNGSTTLDSGQGSGQEAACLGGHDDGMLEGDNASELDALRLLSLSDWPDIVYKVSLQDISVHIPLHRLLSLVLQRALGKCYGETAEQGATSANLSPSVHYDFFGHILGGYHPQGFSAFIMEHTLRVRVFCAQVQAGMWRRNGDAAILSCECYRSVRWSEQCLELDLFLLQCCAALAPADLYISRILERFELSNYLSFNLERPSEYEPALVQEMLTLIIQIVKERRFCGLTSSECLRRELVYRLSISDATHSQLVKSLPRDLAKIDKFQEILDRIATYSNPSGINQGMYKLRLPHWKELDLYHPRWNSRDLQVAEERYMRFCNASALTTQLPGWSKIYPPLGHIAEVATCSTVFQIVRAVVSYAVFSDASNASHAPDGVLLRALHLLSLALDICHAHRESSEHSFYKGDAIPILALACEEISMGKFGDQSLLSLLVFLMRKHKKENYVVETGMLNLVSLVENLLKNFAELQPECMRKLQDLAPDLVNQLSRSFPSGDTNSFISVSDSDKHKAKARERQAAILVRLLKRS

SEQ ID NO: 141 - protein, 603 aa, Capsicum annuum
MGTWSPYMDLEFSPYELTFGVELGPRSILNINKEEEENIVPDKGRQLGTCLNLQLAKYGNNTGIILFDSLTSSMYYMSALQEKMRVQQSKFLASIGSTQDVAADDSKHGEELCNSDGRIRSEDSTPVICSLCHDPNSKSPLSYLILLQKSRLLSFTNRGPPSWEQTRRPGKEPMSCAKQVTDISSERSNLSRSSETISPSWLMQLIQNKVNEFALEGQPKEVEAFLEYIKAKFPSVKNIQPSCASVTVKKKTPSSFEMLEEHMYSLIRDEVDAYSWGWDLLKNDKKLSALSNDGSAESLLLGRYISALSRECSPASVNSRKAQLESSMLLPTYNEFSPSDCDGIYLSSCGHAVHQGCLDRYLSSLTERLYTRRIVFEGGHIVDPDQGEFLCPVCRGLANSVLPALSADAKRSTPSVSTGSSDAVGLPMLRFQEALLLLQSAADVAGSREILQSLPIQQFGQMSVNLESVVRVLCEMYFPDKDKISESGRLSHSLILFDTLKYSLVSTEIAARCEKTSLAPNYSLDALYKELKSSNCFILALLLSIVQNTRTKNLLTVLLRLRGIQLFAESICSGISADEPPNSPTVRGLVPAPLVTFCASIIF

SEQ ID NO: 142 - protein, 338 aa, Capsicum annuum
MQDFLEFSEMELQYPDIQFWKRASDPLLAHDAFSSLMWVLYCLPVPFLSCEESFLSLVHLFYVVTIIQTVITYCRKRQTSLTESGCSDSLVTDIYRIMNENGVAYKYFDSNHMETHDVKDAIRSLSFPYLRRCALLWKLVRSSISVPFNDGSNILDGLPYSMGETSECGENKAVEFNEIEKLEKLFKIPPLDDVISNNTIRFVVPRWLRRFSKQFEAHIPKDVMYSTPAVPFKLMLLPHLYQDLLQRYIKQHCPDCGVLLEDPVLCLLCGRLCSPNWKPCCRESGCQTHAMVCGAGTGVFLLIRKTTLLLQRSARHASWPSPYLDAFGEEVSQWFSYL

SEQ ID NO: 143 - protein, 694 aa, Capsicum annuum
MPLEQTAPIRYLGPSNLVSSPKSSLNDETLQQPQNEYSTIASLLDPPQPEERKDAVTTHTCELCAAMLDMNHLKQCATTKNGKQPVTNFTLRRTQSLYFRERTKNVSTEESKLFLDALDLLNMREELFLKILQDPNSSLASQLHGAHASKGLTKSVSFPSRLSLGKIDRSSNHKSCQDESQIRGELLGTAVFESAEKLNRHLVTRRKEEVVKDVLTRFESFDKVPPSSSGAKHKHHNSKLVLARFKNLKEKITHALKESRKEKHRIVMDGVLHKVPHGHRSSNDVKQGTYLAEKPTADGHDGYHSTFSKSQKKSFSRTSSLNDSLDSYNRLLESCFSTDEKQLSSERSSLRASRSPSPGRSRTIVLERILSLPDLRHYPSFRFEDTPEASYSETLDTAALAASRSSEQNSLHIPLGSERKIQQDSSSDAKIPEDFLDVSENSDDIGDLKTEENSLLVEYNKEGKSSSNSTSDKPIPTTMPDMIIEEATTVPADLSATEGIAENAFGTNEEGISTDEQKRNLLLIQVDERNKAEFNYVKDVLDLSGFRGNEFMNLSAFDDVGGCFLSQPDCSGYDEENGICNQLLLFDLINEVVLQLYDRSSLYWPKALTPRSYIHPMLHVGYHLLEEVWREISWWLSFNLEINDQSLLGDVASRDLAKGDHWMNLQFDAECVGLELEDLIFDDLLDELMFIDIY

SEQ ID NO: 144 - protein, 183 aa, Capsicum annuum
MRNALIATNNEPKGHVNPKRVPQEAYKNKNHEGSSTTCIITLNCKKNTICAANVNDSRFFLFRKGKIIYKSPIQQWSLGCPYQLGKCNDNPNVAHEMELNIKKDDILIVGTDGMLDNMNESDIEEIVQRAIDHKLKADGLARKIGNVALFNSLDRFADTPFARASKRKGGKVGDITVIVAYFQ

SEQ ID NO: 145 - protein, 230 aa, Capsicum annuum
GFWDSYRVWDYHGEVLISGENSNPVGSDQVEDDEIKHDGVIGIIHNACRHINVDDNTNSREGNEDPNVHPKKFYTLVEDVQTELYLGFTKVSKLSFVVKLLLLKCLNHWSNKSMDEFLNFLIEVLPERSFIPKSFYEAKKVLSDLSLGYTKIDACKNDCILYWNDYLNFQSCPKCGESRWKSKEHKGKKFAHKVLRHFPIKPRLQRLYMSKETAKKIRWHKEENIEDGVM

SEQ ID NO: 146 - protein, 238 aa, Capsicum annuum
PDDKQNTNSFGSAEDFERRILGDSTGNKPSPNSFFRKLGKVEKAYDRSGLGSMFGSGNRSSILDGLGESFNTLSDGMDGKLKEAASYFEFDPNEVEKEDYAYRADMTFWPGNTYELKDIDLKKPGVRKPKRRVEFETTTEEVLRKADFRNVRFLANFITEAGILVKRSKTGISAKAQRKIAREIKTARAFGLMPFTTMGTKQFVFGRTMEDLDADYEYEMYDSNFVDTEAGRESPFKA

SEQ ID NO: 147 - protein, 259 aa, Capsicum annuum
MCSIDNFLDMEAQMLEQQKKKRNIVSCREYYCYKIQMRNDENDELLHTRRAFQQYIVDMFIKLETQRLDFFFFNPELFRMKVLQGLLDVLGHGERDAAKIGKQTFLPVTFTGGPRDMRQRYLDAITLVQRFGKPDLFLTMTCNPTWSKIKEHLLPTDETQNRPDLISRVFRAKVEELKTDILKRNVFGKVVAFIYSIEFKKRGLPHAHLLIILANQYKLLTPESYDKFICAELPDSNKDAYLYSVVVHYMIHDDIRDIL

SEQ ID NO: 148 - protein, 291 aa, Capsicum annuum
MAKEKTIEAFDLLLKDLMDTRVLFGRKVIVFGGDFRQTLPIVQNGKKKDFISQTLLYSQIWNQLKKLHLSENIRSRTDPAFSEYLMRIGNGKEKLNSNNKVEIPKSLLIPFTTEEQSLNELFKLTYPNINLLFHDSSALAFRVILTTKNDYVHEINDMLISKFPNTAQTFVAIDETIEPNDQSQFEDYLHTINPAYLPTYRLTLKENCPVMLLRNLNPSEGLCNGTRLTCCNFTIHVISAKIARDDFQIKHVFIPCIPLSASADENLSVPFKRTQFPRRLCFAITINKAQG

SEQ ID NO: 149 - protein, 457 aa, Capsicum annuum
MAASRRLRDLQSQPGNKVCVDCSQKNPQWASVSYGVFMCLECSGKHRGLGVHISFVRSVTMDSWSEIQLRKMELGGNENLIRFMKSYEISKETDIVTKYNSKAASVYRDKIQSLAEGKIWKDPPVVKETLTGPTRPPLSGGGSKSRNSGWDDWDNFDDGGNSNSNSNMKRNQTVSDFRSGGGGGGGPARSKSTDDVTRAQYEASAANKDSFFARKMAENDSRPEGLPPSQGGKYVGFGSSPAPMPRNNNNYNSQGDVFSAVTQGFGRLSMIAASAAQSAATVVQAGSKEFGSKMAEGGYDQKVNETVNVVATKTTEIGQKSWGIMKGVLALASQKVEEFAEGPNSRDNSWKRSENERNGHYKESSQASKGWNSSGGAQSSGRHNNSVSSASWDDWDSNDNRKENSTRGATSSNDDDGWAGWDDGKDDGFDDFQSAPNNKHVAHGGKSESKWTDGGFL

SEQ ID NO: 150 - protein, 455 aa, Capsicum annuum
MDSTVQENSPNSQSPKPNFMVKLKDNLIFKSKWAELNGAMGDLGTYIPIVLALTLASDLNLGTTLIFTGVYNFVTGAIYGIPMPVQPMKSIAAVAISHPEFGIPEVMAAGICTAGILLVLGASQLMQLVYKLIPISVVRGIQLAQGLSFAMTAVKYIRNVQDFAKSKSGKERDWLGLDGLLLALVCAVFILIVNGAGENQNEDQESTESETGYLRKKVRKIIFSLPSAFLIFLLGVVLAIIRGPKAVKGFKFGPSRIEVVSISKQAWKDGFIKGTVPQLPLSVLNSVIAVCKLSTDLFPEKREITATSVSMTVGLMNLIGCWFGAMPCCHGAGGLAGQYKFGGRSGGCVALLGVAKLVLGLVLGSSMVKVLTQFPVGVLGVLLLFAGIELAMCSRDMNTKEDAIVVLVCTAVSLVGSSAALGFLCGIVVHLILKMRNMDSDGHSCSSVLWFHRNP

SEQ ID NO: 151 - protein, 117 aa, Capsicum annuum
MASKLSIVILCLALVVVQTTAARELPSDKSLSDQKNVFGGAGVIGGIGSNGLPYGGVISGVGSNVGGVGTGIGGGAGFGSVGGLGGGLGGLGGLGGGIGGGAGTGVGAGGAGNLPLP

SEQ ID NO: 152 - protein, 132 aa, Capsicum annuum
MAVHDVCKLRFLELKAKRTHRFIVFKIEEKQKQVVVEKVGEPTQSYEDFAESLPADECRYAVYDFDFVTEENCQKSRIFFIAWSPDTARVRSKMIYASSKDRFKRELDGIQVELQATDPTEMGLDVIKSRAN

SEQ ID NO: 153 - protein, 217 aa, Capsicum annuum
MDSNDKSWMNCLIWSDEFQHGVKDFLKKAFERASQGNEILCPCRNCKNHYWHYRDVVEDHLLSRGFRAGYTKWTFHGENASSRKTHHLINDNEGSNIRDDIDGLLHNTFRNVEAKLGHEEGDKEGLPEDAKRFFKLLEDGKQQLYPGCENFTKLSFTIRLFLFKSLYGLSNVAFSDLLVLIKEAFPFAQIPESFNKAKNVTGDLGLDYEKIHACPND

SEQ ID NO: 154 - protein, 312 aa, Capsicum annuum
EMECGGKEPKKVNNCDDQPDVRIDVKEDDMLSEVSNPNLSPRESTSSLQTASSQGVDLLSNNQGVSGDITSFSSGNSSAEESVSEEEHNQIDALKDVAKSSVVLEIPEEFSTTGVRKITFKFSKRKEDYDNASAATALPATAADDGFCEAQTWIESDDMNPFSRPANMELKMSKKVTSDALPTNVKKLLSTGILEGAKVKYISTSGKKELLGIIKDYGYLCGCSLCNFSKILSAYEFEMHAGGKTRHPNNHIYLENGKPIYRIIQELKTAPLSQLEEVVKDVAGSSVNELYLEAWKGSLTLMSILIYLTFHF

SEQ ID NO: 155 - protein, 462 aa, Capsicum annuum
MKGFLLFGLAFVINVFTIHRDNDLHRSLFMPNGLPDGTDLAYYSKGKKVLGGYKQGNGIVCSCCDTEISPSQFESHAGCAAKRQPYRHIYTSNGLTLHDIALMLANGQSIATNNSDDMCTICGDAGELICCEGCPRAFHAACLGLQCTPTSGWLCSYCRDNFVPGRRTAGDAGPIMIRLTRVVKAPESEGGGCVVCRTPDFSVSKFDDRTVMLCDQCEKEYHVGCLRESGLCDLKELPKDKWFCCNDCHKVYVVLQNCVLKGAEVIPAPAAAVVTKKHVQNCLMDTTTNDIQWRILSGKSRFTEHLPLLSNAAVIFRECFDPIVAKSGRDLIPVMVYGRNISGQEFGGMYCVVLIVKSVVVSAGLLRIFGQEVAELPLVATSRQNQGKGYFRALFGCIEILLSSMHVKTLVLPAAEEAESIWTNKLGFRKMTDERYRKYSRDFQLTIFKGTSMLEKEVQQTA

SEQ ID NO: 156 - protein, 238 aa, Capsicum annuum
MAAAAPPPPPSPAPSAISEDLLPTSHNLFSRIRLAKIADVPHIHKLIHQMAVFERLTHLFSATESSLSSTLFPPNPPQPFTSFTVFLLEVSRTPFVSIDQINPSFDPVYKTVMIDVPINDPEAELFKSGAGGGGNDIVVAGFVLFFPNYSTFLAKPGFYIEDIFVRECYRRKGFGKMLLSAVAAQAAKMGYGRVEWVVLDWNVNAIKFYEEMGAEVMKEWRVCRLTGEALQTFANVNI

SEQ ID NO: 157 - protein, 220 aa, Capsicum annuum
MNSNSNSTTPNFDNLLLQSLIGRLQIRPPPPPSSTNPSFLSPKSLEQLLFNNLLDDDDDDDDDNLDDCVNGDGSSKSDLSREESRLEKQIIRTILSGKVDSLKPNSGQAVTIGEHHICVGFHEDSGSDYRVWEWHGHIMLFDEENGYTPEYIYGNYFERVGGKLMTKTTTKKKEEEEEEVEKEEKVGNLGLKELIESGDSNSEGRILRRNMNAGSPRFVE

SEQ ID NO: 158 - protein, 157 aa, Capsicum annuum
MSKISPILLLAIVITAIVFFIFGHLHLLIRYCLKMPSFSSISQSNRFPESSGPQVLQRQLQKLLRQHDSGLDQAVIATLPVFCYKDIRGLKEPFDCAVCLCEFSEHEKLRLLPFCSHAFHIDCIDTWIHGSCQTLLVLYVEVLLLLGFLWKSLVLLY

SEQ ID NO: 159 - protein, 180 aa, Capsicum annuum
DFRQVLPVIPKSTRVEMVNESLVKSYLWNRMEKIKLTRNMRVRTDASFSEFLLRIGNGEKPTIRDDLVLLPRQLVIETKSDRTSLDALIEQIFPSFPGNASSAKYMTERAILASRNKYVDQLNEMLISRFPGETRMFLSFDFAEDDTNNYNQEDYLNTLTPNGLPPHRYVLTYLLICDYV

SEQ ID NO: 160 - protein, 195 aa, Capsicum annuum
MAEFQDATAGRAQTHQFAQRVEFSYFDEGDPHSWLRKYELRWSDFVEETCRRFEEADKNNLNLLGEFKKVEQKGTVSEYLEKFEELKSWVLIKHPTIPEEFFLGFFIEGLKDEIKHTVKMLDPFSLSQTVEKAKHQEKLIETINKKSKSTGVRMAQFNSSRSRNFGQGTKLIEVRRVRGMFKVREKILPGSSMQG

SEQ ID NO: 161 - protein, 301 aa, Capsicum annuum
MPGNFVKMPKLQDKQQPEVFEATSQNMLFETTTSTTIKIMGLYRKKKLVILVDNGSTHSFVASETANQMGCKVQNNIPMRAQLVLYWTNMKTEIGRWVSECDVCQWTKTKNLHPRGLLLPLPIPDLNWQDISMDFVEGLPTSDHKNVVLVVADRLTKYGHFLALKHPFTAKDVAELFLREVHRLHGLPRTIISDRDPIFTSQFWQHLFRSMGTYWSKWLHLVEWWYNSNYHTSFKASPFEALCGYPPPQLPLVSLPYYGNSDVRLSLDQRQHLLQSLKDNLLRAQSRMKFYADKLKLERIL

SEQ ID NO: 162 - protein, 235 aa, Capsicum annuum
SAQEIRPPSLISSSEPPSLFDGTTRLYISYICPFAQRAWITRNVKGLQDKIELVPIDLQNRPAWYKEKVYAQNKVPALEHNNKVIGESLDLIKYIDSNFEGPSLLPDDPEKQKFAEELVTYTDTFLKEIYGSFKGDVEKQAGPHFDYLEKALEKFNNGPFFLGQFSQADIAYASFIERFQIFLQEVFNYDITSWRPKLAKWIEELNKIDGYIQTKADPKKVVEIYKSRFMVVTHT

SEQ ID NO: 163 - protein, 464 aa, Capsicum annuum
MEVNEAAKLPIARPVASRPRCPLYKSFSELLTGAVDISSTNVHSEMAITTIRPKTIRLKPATNHALVGEPSSQVGVSEAPVGFGADNILQSVKKPKVLYKPIAKLAPKKTIPLLENKGSSVSDQQRVIADAEAHIQSANEVKQQHDPTTESKQSLSAKSGQDKKKVRSTIVSGSTEEVAQSLINTSNVDRPSYDGYNWRKYGQKQVKGSEYPRSYYKCTHLKCLVKKKVERSYDGQITEIVYRGEHNHPKPQPPKRNLSDGHRRTAICNDTSKETNNPAWSNQHPQMSEAYVCRRENQNDGGLTIHSSKVPCFYDPIVAAGMHTAVRNSEDSAEGSKKLKATCDEQKSKRRKIKCPSSGAGTSGESTFPYIPNQSTTDSEITEDGFRWRKYGQKVVKGSSYPRRYYRCISPKCNVWKFVERTTDDPRAFITTYEGKHNHGVPNRRPNSEASKTSSKSSAMKEKL

SEQ ID NO: 164 - protein, 221 aa, Capsicum annuum
FDRDWKKIEDFVGSKTVIQILSHAQKYFLKVQKNGSIAHVPPPRPKRKAAHPYPQKAPKNVLVPLQASMGYPSSMNSLPPGYPSRDDTSVLINSPTGGGFFCTYFNVHITDSVDIGSKGATLIGNSSISGIRGSNRVAPNSELPEQSKLGSVPHGIPDFAEVYNFIGSVFDPDTRGHVQKLKEMDPINFETVLLLMRNLTMNLASPDFEPIVSVLLSLYPE

SEQ ID NO: 165 - protein, 81 aa, Capsicum annuum
MVLTVKIFVITSLEDTCYIEILPGSYKSNKTILWSFQAEVHYNSIYSQADHLVGETRKSTKWWWIATLIFFYTLGVFNLFM

SEQ ID NO: 166 - protein, 750 aa, Capsicum annuum
EPQQRGQLTLSIWTNLLCLRFSLVTKTGIPPLSIPSRLTRPLRKMITIQLXSAPKLVYSYDNVIHGFSAVLSKEELESLENSAGFLSAYKDRTVEAHTTHTSEFLKLNPASGLWPVSGFGQDVIIGVLDSGIWPESASFSDDGLPEIPKRWKGICKPGTDFSSSLCNRKLIGANYFNKGILASDPTVNISMNSARDTRGHGTHVASIAAGNFAKGASYFGYANGTARGMAPRARIAVYKFSFEEGTFTSDLIAAMDQAVADGVDILTISYGWVRIPLYQDAIAIASFGAMMKGVLVSASAGNSGPEMGTLNNGAPWIFTVASCYTDRSFYGTLTLGNGVKITGFSLFPVKTIIKDLPVLYNESISPCDSSNPLSRIPNAGRSIMICYSNAVDIEDQMVAISESKFGGAIYISDDPDALSSNFFPNPGVVISTKEGKQVIDYATKSVRPKASISFQETHIDVKPAPVVSGFSSRGPSRSYLRVAKPDIVAPGVLILAAWPSNVSAAVIGVNTFLDSDYRLESGTSMAAPHIAGIAAMLKGAHPDWSPSAIRSAMMTTANPLDNTEKPIKTTDYLETKDATSLAMGAGLVDPNLSVDPGLIYDATPQDYVNLLCSMNFTQDQFKTIVRSLDKHNCSNPSDDINYPSFIALFSPNGNYTWLEQKFRRTVTNVGAGVAKYVAKVIAPKNSTISISPQTLVFEKKNQKQDYTLTIRYKGIADDQAQSGSITWVEENGHHTVRSPIVVAPEIDAWT

SEQ ID NO: 167 - protein, 485 aa, Capsicum annuum
MDQAVADGVDILQLSYGWGSMPLYEDSVAIASFGAMMKGVLVTASAGNNGPGMGTISNGVPWIFTVASGSIDRSFSGTLTLGNGLKITGFRLFPVRTTIKDFVLVNNGSLSTCDSADGYAQVPNAARSIMICYRTAQEELSVSDQMGVISEAKVGGALYVYGDPDVLSSKFFPNPGVVISSKDWEKEIRIAVKPAPVVSAFSSRGPSLSYLRVAKPDIMAPGELILAAWPSNISATVIGVNTFLNSDYSLLTGTSMAAPHISGIAAMLKGVHPDWSPSAIRSAIVTTANPLDNTGKPVKTTDFLETKVATSLGMGAGLVDPNRAVDPGLIYDATPQDYVNLLCSMNFTEKQFKTIARSSAKHKCSNPSDDINYPSFIALFDPHGDYTWMEQTFRRTVTNVGAGAAKYKAKVEAPKNSTISIYPQILVFEKKNQKEDYTLTIRYKGISEDQAQSGSITWVEKNGYHTVRSPIVVAPALDEALDPSS

SEQ ID NO: 168 - protein, 339 aa, Capsicum annuum
MAVLDMKFLVLVLAILGIWASQTTARTLPEEFMMQKYDQWMIQYGHVYRTIAEKESRFKIFKENVERIEAFNDAANQPYKLGINAFADLTNEEFRAARNGLKIPFCEPKITSFKYENVTAVPTAMDWRTKGAVTPIKDQGQCGNCWAFSAIAATEGITKLSTGKLISLSEQALVDCDRTREDQGCEGGYMEDAFEFIVKNKGITTEKTYPYTAADGSCNSKKESSPAADIKGYEKVPANNEKELLNAVANQPVSVSIDASDFSFQFYSSGVFTGACGTELDHGVTAIGYGKAVDGTKYWLIKNSWGTSWGENGFIRMERGIDAKEGLCGIAMDASYPTV

SEQ ID NO: 169 - protein, 170 aa, Capsicum annuum
MDAATFLKSEQHSSTYGECGVFRNSGTITRKTRGMGAGRKLKSHRRRQRWADKSYKKSHLGNEWKKPFAGSSHAKGIVLEKIGIEAKQPNSAIRKCARVQLIKNGKKIAAFVPNDGCLNYIEENDEVLIAGFGRKGHAVGDIPGVRFKVVKVSGVSLLALFKEKKEKPRS

SEQ ID NO: 170 - protein, 624 aa, Capsicum annuum
MSMQTPKARTASVEVTQRTSPSTAKTARKLRTSGTAEVDSVSFPNPASRTPKDRSPKVIGRRSPQSPAIEQKRPGRMSALEDQLAQLQEEVRAAKEQLSLSESLKETSQQEADEVKKQLAAMSEKLEKTEKQLLERSDSEEARFLELHKISQDKDLEWQSELEVVRKQHELDSAALASSLNEIQKLKLQLDRVADSEAIQVRHAESAHAETESLRVQLKETLTLLEQLQNQLNKSKESEAAVLEEVSKAQLELEVAKMTGNTLRSEGLKAMEACNSLSLELEKSKRQVAALEESVNMLQSDQSSKSVNLVDPAESSAAAQENGINAEADELKTEFSNLKNEVNQLQAALEDSERRYQEACIQSTLQIRSAFEMAECTKSESIRSETEWNLRLNAAKADMEELKEKLMHKEAELRMISDENKGLNVQLEEIQAVDREYGLQDELKRSESILADLRASLSDQETELQRFTEENEMLKLEIKKREIESTKVNDELLDLAEAARAAEREALMKLGYLTEEADKTSRKASRIIEELDAVQTTNSEMEAELRKLKVQSDQWRKAAEAAAAMLSTNNNGRCVERTGSLDYHTIGGKLRSPLSEDLDNDYSSKKKNGTMLKKIGSLLKKGHK

SEQ ID NO: 171 - protein, 210 aa, Capsicum annuum
MELDCKSNHRSGYKFVSHVRSGIRILGLDFTVKNDTRKNPAFRAQFHEMCAKVGVDPLASNKVSGQNCWGLVTSIMNLRRKAVRDTISEDDFLCAISKLKVLGSGFEVITMGKRKLVRSVPTELNKDHNEILELAQAQGFVTVDEVQRRLNWSSGRMTDALETLLEEGLAMIHDGHRDGKRRYWFPCVSSVSTYVGAAAFSWFKYENIWL

SEQ ID NO: 172 - protein, 159 aa, Capsicum annuum
MAMLHMEGDVEAWYFSYTLTRRLVRWEEFCEELVMRFEDDHSKNVEAEFKMLTQQGITNDYLQKFEELRAQVLMRNPRVPEDFILECFIGGLKEEIRETVKMYEPINLSQVVSLSRKQERVINAKEEKTRWLNKGSVAPVGKKLIKGSSIGQKIQATLP

SEQ ID NO: 173 - protein, 239 aa, Capsicum annuum
MEEEAICLNAMSGITELNTFKIKGWINKRTVVLLVDSGSTHNFLYTQTAKELGVIAHVDTPMRVTMANNQQMYSHPSVPRVKWKANGVEFEDKFRLLNLGSCGMVLGVDWMRVHTPVTFDYELYELKIKQGGKVVTLKGNTEPVALHQITAKSMGKILKKGQALLAHLFTIDGDNMSLEEMIPGEVQEVLDDFSHIFDEPTDLPPPRAQDHAIPIIPGARPVSLRSYRYNHYQKNELEK

SEQ ID NO: 174 - protein, 214 aa, Capsicum annuum
MDPTSTKPQGECNVSIKLGMKFDSDEHAYECYNEYATSIGFSVRKEYANKNRVQGYVTSTKFTCYKEGYRNIDKRDSMVKKHRKETRTGCLAHIITSRRSNGVFRITSFEDKHSYPLVDPTLSHLLPSQRNIKVVQAYELDLLDDSGIRPKASFEYAARQAGGQFFLGYTKGDHKNYLRDKRQESLKRGVGRSLVNYFEKKNTRRSCFSIFFAV

SEQ ID NO: 175 - protein, 210 aa, Capsicum annuum
MRRIFLSAWQDMLDKYTLKDNSWLKDIFAMRETWSMTYGRNTFSAVSCSCKIFEFMGILCRYALKILDILNVKDTIPAYYILKRWTKDVTNMHEMDINLVTKDSNPKVEVIARYKHLCQTFIQISSEASGSKKGYELAAIGANELITKLNDIKKRKESVEEPIQDNNIQNEANKIVFVDNSNISKVTGLKRKQPTRRSNTRPKIFLKKAL

SEQ ID NO: 176 - protein, 465 aa, Capsicum annuum
MGKKGNWFSSVKKALSPNSKEKSDKKESKSKKKWFGKERHPVPDSSTLVVASVSPPRPVPPVEDVKLAEVEEEQTKHVYSVAAAAEVVRLTTVNQFTGKSKEEIAAIRIQTTFRGYLARRALRALRGLVRLKTLVDGPSVKRQTANTLKCMQSLSRAQSQISSRRSRLLEENRTLQRQLMQKHAKELESLRRGEEWDDTLQSKEQIEASLLGRYEAAMRRERALAYSYSHQQTWKKSSRSTNLLFMDPTNPQWGWSWLERWMGTRSRENQTMSEKELKSDQMSVRSASISIAGGEITKSFARHQLNSELPSSPSSQKPNRPSSRQSPTTPSKPGTARKLKPASARVSAINQDDDARSVFSVQSEMNRRHSIAGSSVRDDESLGSCSSVPSYMASTQSAKARTRLQSPLGVENSTTPAKGSAGSVKKRLSYSPSPAITRRHSGPPKVEITPTNTAIAEEYVNGVVN

SEQ ID NO: 177 - protein, 343 aa, Capsicum annuum
KSSLHVVEQCAWALGNVAGEGEELRNVLLLQGALPALARMLLPNKKSTVRTAAWALSNLIKGPDPRAATELIKIDGIPDRIVRHLKKGDDELATEVAWVIVYLTALSNVATTILAKSDLVEVLIEILGMSTSLQLLIPVLRSLGNLVAGETHITKFVLWYLLFLYLEIVTLFIRISRIFQEAAWVLSNIAAGSVEHKQLIYSSEAMPFLLDLLSTAAFDIKKEVAYVLGNICVAPAEGSGRPNVILDHLVNLVHGGCITGFLDLVRSADVEAARLGLQFIELVSRGMVVVLRGMPNGEGPKHVEREDGIDAMERYQFHENEELRSMANELVDCYFGEEYGLDE

SEQ ID NO: 178 - protein, 196 aa, Capsicum annuum
MGWTADGLPMGHDVGPAAVVGEPIMRRAQWESGLCSCFGKNDEFASSDFEVCLLGTLAPCVLYGSNAERIGTAPGSFANHCLPYTGLFLIGQSFFGWNCMAPWFSYPSRTALRQRFNLEGNCEAATRSCGCYGGFVEDEERREQCESTCDFATHIVCHPCALCQEGREIRRRLPHPGFSAKPVRFMMAPGDQTMGR

SEQ ID NO: 179 - protein, 716 aa, Capsicum annuum
MTSNLESSPVNIFVWFVLLFCFFSSRFTCGTTDFDFDTLTLSSLKLLGDAHLGNNTIRLTRDLPVPNSGAGKVLYSKPVRFRQPGFDFPAGFSTFFSFSVSNLNPSSIGGGLAFVITPGDELVGDAGGYMGILDAKGTQVGTIAVEFDTLMDVEFKDINGNHVGLDLNSMTSSQVGDLDSIDVDLKSGDLINSWIEYSGSNGQLNIFVSYTNLKPKEPFLSVNINLSEYVNDFMFVGFSGSTQGSTEIHNIVWWSFSSSFDASAKSGVGAAEPXTASLVNQTADSVMLPPPSMAPSESNSSTTGVLQDKSYGKCHSNFCKQGPGAVVGVVTAGAFFLAFATLVLVWLYSKKFKRVKNTEILASDVIKMPKEFSYKELKMATKGFNSTRIIGHGAFGTVYKGILSDTGGIVAVKRCSHNGQGKAEFLSELSIIGTLRHRNLVRLQGWCHEKGEILLVYDLMPNGSLDKALFESRMVLPWSHRRKILIGVASALAYLHQECENQVIHRDIKSSNIMLDEGFNARLGDFGLARQVEHDKSPDATVAAGTMGYLAPEYLLTGRATEKTDVFSYGAVVLEVASGRRPIEKETTRVEKVGVNGNLVEWVWGLHREGNLLNVADSRLCGEFDEPEMRRVLMVGLACSHPDPMSRPTMRGAVQMLEGESEIPIVPRTRPSMTFSTSHLLMTLQDSVSDLNGLITLSPSSSECSYTGGGNGMDLV

SEQ ID NO: 180 - protein, 222 aa, Capsicum annuum
MRNFKYIETIKQALENECPNTVSCADIVLSARDGVVMLGGPHIEMKTGRRGESKESYLAEVDNFIPNHNDSMSLVLSRFKTIGVDAQGTVALLGAHSVGRVHCVNIIHRLYPTVDPTLDPDYATYLKTRCPSPQPSPNVTEFARNDRETPMVWDNLYYKKGLLSVDQQLVSDPITYPFVEKFAASNSYFHAQFGKALIILSENNPLIGDEGEIRKVCHKVNK

SEQ ID NO: 181 - protein, 464 aa, Capsicum annuum
MEVNEAVKIPIARPVASRPRCPVYKSFSELLTGTVDISSTNVHSEMAVTAIRPKTIRLKPATNHALVGEPSSQVGVSKAPVGFGSDNILQSVEKPKVLYKPIGKLAQRKTIPLLENKGSSVSDQQRVIADSEAHVQSANEVKQQHDPTTESKQSLSEKSGQDKKKVRSTIVSGSTEEVAQSLINTSNVDRPSYDGYNWRKYGQKKVKGSEYPRSYYKCTHLKCPVKKKVERSYDGQITEIVYRGEHNHPKPQPPKRNLSDGHRQTAICNDTSKETNNPAWGNQHPQMSEAYVCRIENQNDRGLTIHSSKVPCFYDPIVAAGMHTAVRNSEDSAVGSKKLKATCDEQKSKRRKIKGPSSGAGTSGESTFPYIPNQSTTDSEITEDGFRWRKYGHKVVKGSSYPRSYYRCTSPKCSVRKFVERTTDDPRAFITTYEGKHNHGVPNRRPNSEASKTSSKSSAMKEKL

SEQ ID NO: 182 - protein, 264 aa, Capsicum annuum
MANNISTPTDASSKKVRKPYTITKSRESWTEEEHDKFLEALQLFDRDWKKIEDFVGSKTVIQIRSHAQKYFLKVQKNGSIAHVPPPRPKRKAAHPYPQKAPKNVLVPLQASMGYPSSMNSLPPGYPSWDDTSVLINSPTGGVMSSQDEYHLQRIQADIGSKGATLIGNSSISGIRSSNRAAPNSELPEQSKLGSVTHGIPDFAEVYNFIGSVFDPDTRGHVQKLKEMDPINFETVLLLMRNLTMNLASPDFEPIVSVILSLYPE

SEQ ID NO: 183 - protein, 249 aa, Capsicum annuum
MCSIDNFLDMEAQMLEQQKKKRNTVSCREYYCYKIQMRNDENDELLHTGRAFQQYIVDMFIKLETQRLDFFFFNPELFRMEVLQGLLDVLRHGERDAAKIGKQTFLPITFTGGPRDMRRLYRDAIALVQRFRKPDLFLTMTCNPTWLEIKEHLLPTDETQNRLDLINKIFRAKVEELKSNILKRNVFGKVVAFIYSIEFQKCGLPHAHFLIILANQYKLLTLESYDNLFVLNYLILTKMLTYIQLLYII

SEQ ID NO: 184 - protein, 147 aa, Capsicum annuum
MDFSCKESDMEESLPPGVFEIPGEPAVVINGLPPVSSSADTFIPCPVVTSAESHKNAGFAQWLEGREVRKLFGGQYYYGKVTGFDEEVGWFRVKYEDGDIEDLEWHELEQILRPLDITIPLKTIATKIIKRKQRSIQKSGGRTRNKV

SEQ ID NO: 185 - protein, 201 aa, Capsicum annuum
LVRAAVVTLDSIEIYKTHELFGSKPILFFQCKGENKTILPDVKKKHVLYIFKGEESWQPLTALTYKKCKRCGIYEKDSIKPDDTFDEWELCASDFTSPHGKCSHYIKKEFKATFLCPDCVHRRDASNHASGSEKGGQRMHWAFLLLICALVSIVLVLGAATAYKFWKKKKRQHEQARFLKLFEEDDDIEDELGIGPLSHVI

SEQ ID NO: 186 - protein, 211 aa, Capsicum annuum
MLENYIILVGRLEQSMHIPSEPPEIIQGNRPDPSCPYTGKLEIIDLKKKNHTKFIFLVRYQPKASLVLQGISCIIEVGYKIGIIGRTGSGKKNLISSLFLLVEPIEVRMIIDGLNISTIGILELQSSLSIIPQDPTLFSGTVRYNLDPLSEHTDQDIWEVTFLYKHCQNLSFCSIASFSLLQYFSFFPSKGLFYCSNRSVIITFYQIHQFL

SEQ ID NO: 187 - protein, 215 aa, Capsicum annuum
MYCTKNFQYSQCTVVGIVKLRLYCLSFKDVCEKEEDNEKSSSSPPKERPTRLNLVTTRNRSHSSSPATNHKRYSRIGRYLKDTGNAMSYKSLNELEFEEVKGFMDLGFIFRRGHISKNLVSVLPGLQKLEIVMSKDEDKICEESEAIHIDGIVAEVEEDDKREIVRPYLSEAWLIKRPDSPLLNMKIPRISAASDMKKHLRYWAKTVANVVLQES

SEQ ID NO: 188 - protein, 807 aa, Capsicum annuum
MSNTMTVKAKPTTTEQLAISTIVKNHDQNHNHHDEEGEPNVGLEFDTAEDAQEWYSNYATKKGFRIRIGQLYRSRIDGSVMSRRYVCSKEGFQTSSRTGCPAFIRVQRVDDSGKWVLANVNKEHNHAMENADETCVPKIVQRKVVVDRTVKLKSKSSSGLRSFDDDGPSSRSGVVDFKRVVNQTTKLKSSTGLRSLDEDDGLSGRCGVIDFKRVRRERVNGESRGGEPYKGLEFVTAGEAYEFYHSYAVNTGFRVRIGQLFRSKNDGSITSRRFVCSKEGHQHPSRVGCGAFMRIQRQATGRWLVDRCHNEHNHELGPPGDVSERVSLKGFKEEGSCELENMDLVESNGGLSLVTRSRESRIGSDWYNELFDYFQSRQAEDMGFFYAAEVHKGRAMSVLWADSRSRFACTQFGDAVVFDTTYRRGSCLVPLALFVGVNHHRQPVLLGCALIAEESEESFTWVFQAWLRAMSGRCPISIVADQDRAIQHSVAQVFPGTHHRFSPWQVVAKEQEAIGALRSMNPEFKYEYETCISQSQTPNEFEAAWNVLINKYNLRENTWLKEMYNMRKSWVPLYIRGTFFAGIPIDGSFKSYLGNILTSQAPLCEFIIRYEKSLEQRREEERKEDFNSFNLQPVLHTKDPIEDQGRRLYTITMFKVFQKELLECYSYVAIKINVEGAISRYLVQKCGNGDERNTVAFNASNLNISCSCKMFEFEGVLCRHALKVFQIMNIRELPSRYILHRWTKDAKYGILRDVDSGGTSQDYKALMLWSLREEAKNYIEAGTTSLERYKLAFEIMQEGRRNLCWQN

SEQ ID NO: 189 - protein, 260 aa, Capsicum annuum
MSVIDILTRVDSICKKYDKYDIDKQKDSNVAGDDAFARLYSSVESDIESALQKAETAANEKNRASVVAINAEIRRTKARLLEEVPKLQRLAVRKVKGLSGEELAARNDLVLALPDRINAIPDGSAAPPKQSGGWRASGSRTEIKFDSDGQFDNEYFQQTEESSQFKQEYEMRKMRQACLEVIAEGLDTLKNMASDMNEELDRQVPLMDEIDTKVDRATSDLKNTNVRLKHTVNQLRSSRNFCIDIILLCIILGIAAYLYK

SEQ ID NO: 190 - protein, 480 aa, Capsicum annuum
MMSGDVFSFPSSVKAVLPHHQELQTNPNPKPNPSTKKRRNLPGTPDPDAEVIALSPKSLMATNRFICEICNKGFQRDQNLQLHRRGHNLPWKLKQRNKLEQVKKKVYICPEKTCIHHDPSRALGDLTGIKKHFSRKHGEKKWKCEKCSKKYAVQSDWKAHSKTCGTREYKCDCGTLFSRKDSFITHRAFCDALAEESSRITSVGTNNLINFPNNATLLNQQANLHGGFTGFRPDYGYPQQQKPRLSLWLDQQAPNHNNHLQISSSSNLFDSSNTTGFQEMIQMQSQNHVFGGSNSTTSNFSLSSPVQGNSIADQSLNSLYSDSQNSHQLTKANSTPLPMSATALLQKAAQMGSTKSNPTTTFFTNNFNKLNSSSSSSSPTTPTFIGLQSIDQTTDQLFTQKSEDYTTSLLQCNNGLMQSVGMQNNPAISMNMEQGINGLANCLTRDFLGIKHEGNCQFLPQEIVKFASLSNSAMGLSHLQ

SEQ ID NO: 191 - protein, 475 aa, Capsicum annuum
VLAELVKLAQKRGLKGSKGRWKDFLRSYDKKFGVSLSDPSKRSIDVLLAFLKTFSQNDLKYTYKVFQCHSNQDAVEQLQKSFPDSESAEQGLVYLTLEHPEYPRDYSFPSHEEGWLVTRRSYFDQSKAMVAIDCEMVLCQDGTEALVRICAVDRNLEVKLNEFVNRSKPIADYRTEITGITAGDLDGVSCSLADVQTSMKKLLSHGTILIGHSLHNDLRALKIDHARVIDTSYAFKYQDEPYYKPSVSNLCKSVLRFKLRKMGSPHICLDDASAAMKLVLAKVENGVDNIIPLVHEEVAALLVHRIPVAVHSQELHKVIPGEFTVAVKANRKGQADKYTAYVNFKNQHEANEAFDKLEGNQEKDTWGRPQKLVSFHLDSGISGSLCVRKMVDNTYSKEVTPMKRSFEEDKETVEESKKPRTDYQCTEIKEFSAGCNQCETHSKEIERLKKELSHRDEEISSLNRLIVNLVRKHAL

SEQ ID NO: 192 - protein, 224 aa, Capsicum annuum
DWLVTRRSKKPKHIQSKAMVAIDCEMVLCQDGTEALVRICAVDRNLEVRLNEFVNPNKPIADYRTEITGITAGDLDGVSCSLADVQTSMKKLLSHGTILIGHSLHNDLRALKIDHAKVIDTSYVFKYQDQPSNRRPSLSNLCKSVLGFELRKTGSPHNCLDDACTAMKLVLAKVEGGVDNIIPLVREEVQETETAKLLVHRIPAAVHSQELHKVIPGDFTVEVK

SEQ ID NO: 193 - protein, 191 aa, Capsicum annuum
MQHLDVIFYHLRKKLKLRLCDNYRYTTINCFFKTYIDKTYSRYYPADQTYQISTQEDYEKSYVVAKNEDAITNIINRFCISAGLPWHMIDEVYVPVNRGKEFHWVLVVIVLKERVIRVYDLLSSKIKKKSPNKIQKLAVMLPAYLSDSHFFKKTKRTDWSTLKAYKGKLGQQTDLIIQNLFDVEYVQNIPQ

SEQ ID NO: 194 - protein, 435 aa, Capsicum annuum
MTFPTKSEINTSSLPRKNLCLSVVEPNIKDKASSQELDMILTQYLESLYQRKKYHIGYPTNMSYEHHAILAPLLQFSLNNCGDPFTQSPVDFHSKDFEIAVLDWFAQLWEIEKDEYWGYITNGGTEGNLHGLLVGRELLPGGMIYASADSHCSIFKAARMYRMEIETLNTLVSGEIDYEDLRTKLLLNQNKPAIININIGTTFKGAIDDIDLVVQALEYCGYSNDRYYIHCDAALCGLIVPFIKHAKTITFKKPIGSISISGHKFLGCPIPCGIQITRKSYVNSLSKVEYIASADITISGSRNGLAPIFLWYNLSMKGHAGLKQDSKMCIENAQYLKDRLLKEGISAMLNEFSSTVVFERPCDHKFIRRWQLCYLSGMTHVVVMPGITREIIDSFLNDLMQERKNWYQDETTQPPCLADDIGSQNCLCSYQKMHY

SEQ ID NO: 195 - protein, 106 aa, Capsicum annuum
MELIPCQGFQVYYPNLKVLKLSKANSITALCSHQLPTAYFNKLVTLEVRDCEKLRNLMSPSMARDVLSPQKLRIEKCQSMEEVITEEEQQGEEIMTNKPLFSGKKS

SEQ ID NO: 196 - protein, 123 aa, Capsicum annuum
MRGRIEVERGCFYGWCPKLKSRYRLSKRAKKITLDVIELQNEGNKHDVFCYPVLAVEIEAIPINSVEKFDSRIKKEEEGMAALIDEGVTVIGICGMGGVGKTTLAEKVRARENKQSCLMMLSW

SEQ ID NO: 197 - protein, 244 aa, Capsicum annuum
MVISYIPYHAHPYLLVEVTLFKCGGMAICICVFHKIADEPILYGLAKASATTARGGYNDLPEFIAAAKFLAPPIPYVSNKPTLQSQFANFCHNNQRCVAKLFTFNASTIALLRAKAMSDDVLMPTWVEAVTTLIWKCVILVGVDTGTSKLVNQVNIRRRFVPPLPNHCVGNVVAVATTCKDENDGSDLTTLIYYIRKSLSELSFKYVDKESRDEAIFAIPHDFMELTKGQILGRSLYKLVACVV

SEQ ID NO: 198 - protein, 434 aa, Capsicum annuum
MTKLPLPFLLLLFLVSFTTGEIKKLKISSDPRAMILFERFGFTHTGHAAISVSSVSVISTLASPDPARLGFFLLSEEALLQVLLELQQNPTFCVLDSKFVNLLFTFRELSPPPNSSFNRSYPVTSPNEYSLFFANCAPESKVSMDVRTELYNMDTNQVKDYLSAGLTVLPGLYFLFSFAYIGFLGVWLYVCAKNRKSVHRIHMLMAALVVMKGLNLIFAAEDKHYVKVTGTAHGWDVLFYIFQSIRVVLLFTVIVLIGTGWSFLKPFLQEREKKVLMIVIPLQVLANVASIVIGETGPFIRDWVTWNQVFLIVDIICCCAIIFPIVWSIRSLRETSKTDGKAARNLSKLTLFRQFYIVVIGYLYFTRIVVFALRTIAAYKYQWVANAAEEIASLAFYMVMFYMFRPVEKNEYFVLDEEEEEAAELALRDEEFEL

SEQ ID NO: 199 - protein, 868 aa, Capsicum annuum
MNVVGKVSSFITQGVYSVATPFHPFGGAVDIIVVKQNDGTFKSTPWHVRFGKFQGVLKRAEKVVRIEVNGVGADFHMYLDNSGEAYFIREVTADNEREVNEDSRDSFEKEEVDTSDLDNANNNEGVKEGDLSRNEDNDSNKAVLSSRDEGVTLDRGGSDDDRRCYEFLDKQTSLEDLVLEEFGSSRHENSDNVEEVLESLDSSSEVVMVSVDGHILKAPILSSERNVEDVELDTPRFHLGPGQGTDFSDGNSEFISGEATWADEYSSYQASPKVAPADACNVKKESSKVEHQRDFSKVDGQFPENLDGKNQENVNVEVTSCGIKKDEVSNSCLELPASGTHDGNEINQPDVVSPVKIREVVDDLEKKPPGKLGDNSSLNPIKSLQQGESVMDSNGFDGSRNHHIVSGKHPGEKNDVSLASGEIKSCHQESCECGQCKNVRSLIEASLKVIVTGVEISLCGNLLHAGMGSSSAREAFDANRISEEEFRSSSESIIKNENLVVRIRGNYIFWDRAAPLILGMSECDMELPVEYADVIPVEPKETSKSGQDDSGISSVPSGRRWTLWPNPFRRVKTLDNSNCNSSNEEVIVDSESNSICQPMEQTAITKGGKETPQVQLVRTNNPTSDQITSLNLKEGQNMVTFIFSTRVLGEQKVESHIYLWKWNSKIVISDVDGTITKSDVLGQFMPLVGKDWTHFGTARLFSAIKENGYQLLFLSARAIVQAYLTKNFLFNLKQDGKTLPTGPVVISPDGLFPALYREVIRRAPHEFKIACLEDIKALFPQDHNPFYAGFGNRDTDEFSYRKIGIPKGKIFIINPKGEVSINNQIDVKSYTSLHSLVDDMFPPTSMAEQEDFNIWNYWKMPISDINYL

SEQ ID NO: 200 - protein, 680 aa, Capsicum annuum
MNKPSSLLCQQKNFTLSLLKSPHKISTLNHLKSLHLHLLRTGLNHSNYAIGNFITRCACLGLMSYAHQLFDKMLQPNSFVWNTLIRGYQQNQLPKHTLYCFDQMRVNNVQPDRFTYPFVIRACSDLHEFEKGKSFHGQVFKIGVNLDVFVGTKLIEFYTTMGDLNMTKRVFEGMKDKDEVTWYTMLSSYVNIFNDMGKARDLFEKIPCKDVVIWHTMILGCVKAGELEAAKEYFDRAPVKDLLMYNTILGCLAKNGEVECLLRFFREMTCRDLVSWNTVIGGVVRDGRINEAMRLFYEMQRVNLSPNDVTLASLLSACAQAGALDTGKRLHSYIDRRGSELNAVIGTALLDMYCKCGDLETAADVFNKMSERDVVAWSAMIMGSSMNGQSRTALNFFYRMKDESERPNDATILGVLCACVHAGLVDEGRMCFYGMSEEFGLTPKLEHYGCMVDLLGRAGLLDDAYNLIQSMPYEPHTGSWGALLGACKIHGNVELAEKAIEHLIQLDLDDGGYLAIMSNIYANAGRWEDVSKIRKLMKEKGIGKSRGISCIEVNGVIHEFGVQEKKHTQAREIYDMIDEIYRRLKRAGHVASTREVFFDVEEEEKEKALFFHSEKMAVAFGLIATDKTAIIRVVKNLRICPDCHAAMKLISASFEREIVIRDRHRFHHFKNGFCSCRDYW

SEQ ID NO: 201 - protein, 601 aa, Capsicum annuum
MDLEMGLPPAPHPISNMNYPGDPSPSPSPRAPALVVSNSSKALMPSNSSKALFVSNSGKALLLSNSGKRIDPTGKKKYVKQVTGRHNDTELHLAAQRGDVGAVREILGEIDSQMLKTMSGAEFDAEMAEIREAMVNEVNELGETALFTAAEKGHIDVVKELLPYSTKEGITTKNRSGLDPLHIAANQGHQDIVKLLLEHEPELSKTLGQSNATPLVSAATKGHTAVVHELLSKDSSLLEISRSNGKNALHLSARQGHVDIVRALLEKDPQLARRTDKKGQTALHMAVKGISSEVVKLLLQADPAIVMLPDKFGNTALHIATRKKRSEVVHELLLIDDTNVNALTREHKTALDIAEGLPMSEESTELKESLMRYGACRANELNQPRDELRKTVTEIKKNVHSQLEQARRTNKNMTGIAKELEKLHREGVNTATNSVTIVASLFATVAFAAIFTVPGGDEDSGYAVASNTPAFKIFYFTNALALFTALAVVVVQITVVRGETKTEKRVVSVINKLMWLAAVFTNLAFVSASYVVIGRRNLWAAIFTTVVSGIIMGGVLGAMTYYVIKSKRNHKMRKRQKHTRTRTSSFQTNLSDSDYNPMFAL

SEQ ID NO: 202 - protein, 286 aa, Capsicum annuum
NDGPDKGSVVNEAPWILTVGASTTDRKIKAVAVLGNGAEFEGESAFQPTNFSRKLLPIVNGNACELLNTSDVKGKIVLCKTHGYRSGKDKGVVVKMAGGVGMILMTEKNLGSTTLSENDVLPTTNVDYNDEQMIINYTKSTSTRVATISFKGTRTGNKHAQTVAYFSSRGLSMQSRGILKPDVIGPGVNILAAWSASVAAAITSSTAASSTFFFQSGAVLQSMSSPHLAGVAALLKSAHPEWSPAAIKSTIMTTADYINLGNDLIQDETLKHADLLTLGSGHVNPS

SEQ ID NO: 203 - protein, 142 aa, Capsicum annuum
MHTTHCPSFLGLHQNVGLWNASNSGKGAIIGVFDTGIAPQHTSFNDNGMPSPPVKWKGKCEFNVTTYCNKKLIGAMNFAQLDKSQWDKDGHGTHTSSIATGNFVDGANFLGNANGTAVGIAPRAHVQSLLFLLSRNRCHSRS

SEQ ID NO: 204 - protein, 128 aa, Capsicum annuum
MIINYTKSTSTPVATILFKGMRTGNKNAPTVAYFSSRGPSMQSRGILKPDIIGPGVNILAAWTTPVGGAITSATAVSLTFNILSGTSMSCPHLAGVAALLKSAHPNWSPAAIKSAIMTTADYINPGII

SEQ ID NO: 205 - protein, 148 aa, Capsicum annuum
MYKVCYSYCLEADVVAGLDAAIDDGVDVISMSFGLVVSPPLYDDYVAVGAYSAMKKGIFVSCSAGNGGPGNGSVINGAPWILTIGASTTDRKISAVPVLDNGAEFEGESAFQSTNFSRKLLPIVKGNACESLNTSDVKSKIVLCDTSG

SEQ ID NO: 206 - protein, 771 aa, Capsicum annuum
MSLLTFSWYLLCLSLFLGTSAERSTYIVHLDKSFMPTIFASHHNWHSSIIDTIKIETPTTQNGHHPVPKLLYSYDNVLHGFSAVLSKNEFEALKKSPGFLSAYKDRTVEAHTTHTPEFLKLNPTSGLWPASGFGQDVIIGVLDSGVWPESASFRDDGLSAIPKKWKGICKPGIAFNSSLCNRKLIGANYFNKGLLASDPTIILSMNSARDMRGHGTHVASIAAGSFVEGVSYFGYAPGTARGIAPRARIAVYKFSFEEGTVTSDLIAAMDQAVADGVDILQLSYGWGSMPLYEDSVAIASFGAMMKGVLVTASAGNNGPGMGTISNGVPWIFTVASGSIDRSFSGTLTLGNGLKITGFSLFPVRTTIKDFVLVNNGSLSTCDSADDYAQVPNAARSIMICYSTAQEDLSVSDQMGVISEAKVGGALYVYGDPDVLSSKFFPNPGVVISSKDWEKVTKYADNSAKPKVSISFQETSIAVKPAPVVSAFSSRGPSLSYLSVAKPDIMAPGELILAAWPSNVSAAVIGVNTFLDSDYSLLTGTSMAAPHISGIAAMLKGVHPDWSPSAIRSAIMTTANPLDNTGKPIKTTDFLETKVATSLAMGAGLVDPNRAVDPGLIYDATPQDYVNLLCSMNFTEKQFKTIARSSAKRKCSNPSDDINYPSFIALFDPYGDYTWMEQTFRRTVTNVGAGAAKYKAKVKAPKNSTISISPQILVFEKKNQKQDYTLTIRYKGIAEDQAQSGSITWVEKNGHHTVRSPIVVAPALDEALDPSS

SEQ ID NO: 207 - protein, 285 aa, Capsicum annuum
MAKDIEYGNDQYAPNKDYQDPPPAPLIDPEELGKWSFYRAIIAEFIATLLFLYITVLTVIGYKSQSDTKHNGDQCGGVGILGIAWAFGGMIFILVYCTAGISGGHINPAVTFGLFLARKVSLVRAVMYIVAQCLGAICGCGLVKSFQKAYYVKYGGGANELADGYNTATGLGAEIIGTFVLVYTVFSATDPKRNARDSHVPVLAPLPIGFAVFMVHLATIPITGTGINPARSFGAAVIYGKEKSWDDQWIFWVGPFIGAAIAALYHQFILRAGAAKALGSHRSNA

SEQ ID NO: 208 - protein, 378 aa, Capsicum annuum
MAYILFLKIIVIIVETGGERIQKFSVFSYKELKAATDGFRASSRIGEGGFGSVYKGRLEDGSFVAVKVLSVELESTRGEREFISEIASLSNIRHQNLVTLRGYCVDGNKRLLVYGYMQNNCLSQTLLGEEQNRRKFTWELRREISKGIANGLSYLHEEVNPHVVHRDIKASNILLDQNFTPKIADFGLSRLFSKNISHITTRVAGTLGYLSPEYAISGHLTRKSDVYSFGVLLLEIVSGCPVIAFDIERGEHFLVNKAWEIYNTGRLLELVDPMLNGEFHGDEAVRFLKIGLLCVQEIASLRPKMSTVIKMLSNANYMELEDINITQPGILADLGDVKIGQKQSTHSFLSSAVDIARKCWYKVYVSHTQSWPILFDKI

SEQ ID NO: 209 - protein, 316 aa, Capsicum annuum
MGNAGSSXXXXXXXLPPPPPPEINLNRYLFAAATPYPSRFPHGPPYYQYAGYFPPPPAPAMRRRYVKYQKAVMIRNDVNVKKESLRVEHDEQNPGKYLVAFTFDATVSGFLILLIVIFFGKEGEDCSLTPMKESLLPSITVEFQQDLDQKFRQPSGTGIDLSMFDEAESFKDGDADLYPLVVKAEAFPGGLSVLEDGKAASVSTTSQITQAVFEKYKGEYHVRVVKQILWINGMRYELQEIYGIDNSIDNNLDENDPGKECVICLSEPRDTTVLPCRHRCMCSGCAKVLRFQTYRCPICRHPVERLLEIKVNEGAE

SEQ ID NO: 210 - protein, 290 aa, Capsicum annuum
MTYNLVLSFLFLFLFLLQFTCILAQAPAMAPMPPTQSLAPASLPPAQPSAPASPPPTISSPPAPISSPIVASAPGPPGPPNVIKILAKAGSCSTFIKLLQITQEVSEITSLLNNSNRITMFVPSDGAFLNLKTGTLNSFSDQRKAELVKFHVLTRYFSLLQFQTASNPLQTQAGGTTNREFPINITTNGTAVDIATGIVNASVTSTIFNDNQLAIYQVDKVLLPLQFFVPPLPPAPAPAPPKPKKDVPSTDSSPVNVVPSDATSLLPHILQGIVYPGSFFFFSLCFEYMS

SEQ ID NO: 211 - protein, 264 aa, Capsicum annuum
MAPMPPTQSSAPASLPPSQPSAPASPPPTISSSPAPILSPVVASAPAPGPPGLPNVIKILAKAGSCSTFIKLLQITHEVGEITSLLNNSNSITMFVPSDGAFLNLKTGTLNSFSDQRKAELVKFHILPSYFSLSQFQTASNPVQTQAGGTTNREFPINIATNGTAVNITTGIVNASLTSTIYTDSQLAIYQVDKVLLPLKFFVKPLPPAPAPEPSKPKKDDPSSDSSSVNVVPSGATSLLPHTFQGMVYPGCFFFFSVCFVYMS

SEQ ID NO: 212 - protein, 292 aa, Capsicum annuum
MDYNLASPFLVLILFLLQFTCILAQAPAMAPMPPTQSSAPASLPLAQSSAPASPPPTISSPPAPILSPIVVSAPAPGPSGPPNVIKILGKAGSCSTFIKLLQITHEVGEITSYLNKSNSLTMFVPPDGAFLNLKTGTLNSFSDQRKADLVKFHILPSYFSLSQFQTASNPVPTQAGGTTNREFPINITTNGTAVNITTGIVNASLTSTIYTDNQLAIYQVDKVLLPLKFFVPPLPPAPAPEPSKPQKDDPSSDSSSVNVVPSGATSLLPHMFRGMVYPGSFFFFSVFSVYMS

SEQ ID NO: 213 - protein, 606 aa, Capsicum annuum
MSTPKFYQTAKHLFPKKTNQEIAKLVCSTLINLNSTNKKIPPSLISYINPQITHLVLSNPLVPTQSCLTFFKFLQENQCINHQKPNLESYIVLFLRLFTARKFAESKNVLNNVVANDALRRPFLEIVSFENHVEHKVMGKILDMLFRVYVDNMRFKEGLEVFEYMRNEGFVIDYRSCMVYLLAMKRGKEYELLIEFFDKMVEFDVRITVYSMTMVVDALCKVGEVRKARKVVDEMVSKGVKPNVYTYNTLLDAFMKKQDFVGVKEVLMTMEKEGFDLNVTSYTLLIDGYCNFGNVKEVERLFREIEGKGVEADVHLYTSMISGCSRLCNAKRAFSLFDEMVDKGLVPNAHTYGALINCLCKAGQMKAAEVLLNEMQSKGIDINQVVFNTMMDGYCKQGNVDEAWRMQKIMEGKGYESNVYVYNIIATGLCKLDRREEAKTWLFSMVDRGVAPNEVAYTTLIGIYTKEGNFVEAKRILREMETKGVKPNTTTYNTLIDGYCKKGMMKEAYKLKTVMECKGLIPDVYTYTSLVHGECISGKVDDALKLFNEMPREGVVPNVVTYTAMISGLSKEGRSDEAFRLYDEMIEAGLKPDASTYSALVGSLHG

SEQ ID NO: 214 - protein, 171 aa, Capsicum annuum
MDCLRFIYRGYMEPEYLACGQLTKKEDVYSFGVLRLEIVTGRQSNQRNNVVYTVNLVSTVTWEHFQRGTVEKIFEPNLMLQNSHTINMKNEVTKLLHIGLLCTQEIPMLRPSMSKALQMLVKKKEELPSPTTPPFIDEKTMELYDLWEKYSLKLGNSSSIAMLSHSSFNPR

SEQ ID NO: 215 - protein, 72 aa, Capsicum annuum
MLGCLPWLKGRALYIRCFMRYFDTNFLNAILTSGGSSSRGTSRKVVVIVIVVSFVIVLGVGAFIGIGVWKNK

SEQ ID NO: 216 - protein, 237 aa, Capsicum annuum
MQRCRTSHQIAQVQHIKTIGVGKHGTRYKVFWDLKQDGKYSNKSTWKLIRERKPTDHNIAQIQRNMYGITLEGHWESEFRHTIRHTIYNRCNQNSNNSIHKLVLQTMPGLIYWEIWKQRCECNHGGKMNFNRYMIIQHIGWNIQNTITKAYAEVEKLVKLKPVPNLCIVCWNFYENGVLKINNDGSCYPASGKSGMGGFLRDDKGNLILAFSSLSSCCSSTQAEASTASYGLSLCLL

SEQ ID NO: 217 - protein, 487 aa, Capsicum annuum
MITCSNFLRRCIVEYKVKFTVYSMTLVIDGLCKMGEFCKAKKFMDENISKPNDLTYKKILNIYINKKPDFVIVKEILRTMEKEGWGFDVKSYTQLIKWYSTFGEIEEMKSLFMEIEEKGIKPDVYLYTCMVSGYCKMGDLVKACKVFDEMSDRGLVPDGYIYGALINGLCKNGLMEAAEVRFNEMRSKGVWVNRCVFNAMMDGYCKLGNVEEALRVQRVMEREGYEPDANVYNIIATGLRKLDKHDKAKRLLLLMLDRGVVPNALAYTTLIDIYCKQGDFVEAKVIFSEMESKGIKPNTGTYNALIDGYFKHGNFIDAKRALREMETKGVKPNTKTYTVLIDGYCKQGDFVKAKRTLIEMESNGVKPNTNTYTVFIDSYCKKGMMNEAYKLIMIMESKDLKPNVYTYTSLVHGECKLGKVDNALKLFNVMSTKGLVPNVVTYTAVISGLSQEGRLDEALRLYNQMIEAGVIPDAAAYSALSVRNSSL

SEQ ID NO: 218 - protein, 538 aa, Capsicum annuum
MLVGPNLHQDMLVILDKCRSSTQLKQLQGHLITTGHGQTQLYAFKLVRFCTISLSDLNYARLIFNYINFPNVYLYTAMITAYTSLPDHKSSLLLYREMVRSGLSKPNHFVFPIILKSFPQVTKHCGIEMAHAHIERMGFGKYPVVQTALLDAYSRFTSDVRVARQLFDEISDKNVVSWTAMISGYTRVGRMGDAVLLFEEVPQHIRDTPSWNSIIAGCTQNGLFSEAISLFRRMIVEEGMVQGNKPNDITFACVLAACGHTGMLQLGKCIHGYTYRNSLHLNTLTLNALIDMYGKCGSLKQARNLFDKANRRRGSLACWNSMINCLALQGHWKGAIGVFKDMLQHGDDVKPDTVTFIGLLSACTHGGLVEEGLSYYDLMTRVYGINPEIEHYGCLIDLLGRAGRFEEIMKIVRDMHLTPDAVIWGSLLNGCKIHGRIDLAEYALEKLISIDPNNGSYYSVLANLYGELGKWDEARNIRKILNQQNAYKAPGCSWIELHSQVHQFYSVEKSHPTMEEINSTLESLSTVLSTSQIYYEII

SEQ ID NO: 219 - protein, 119 aa, Capsicum annuum
MESGELSCDQSEVVISYLMNGRGEIHPTFIRNDRHVELYILCVDFDNSKPILRVKVVERSREEASTSAPTPPSPSPPPSPLTVDNTSMEYDCMGGDEWDNTEDYEEEKCGGDEDGQLEP

SEQ ID NO: 220 - protein, 185 aa, Capsicum annuum
MRLLLIRKERRKNQKKMSRESCLLLDPLVEKQGWNTLLKGNKLGDRGPYMITNRSIIMTKRATDFDFNAEILQTISIWVRLSNLPLNCWGMNSLSRIGSGLGVPLFAADCTTSVEKIFYARIFLDMNVTQPLSTTIKVVDPTGRSFNQKILYDLVPKYYPTCMQVGHTCKTKPPNPTGPLVVEQP

SEQ ID NO: 221 - protein, 533 aa, Capsicum annuum
MAENGGVGSPGSSMHGITGKEQTFTFTVTSDPKIPTDTTAKFALPIDSEHKSKVFKIFSFAKPHMTTFHLSWTSFFTCFISTFAAAPLVPIIRDNLNLTKSDIGNAGIASVSGSIFSRLVMGAVCDLLGPRYGCAFLMMLTAPTVFCMSFVSSAGGYVAVRFMIGFSLATFVSCQYWMSTMFNSKIIGVVNGTTAGWGNMGGGVTQLLMPSLYELIRRAGATPFTAWRITFFVPGWLHVIMGLLVLGLGQDLPDGNRGALQKKGDVAKDKFSKILWYAVTNYRTWIFVLLYGMSLGVELTTDNVIAEYFFDRFNLKLRTAGTIAATFGMANIFARPLGGLTSDILAKRFGMRGRLWNLWILQTFGGIFCIVLGKSEKLEISILSMIFFSIGAQAAAGATFGIIPFITRRSLGVISGLTGAGGNFGSGLTQLLFFTNASYSTSTGLLYMGVMIVVCTLPVMLVHFPQWGSMFLPPSKNVDKFNEEYYYGSEWNDEEKLKGLHEGSLKFAENSRSERGRQVVSVTTPPDATPPVV

SEQ ID NO: 222 - protein, 809 aa, Capsicum annuum
MTNKAESSDSKGTKRDFSTAILERKKSPNRLVVDEATNDDNSVVALHPDTMEKLQLFRGDTILIKGKKRKDTICIALADDTCDVPKIRMNKVVRNNLRVRLGDVVSVHQCPDVKYGKRVHILPIDDTIEGVTGNLFDAYLKPYFLEAYRPVRKGDLFLARGGMRSVEFKIIETDPPEYCVVAPDTEIFCEGEPVSREDENRLDEVGYDDVGGVRKQMAQIRELVELPLRHPQLFKSIGVKPPKGILLYGPPGSGKTLIARAVANETGAFFFCINGPEIMSKLAGESESNLRKAFEEAEKNAPSIIFIDEIDSIAPKREKTNGEVERRIVSQLLTLMDGLKSRAHVIVMGATNRPNSIDPALRRFGRFDREIDIGVPDEVGRLEVLRIHTKNMKLAEEVDLERICKDTHGYVGADLAALCTEAALQCIREKMDVIDLEDETIDAEILNSMAVTNEHFQTALGTSNPSALRETVVEVPNVSWEDIGGLENVKRELQETVQYPVEHPEKFEKFGMSPSKGVLFYGPPGCGKTLLAKAIANECQANFISIKGPELLTMWFGESEANVREIFDKARQSAPCVLFFDELDSIATQRGSSSGDAGGAGDRVLNQLLTEMDGMNAKKTVFIIGATNRPDIIDPALLRPGRLDQLIYIPLPDEDSRNQIFKACLRKSPLSKDIDLRALAKFTQGFSGADITEICQRACKYAIRENIEKDIERERRRSENPEAMEEDIEDEVPEITPSHFEESMKYARRSVSDADIRKYQAFAQTLQQSRGFGSEFRFSETSTAGTTGTAAADPFATSTGAADDDDLYS

SEQ ID NO: 223 - protein, 136 aa, Capsicum annuum
MSLNTFTIKKQQLALSQHIRRKRMGKNSQKRKVICGDYFPKVKKIMKLFENMFLHNISNWKKWKAQVTTSTKKGHFSVLALGENAEPRKFSVALDYLSHPTFIKLLEEAEREFGYCHQGVIVLPCDAREIQRLLLR

SEQ ID NO: 224 - protein, 218 aa, Capsicum annuum
MLAYFGSEFSAGSALVYFIRPSCWFSSPLANVVNYSISHLHRTEEEQVVQCLRMLTEKSTTIVGVGVAEGKVGVNKPELLPKEFTPVIDVAGFLSDGQEKRLAQEITDIEKDTGFKLRVLAQNYPDTPGLAIKDFWQVDDRTIVFVADPTFGNILNFNVGVTVDLDIPRSFWSRLAGTYGNMFYWKEKGEDASIEAAVMAISSCLREPVGPNNCSEIK

SEQ ID NO: 225 - protein, 128 aa, Capsicum annuum
MFKIIGGERIKGLIILKLLIKKLMNFHFSPRYDDENGTQFVATSIEDDEEVVPCDVKEGHFAIFAVNTKDEERKRFILELNWLNNPALLRLLNLAEEEYGFTQRGVLKIPCPHEELEKIILQLKIERN

SEQ ID NO: 226 - protein, 104 aa, Capsicum annuum
MGKIKNNLLQLVPKLDDVQIVETPRAKDEDIPNDVKEGYFAVFSVNAEEQPTRFIVELHWLTDPSFLKLMKQTEDEYGFGQKGVLEVYCLAKELQKILEIKFAV

SEQ ID NO: 227 - protein, 101 aa, Capsicum annuum
MGKLKNNLVQLIIPQVEYVETQRANEEVLPNDVKEGHFAVFSVNPEEEPKRFIVELHWLTDPSFLKLLKQAEDEYGFGQKGVLEIPCRAEELQKILELKIG

SEQ ID NO: 228 - protein, 86 aa, Capsicum annuum
MPDELFALNENSTWDFIDKPTDGVLIGNKWVYSIKMKADGTIDLYKSRLVSQGYKQQYEVDYEKTFAPVAKMTTVRTLLAFALIKG

SEQ ID NO: 229 - protein, 136 aa, Capsicum annuum
MGKERKSNCKGIMINHLILKLKSITHLQLIPKYSNRYQFDVVESVDTPRANEEVVPNDVKEGYFAVFSVNPKEEPKRFIVELHWLTDPSFLKLMKQTEDEYGFEPKGVLEVPCLAEELQKILELNIGSNVSANFVV

SEQ ID NO: 230 - protein, 112 aa, Capsicum annuum
MVMFKVIMRKLKSHIQLIPKYTNRHQLGVVETPRANEEVLPNDVKEGYFAVCSVSPEEEAKRFIVELHWLNDPSFLQLLKQAEDEYGFQQKGVLEVPCRAAELQKILELNIE

SEQ ID NO: 231 - protein, 150 aa, Capsicum annuum
MKAVDTMKSQVQIPSEEQKYYLVISFYLSKLVDVAGTTSPKHIAKMLKKILQFTRRSSNFRHAIDDWDEDASSLPPPENVKEGHFVVHAIDDGKLRRFVVKLSYLADPGFMRLLEQAEEEFGFKQEGVLAVPCRHSEMEKILESRKKNKK

SEQ ID NO: 232 - protein, 89 aa, Capsicum annuum
MCHYLGVEKENLCLYGQPNETWEVNLPAEEVPPELPEPALGINFARDGMNRRDWFSLVAVHSDCWLLSVSFYFGARLNRNERYSFLIIN

SEQ ID NO: 233 - protein, 216 aa, Capsicum annuum
MQPCTGYRRKRSLCSLLMDSFKSGATWSSEKCLHLMYAYFCPILGGRISSVRVSSHYSEHKGINEYSFLYFLVVNMAHSLNLYTLYGKKQGKLKVSLRSQRFRESSYMDPNQSLCLGALFDIAATNGLDMGRKLCIIGFCRSIEMLSDVVEDTVLEHGGEIVSAEKASKDNLQEKLTMTVAVPLLWGVPPASETIHLAVRNGGGIVDKVYWRWDFL

SEQ ID NO: 234 - protein, 1475 aa, Capsicum annuum
MSLNTSHLFEELQCVAKKWASQENARLIQNLRQIEREYSSVCKVAEKDLSWLNHYLDELQRVEEKYGSFHVFSDNNHLLQTANRIQQGCYSFRIFVEGPNVTSSLYAIEDKCKSFYTVLNEYNFIEEYHLLGTLKRIEEDLGSARVFSDQYLSKSSHLLQTLQQTEAECYQSCEFLAELQSKKSDLLQSLQHIEQVWGSFRKYSDELQSKISNFLETTARFVEECGSFLVFLETYLSAEDSVQRLEYGWAFPVDQFMVLKRDFKFLGIILNLQNLKDEPNFLKQARDSIQRTERHIVRIGQENRISEMRYIQRVFHATKSEIRIRYSFPEMYFQLFTNMRGIVIPHFVMEFIDTVVENLSDLLKYNDPSSLLCIGEAMMGQIEKALEGLKFLRSFLCFVSDRCIEPQRQYTFLNHILQVAWLTTMFAWLYLPSTQGYADYPPDEINYLFSKLRREMIQPIDPSTCKIYIGVLQALKLVQSQWYPVIQIKYIVDCEVGFVDTLLHSLKELPVPSNFTAIKKTLQEMLNFLKTNLINLPVRALVFHLQYINSVIVDVGLLVYSLNYKENDKEDETLGEAKNDKEDETLGEANQVLVLDFPGRIQSMQAMIYLISRKLFLFQYGIDEQGTADFTVDILMDLLCHYSNSISSIKSQLQKTQKELMCFQKVVEQDDRCRQFATPVIHLERKLEYVTSSSIEESFPDWCLFLWILHIGADIRLLKAEVAEILEKNVLDLVLHNTKDADIAHSSSQHSRNPAKNEEMVGHKDVMDELRGKLTKGSSGFDVISIVGMPGVGKTTLANNLYFDQSVVSHFDRRAQCCVSQEYSRKGLLLSLLGDITENTTKLEGESEDVLADKLRKLLMFKRYLIFIDDIWEASVWDYLNLCFRNANNGSRIILTTRHYDVASYAKHVSDPLVLRLLSNEESWILLQNKVFNTETCPLALEDVGKSIAQKCGGLPLSIVLVAGILAKMEKERHRWEQVTTKLSQHIQDLSEHTIDLSYQDLPHHLRTCFLYFAVFSEDEEIQVSKLTWLWISEGFVKTHMEKLLEDIAEDYMDNLVERNLVMVSKRSFDGKIKACHIHDLLLEFCRKKAELNNFMQRIKGYPDFTNFCTGRNEVFLPFHVFLPKCKTPRRLSLQSQCNNVEKWCFWFSHVKSFQFREARKIAFSLIDHASYIFNSFKFLRVLDLEFTIIDFLPQELTLLRYLAFRTAKDMLLLPANLCNLETLIVQGMNGRVSLPDTIWKMVKLRHLHIYNQAFFTLNSTQEISESTSKMDDLQTLASAYFSCMDNADKILAKMPNLRKLRCEVLKFTGSFTAFMNLAKLKMLKISSGPTFTLINQLKIPSHLKKLTLTNFRINLNEVTTLSNLQVLKLLGVTISSNTWVVNDGQFGKLKCLKLENLSFSEWDVSDDAFPCLEHLVLKRCRYLEGIPSCFGYMSSLKSIEVKSCKGSLAESATVIKEMQVEEMGYSAFEVFIHK

SEQ ID NO: 235 - protein, 310 aa, Capsicum annuum
MKVPTIDFRNPELKPSTKQWDFTKSQVTQALQEYGCFEAIYDKLPDDTREAMLGTLKEIFEFPLETKMKNVSKKSFNGYLGQLPTLPLYESVCIDNLLQPGSVETFANIFWPDGNPDFCNVVKSYSKPLVELDEMVKRMVLENLGLKDYVDEFLDLSSFLLRLTNYKAAEGEDGNKPGIGDHTDGGFLTIISQNEVNGLQILKKNGEWIDVNISSNSFVVLSGDSFMAWTNGRLHSPLHRVTLSGDKDRYSIQLFADPKPDCIIEAPKELVDEEHPLLFKPYDFLGYFEFFGTKAGREAGANVLRAYCGV

SEQ ID NO: 236 - protein, 390 aa, Capsicum annuum
METFLFTSESVNEGHPDKLCDQVSDAILDACLEQDPESKVACETCTKTNMVMVFGEITTKATVDYEKIVRDTCRGIGFTSADVGLDADHCKVLVNIEQQSPDIAQGVHGHLTKKPEEIGAGDQGHMFGYATDETPELMPLTHVLATKLGAKLTEVRKNKTCPWLRPDGKTQVTVEYKNDNGAMVPIRVHTVLISTQHDETVTNDQIAQDLKEHVIKPVIPAKYLDDNTIFHLNPSGRFVIGGPHGDAGLTGRKIIIDTYGGWGAHGGGAFSGKDPTKVDRSGAYIVRQAAKSVVAAGLARRCIVQVSYAIGVAEPLSVFVDTYKTGTIPDKDILALIKENFDFRPGMMSINLDLKRGGNFRYQKTAAYGHFGRDDPDFTWETVKPLKPKA

SEQ ID NO: 237 - protein, 134 aa, Capsicum annuum
MALLPEGITELLHHEQLSVPMIKCGKVIILTATNVTELDLQWNCLIDSLKSNGDLVVMTPFVSKCLATTLSDVEVARPLSKLCLQFPDLYVGGYRGSRKGPLMIRFEGKDLSRIEAACQSLCQNFYPGAFSETE

SEQ ID NO: 238 - protein, 341 aa, Capsicum annuum
VSHPLPFCLNRGPLNSIQEVALSFNGGKDSAVLLHLLKAGYFLHKAEESSSQDVADGENTLPIRTIYFETPNAFPEINSFTYETAATYNLQMDILRLDFKSGLEALLKANPIRAIFLGVRIGDPTAVGQEQFSPSSPGWPPFMRVNPILDWSYRDVWAFLLVCKVRYCSLYDQGYTSIGSIHDTVPNALLGIRDDNNSKKKFRPAYLLADGRLERAGRVKKYTLPVSGKLSSVSNGLKIEDLNSNNMLNASIIAVGDEILFGTVEDKLGSVLCRKLHSIGWAASLLVVARNDIDSVAEEVERRKSTSDMVFIYGGVGPLHSDVTVAGVAKAFGVRMVCNIH

SEQ ID NO: 239 - protein, 63 aa, Capsicum annuum
MISSGDAQTLINHFMHLQSEDSNFFYAFQVDEDGRLCNFFWRDSISKWHYECFGDVIVQIDMT

SEQ ID NO: 240 - protein, 569 aa, Capsicum annuum
MEGFGLHMLVGRWFMVFASLLILSVAGGTYIFGLYSEEVKIALGYDQTTLNLLGFFKDLGANVGIISGLINEVTPPWVVLLLGAFMNFSGYFSIWLAVTGRLSKPKVWQMCFCIFIGANSQTFANTGVIVTCVKNFPQSRGIVIGLLKGFVGLSGAILTQLYHAFYGSNGKSLILLIAWLPAVVSCVFLRVIRTMQVTRQENELKIFYQLLFMSLGLAGFLMAIIIVQNRIHFTSVGYWSTAGTILVLLFAPIILVVREELRLWGTKKQDLSQDVEIKVAEIETPLQSSATVVPINDQKEPVSFFQNVFKPPERGEDYTILQAVLSIDMLILFIATIFGAGGLLTAMDNLGQIGKALGYPKTSITTFVSLVSIWGYLGRVGSGFASEIFLEKYKFPRPLMLTIVLFLSCAGHVLVAFGVPNTLYIASVLMGLCFGALWPLIFAIISEIFGLKHYSTLLNFGGAASPIGAYIFNVKVAGNLYDREALKQMAAHGFIRKPGEDLHCTGVECYKLSFLIIAASTFVGCIVSLVLVVRTFKFYKGDIYKKFREQAKALDAAESQSRTNGDAPS

SEQ ID NO: 241 - protein, 204 aa, Capsicum annuum
MVGFSIHLLMSRWFMVFACLLIMSMAGATYLFSLYSNEIKSSLGYDQTTLNLLSFFKDLGGNVGMISGFINEVTPPWVVLFMGAIMNLFGYFMRWFTVVGRIAKPPVWQMCFYICIGASSQTFGNTGVLVTCVKNFPESRGIVLGLLKGFVGISGAIITQLYHAFYRNDVKSLVLLIGWLPSVVSCFFLRTVRVLKVDRQANEL

SEQ ID NO: 242 - protein, 193 aa, Capsicum annuum
HGSKVPGEIFSLKSGSWRTIDNHPRGIENLLHCSGSALALVNDAFHWVCISGNYFDDSRAFSLVSFSISKEVYADIPLPGQLLRLGGNIGIGVSELDGMLCAYSICEHQRKRTFKLWVLKDYGVGECNYLHLDCVIGIGISELDGMLCAHSICEHQRKRTFKLWVLKDYGVGESWIALFVIDASDILRAVPKY

SEQ ID NO: 243 - protein, 225 aa, Capsicum annuum
MRHQLWFDPNSLSFLTNSNPNSQSSTPPTSPSAQSNPDSTTHPGVLLDLLFNDSELILNPLINVASSGHLKIVEAALDAVQQLIAYGYLRGEVDPTWGPDAKLLSKLIGFVCKCHDLGDDAVELLVTKCILSVVTSVLIRIHGDSLLQVVRTCYDIYMESKNVVNRTTAKASLVQMLVIVFRRMEADSSTVPWQPTVVAELMEPAEKDDTDGSMTLFVQGFITKV

SEQ ID NO: 244 - protein, 226 aa, Capsicum annuum
MARIKPQALLLQSKKKKGPSGVSAPTIIVYILIVAVMVFSLFFTYRYWSRRSIVQTQDAISTSKAIERKKSDIPNYALISTSKGLITVELYKDGSSDIVDDFIDFSQKGHFKGMQFTRVIKNFVIHGSKVERPEATEDWTSRGKLDTSLKHEAFMLGTSKVKHEGGGFDLFITTAPIPDLNNKINVFGRVIKGEDVVQGIEKVDTDDHYRPKTHVQINEVTLKHNS

SEQ ID NO: 245 - protein, 233 aa, Capsicum annuum
AMASIEEVQNIEESLNINLGVRKHGWGSFLEGNIYILDCLTVRLKTARAYDKAAITRIGRDAVTNFDPSTYEGVLSSEGDIGGLSHNIDLNLGISPSSYADNQHGKTTQIGNFQFQHGSTGLPEHHREVLGSAMVKGLEVDTPLKWIWQYQNTYGGSPTLPLFFSAASSGFGNSAIIALSTATHQLYYSSCLSNKSFSHYCSSWPINLSQRPISFLCTSSTTHSITNKDRVYN

SEQ ID NO: 246 - protein, 192 aa, Capsicum annuum
MQLNSLFWVRQDQLLRSALVASLTLKVAAYVLSDTTSYDVWITLHNLYARPSRARLLTLKESLTRVNKGTKSMASYLQQVKQFVTTLNAAGENLTMDDVTIHVLNGFPSEYKILSAIIHARETPLSFDELHEKLCYYKSSVAHEASTIFGPIKENYTSHNGGLNRHTNRTNHYSASKVQPNPNSRQNYFAPS

SEQ ID NO: 247 - protein, 428 aa, Capsicum annuum
MTSASASGNMENLPLFRDVPVSERQVLFLRKLQICCYQFDFNDTMKMVREKEIKRQTLVELVDFVQSGAGKISESNQEEMIRMISGNIMRCLPPSSHENTGSENMEHEEDEPCLEPAWPHLQLVYELLLRYVVSSDTDTKVAKRFIDHSFVLKLLDLFDSEDPREREYLKTILHRVYGKFMVHRPFIRKGINNIFYRFIYETERHSGIGELLEILGSIINGFALPMKEEHKLFLVRALIPLHKPKTVAMYHQQLSYCIVQFVEKDYKLADTVIRGLLKYWPVTNCQKEVLFLQELEEVLETTQAAEFQRCMVPLFRQIARCLNSPHFQVAERALFLWNNEHIVGLIAQNRNVIIPIIFDSLQKNIDNHWNQAIHGLTVNVRKMFMEMDADLFDECQRQYSVKAARATELEEQRELRWRRLADAAAQGC

SEQ ID NO: 248 - protein, 186 aa, Capsicum annuum
MARSATKKISLPNRKKSHGGTKSISLSKEEKQRIYAPWKCSVIIKCVGRKFNRNYLRSKLTDLWKLEEKLILIDLGDELFTTKLILEESQKKILQEGPWFVAGSYVSTRKWEPNFVPIQSKIPSTAIWLRLPFLSTEFYDRSILEKICKHIDSLLKIDSCTSATLRGRYARICVQIPMDTPVCTSI

SEQ ID NO: 249 - protein, 155 aa, Capsicum annuum
MDFVKGLFLLIIEYAITYKSKVIVLPQNKKDLFNLCHASLRSSVERVFAILKRRFRVIDNKAFWDFKTQVNVVLASYILHNHIVDLDPNDMIIQEVDIEAVIQPQNVQISQNSQGIQSYQTQRERREEVRQLSSKRDAIAHAMFINYDRWRNEGL

SEQ ID NO: 250 - protein, 125 aa, Capsicum annuum
MGGGGAAALQKDAPWRASSGAKPIPKIHYSPILRIPQNPHSDYALSIMKHPDPIGSGLGTEAIVEAAGPDCIVPGQNPPIKLLGFKVWPIEVDLKFMEPVGRELKSVGKFMDSAVDLMNKSFIDR

SEQ ID NO: 251 - protein, 129 aa, Capsicum annuum
MSSQLTMQEEELRRGQWLEEEDERLAMIVAISGERRWDALAKDSGLKRSGKSCRLRWLNYLRPNLKHGHITADEERLIIRLQKQFGNKWSKIAKQLPGRTDNEIKNYWRSHLRKKAPVYEQGLLKYSAC

SEQ ID NO: 252 - protein, 260 aa, Capsicum annuum
MACSTQCFLHYHLLSKTPLVTLKGVSDLKPNKLVYLAQRDQQDNGNNVFVSRRLALTILFGAAAIGSKVSPADAAYGESANIFGKPKTNTDFLPYNGDGFKLQIPSKWNPSKEVEYPGQVLRYEDNFDSTTNVIVTITPTDKKSITDYGSPEQFLSQVDFLLGKQVYFGKTDAEGGFESDAVATANLLETSSATVGGKQYYYLSVLTRTADGDEGGKHQLITATVNDGKLYICKAQAGDKRWFKGARKFVENAASSFNLA

SEQ ID NO: 253 - protein, 263 aa, Capsicum annuum
MAWLLSVFPLQLLIKTPDISYGTPSTRESILLPQPIFPVQTGYSLGLGYDSTSGDYKILNICSHSHGSKVPGEILSLKSGSWRTIDNHPRGIENLLHCSGSALALVNDAFHWVCISGNYFEDSRAFSLVSFSISKEVYADIPLPEQLLRLEGNIGIGVSELDGMLCAHSICEHLRKRTFKLWVLKDYGVNESWIALLVIDAWDILRSVPKYRFADGEVLFSSTNLLGKPTHSFWTSRESFGMCDAIRGGFAFTESLISPKSLI

SEQ ID NO: 254 - protein, 230 aa, Capsicum annuum
MCSETSVAMRWHATEQPKDGNLRHPADGEARQHFDSMYPDFAKDIHNVRLGLSSDGFNPFRTMSISHSTWPVMLMNYNLSPWICMKPEYIMLSMIILGPSSPGNDIDVYLQPLITELNELWKVGVEIFDAETNQTFNMRATLLWTVSDFPVLAMLSGWSTKGRLACPTCNHNNCSQYLKHSHKMCYLGHRTFFPSDHPYRSDKKSFNGKEEHKLDLLPYQEKKSLKKYVN

SEQ ID NO: 255 - protein, 226 aa, Capsicum annuum
MWGELKKEWEACKNGEMQSPIDMSHERVRIIRKPEKRHYKLCNATVKNRGHDISLQWHGDAGSVLINGTNYPLQQAHWHSPSEHTVNGRRYEMEMHMVHFNENATNTIAVTAVLYKFGKPDRFLSKLIRNISSMIDKKDEVKNAGMVNPREIKIGSRKYYRYMGSLTVPPCTEGVIWTIKKKVRTVSRDQVKMLREAVHDYAERNVRPLQSINKRPIYLMAPEDTA

SEQ ID NO: 256 - protein, 108 aa, Capsicum annuum
MVDKQIKKKLGLSYARLLVEVKVGKALPEEVKFQNEKGVKLTQNVIYDWKPSLCDHCHKYEHERDDCRKLKVEKKPETTQEEEKRPRERKQKDRVRRRYGIKGKIIQR

SEQ ID NO: 257 - protein, 245 aa, Capsicum annuum
MPINRIAIGRADEATQPDALKAALAEFISTLIFVFAGSGSGVAFSKLTSGXXAGLIAAAVAHAFGLFVAVAVGANISGGHVNPAVTFGAFVGGNITLLRGLLYWVAQLLGSVVACALLKFSTGGMEIGAFSLSKGVGVSNALVLEIVMTFGLVYTVYATAVDPKKGSLGTIAPIAIGFIVGANILAGGAFDGASMNPAVSFGPAVVSWSWNNHWVYWVGPLIGGGLAGLVYELFFINQTHEPLPQ

SEQ ID NO: 258 - protein, 220 aa, Capsicum annuum
MVLGFKPSLQIASAGFFTKTDMAVPRGRNGEEEGGDIFEEEFEFEDPNSVPPHLLNLFDAAENGNLDALRHALDNLTGSIDQPLEDGDTALHLTCLYGHLSCVQLLLERGASLEAKDEDGALPLHDACAGGYTEIAQLLINSAPNPDCVKRMLESVDVEGDTPLHHAARGEHVNVIRLLLASGACPTKTNIYGKSPGELAAPDTEPQRILEEAASAVPSH

SEQ ID NO: 259 - protein, 494 aa, Capsicum annuum
MAAQNSQLHFVLVPLMSPGHLIPMVDFAKLLAQHGVIVSIISTPLNTVRFKSGIDPSIKSGLQIRVYELEFPAVLAGLPEGCENMDSLPSRDSIRDFFAAASMLQEPFEELFSDLRPSPCCIISGKNMAWTVDSARKFKVPRIFFDGMGCFSATCTQKLQSSKVHENVSKFESFVVPGLPHRVELTKAQLPENLNPGSPDLVDVRNKMVAAESFSDGIIVNTFEELELEYVKEFKKIKGGKVWCIGPVSACNKSESEKAARGKNVSLEQNQCLKWLDLQEPNSVVYASLGSICGLTCSQLVELGLGLEASNRPFIWVMRGGEKSKELEKWIEEERFEERIQGRGFLIKGWSPQILVLSHPSVGAFLTHCGWNSTLEGCCSGLPIITCPLFAEQFINEKLITQVLGNGISVGVQAAVTWGMEEKSGLVMKRGDVKNAIEKIIDEGVEGEDRRRKAKEIGKMANRALEEGGSSYLNIKALIQDIMQQSLSSVEASS

SEQ ID NO: 260 - protein, 497 aa, Capsicum annuum
MATQLVQQLHFILFPLMAPGHMIPMIDIAKILASRGAITTIITTPVNANRFSSTISRATQTGSKIQIVTLNFPSEEVGLPKGCENLDMLPSLDLASKFFAAISMLKQQVENLVDTLNPKPSCIISDMGLPWTTQIAESNHIPRIVFHGTCCFSLLCNYKILTSKILENLTSDSEYFVVPGLDDKIELTKAQVSGSAKTNSSVSSSVLKEVTEQIRLAEELSYGVIVNSFVELEPTYEKEYRKVKGKKVWCVGPVSMCNKDSEDLVTRGNKSTIVNQDCLKWLDSKEPRSVIYASLGSLSRLTVLQMVELGLGLEESNRPFVWVLGGGDKLHDLEKWIVENGFEERNKERGLLIRGWAPQVLILSHPAIGGILTHCGWNSTLEGISAGLPMVTWPLFAEQFCNEKLVVQVLNIGISLGVKVPIKWGDEENVGILVKKDDVKKALDKLMDEGKEGQIRRTKAKQLGELAKRAFEEGGSSYVSLTAMIEDIIEQQKSQEK

SEQ ID NO: 261 - protein, 276 aa, Capsicum annuum
MILIKNSTDNTIILTAEEKNRIYHPWSYSVIIKLTRRKINHAHLKNRLSVLWKPMEKSILIDLGSDYFIVKFLKEDSMQIALQKDLWFINGAFLSVRRWHPNFVASKAHKTVSVIWIRLPELPTEYYDHSILSKISRKLGKLVKTYICTSATLRGRYARICIEVPLGVPVKTHVYIGSLKYTIVYEGDDILCTNCGMIGYTHGMCHNRCAILPTTLPEDTVDTTPQTQNTHNECGKEDWLVVKFKKRTNNSQSKVASCSVDEDTSSKRLLCQRERV

SEQ ID NO: 262 - protein, 383 aa, Capsicum annuum
MAEQINSAAGDTTGSGAKRWSFWPSILRWIPTSTDRIIASEKRLLSLVRTPYTQEQVNIGTGPPGSKVRWFRSVSNEPRSINTVTFDSKEGSPTLVMVHGYGASQGFFFLNFDALAKHFKVIAIDQLGWGGSSRPDFTCRSTEETEDWFIDSLEEWRKAKNLSSFILLGHSFGGYVAAKYALKHPEHVQQLILVGPAGFSSETEHMSERMTQFRATWKGAVLNQLWESNFTPMKLIRGLGPWGPNLVRRYTNARFTDLTEESSGLLSDYIYHTLAAKPSGELCLKYIFSFGAFAKSPLLYRAPDWKVPTAFIYGHQDWMNYRGAQQARKNMKVPCEIIRVPQAGHFVFMENTSAFHSAVLYACRRFISPQKDSDSLPEGLVSV

SEQ ID NO: 263 - protein, 195 aa, Capsicum annuum
MHNESCSRVFSLSLHTEAEKVIAFKSIVAYRTGLAINTEVGMQSESQTRTSLIYLHVCFGGGSNFEEKDLDLRLANPLDLRNDTVFSLPFSFGEKDLDLRLANPLNLRNLLEDKKFTKTRLVLLHVAYPFAKEASYLVSVYPQVYLDFGLAIPKHRYHRMISSVKELMAIAPMNKVVMFSTDGFAFAESFYLGLA

SEQ ID NO: 264 - protein, 262 aa, Capsicum annuum
MVIPQERFYSSVKKHGLGLTIECMGMTSLSDDIAQDTWISPTGKVRTVPDLSTKCRVPWAKHQEMVLADMLTEPGKPWQYCPRDVLRRFSKILEDEYGLVMTVGVEVEFYILKTVLIDGKEEKRIFDRTPYCSTAAFDAASSVLDEIVASLKYLNITIEQTHSEAGKGQFEIVLGHTDAITQANNLVYTHEVIKGVARKKWSARYVYAKVGSNVFRCSFLFYLLILYAFASFRAETAGLFWILLSLFLFINISFLLKHEYII

SEQ ID NO: 265 - protein, 83 aa, Capsicum annuum
MHTWYNQNGYGVTASRKYTISDSYAKLLGVKNSWRGAELIWSRIALPKHRLIMWLAMQDKLLTKERLQRLGLGKDSNCCVLCE

SEQ ID NO: 266 - protein, 294 aa, Capsicum annuum
MVFXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXVAIDSTFPFINCFSEADGDALSHVPHTINFKVSLFFHSLTFNLLH

SEQ ID NO: 267 - protein, 630 aa, Capsicum annuum
MIFFSFCNILPVLISNTVLFLHFSFGDKDLDLRLANPLHLRNLLEDKRFMKSRLVLLHASYPFSREASYLASVYSQVYLDFGLAIPKLSFHGMVSSVKELLELAPMNKVMFSTDGYAFAETFYLGAKKAREVVFSVLRDACVDGDLSIPEAIAAVKDIFAENAKQFYKLDVSSRYSDVKPFLSSSFQEEELNGSLKDVTFVRILWIDTSGQHRCRVVPQQRFYSSVEKHGVGLTCACMGMSSTSDGPAVDTNLTASGEIRIVPDLSTKCRLPWARQQEMVLADMYIEPDKIWEYCPREALRRVSKILREEFDLAVNAGFENEFYLLKSVLRDGKEELAPFDRTPYSSTSAFDAASPILEEVFASLQSLDIIVEQLHAEAGKGQFEIALKYTECFRAANSLIFTREVIRAVARKHGLLATFVPKYALDDIGSGSHVHISLSRNGENVFMASGESKYGMSKIGEAFMAGVLNHLPSILPFTAPLPNSYDRIQPNMWSGAYLCWGKENREAPLRAASPPGVPHGFVSNFEIKAFDGCANPYLGLAAIIAAGIDGLRRNLSLPEPVDGDPDILKENLQRLPVSLAESIEALERDTLFRDMIGEKLLVAIIGVRKAEVKYYSDNKEGYKDLIFKY

SEQ ID NO: 268 - protein, 208 aa, Capsicum annuum
MISAPAASPGDVGGQALALRITGDEGAFYGCGFYGAQDTLNDDKGRHYFKECFIQGSIDFIFGSARSLYEDCTINSIAKEGTSGEIGGSITAHGRNSKNENSGYSFVNCEISGTGKIWLGRAWGAYATVIFSKTYMSDVISSDGWDDWRDPNKDQTVLFGEYDCYGPGANYTDRVPYAKQLKQNEAAQYMDISFIDGQQWLSNGRSDT

SEQ ID NO: 269 - protein, 415 aa, Capsicum annuum
MVYLSVQFQRYWLWAKRQNHTYRPNRPLTAQEEVQSVGQLREVSNKANNAELKLFLEVEFGLELQPVPPPEKIKEDILLFFKLYDPSKEEIRYAGRLFVKGSGKPLEILAKLNELAGFLPDEEIELFEVNFSSSFAASSSTQNIGCDFYLCYLQEIKFEPNVMCEHINRNLSFRGCQLEDGDIICFQKSLRKQGNEQYRFPEVPSFLEYVHNRQVVRFCSLEKSKEDEFTLELSKQNNYDEVVECLARHLHLDDPSKVRLTSHNCYSQHPKPQPIRYHGVDRLTEILSHYNQTSDILYYEVLDIPLRELQGLKTLKVTFYHAAKDEVTIHTIRLPKQSTIGDVLNDLKTKVELSRPDAELRLVEVFYHKIYKVLRCCIASFYTVVLGLLRCFSLLSTQISSLVVNLISFTYFFVF

SEQ ID NO: 270 - protein, 727 aa, Capsicum annuum
MGKNKKTDEAGVSGKAKGSSKDSSKEGKKEKLSVSAMLASMDQKPDKPNKGSSATASSKPKPKAAAKASSYTDDIDLPPSEDEEEEEHFLDEEEDLRRQRRNETGGALDTSISHKEVKKREKKGMIAVQAAEVAKKEALRDDHDAFTVVIGSRTSVLEGQDDADANVKDISIENFSVAARGKELLKNTSVKISHGKRYGLVGPNGMGKSTLLKLLAWRKIPVPKNIDVLLVEQEVVGDDRTALEAVVSANEELIKLREEAASLQNAAASVGETEDDVDGDDVVERLSELYEKLQLMGSDAAEAQASKILAGLGFTKEMQGRSTRSFSGGWRMRISLARALFVQPTLLLLDEPTNHLDLRAVLWLEEYLCRWKKTLVVVSHDRDFLNTVCSEIIHLHDMKLHFYRGNFDDFESGYEQRRKEMNKKFEVYDKQVKAAKRSGSRAQQEKVKERAKFVAAKESKKKGKDKVDEDETPTEAPKKWRDYTVEFHFPEPTELTPPLLQLIEVSFSYPNRPDFRLSNVDVGIDMGTRVAIVGPNGAGKSTLLNLLAGDLVPTEGEVRRSQKLRIGRYSQHFVDLLTMDETPVQYLLRLHPDQVGPSKQEAVRAKLGKFGLPSHNHLTPILKLSGGQKARVVFTSISMSKPHILLLDEPTNHLDMQSIDALADALDEFTGGVVLVSHDSRLISRVCDDEERSEIWIVENGTVEKFPDTFDEYKAELIREIREEVDD

SEQ ID NO: 271 - protein, 148 aa, Capsicum annuum
MGDKSFTEDLTIIKLGGSDIVLGNDWMKKFNPTIFDLEHHCVTVGKNADYVILHALPEEGQLNMITSTSMGKILRKGQTLLGHLFLMAGATEVEQTEIDRAIQKILVKYEMLFAKPKSLLPERKLDHAIPLKQNALPVSLRPYRYNYH

SEQ ID NO: 272 - protein, 259 aa, Capsicum annuum
MNYEFEEIWEAWDIRVVILFILTLQIILIFFSPQRKSFLIVHLGGPDTISAFALEDNALWHRYFLGLTTQGSSVAYVFFQTVPRNKLWIPTVLIYVARVIKYGKHIWALYLASLDRFCESIMKDPDPDPNYAKLIDEYSSKIAAKLSSNIEMIHSTMALWKQKNSGEAHYLFEKFKGLIVDLIFDFCQRNEIQNFFLILEAIDAFRVIEVEHNFIYDVLFTKMIVANSYSGYLFRLICFLLEVVAAILFYRWDKRGLPR

SEQ ID NO: 273 - protein, 252 aa, Capsicum annuum
MIGGGSSERASDDGQSKIIIKSHERWSYRTVDRNRVFVFNEKENFMERFLIRRDNEVKGFKALGIYGVAGVGKTTLCQLTFNNKQVMNHFFPQIWVCKHRGLRGLLFALRSVLKGRRYLIVLDDAFYIDNFFDNLWSEPKLEDNCTEKLALGLPKQQSGAVIVMTWSEELEKEMVGEENVHPLLPLQDKSCHEIFEDSSGEKVKQLLNFQTDHDLEDTFMSKFGGLPLAAKIMGKIMEQELTKMPANTTSLD

SEQ ID NO: 274 - protein, 145 aa, Capsicum annuum
MDSEDKSWMNCLIWSDEFERGVKYFLEKAFERASQGNEILCPCRVWKNQYWRYRDVVEDHLLSRGFLANYTKWTFHGESASLRKTHHSINDDEDSNMCDDIDGLLHDTFRNVDAESRYGQGVGEGLPEDATKFFKLLEEGKQQLQ

SEQ ID NO: 275 - protein, 114 aa, Capsicum annuum
LNLALLPEANETVQLLERFLKKNGILKNLKPSQIDNFQHKTHPLVLLNNTNKDKLVCDGCISCQQCSYFMHLTCSQLPGELEPYHPVSPGTRTLHKASGVLLFLPAMQSLQIRH

SEQ ID NO: 276 - protein, 515 aa, Capsicum annuum
MAKYMCADDTLRYFEWLENYRKLADPATINVLKMCLRFLKVFLVAAEKLPTEQKDLISYVQVWVQNTGKAIQSLCANAVEESKVTEVWHGKIEYLTSKISTSSRSVLQLGARVSTGCLVELIDSVLNNLNDLLGPETKQPIDSTKKQIEVLEAKFRFLRNFAWFSSQSCIEHHKLEDLLRIIEVVATTVACVLFQWLFEDMDTIMETKFSQVLVTIQLIEPELRYAYFRSLKLSLPSRLKENIPMVVSIGFLCCFHNDLKELLSPKAYLMASARDHMCNLCEELNFVIAFLRDVQKMDDSKSVEDLLEHTKGMTTKTAHVIHLLLVSSRNDDDSSQPGSSVLYDLLNEIDFTKKEMITDKYLDLWKSIRSNLIPKVNSLGLIDILLGTINVVLDNKNDLIAFFGHQLEIVKTEVLSLRSLLCVNDIKTEITDVVHEAKHAIGSLIIRESPCWHCMLQLSSVIIKIQAVINKVASCFVSDQIGEIGMGTSAQNSDSSSEVHMPPVKSEMERETTEV

SEQ ID NO: 277 - protein, 283 aa, Capsicum annuum
MSDTMRGADLSGTISVTSESSKATGFDANRIAEVKAWLTSQFDAAGKDVPDFKYTPRSINELQKIATLSQAKTQAAVIVANDHPQKAAEYRSQAATIREILESVGLAQESLPSNVVSSSHVLAKVANLLDVRDTELSSFLVAVAELSLRKTAVEEKRAKVQQESKLEDDIAPCEAQMENWKTNLAIMESKERHYLQKYGYYKAALNHVGYTPEISHGVLVEMAEHKKDLEKKTKQILDTLRSYQDLPPDKALAALAIEDKKRQYAAAEKYLEDVLQSALASSE

SEQ ID NO: 278 - protein, 96 aa, Capsicum annuum
MKHDKADCPADEITDEMEHQPCPRGGITPAPASRFAQYPKLLDKCDIDINTIIEGVILPICVHDRVTTLVITNYTLSQHARYLNIQFQAYWSWSLH

SEQ ID NO: 279 - protein, 470 aa, Capsicum annuum
MREAPEEEEDGDSEGDASFSLRGLGDRVGREFHYLSDSKSHPDGSPGDFGKSLEMSPRSMYRAISLKYRYSVDGVNDGCIGLPGRAVADLHRSREFGKFPEWTPNVQYYSSKEFPQLDHALNYNIRGTFALPVFERENCVGVLELVMTSQKINYAPDFDKVCKALEATNLKSTGTFDHQDTWSEILELLYAVWETHKLPLAQTWLPQRHQNGMICGKRFSVLTCVPSLAFYATDASMWSFREACLEHHLQDEQGLPGRAFSSLNACFCVDITLFRKSDYPLVLYVRMFGLQSSLTIPVCSNHSEKDTYALEFFLPHSSTDHKIQLQITASILATTMEILQNSKVIQWIEPKVGHFKIIDLSIGKKLRFSPVPELKSQTMETTHGLETSQSGLVSKEQDFSLQQRSECDVALLKVSDAIAENITFSTFEKDGKMTLQFLKQLFGKNLNDAANCLGNLNFRSHHCWKNWKTR

SEQ ID NO: 280 - protein, 880 aa, Capsicum annuum
MTAKTNREFATSSAAPSATETASAKSESESGDLNRNKSDSSRNGVVVYRRNKRLKMADSSGKVVNTDVAKGNLGIPVEESKKSRRLTRSAVVGRKRELVEILNGKSGSEVDDERSSVTKKLEMKMSKKISITVIPATVKELFETGLLEGYPVFYAGKKGIPLRGIIKDTGILCSCELCKGTTVIPPSKFEIHACNSYRRASEYICLENGKSLLDVVKECSKGSLKTLEETIQSVIGPVPMKKSLICRDCKGSFVATLAGKDEQLCDPCIISLRSGATPTQLINTENGVFEPVLNLKSSGTSTMSSVLPRGIRGRKKKKAVMVHSSSQSPSVHTLSRNKWKTPNKLMNPVFSPKSDGAVTMGSSLTNNMQGNISEKLSKSVPVSKYSKVSPGVSVHSRNQWKMTKRDQKMHKLVFEEGGLPEGTEVAYYSRGKKLLVGYKKGSGIFCSCCNTEVSPSQFEAHAGWASRKKPYGYIYTSNGVSLHEFAISLLKGRKSSLKDSDDLCIICADGGKLVLCDGCPRAFHKECASLSTIPRGKWYCKYCESMLQREKFAEHNANALAAGRVSGVDSIEQITNRCIRSVKNAEEAEFIACVLCRAYDFSKSGFGPRTVILCDQCEKEYHVGCLKKSKISDLKELPKGKWFCSTNCKRIYSALQNLLNSGEERLPDSCLDATRVKEKEKSLVAGGEFDVRWRLLSGRVSSRETRRLLAEAVSIFHDGFDPIVDSVTGRDFIPSMVYGRNIRGQDFGGMYCTILTVNSTVVSAGILRIFGQDMAELPLVATRVGSKGKGYFQVLFSCIEKLLAFLGVRRFVLPAAVEAMSIWTKKFGFVELSPDQIANYRKTCWQMISFKGTSMLEKMVPKCRIIQRGEAEAEADVPDE

SEQ ID NO: 281 - DNA, 101 bp, Capsicum annuum
  source 1..101: mol_type = genomic DNA
aattttgttttttcctattcctaatcaaattgggattagagttttattagaactataggccttgtaaatatggatggcctgacatttcgagttaatggtaa

SEQ ID NO: 282 - DNA, 106 bp, Capsicum annuum
  source 1..106: mol_type = genomic DNA
gtcttattgtagcacagagaaaagggcataaataatcccttatctataggagcaagtattatctaaaagtttattaatattgatctttggatattcttatacaaac

SEQ ID NO: 283 - DNA, 123 bp, Capsicum annuum
  source 1..123: mol_type = genomic DNA
gtttttttataatttgatatctattatgcatggttgtgaatgtataacaaatttatacatatcatatatataccttctattcgaattgaacctcagttattgttggacattcagtatggatgt

SEQ ID NO: 284 - DNA, 101 bp, Capsicum annuum
  source 1..101: mol_type = genomic DNA
tttcggactgtataattggactggacactaaatttaggattgtttggttttgcttgcttgctttgttttgtatgttttgtgtggtttatacatttgtagtg

SEQ ID NO: 285 - DNA, 101 bp, Capsicum annuum
  source 1..101: mol_type = genomic DNA
cagttaatcattaattgtccaatttggacgaactacgcatacctgattcccttccctcggggtgggatatgtaggcaaccctcgatgggttcggtaatatt

SEQ ID NO: 286 - DNA, 101 bp, Capsicum annuum
  source 1..101: mol_type = genomic DNA
atcagcaaaaggtaggtctttggggtcggcgggaccgtttgggtagtcaaccgggagaaacggcacgaacaaaccgtatttaggccaaattggtgtgctat

SEQ ID NO: 287 - DNA, 102 bp, Capsicum annuum
  source 1..102: mol_type = genomic DNA
tgtcttccctcatatgtgaatctgattcttttttttttaggctgaacacgtaaaaaaaaaaatttagataatgaatgagtgacatttaacacttatatagga

SEQ ID NO: 288 - DNA, 106 bp, Capsicum annuum
  source 1..106: mol_type = genomic DNA
catatgtgaatctgattcttttttttttaggctgaacacgtaaaaaaaaaattttttttagataatgaatgagtgacatttaacacttatataggatatgcatctt

SEQ ID NO: 289 - DNA, 101 bp, Capsicum annuum
  source 1..101: mol_type = genomic DNA
ctggtattgccagttctaacagaatcaagtctgttcaacaaatgaaagtggtatgcttgaacgtacacaatgtggccttttttgcacttcatcataataca

SEQ ID NO: 290 - DNA, 103 bp, Capsicum annuum
  source 1..103: mol_type = genomic DNA
taaagagggtgaactctcagattaaattactctttcatggttgcaaaaattaaaaaaaaaagaattgacatattattaaacaaattaaataacactatattaa

SEQ ID NO: 291 - DNA, 155 bp, Capsicum annuum
  source 1..155: mol_type = genomic DNA
tgcagggattgatttccttatgtttcctaacataccacctggagatcgttgggaggccaaaggtacttattaaaaaaattagtttggcaagtttttggagaaaataagataccatgatgaagtaacgattatcattatctctttcggaggaaaga

SEQ ID NO: 292 - DNA, 103 bp, Capsicum annuum
  source 1..103: mol_type = genomic DNA
atggcgatatttggttgactcttacatattagaaaactaattatatttaataaaaaaaaaaaaaagataaaatagacatttatgacatgtcttctacatctgc

SEQ ID NO: 293 - DNA, 101 bp, Capsicum annuum
  source 1..101: mol_type = genomic DNA
cttcacaaagtgggtaaaagtagtaactttcaagtcagtgacaaagaaggttgtggtagattttgttcatgccaatatcatttgtagatttggaattccta

SEQ ID NO: 294 - DNA, 100 bp, Capsicum annuum
  source 1..100: mol_type = genomic DNA
caaaatatctcaaatattaaacatcaacgtgatcgtaacacacactttctaaaaaaaaatagaatttccaatgggaattcacgacccaaatatcaaaatc

SEQ ID NO: 295 - DNA, 101 bp, Capsicum annuum
  source 1..101: mol_type = genomic DNA
atcataggggtgcatggtttaagatgagtataaagaagggaaaaataaaggaaaaataataaaagttcggtgctattcacaaaaaaaataaaataataata

SEQ ID NO: 296 - DNA, 133 bp, Capsicum annuum
  source 1..133: mol_type = genomic DNA
gggtttgaatattgattcaaaaaacatttcatgaaatcattttgatgaaagtatatatatatatatatatatatatatatatatatatatattgtattgaactctttttcatggaaattgaattatagcccct

SEQ ID NO: 297 - DNA, 102 bp, Capsicum annuum
  source 1..102: mol_type = genomic DNA
tccactttaataatgaaaaccctttacaaactaaaatatgatctttttggtaaaaaaaaaaaaagggtgttgatagctctagcaactctgctggggactttt

SEQ ID NO: 298 - DNA, 101 bp, Capsicum annuum
  source 1..101: mol_type = genomic DNA
aaacatcaaaaagatttttcttttgtttctttagtcagcattcataattttaaaaaaaaaaaaatttattaatagaaactttttataaaagaaaaattcaa

SEQ ID NO: 299 - DNA, 101 bp, Capsicum annuum
  source 1..101: mol_type = genomic DNA
atctcattatatgtcatctcaagagcttgaccacctgcatctaaatctagaaaaatctttgtattttgatcaagagcctctacaaagtatgagcaagtacc

SEQ ID NO: 300 - DNA, 101 bp, Capsicum annuum
  source 1..101: mol_type = genomic DNA
aaacaaagattatgataaagcaagattttattattctgaaaataatgtccaatttttcatgagggtcctgaaaataaagtaaactcccattttttaggagg

SEQ ID NO: 301 - DNA, 117 bp, Capsicum annuum
  source 1..117: mol_type = genomic DNA
gaaatccttccaaatgcagcatctcttgtttgactcttgacattggtgtttggcattctgtagatacgcaatgtttgtattaggaagttggctaattttataatcaggtggcatggt

SEQ ID NO: 302 - DNA, 101 bp, Capsicum annuum
  source 1..101: mol_type = genomic DNA
ttggatatcgaagtaaaacgttatggttgatctacttcagactatcaaaaaagtccaccaaaggatagattcgagaattattgaatttttaggggcgtttt

SEQ ID NO: 303 - DNA, 101 bp, Capsicum annuum
  source 1..101: mol_type = genomic DNA
ttatgtagtataatgctagtattcgttgtttggaaagacgtaacattgatgtagtgagattgaataaataaaaatacatctatttttctataatgttaata

SEQ ID NO: 304 - DNA, 101 bp, Capsicum annuum
  source 1..101: mol_type = genomic DNA
atatctacatgttcaatgtgttctatccataattgtcgttgattttcgttgtaaggtggtgtataatctcatttagtactgaatatatgtatagaagaata

SEQ ID NO: 305 - DNA, 103 bp, Capsicum annuum
  source 1..103: mol_type = genomic DNA
tatattttaatattataatagttgaaaacatctagtacattttaaaaagacaaaaaaaaaaaagaaaaaagacttattaggctaaagcccagcagaagcaacc

SEQ ID NO: 306 - DNA, 102 bp, Capsicum annuum
  source 1..102: mol_type = genomic DNA
ttatgtttttcaaaaagtttttttactattcacctccgttagttaaccgttaaaaaaaaaaaaagagtaagccgtaagaatttattacctttatcctatttt

SEQ ID NO: 307 - DNA, 101 bp, Capsicum annuum
  source 1..101: mol_type = genomic DNA
cctaaagactaggacaaagctggaaatagattgaaaataggactttattctctgactttatttgctaacaagcatgatataaggacaggtaagcaaataac

SEQ ID NO: 308 - DNA, 102 bp, Capsicum annuum
  source 1..102: mol_type = genomic DNA
gataagcatcccttataatagatgttattgaagagaattttaagaatagataaaaaaaaaaactctaatatatttgtcatttctgttataaaatgtatagta

SEQ ID NO: 309 - DNA, 103 bp, Capsicum annuum
  source 1..103: mol_type = genomic DNA
atatgttgcatcagataatttatttattgcaacatataatctactttttttaaaaaaaaaaataactatcatcatatcattaatccaaatttgcttacttttt

SEQ ID NO: 310 - DNA, 107 bp, Capsicum annuum
  source 1..107: mol_type = genomic DNA
aatcattaattaaaataaaaacttgattagaattggacttactgctttcttgggggggggggggttgaaatgatcaagaaattttgtatttttataggttttggccg

SEQ ID NO: 311 - DNA, 114 bp, Capsicum annuum
  source 1..114: mol_type = genomic DNA
tacttatctatatatatgtggggtgtaaaataataatttagtataatcttattggattatcagtttacccaataacccaataaaaaaatcgaaattgaatcaataacccaataa

SEQ ID NO: 312 - DNA, 103 bp, Capsicum annuum
  source 1..103: mol_type = genomic DNA
ttaaaagttgaggacataaaatcatatttaaaaaagatttttcaaaaatctaaaaaaaaaaaattcaaaaagacatggtcaaacacaactccaactttatctt

SEQ ID NO: 313 - DNA, 101 bp, Capsicum annuum
  source 1..101: mol_type = genomic DNA
atctttaacttcaactctaactccaaagaaagtaatttttatgaccaaatcgctattaatgtgttgttatatgtgacaattttagaaccaattgctaccca

SEQ ID NO: 314 - DNA, 101 bp, Capsicum annuum
  source 1..101: mol_type = genomic DNA
tgttgacaaaatttttgtatgccttatttcggctcataattatatcttttacatgtgctggcccttttgtcttgctttgttcaattgaagaagctggtagc

SEQ ID NO: 315 - DNA, 101 bp, Capsicum annuum
  source 1..101: mol_type = genomic DNA
atatagaagctactgctgaacaatattgataatatgctactctttttatggaaatacggagaacagaaaatgcagaatcgtacgtaagcgacaataaatat

SEQ ID NO: 316 - DNA, 101 bp, Capsicum annuum
  source 1..101: mol_type = genomic DNA
ataaagaggtttggcaagaagggaaactcagtcctcgatacatcggtccttaaagaattctcagctattttaaaaaggtagcttatgaacttgagttgtct

SEQ ID NO: 317 - DNA, 101 bp, Capsicum annuum
  source 1..101: mol_type = genomic DNA
caattaggaaaatgactcatattgtcattaccagtaaaaattttattttttaaaaaaaaaaaaaaaagggtaaacaggctcttgctctctgttgagtatag

SEQ ID NO: 318 - DNA, 101 bp, Capsicum annuum
  source 1..101: mol_type = genomic DNA
ctaatacttccttcgtttcgttttagttgatcctttttctaaaaatatttattttaatttagttgtctctttgatgaaatctagagaattttattatattc

SEQ ID NO: 319 - DNA, 103 bp, Capsicum annuum
  source 1..103: mol_type = genomic DNA
atttaggacatccaacggtccaattgaaatatgtggtttcatttaggatgatttttttttttttacaggaagcttgattttttcaaaataacttatttagtgt

SEQ ID NO: 320 - DNA, 101 bp, Capsicum annuum
  source 1..101: mol_type = genomic DNA
gaatctacaaccccataagctattgacaatatggaacctacaatataaaagaatcatattgtatcaattgttatataattatacaaaataaactaaaataa

SEQ ID NO: 321 - DNA, 113 bp, Capsicum annuum
  source 1..113: mol_type = genomic DNA
tgtactggtatgcatgtgaggtaggctaaatgcatagggttcatatgcatgaataataataataataataataataataataataactgactaactaacatgagtgtgagaat

SEQ ID NO: 322 - DNA, 101 bp, Capsicum annuum
  source 1..101: mol_type = genomic DNA
atccaccaaatcattcaatctcaaatatagcaaattcaacaaattatgattaaaaaaaaaaagagaacggagtaaagaaatgaaccatttgataatcaatt

SEQ ID NO: 323 - DNA, 101 bp, Capsicum annuum
  source 1..101: mol_type = genomic DNA
atcacttgtgacattttaataacacagcatatgtgtcatttctttcttcttcatttgtcaaacatataactctacctttgttatacatctctcacatggaa

(19) United States
(12) Patent Application Publication
(10) Pub. No.: US 2025/0024809 A1
(43) Pub. Date: Jan. 23, 2025

(54) PEPPER PLANT WITH IMPROVED YIELD

(71) Applicant: BREEDX LTD., Kanot (IL)

(72) Inventors: Binyamin NIR, Sderot (IL); David JOLLES, Eliav (IL); Moshe BAR, Rishon-LeZion (IL)

(73) Assignee: BREEDX LTD., Kanot (IL)

(21) Appl. No.: 18/790,617

(22) Filed: Jul. 31, 2024

Related U.S. Application Data
(63) Continuation-in-part of application No. PCT/IL2023/051209, filed on Nov. 23, 2023.
(60) Provisional application No. 63/384,806, filed on Nov. 23, 2022.

(51) Int. Cl. A01H 6/82; A01H 1/00; A01H 1/04; A01H 5/08

(52) U.S. Cl. CPC A01H 6/822; A01H 1/045; A01H 1/121; A01H 5/08

(57) ABSTRACT

A cultivated pepper plant or seed capable of producing high yield properties of parthenocarpic fruits. The plant or seed comprises at least one Quantitative Trait Locus (QTL) for an Excellent Parthenocarpy (EP) trait, the EP trait conferring production of elevated yield properties of parthenocarpic seedless fruits, as compared to a pepper plant having the same genetic background and lacking this EP trait, and the QTL is selected from QTL1 located 09.0-13.5 Mbp on chromosome 1, and QTL2 located 199-218 Mbp on chromosome 10.

### CROSS-REFERENCE TO RELATED APPLICATIONS

This application is a continuation-in-part of PCT Application No. PCT/IL2023/051209 filed on Nov. 23, 2023 which, in turn, claims the benefit of U.S. provisional application Ser. No. 63/384,806 filed on Nov. 23, 2022, the disclosures of which are hereby incorporated in their entirety by reference herein.

### REFERENCE TO AN ELECTRONIC SEQUENCE LISTING

The contents of the electronic sequence listing (Sequence_Listing_Pepper_Plant_with_Improved_Yield.xml; Size 370,173 bytes; and Date of Creation: Jul. 30, 2024) is herein incorporated by reference in its entirety.

### FIELD OF THE INVENTION

The present invention relates to pepper plants or seeds with high yield properties of parthenocarpic fruits, methods of producing said plants and seeds and to use thereof.

### BACKGROUND OF THE INVENTION

Crop improvement is a common approach that has been used for many years seeking the development of new and improved cultivars (varieties) comprising the desired trait or traits. The better traits or properties include yield performance, disease resistance and market preference.

Regarding the fruit market, the consumers will tend to buy fruits according to its aspect (color, weight, size), taste, and with some proprieties, such as seedless fruits, since it is easier and more convenient to prepare for eating. Not only for the consumers, but seedless fruits save time and money in food establishments and industries.

Therefore, new technologies and new seedless plant varieties have been developed in this sense. Hormonal treatments, such as colchicine for watermelons and gibberellin for grapes, are used to produce seedless fruits. The clear drawback of treating fruits with hormones is that each fruit needs to be individually treated, thus demanding extensive work.

There are also breeding methods for producing seedless fruit varieties, such as tomato and peppers. The production of hybrids is a well-developed industry, involving the isolated production of both the parental lines and the hybrids which result from crossing those lines.

A few efforts have also been made for producing seedless fruits. US20210037779A1 discloses parthenocarpic plants and methods of producing same, wherein the plant, selected from the group consisting of tomato, pepper and eggplant, exhibits a facultative parthenocarpy and comprises a loss-of-function mutation in an AGL6 gene. This prior art is not related to introgressed traits but to using mutagenesis methods and treating the plant with a mutagen. In addition, it did not achieve high yields, but it kept plants with an average fruit weight/plant as that of a non-parthenocarpic tomato of the same genetic background under fertilization permissive conditions of the non-parthenocarpic tomato.

U.S. Pat. Nos. 8,492,619, 9,303,271 and 10,143,174 relate to a method of producing a seedless pepper plant comprising crossing a seedless pepper plant which is male sterile as a female line and a male-fertile pepper plant as a male line. This publication teaches that the seedless trait is controlled by a genetic determinant. U.S. Pat. No. 8,957,286 B2 provides a pepper plant capable of bearing seedless fruits by a crossing method of a male sterile line and a plant of a parthenocarpic line. However, this prior art does not teach cytoplasmic male sterility trait and does not teach or achieved a hybrid plant with cytoplasmic male sterility combined with production of parthenocarpic fruits. In addition, this publication does not solve the problem of generating high yield and high fruit quality of commercial products setting seedless parthenocarpic fruits under hot conditions.

U.S. Pat. No. 10,499,578 B2 discloses seedless pepper plants and methods to produce the same, wherein the method comprises crossing as a female parent a first pepper plant comprising a non-Peterson cytoplasmic male sterile (CMS) trait, which is derived from Capsicum baccatum, with a second plant which is parthenocarpic to produce at least a first seed of a parthenocarpic seedless pepper plant. U.S. Pat. No. 10,499,578 B2 also discloses a method for increasing the number or yield of fruit of pepper plants grown under cold conditions. However, the pepper plant of this prior art merely comprises a non-Peterson CMS trait from Capsicum baccatum, and the female parent is non-parthenocarpic. In addition, the higher number of parthenocarpic fruits are not achieved under hot conditions, but only under low temperatures.

It is a long felt need to have means and methods of obtaining cultivated pepper lines with enhanced parthenocarpy traits that will keep and/or improve the yield properties of the pepper fruits.

### SUMMARY OF THE INVENTION

It is a principal object of the present invention to provide new Capsicum annuum pepper plant or seed with an Excellent Parthenocarpy (EP) trait capable of producing high yield properties of parthenocarpic fruits.

It is an object of the present invention to provide a cultivated pepper plant or seed capable of producing high yield properties of parthenocarpic fruits, wherein said plant or seed comprises at least one Quantitative Trait Locus (QTL) for an Excellent Parthenocarpy (EP) trait, said EP trait conferring production of elevated yield properties of parthenocarpic seedless fruits, as compared to a pepper plant having the same genetic background and lacking said EP trait, said QTL is selected from QTL1 located 09.0-13.5 Mbp on chromosome 1, and QTL2 located 199-218 Mbp on chromosome 10.

It is another object of the present invention to provide the pepper plant or seed as defined above, wherein said plant is male sterile, the male sterility is selected from a cytoplasmic male-sterile (CMS) trait, a genomic male sterile trait (GMS) or a combination thereof.

It is another object of the present invention to provide the pepper plant or seed as defined in any of the above, wherein QTL2 is located 204-218 Mbp as indicated in pepper genome Capsicum annuum cv CM334 v1.55 or at position 199-214 as indicated in Capsicum annuum cv Maor genome ASM2707369v1 NCBI, on chromosome 10.

It is another object of the present invention to provide the pepper plant as defined in any of the above, wherein QTL1 located on chromosome 1 is associated with the property of elevated fruit number per plant and/or elevated fruit weight per plant.

It is another object of the present invention to provide the pepper plant or seed as defined in any of the above, wherein QTL2 located on chromosome 10 is associated with the property of early fruit setting and/or setting parthenocarpic fruits at high temperature and/or setting fruit at high temperature.

It is another object of the present invention to provide the pepper plant or seed as defined in any of the above, wherein said plant comprises at least one allele, haplotype, molecular marker, single nucleotide polymorphism (SNP), gene and/or genetic determinant associated with the at least one QTL.

It is another object of the present invention to provide the pepper plant or seed as defined in any of the above, wherein the genome of said plant comprises at least one molecular marker and/or gene associated with the at least one QTL, the at least one molecular marker and/or gene is selected from the group consisting of: (a) a molecular marker selected from (i) SEQ ID NO: 281-290 or any combination thereof, (ii) allele A at position 9570115 as indicated in pepper genome Capsicum annuum cv CM334 v1.55 or at position 12,239,886 as indicated in Capsicum annuum cv Maor genome ASM2707369v1 NCBI, and/or (iii) a gene encoding a sequence selected from SEQ ID NO: 1-93 or any combination thereof, (iv) any combination thereof, associated with QTL1 on chromosome 1; (b) a molecular marker selected from (i) SEQ ID NO: 291-323 or any combination thereof, (ii) an allele selected from the group consisting of:

| | Position (Capsicum annuum cv |
|---|---|
| Allele | CM334 v1.55 reference genome) |
| C | 204079966 |
| T | 206006853 |
| T | 206040933 |
| C | 207234334 |
| T | 207311998 |
| A | 208017242 |
| C | 209904182 |
| T | 209904264 |
| G | 210564835 |
| T | 210564909 |
| C | 210743072 |
| G | 210819645 |
| A | 210819791 |
| T | 210820018 |
| G | 210820058 |
| C | 210909279 |
| A | 213685989 |
| A | 213686017 |
| G | 214398109 |
| C | 214593001 |
| T | 214722404 |
| C | 214853116 |
| A | 215238239 |
| A | 215250773 |
| T | 215250835 |
| A | 215940270 |
| G | 216217080 |
| T | 216959296 |
| A | 217149552 |
| C | 217149565 |
| T | 217149575 |
| T | 217245544 |
| G | 217444730 |
| A | 217492476 |
| A | 217492488 |
| A | 217501066 |
| C | 217501349 |
| C | 217504354 |
| C | 217524569 |
| G | 217538084 |
| G | 217538473 |
| A | 217613274 |
| C | 217613308 |
| T | 217613345 |
| G | 217613384 |
| G | 217613401 |
| A | 217613486 |
| T | 217613553 |
| A | 217613566 |
| C | 217613583 |
| C | 217613587 |
| C | 217613736 |
| G | 217613791 |
| A | 217613899 |
| A | 217613910 |
| A | 217629488 |
| G | 217629537 |
| A | 217629540 |
| T | 217631692 |
| C | 217631722 |
| A | 217631735 |
| T | 217631744 |
| G | 217631746 |
| C | 217802876 |
| T | 217802908 |
| G | 217874088 |
| T | 217874098 |
| G | 217874137 |
| C | 217874624 |
| T | 217874686 |
| C | 217874705 |
| A | 217875456 |
| G | 217875502 |
| T | 217875804 |
| C | 217876561 |
| T | 217876777 |
| A | 217876872 |
| G | 217876940 |
| A | 217876961 |
| G | 217876970 |
| C | 217877084 |
| T | 217877116 |
| T | 217877164 |
| G | 217877182 |
| T | 217933099 |
| G | 217933230 |
| G | 217933311 |
| G | 217933467 |
| A | 217933523 |
| C | 217933527 |
| G | 217933778 |
| A | 217933882 |
| A | 217933937 |
| G | 217934044 |
| A | 217949286 |
| T | 217950632 |
| A | 217954424 |

or any combination thereof, and/or (iii) a gene encoding a sequence selected from SEQ ID NO: 94-280 or any combination thereof, (iv) any combination thereof, associated with QTL2 on chromosome 10; and (c) any combination thereof.

It is another object of the present invention to provide the pepper plant or seed as defined in any of the above, wherein the high yield properties of parthenocarpic fruits comprise at least one of: early fruit setting, setting fruit at high temperature, setting parthenocarpic fruits at high temperature, elevated fruit number per plant and/or elevated fruit weight per plant.

It is another object of the present invention to provide the pepper plant or seed as defined in any of the above, wherein the high yield properties comprise (i) increased parthenocarpic fruit number per plant of at least about 5%, particularly 10-25%, and/or (ii) increased fruit weight per plant of at least about 15%, particularly 20-35%.

It is another object of the present invention to provide the pepper plant or seed as defined in any of the above, wherein the property of elevated fruit number per plant and/or elevated fruit weight per plant is associated with molecular markers and/or genes as defined above, associated with QTL1 located on chromosome 1.

It is another object of the present invention to provide the pepper plant or seed as defined in any of the above, wherein the plant comprising the at least one molecular marker and/or gene associated with QTL1 have 15-20% more fruits compared to a plant lacking said at least one QTL1 associated molecular marker and/or gene.

It is another object of the present invention to provide the pepper plant or seed as defined in any of the above, wherein the property of early fruit setting and/or setting fruit at high temperature and/or setting parthenocarpic fruits at high temperature, is associated with molecular markers and/or genes as defined above, associated with QTL2 located on chromosome 10.

It is another object of the present invention to provide the pepper plant or seed as defined in any of the above, wherein the plant comprising the at least one molecular marker and/or gene associated with QTL2 have an average EP value of above 3, particularly in the range of 3-8.

It is another object of the present invention to provide the pepper plant or seed as defined in any of the above, wherein the plant comprising the at least one molecular marker and/or gene associated with QTL2 exhibits an increase in EP value of about 20-40% compared to a plant lacking said at least one QTL2 associated molecular marker and/or gene.

It is another object of the present invention to provide the pepper plant or seed as defined in any of the above, wherein said plant is capable of forming fruits from at least about 90% of the flowers on said plant.

It is another object of the present invention to provide the pepper plant or seed as defined in any of the above, wherein said plant produces at least 38 fruits per plant, such as at least 65 fruits per plant, particularly at least 90 fruits per plant.

It is another object of the present invention to provide the pepper plant or seed as defined in any of the above, wherein said plant has fruit yield of at least about 1.5 kg per plant, preferably at least 3.6 kg per plant, more preferably at least 5.0 kg per plant.

It is another object of the present invention to provide the pepper plant or seed as defined in any of the above, wherein said plant produces a fruit type selected from the group consisting of: bell pepper, pointed pepper, half long pepper, Como di Toro pepper, sweet pepper including a dolce-type pepper, a big rectangular pepper, a conical pepper, a long conical pepper and a blocky-type pepper.

It is another object of the present invention to provide the pepper plant or seed as defined in any of the above, wherein the mature fruit of the plant is green, yellow, orange, red, ivory, brown, or purple.

It is another object of the present invention to provide the pepper plant or seed as defined in any of the above, wherein, from at least 60% of the fruits grown on said plant, at least 95% are seedless.

It is another object of the present invention to provide the pepper plant or seed as defined in any of the above, wherein the pepper plant or seed is an inbred, a dihaploid or a hybrid.

It is another object of the present invention to provide the pepper plant or seed as defined in any of the above, wherein said pepper plant or seed is a genome edited plant, such as a plant or seed produced using the CRISPR/Cas system.

It is another object of the present invention to provide the pepper plant or seed as defined in any of the above, or a progeny thereof, wherein said at least one QTL for an Excellent Parthenocarpy (EP) trait is as found in seeds of Capsicum annuum CM-192-539, representative seeds of which was deposited with NCIMB Aberdeen AB21 9YA, Scotland, UK under accession number NCIMB 44203 on 04/08/2023.

It is another object of the present invention to provide the pepper plant or seed as defined in any of the above, wherein said plant further comprising within its genome at least one additional trait selected from the group consisting of, taste, nutritional value, insect resistance, resistance to bacterial, fungal or viral disease, and resistance to a non-biotic stress, wherein the additional trait is introduced by a method selected from the group consisting of breeding, genome editing, genetic determinant introgression and transformation.

It is another object of the present invention to provide a plant part comprising at least one regenerable cell, pollen, ovule, fruit or seed of the cultivated pepper plant or seed as defined in any of the above.

It is another object of the present invention to provide a plant part of a cultivated pepper plant or seed as defined in any of the above, wherein said plant part is defined as a leaf, a bud, a meristem, an embryo, a root, a root tip, a stem, a flower, a fruit, seed or a cell.

It is another object of the present invention to provide a pepper seed obtained from a crossing in which at least one of the parents is the cultivated pepper plant or seed as defined in any of the above, or which produces the cultivated pepper plant or seed as defined in any of the above.

It is another object of the present invention to provide a tissue culture of regenerable cells, protoplasts or callus obtained from the cultivated pepper plant or seed as defined in any of the above.

It is another object of the present invention to provide pepper fruit or processed pepper fruit derived from a cultivated pepper plant or seed as defined in any of the above.

It is another object of the present invention to provide a method for producing a pepper plant or seed capable of producing high yield properties of parthenocarpic fruits, wherein said method comprises steps of: (a) producing and selecting a first pepper plant as a donor male parent, comprising at least one Quantitative Trait Locus (QTL) for an Excellent Parthenocarpy (EP) trait, said EP trait conferring production of elevated yield properties of parthenocarpic seedless fruits, as compared to a pepper plant having the same genetic background and lacking said EP trait, said QTL is selected from QTL1 located 09.0-13.5 Mbp on chromosome 1, and QTL2 located 199-218 Mbp on chromosome 10; (b) crossing said first pepper plant with a second pepper plant as a female parent comprising a cytoplasmic male sterile (CMS) trait, to produce progeny hybrid pepper plants; (c) selecting at least one progeny hybrid plant showing elevated yield properties of parthenocarpic seedless fruits as compared to the yield of a pepper plant having the same genetic background and lacking said EP trait; and (d) optionally, backcrossing said at least one selected progeny plant with said male parent plant and/or repeating steps c-d.

It is another object of the present invention to provide the method as defined above, wherein QTL1 located on chromosome 1 is associated with the property of elevated fruit number per plant and/or elevated fruit weight per plant.

It is another object of the present invention to provide the method as defined in any of the above, wherein QTL2 located on chromosome 10 is associated with the property of early fruit setting and/or setting parthenocarpic fruits at high temperature and/or setting fruit at high temperature.

It is another object of the present invention to provide the method as defined in any of the above, wherein said step of producing and selecting a pepper plant as a donor male parent comprises steps of screening F2 seeds of various pepper genetic sources for being the donor male parent comprising the EP trait by: (a) crossing F2 plants of the F2 seeds as a male parent, with a first preselected CMS line plant as a recurrent female parent, for producing a first parthenocarpic hybrid progeny set; (b) selecting from said first hybrid set a first parthenocarpic hybrid plant producing the highest parthenocarpic fruit yield relative to the other hybrid progeny plants derived from the same first recurrent female parental line; (c) selfing the F2 male parent plant of the first selected parthenocarpic hybrid to produce F3 seeds; (d) crossing plants of the F3 seeds as a male parent with the first CMS recurrent female parent to produce a second parthenocarpic hybrid progeny set and selecting from said second hybrid set a second parthenocarpic hybrid plant producing the highest parthenocarpic fruit yield relative to the other hybrid plants; (e) repeating steps a to d with the relevant/next generation seeds or plants to produce a fertile pepper plant as a donor male parent comprising the EP trait, conferring production of elevated yield of commercially acceptable parthenocarpic seedless fruits.

It is another object of the present invention to provide the method as defined in any of the above, wherein said step of screening comprises steps of producing a doubled haploid (DH) genotype plants from haploid cells derived from various pepper genetic sources.

It is another object of the present invention to provide the method as defined in any of the above, wherein said method comprises steps of inbreeding a pepper plant which is characterized by said EP trait until the genetic composition of the progeny of such inbreeding becomes substantially stable.

It is another object of the present invention to provide the method as defined in any of the above, wherein the genome of the donor pepper plant comprising the EP trait, comprises a molecular marker and/or a gene associated with the at least one QTL, said molecular marker and/or gene is selected from the group consisting of: (a) a molecular marker selected from (i) SEQ ID NO: 281-290 or any combination thereof, (ii) allele A allele A at position 9570115 as indicated in pepper genome Capsicum annuum cv CM334 v1.55 or at position 12,239,886 as indicated in Capsicum annuum cv Maor genome ASM2707369v1 NCBI, and/or (iii) a gene encoding a sequence selected from SEQ ID NO: 1-93 or any combination thereof, (iv) any combination thereof, associated with QTL1 on chromosome 1; (b) a molecular marker selected from (i) SEQ ID NO: 291-323 or any combination thereof, (ii) an allele selected from the group consisting of:

| | Position (Capsicum annuum cv |
|---|---|
| Allele | CM334 v1.55 reference genome) |
| C | 204079966 |
| T | 206006853 |
| T | 206040933 |
| C | 207234334 |
| T | 207311998 |
| A | 208017242 |
| C | 209904182 |
| T | 209904264 |
| G | 210564835 |
| T | 210564909 |
| C | 210743072 |
| G | 210819645 |
| A | 210819791 |
| T | 210820018 |
| G | 210820058 |
| C | 210909279 |
| A | 213685989 |
| A | 213686017 |
| G | 214398109 |
| C | 214593001 |
| T | 214722404 |
| C | 214853116 |
| A | 215238239 |
| A | 215250773 |
| T | 215250835 |
| A | 215940270 |
| G | 216217080 |
| T | 216959296 |
| A | 217149552 |
| C | 217149565 |
| T | 217149575 |
| T | 217245544 |
| G | 217444730 |
| A | 217492476 |
| A | 217492488 |
| A | 217501066 |
| C | 217501349 |
| C | 217504354 |
| C | 217524569 |
| G | 217538084 |
| G | 217538473 |
| A | 217613274 |
| C | 217613308 |
| T | 217613345 |
| G | 217613384 |
| G | 217613401 |
| A | 217613486 |
| T | 217613553 |
| A | 217613566 |
| C | 217613583 |
| C | 217613587 |
| C | 217613736 |
| G | 217613791 |
| A | 217613899 |
| A | 217613910 |
| A | 217629488 |
| G | 217629537 |
| A | 217629540 |
| T | 217631692 |
| C | 217631722 |
| A | 217631735 |
| T | 217631744 |
| G | 217631746 |
| C | 217802876 |
| T | 217802908 |
| G | 217874088 |
| T | 217874098 |
| G | 217874137 |
| C | 217874624 |
| T | 217874686 |
| C | 217874705 |
| A | 217875456 |
| G | 217875502 |
| T | 217875804 |
| C | 217876561 |
| T | 217876777 |
| A | 217876872 |
| G | 217876940 |
| A | 217876961 |
| G | 217876970 |
| C | 217877084 |
| T | 217877116 |
| T | 217877164 |
| G | 217877182 |
| T | 217933099 |
| G | 217933230 |
| G | 217933311 |
| G | 217933467 |
| A | 217933523 |
| C | 217933527 |
| G | 217933778 |
| A | 217933882 |
| A | 217933937 |
| G | 217934044 |
| A | 217949286 |
| T | 217950632 |
| A | 217954424 |

or any combination thereof, and/or (iii) a gene encoding a sequence selected from SEQ ID NO: 94-280 or any combination thereof, (iv) any combination thereof, associated with QTL2 on chromosome 10; and (c) any combination thereof.

It is another object of the present invention to provide the method as defined in any of the above, wherein the high yield properties of parthenocarpic fruits comprise at least one of: early fruit setting, setting fruit at high temperature, setting parthenocarpic fruits at high temperature, elevated fruit number per plant and elevated fruit weight per plant.

It is another object of the present invention to provide the method as defined in any of the above, wherein the high yield properties comprise (i) increased parthenocarpic fruit number per plant of at least about 5%, particularly 10-25%, and/or (ii) increased fruit weight per plant of at least about 15%, particularly 20-35%, as compared to a pepper plant having the same genetic background and lacking said at least one QTL associated with the at least one molecular marker and/or gene sequence.

It is another object of the present invention to provide the method as defined in any of the above, wherein the property of elevated fruit number per plant and/or elevated fruit weight per plant is associated with molecular markers and/or genes as defined above associated with QTL1 located on chromosome 1.

It is another object of the present invention to provide the method as defined in any of the above, wherein the plant comprising the at least one molecular marker and/or gene associated with QTL1 have 15-20% more fruits compared to a plant lacking said at least one QTL1 associated molecular marker and/or gene.

It is another object of the present invention to provide the method as defined in any of the above, wherein the property of early fruit setting and/or setting fruit at high temperature and/or setting parthenocarpic fruits at high temperature, is associated with molecular markers and/or genes as defined above associated with QTL2 located on chromosome 10.

It is another object of the present invention to provide the method as defined in any of the above, wherein the plant comprising the at least one molecular marker and/or gene associated with QTL2 have an average EP value of above 3, particularly in the range of 3-8.

It is another object of the present invention to provide the method as defined in any of the above, wherein the plant comprising the at least one molecular marker and/or gene associated with QTL2 exhibits an increase in EP value of about 20-40% compared to a plant lacking said at least one QTL2 associated molecular marker and/or gene.

It is another object of the present invention to provide the method as defined in any of the above, wherein the pepper plant or seed is an inbred, a dihaploid or a hybrid.

It is another object of the present invention to provide the method as defined in any of the above, wherein said pepper plant or seed is a genome edited plant, such as a plant or seed produced using the CRISPR/Cas system.

It is another object of the present invention to provide a method for producing a pepper plant or seed exhibiting high yield properties of parthenocarpic fruits, wherein said method comprises steps of introducing at least one QTL as defined in any of the above, in a Capsicum annuum plant.

It is another object of the present invention to provide the method as defined in any of the above, wherein said at least one QTL for an Excellent Parthenocarpy (EP) trait is as found in seeds of Capsicum annuum CM-192-539, representative seeds of which was deposited with NCIMB Aberdeen AB21 9YA, Scotland, UK under accession number NCIMB 44203 on 04/08/2023.

It is another object of the present invention to provide a pepper plant obtained by the method as defined in any of the above.

It is another object of the present invention to provide a pepper seed or fruit produced by the method as defined in any of the above.

It is another object of the present invention to provide a method for detecting cultivated pepper plant or seed as defined in any of the above, capable of producing high yield properties of parthenocarpic fruits, comprising the steps of detecting at least one of the following molecular markers: (a) a molecular marker selected from (i) SEQ ID NO: 281-290 or any combination thereof, (ii) allele A at position 9570115 as indicated in pepper genome Capsicum annuum cv CM334 v1.55 or at position 12,239,886 as indicated in Capsicum annuum cv Maor genome ASM2707369v1 NCBI, and/or (iii) a gene encoding a sequence selected from SEQ ID NO: 1-93 or any combination thereof, (iv) any combination thereof, associated with QTL1 located 9-13 million bp on chromosome 1; (b) a molecular marker selected from (i) SEQ ID NO: 291-323 or any combination thereof, (ii) an allele selected from the group consisting of:

| | Position (Capsicum annuum cv |
|---|---|
| Allele | CM334 v1.55 reference genome) |
| C | 204079966 |
| T | 206006853 |
| T | 206040933 |
| C | 207234334 |
| T | 207311998 |
| A | 208017242 |
| C | 209904182 |
| T | 209904264 |
| G | 210564835 |
| T | 210564909 |
| C | 210743072 |
| G | 210819645 |
| A | 210819791 |
| T | 210820018 |
| G | 210820058 |
| C | 210909279 |
| A | 213685989 |
| A | 213686017 |
| G | 214398109 |
| C | 214593001 |
| T | 214722404 |
| C | 214853116 |
| A | 215238239 |
| A | 215250773 |
| T | 215250835 |
| A | 215940270 |
| G | 216217080 |
| T | 216959296 |
| A | 217149552 |
| C | 217149565 |
| T | 217149575 |
| T | 217245544 |
| G | 217444730 |
| A | 217492476 |
| A | 217492488 |
| A | 217501066 |
| C | 217501349 |
| C | 217504354 |
| C | 217524569 |
| G | 217538084 |
| G | 217538473 |
| A | 217613274 |
| C | 217613308 |
| T | 217613345 |
| G | 217613384 |
| G | 217613401 |
| A | 217613486 |
| T | 217613553 |
| A | 217613566 |
| C | 217613583 |
| C | 217613587 |
| C | 217613736 |
| G | 217613791 |
| A | 217613899 |
| A | 217613910 |
| A | 217629488 |
| G | 217629537 |
| A | 217629540 |
| T | 217631692 |
| C | 217631722 |
| A | 217631735 |
| T | 217631744 |
| G | 217631746 |
| C | 217802876 |
| T | 217802908 |
| G | 217874088 |
| T | 217874098 |
| G | 217874137 |
| C | 217874624 |
| T | 217874686 |
| C | 217874705 |
| A | 217875456 |
| G | 217875502 |
| T | 217875804 |
| C | 217876561 |
| T | 217876777 |
| A | 217876872 |
| G | 217876940 |
| A | 217876961 |
| G | 217876970 |
| C | 217877084 |
| T | 217877116 |
| T | 217877164 |
| G | 217877182 |
| T | 217933099 |
| G | 217933230 |
| G | 217933311 |
| G | 217933467 |
| A | 217933523 |
| C | 217933527 |
| G | 217933778 |
| A | 217933882 |
| A | 217933937 |
| G | 217934044 |
| A | 217949286 |
| T | 217950632 |
| A | 217954424 |

or any combination thereof, and/or (iii) a gene encoding a sequence selected from SEQ ID NO: 94-280 or any combination thereof, (iv) any combination thereof, associated with QTL2 located on chromosome 10; and (c) any combination thereof.

It is another object of the present invention to provide a method for selecting cultivated pepper plant or seed as defined in any of the above, capable of producing high yield properties of parthenocarpic fruits, said method comprising the steps of: (1) assaying pepper plants for at least one molecular marker genetically linked to at least one QTL selected from QTL1 located 09.0-13.5 Mbp on chromosome 1 and QTL2 located 199-218 Mbp on chromosome 10, said molecular marker is selected from: (a) a molecular marker selected from (i) SEQ ID NO: 281-290 or any combination thereof, (ii) allele A at position 9570115 as indicated in pepper genome Capsicum annuum cv CM334 v1.55 or at position 12,239,886 as indicated in Capsicum annuum cv Maor genome ASM2707369v1 NCBI, and/or (iii) a gene encoding a sequence selected from SEQ ID NO: 1-93 or any combination thereof, (iv) or any combination thereof, associated with QTL1 on chromosome 1; (b) a molecular marker selected from (i) SEQ ID NO: 291-323 or any combination thereof, (ii) an allele selected from the group consisting of:

| | Position (Capsicum annuum cv |
|---|---|
| Allele | CM334 v1.55 reference genome) |
| C | 204079966 |
| T | 206006853 |
| T | 206040933 |
| C | 207234334 |
| T | 207311998 |
| A | 208017242 |
| C | 209904182 |
| T | 209904264 |
| G | 210564835 |
| T | 210564909 |
| C | 210743072 |
| G | 210819645 |
| A | 210819791 |
| T | 210820018 |
| G | 210820058 |
| C | 210909279 |
| A | 213685989 |
| A | 213686017 |
| G | 214398109 |
| C | 214593001 |
| T | 214722404 |
| C | 214853116 |
| A | 215238239 |
| A | 215250773 |
| T | 215250835 |
| A | 215940270 |
| G | 216217080 |
| T | 216959296 |
| A | 217149552 |
| C | 217149565 |
| T | 217149575 |
| T | 217245544 |
| G | 217444730 |
| A | 217492476 |
| A | 217492488 |
| A | 217501066 |
| C | 217501349 |
| C | 217504354 |
| C | 217524569 |
| G | 217538084 |
| G | 217538473 |
| A | 217613274 |
| C | 217613308 |
| T | 217613345 |
| G | 217613384 |
| G | 217613401 |
| A | 217613486 |
| T | 217613553 |
| A | 217613566 |
| C | 217613583 |
| C | 217613587 |
| C | 217613736 |
| G | 217613791 |
| A | 217613899 |
| A | 217613910 |
| A | 217629488 |
| G | 217629537 |
| A | 217629540 |
| T | 217631692 |
| C | 217631722 |
| A | 217631735 |
| T | 217631744 |
| G | 217631746 |
| C | 217802876 |
| T | 217802908 |
| G | 217874088 |
| T | 217874098 |
| G | 217874137 |
| C | 217874624 |
| T | 217874686 |
| C | 217874705 |
| A | 217875456 |
| G | 217875502 |
| T | 217875804 |
| C | 217876561 |
| T | 217876777 |
| A | 217876872 |
| G | 217876940 |
| A | 217876961 |
| G | 217876970 |
| C | 217877084 |
| T | 217877116 |
| T | 217877164 |
| G | 217877182 |
| T | 217933099 |
| G | 217933230 |
| G | 217933311 |
| G | 217933467 |
| A | 217933523 |
| C | 217933527 |
| G | 217933778 |
| A | 217933882 |
| A | 217933937 |
| G | 217934044 |
| A | 217949286 |
| T | 217950632 |
| A | 217954424 |

or any combination thereof, and/or (iii) a gene encoding a sequence selected from SEQ ID NO: 94-280 or any combination thereof, (iv) any combination thereof, associated with QTL2 on chromosome 10; and (c) any combination thereof; and (2) selecting a plant comprising at least one of the molecular markers associated with at least one of the QTLs for EP trait conferring production of elevated yield of parthenocarpic seedless fruits, as compared to a pepper plant having the same genetic background and lacking said EP trait.

It is another object of the present invention to provide a Method for the production of a pepper (Capsicum annuum) plant exhibiting high yield properties of parthenocarpic fruits, by using a doubled haploid generation technique on plant material comprising the QTL as defined in any of the above, to generate a doubled haploid line comprising said trait and the QTL as defined in any of the above.

It is another object of the present invention to provide an allele, haplotype, QTL, molecular marker or gene being inherited to progeny plant, wherein said allele, haplotype, QTL, molecular marker or gene is associated with an Excellent Parthenocarpy (EP) trait capable of conferring production of elevated yield of parthenocarpic seedless fruits as compared to a pepper plant having the same genetic background and lacking said allele, haplotype, QTL, molecular marker or gene, said molecular marker and/or gene sequence is selected from the group consisting of: (a) a molecular marker selected from (i) SEQ ID NO: 281-290 or any combination thereof, (ii) allele A at position 9570115 as indicated in pepper genome Capsicum annuum cv CM334 v1.55 or at position 12,239,886 as indicated in Capsicum annuum cv Maor genome ASM2707369v1 NCBI, and/or (iii) a gene encoding a sequence selected from SEQ ID NO: 1-93 or any combination thereof, (iv) or any combination thereof, associated with QTL1 located 09.0-13.5 Mbp on chromosome 1; (b) a molecular marker selected from (i) SEQ ID NO: 291-323 or any combination thereof, (ii) an allele selected from the group consisting of:

| | Position (Capsicum annuum cv |
|---|---|
| Allele | CM334 v1.55 reference genome) |
| C | 204079966 |
| T | 206006853 |
| T | 206040933 |
| C | 207234334 |
| T | 207311998 |
| A | 208017242 |
| C | 209904182 |
| T | 209904264 |
| G | 210564835 |
| T | 210564909 |
| C | 210743072 |
| G | 210819645 |
| A | 210819791 |
| T | 210820018 |
| G | 210820058 |
| C | 210909279 |
| A | 213685989 |
| A | 213686017 |
| G | 214398109 |
| C | 214593001 |
| T | 214722404 |
| C | 214853116 |
| A | 215238239 |
| A | 215250773 |
| T | 215250835 |
| A | 215940270 |
| G | 216217080 |
| T | 216959296 |
| A | 217149552 |
| C | 217149565 |
| T | 217149575 |
| T | 217245544 |
| G | 217444730 |
| A | 217492476 |
| A | 217492488 |
| A | 217501066 |
| C | 217501349 |
| C | 217504354 |
| C | 217524569 |
| G | 217538084 |
| G | 217538473 |
| A | 217613274 |
| C | 217613308 |
| T | 217613345 |
| G | 217613384 |
| G | 217613401 |
| A | 217613486 |
| T | 217613553 |
| A | 217613566 |
| C | 217613583 |
| C | 217613587 |
| C | 217613736 |
| G | 217613791 |
| A | 217613899 |
| A | 217613910 |
| A | 217629488 |
| G | 217629537 |
| A | 217629540 |
| T | 217631692 |
| C | 217631722 |
| A | 217631735 |
| T | 217631744 |
| G | 217631746 |
| C | 217802876 |
| T | 217802908 |
| G | 217874088 |
| T | 217874098 |
| G | 217874137 |
| C | 217874624 |
| T | 217874686 |
| C | 217874705 |
| A | 217875456 |
| G | 217875502 |
| T | 217875804 |
| C | 217876561 |
| T | 217876777 |
| A | 217876872 |
| G | 217876940 |
| A | 217876961 |
| G | 217876970 |
| C | 217877084 |
| T | 217877116 |
| T | 217877164 |
| G | 217877182 |
| T | 217933099 |
| G | 217933230 |
| G | 217933311 |
| G | 217933467 |
| A | 217933523 |
| C | 217933527 |
| G | 217933778 |
| A | 217933882 |
| A | 217933937 |
| G | 217934044 |
| A | 217949286 |
| T | 217950632 |
| A | 217954424 |

or any combination thereof, and/or (iii) a gene encoding a sequence selected from SEQ ID NO: 94-280 or any combination thereof, (iv) any combination thereof, associated with QTL2 located 199-218 Mbp on chromosome 10; and (c) any combination thereof.

It is another object of the present invention to provide the allele, haplotype, QTL, genetic marker or gene as defined in any of the above, having at least 90% sequence identity with the allele, haplotype, genetic marker or gene as defined above, wherein said allele, haplotype, genetic marker or gene is associated with an Excellent Parthenocarpy (EP) trait capable of conferring production of elevated yield of parthenocarpic seedless fruits as compared to a pepper plant having the same genetic background and lacking said EP trait.

It is another object of the present invention to provide use of a molecular marker as defined in any of the above, to identify pepper plant or seed capable of producing high yield properties of parthenocarpic fruits, wherein said plant comprises at least one Quantitative Trait Locus (QTL) for an Excellent Parthenocarpy (EP) trait, said EP trait conferring production of elevated yield of parthenocarpic seedless fruits, as compared to a pepper plant having the same genetic background and lacking said EP trait.

It is another object of the present invention to provide use of isolated sequences, or sequences having at least 90% sequence identity with the sequences as defined in any of the above, for detection and/or production of a pepper plant comprising an Excellent Parthenocarpy (EP) trait conferring production of elevated yield of commercially acceptable parthenocarpic seedless fruits, as compared to a pepper plant having the same genetic background and lacking said EP trait.

It is another object of the present invention to provide use of a molecular marker selected from the group consisting of (a) a molecular marker selected from (i) SEQ ID NO: 281-290 or any combination thereof, (ii) allele A at position 9570115 as indicated in pepper genome Capsicum annuum cv CM334 v1.55 or at position 12,239,886 as indicated in Capsicum annuum cv Maor genome ASM2707369v1 NCBI, and/or (iii) a gene encoding a sequence selected from SEQ ID NO: 1-93 or any combination thereof, (iv) any combination thereof associated with QTL1 located 09-13.5 Mbp on chromosome 1; (b) a molecular marker selected from (i) SEQ ID NO: 291-323 or any combination thereof, (ii) an allele selected from the group consisting of:

| | Position (Capsicum annuum cv |
|---|---|
| Allele | CM334 v1.55 reference genome) |
| C | 204079966 |
| T | 206006853 |
| T | 206040933 |
| C | 207234334 |
| T | 207311998 |
| A | 208017242 |
| C | 209904182 |
| T | 209904264 |
| G | 210564835 |
| T | 210564909 |
| C | 210743072 |
| G | 210819645 |
| A | 210819791 |
| T | 210820018 |
| G | 210820058 |
| C | 210909279 |
| A | 213685989 |
| A | 213686017 |
| G | 214398109 |
| C | 214593001 |
| T | 214722404 |
| C | 214853116 |
| A | 215238239 |
| A | 215250773 |
| T | 215250835 |
| A | 215940270 |
| G | 216217080 |
| T | 216959296 |
| A | 217149552 |
| C | 217149565 |
| T | 217149575 |
| T | 217245544 |
| G | 217444730 |
| A | 217492476 |
| A | 217492488 |
| A | 217501066 |
| C | 217501349 |
| C | 217504354 |
| C | 217524569 |
| G | 217538084 |
| G | 217538473 |
| A | 217613274 |
| C | 217613308 |
| T | 217613345 |
| G | 217613384 |
| G | 217613401 |
| A | 217613486 |
| T | 217613553 |
| A | 217613566 |
| C | 217613583 |
| C | 217613587 |
| C | 217613736 |
| G | 217613791 |
| A | 217613899 |
| A | 217613910 |
| A | 217629488 |
| G | 217629537 |
| A | 217629540 |
| T | 217631692 |
| C | 217631722 |
| A | 217631735 |
| T | 217631744 |
| G | 217631746 |
| C | 217802876 |
| T | 217802908 |
| G | 217874088 |
| T | 217874098 |
| G | 217874137 |
| C | 217874624 |
| T | 217874686 |
| C | 217874705 |
| A | 217875456 |
| G | 217875502 |
| T | 217875804 |
| C | 217876561 |
| T | 217876777 |
| A | 217876872 |
| G | 217876940 |
| A | 217876961 |
| G | 217876970 |
| C | 217877084 |
| T | 217877116 |
| T | 217877164 |
| G | 217877182 |
| T | 217933099 |
| G | 217933230 |
| G | 217933311 |
| G | 217933467 |
| A | 217933523 |
| C | 217933527 |
| G | 217933778 |
| A | 217933882 |
| A | 217933937 |
| G | 217934044 |
| A | 217949286 |
| T | 217950632 |
| A | 217954424 |

or any combination thereof, and/or (iii) a gene encoding a sequence selected from SEQ ID NO: 94-280 or any combination thereof, (iv) any combination thereof, associated with QTL2 located 199-218 Mbp on chromosome 10; and (c) any combination thereof; to identify or develop pepper plants comprising an Excellent Parthenocarpy (EP) trait conferring production of elevated yield of commercially acceptable parthenocarpic seedless fruits, as defined in any of the above, and/or identify the at least one QTL, and/or to develop other markers linked to the at least one QTL.

It is another object of the present invention to provide Pepper genetic markers, sequences or elements, plants, seeds, fruits as described in any one of the above and plant products thereof, for the use in multiple geographical- and/or weather-related environments and growth conditions.

It is another object of the present invention to provide use of a seed deposited under NCIMB accession number 44203, with NCIMB Aberdeen AB21 9YA, Scotland on Apr. 8, 2023, for the production of the pepper plant as defined in any of the above.

It is another object of the present invention to provide use of a pepper plant, which plant carries the at least one QTL as defined in any of the above, that confers Excellent Parthenocarpy (EP) trait, said EP trait conferring production of elevated yield of parthenocarpic seedless fruits, as compared to a pepper plant having the same genetic background and lacking said EP trait, said QTL is selected from QTL1 located 09.0-13.5 Mbp on chromosome 1 and QTL2 located 199-218 Mbp on chromosome 10, which the at least one QTL is obtainable from a Capsicum annuum pepper plant carrying the QTL as defined in any of the above, in particular a pepper plant grown from seed of which a representative sample was deposited under accession number NCIMB 44203, deposited with NCIMB Aberdeen AB21 9YA, Scotland on Apr. 8, 2023, as a crop.

It is another object of the present invention to provide a method for increasing parthenocarpic seedless pepper fruit yield production to a commercially relevant extent in multiple geographical- and/or whether-related environments or areas or growth conditions comprising growing in said geographical area pepper plant or seed as defined in any of the above.

It is another object of the present invention to provide a genome modified pepper plant or seed capable of producing high yield properties of parthenocarpic fruits, wherein said plant or seed comprises at least one Quantitative Trait Locus (QTL) for an Excellent Parthenocarpy (EP) trait, said EP trait conferring production of elevated yield properties of parthenocarpic seedless fruits, as compared to a pepper plant having the same genetic background and lacking said EP trait, said at least one QTL is selected from QTL1 located 09.0-13.5 Mbp on chromosome 1, and QTL2 located 199-218 Mbp on chromosome 10, and comprises genome modifications selected from: (a) at least one genome modification associated with QTL1 on chromosome 1 selected from (i) SEQ ID NO: 281-290 or any combination thereof, (ii) allele A at position 9570115 as indicated in pepper genome Capsicum annuum cv CM334 v1.55 or at position 12,239,886 as indicated in Capsicum annuum cv Maor genome ASM2707369v1 NCBI, and/or (iii) a gene encoding a sequence selected from SEQ ID NO: 1-93 or any combination thereof, (iv) any combination thereof; (b) at least one genome modification associated with QTL2 on chromosome 10 selected from (i) SEQ ID NO: 291-323 or any combination thereof, (ii) an allele selected from the group consisting of:

| | Position (Capsicum annuum cv |
|---|---|
| Allele | CM334 v1.55 reference genome) |
| C | 204079966 |
| T | 206006853 |
| T | 206040933 |
| C | 207234334 |
| T | 207311998 |
| A | 208017242 |
| C | 209904182 |
| T | 209904264 |
| G | 210564835 |
| T | 210564909 |
| C | 210743072 |
| G | 210819645 |
| A | 210819791 |
| T | 210820018 |
| G | 210820058 |
| C | 210909279 |
| A | 213685989 |
| A | 213686017 |
| G | 214398109 |
| C | 214593001 |
| T | 214722404 |
| C | 214853116 |
| A | 215238239 |
| A | 215250773 |
| T | 215250835 |
| A | 215940270 |
| G | 216217080 |
| T | 216959296 |
| A | 217149552 |
| C | 217149565 |
| T | 217149575 |
| T | 217245544 |
| G | 217444730 |
| A | 217492476 |
| A | 217492488 |
| A | 217501066 |
| C | 217501349 |
| C | 217504354 |
| C | 217524569 |
| G | 217538084 |
| G | 217538473 |
| A | 217613274 |
| C | 217613308 |
| T | 217613345 |
| G | 217613384 |
| G | 217613401 |
| A | 217613486 |
| T | 217613553 |
| A | 217613566 |
| C | 217613583 |
| C | 217613587 |
| C | 217613736 |
| G | 217613791 |
| A | 217613899 |
| A | 217613910 |
| A | 217629488 |
| G | 217629537 |
| A | 217629540 |
| T | 217631692 |
| C | 217631722 |
| A | 217631735 |
| T | 217631744 |
| G | 217631746 |
| C | 217802876 |
| T | 217802908 |
| G | 217874088 |
| T | 217874098 |
| G | 217874137 |
| C | 217874624 |
| T | 217874686 |
| C | 217874705 |
| A | 217875456 |
| G | 217875502 |
| T | 217875804 |
| C | 217876561 |
| T | 217876777 |
| A | 217876872 |
| G | 217876940 |
| A | 217876961 |
| G | 217876970 |
| C | 217877084 |
| T | 217877116 |
| T | 217877164 |
| G | 217877182 |
| T | 217933099 |
| G | 217933230 |
| G | 217933311 |
| G | 217933467 |
| A | 217933523 |
| C | 217933527 |
| G | 217933778 |
| A | 217933882 |
| A | 217933937 |
| G | 217934044 |
| A | 217949286 |
| T | 217950632 |
| A | 217954424 |

or any combination thereof, and/or (iii) a gene encoding a sequence selected from SEQ ID NO: 94-280 or any combination thereof, (iv) any combination thereof; and (c) any combination thereof.

It is another object of the present invention to provide the genome modified pepper plant or seed as defined in any of the above, wherein said genome modifications are produced using targeted genome editing technique, such as using the CRISPR/Cas9 system.

It is another object of the present invention to provide the genome modified pepper plant or seed as defined in any of the above, wherein the genome modifications located on chromosome 1 are associated with the property of elevated fruit number per plant and/or elevated fruit weight per plant and wherein the genome modifications located on chromosome 10 are associated with the property of early fruit setting and/or setting parthenocarpic fruits at high temperature and/or setting fruit at high temperature.

### BRIEF DESCRIPTION OF THE DRAWINGS

FIG. 1 presents a breeding scheme describing the resultant hybrid varieties and their parental lines;

FIG. 2 presents a breeding scheme for generating F2 population for genotyping markers associated with the high yield trait of the present invention;

FIG. 3 illustrates Manhattan PLOTS for the F2 population individuals according to fruits number parameter; and

FIG. 4 illustrates Manhattan PLOTS for the F2 population individuals according to EP visual evaluation.

### DETAILED DESCRIPTION OF THE INVENTION

The present invention is now described more fully hereinafter with reference to the accompanying examples and drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

According to one embodiment, the invention provides a cultivated pepper plant or seed capable of producing high yield properties of parthenocarpic fruits. The plant or seed comprises at least one Quantitative Trait Locus (QTL) for an Excellent Parthenocarpy (EP) trait, said EP trait conferring production of elevated yield properties of parthenocarpic seedless fruits, as compared to a pepper plant having the same genetic background and lacking said EP trait, said QTL is selected from QTL1 located 09.0-13.5 Mbp on chromosome 1, and QTL2 located 199-218 Mbp on chromosome 10.

It is within the scope of the present invention that the cultivated pepper plant or seed as defined above, comprises at least one allele, haplotype, molecular marker, single nucleotide polymorphism (SNP), gene and/or genetic determinant associated with the at least one QTL (molecular markers and/or genes identified by the present invention, to be associated by the EP trait are defined and detailed below).

The EP (excellent parthenocarpy) trait is shown to lead to:
a. Yield increase (fruit number and/or weight), for which unique genes/DNA sequences associated with the trait were identified on QTL1 of chromosome 1 (SEQ ID NO: 1-93, SEQ ID NO: 281-290 and Table 8); and
b. Early fruit setting/setting fruit at high temperature, and/or setting parthenocarpic fruit at high temperature, for which unique genes/DNA sequences associated with the trait were identified on QTL2 of Chromosome 10 (SEQ ID NO: 94-280, SEQ ID NO: 291-323 and Table 9).

It is further within the scope that the pepper plants or seeds of the present invention are produced using targeted genome editing, e.g. using the CRISPR (Clustered Regularly Interspaced Short Palindromic Repeats)/Cas (such as Cas9) technology or system, or any other genetic modification method known in the relevant art to generate the herein described lines/variants/sequences/SNPs.

Thus, the cultivated pepper plant or seed of the present invention, may be a genome edited plant or seed, such as a plant or seed produced by the CRISPR/Cas system.

It is further within the scope of the present invention that the cultivated pepper plant or seed is an inbred, a dihaploid or a hybrid.

According to further aspects, the present invention provides a pepper plant or seed capable of producing high yield properties of parthenocarpic fruits. The plant comprises an introgressed Excellent Parthenocarpy (EP) trait, said EP trait conferring production of elevated yield of commercially acceptable parthenocarpic seedless fruits, as compared to a pepper plant having the same genetic background and lacking said EP trait.

In the context of the embodiments of the invention, the term “plant” is meant to be understood as whole plant, grafted plant, ancestors and progeny of the plants, or any parts or derivatives thereof. According to the present invention, a non-liming list of plant part includes plant cells, plant protoplasts, plant tissue, plant cell, plant organ, suspension cultures, plant cell or tissue culture from which pepper plants can be regenerated, plant callus or calli, meristematic regions, meristematic cells, gametophytes, sporophyte, microspores, embryos, immature embryos, pollen, ovules, egg cells, zygotes, anthers, fruit (e.g. harvested pepper fruit), flowers, flower parts, scion, leaves, cotyledons, pistil, stem, anther, seeds, seed coat, cutting, seed coat, roots, root tips, rootstock, shoot, bud, meristem, and the like. As used herein, the term “plant part” is interchangeable with “plant material”.

In the context of the embodiments of the invention, the term “plant cell” refers, without limitation, to a structural and physiological unit of a plant, comprising a protoplast and a cell wall. The plant cell may be in the form of an isolated single cell or a cultured cell, or as a part of higher organized unit such as, for example, plant tissue, a plant organ, or a whole plant.

In the context of the embodiments of the invention, the term “plant cell culture” refers, without limitation, to cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, fruit, seeds, seed coat or any combination thereof.

In the context of the embodiments of the invention, the term “plant organ” refers, without limitation, to a distinct and visibly structured and differentiated part of a plant such as a root, stem, leaf, flower, flower bud, embryo, and the like.

In the context of the embodiments of the invention, the term “plant tissue” refers, without limitation, to a group of plant cells organized into a structural and functional unit. Any tissue of a plant in planta or in culture is included. This term includes, but is not limited to, whole plants, plant organs, plant seeds, tissue culture, protoplasts, meristematic cells, calli and any group of plant cells organized into structural and/or functional units. The use of this term in conjunction with, or in the absence of, any specific type of plant tissue as listed above or otherwise embraced by this definition is not intended to be exclusive of any other type of plant tissue.

In particular embodiments of the present invention, the plant part is further defined as a pepper fruit that lacks internal carpelloid structures. In the context of the embodiments of the invention, the term “carpelloid structures” refers to, without limitation, to a structure that mimics the role of seeds and support parthenocarpic fruit growth.

In the context of the embodiments of the invention, the term “parthenocarpic” refers, without limitation, to seedless fruits, i.e., fruits that have been developed without pollination and/or fertilization. Usually, fruits generated through parthenocarpy are seedless. Some types of parthenocarpy are genetically determined while other types of parthenocarpy are determined by cultivation environments. The parthenocarpy referred to as in the present invention is the genetically determined type. Plants having a genetically determined parthenocarpic trait are preferred for the case of edible cultivation plants, because they offer high reliability and reproducibility as well as enabling the reduction of the labor for managing the cultivation environment. As used herein, the term “parthenocarpic” is interchangeable with “parthenocarpic fruit”, “parthenocarpy”, “seedless fruits”, or “parthenocarpic seedless fruits”.

In the context of the embodiments of the invention, the term “pepper” refers, without limitation, to the common name given to many plants, their fruits and to the spices obtained from their fruits, usually with a spicy flavor (resulting from the capsaicin). The pepper plants can include plants from the genus Capsicum, Pipper and Pimenta. Capsicum is a genus of flowering plants (angiosperms) comprised of the nightshade family Solanaceae. It is generally accepted that the Capsicum genus originated in Bolivia and consists of 25-30 species. In the context of the embodiments of the invention, the pepper plant refers, without limitation, to the cultivated species Capsicum annuum, Capsicum chinense, Capsicum baccatum, Capsicum frutescens and Capsicum pubescens, and to the wild species. Capsicum annuum comprises both non-pungent and pungent (chili) peppers¹. Furthermore, the term “pepper” also includes, without limitations, plants called by names other than “pepper”, e.g., horticultural crops called “piment”, “paprika”, and “sweet pepper”. In the context of the embodiments of the invention, the pepper plant is preferably Capsicum annuum. In the context of the embodiments, the Capsicum annuum is selected from a fruit type including, but not limited to, bell pepper, pointed pepper, half long pepper, Como di Toro pepper, sweet pepper including a dolce-type pepper, a big rectangular pepper, a conical pepper, a long conical pepper and a blocky-type pepper.

In the context of the embodiments of the invention, the term “introgressed” refers to incorporation (usually via hybridization and backcrossing) of alleles from one species into the gene pool of a second, divergent species. The introgression is made by means of repeated backcrosses between and hybrid and the first plant generation. As used herein, the term “introgression” is interchangeable with “introgression”, “introgressing” or “introgressive hybridization”. An introgression may also be described as a heterologous genetic material stably integrated in the genome of a recipient plant.

In the context of the embodiments of the invention, the term “backcrossing” refers, without limitation, to the repeatedly crossing of a hybrid with one of its parents, or an adult genetically identical to the parent, to achieve offspring with a genetic identity closer to parents. In some embodiments, the backcrossing process refers to the repeated crossing of a hybrid progeny back to one of the parental pepper plants. The parental pepper plant, which contributes the gene for the desired characteristic, is termed the nonrecurrent or donor parent. This terminology refers to the fact that the nonrecurrent parent is used one time in the backcross protocol and therefore does not recur. The parental pepper plant to which the gene or genes from the nonrecurrent parent are transferred is known as the recurrent parent as it is used for several rounds in the backcrossing protocol. In a typical backcross protocol, a plant from the original varieties of interest (recurrent parent) is crossed to a plant selected from second varieties (nonrecurrent parent) that carries the single gene of interest to be transferred. The resulting progeny from this cross are then crossed again to the recurrent parent and the process is repeated until a pepper plant is obtained wherein essentially all of the desired morphological and physiological characteristics of the recurrent parent are recovered in the converted plant, in addition to the single transferred gene from the nonrecurrent parent. Backcrossing methods can be used with the present invention to improve or introduce a characteristic into the parent lines.

In the context of the embodiments of the invention, the term “trait” refers, without limitation, to the appearance of other detectable characteristic or phenotype of an individual, resulting from the interaction of its genome, proteome and/or metabolome with the environment. A trait may be inherited in a dominant or recessive manner, or in a partial or incomplete-dominant manner. A trait may be monogenic (i.e. determined by a single locus) or polygenic (i.e. determined by more than one locus) or may also result from the interaction of one or more genes with the environment. A dominant trait results in a complete phenotypic manifestation at heterozygous or homozygous state; conventionally, a recessive trait manifests itself only when present at homozygous state. For example, in the context of the present invention, the Excellent Parthenocarpy (EP) trait confers the production of elevated yield of commercially acceptable parthenocarpic seedless fruits on the pepper plants or seeds as described herein.

The EP (excellent parthenocarpy) trait is shown to lead to:
a. Yield increase (fruit number and/or weight), for which unique genes/DNA sequences associated with the trait were identified on QTL1 of chromosome 1 (SEQ ID NO: 1-93, SEQ ID NO: 281-290 and Table 8); and
b. Early fruit setting/setting fruit at high temperature and/or setting parthenocarpic fruit at high temperature, for which unique genes/DNA sequences associated with the trait were identified on QTL2 of Chromosome 10 (SEQ ID NO: 94-280, SEQ ID NO: 291-323 and Table 9).

According to a further embodiment, the cultivated pepper plant or seed of the present invention produces parthenocarpic seedless fruits which are commercially acceptable or marketable as defined by different market, segment and fruit type.

In the context of the embodiments of the invention, the term “phenotype” refers, without limitation, to distinguishable characteristics from a genetically controlled trait.

In the context of the embodiments of the invention, the term “homozygous” refers, without limitation, to a genetic condition or configuration existing when two identical or like alleles reside at a specific locus but are positioned individually on corresponding pairs of homologous chromosomes in the cell of a diploid organism. Conversely, as used herein, the term “heterozygous” means a genetic condition or configuration existing when two different or unlike alleles reside at a specific locus but are positioned individually on corresponding pairs of homologous chromosomes in the cell of a diploid organism. In specific embodiments, the hybrid pepper plants of the present invention comprise heterozygous configuration of the genetic markers associated with the high yield characteristics.

An important way to measure parthenocarpy traits is through Parthenocarpy degree. The currently available commercial seedless pepper variety has an EP between 2.9 and 3.1.

In the context of the embodiments of the invention, the term “Excellent Parthenocarpy (EP) trait” refers, without limitation, to pepper fruit yield from selected lines that have an Excellent Parthenocarpy (EP) phenotype of level above 3, preferably above 5, and more preferably about 8, considerably higher than the known or commercially available lines, with an EP phenotype level around 3.

It is within the scope that Excellent Parthenocarpy (EP) evaluation is a qualitative trait, measured visually. It is an internal grade (a value between 0-10) given to each of the tested lines for evaluating their fruit yield. In certain embodiments, the EP grade or level or value herein means a grade given to each plot by visually evaluating the fruit yield in the particular plot. EP grade is a relative evaluation, where the relative point used is a commercial seedless pepper variety that has EP in the range of 2.9-3.1. The EP grade represents the early fruit set; it means fruits that are observed on the plant up to 60-80 cm from the ground.

According to some embodiments of the above pepper or plant seed, the plant is male sterile, the male sterility is a cytoplasmic male-sterile (CMS) trait, a genomic male sterile (GMS) trait or a combination thereof.

In the context of the embodiments of the invention, the term “cytoplasmic male-sterile (CMS)” refers, without limitation, to total or partial male sterility in plants as the result of specific nuclear genes alone (GMS) or of specific mitochondrial and nuclear genes interactions (CMS). In the context of the embodiments of the invention, male sterility is the failure of plants to produce functional anthers, pollen, or male gametes.

The pepper Cytoplasmic Male Sterility (“CMS”) system or Cytoplasmic Genic Male Sterility (“CGMS”), for which the male sterility factors are coded in the mitochondrial DNA, can be used for the production of fully male sterile progeny. The cytoplasm, including the mitochondria, is passed from the female parent to its progeny. Therefore, the progeny plants coming from a cross between two parental plants, one used as female and one used as male, will carry the cytoplasm of the female parental plant. Therefore, if this female parental plant displays the CMS trait, then it may be expected that all progeny will also carry that trait. According to some embodiments, plants with genetic male sterility obtained via CMS or GMS, refers to plants that are not usually capable of breeding from self-pollination, but are capable of breeding from cross-pollination.

According to some embodiments, a “female parent” refers to a pepper plant that is the recipient of pollen from a male donor line, which pollen successfully pollinates an egg. A female parent can be any pepper plant that is the recipient of pollen. Such female parents can be male sterile, for example, because of genetic male sterility, cytoplasmic male sterility, or because they have been subject to manual emasculation of the stamens. Genetic or cytoplasmic male sterility can be manifested in different manners, such as sterile pollen, malformed or stamenless flowers, positional sterility, and functional sterility.

It is further within the scope of the present invention that “male parent plant” refers to a parent plant that provides pollen to (i.e. is a pollinator for) a female line. They may be useful for breeding of progeny pepper plants, such as parthenocarpic seedless progeny plants.

According to some embodiments of the above pepper or plant seed, the male sterility is preferably a cytoplasmic male-sterile (CMS) trait.

According to some embodiments of the above pepper or plant seed, the plant comprises at least one allele, haplotype, genetic marker, gene encoding sequence or genetic determinant associated with said EP trait.

In the context of the embodiments of the invention, the term “allele” refers, without limitation, to one or more variant forms of DNA sequence (a single base or a segment of bases) at a given genomic location (gene locus) and relates to a trait or characteristic of an individual. Diploid cells or organisms inherit two alleles, one from each parent, for any given genomic location (locus, or loci in plural), on a pair of homologous chromosomes, where such variation exists. One allele is present on each chromosome of the pair of homologous chromosomes. If the two alleles are the same, the individual is homozygous for that allele. If the alleles are different, the individual is heterozygous. A diploid plant species may comprise a large number of different alleles at a particular locus. Such alternative or variant forms of alleles may be the result of single nucleotide polymorphisms, insertions, inversions, translocations or deletions, or the consequence of gene regulation caused by, for example, by chemical or structural modification, transcription regulation or post-translational modification/regulation. An allele associated with a qualitative trait may comprise alternative or variant forms of various genetic units including those that are identical or associated with a single gene or multiple genes or their products or even a gene disrupting or controlled by a genetic factor contributing to the phenotype represented by the locus.

In the context of the embodiments of the invention, the term “locus” (loci plural) refers, without limitation, to a specific place or places or region or a site on a chromosome where for example a gene or genetic marker element or factor is found. In specific embodiments, such a genetic element is contributing to a trait.

In the context of the embodiments of the invention, the term “haplotype” refers, without limitation, to a physical grouping of alleles (DNA sequences) from adjacent loci (locations) on a chromosome that tend to be inherited together. A haplotype may be one locus, several loci, or an entire chromosome depending on the number of recombination events that have occurred between a given set of loci. A specific haplotype typically reflects a unique combination of variants that reside near each other on a chromosome. “Haplotype” further refers to a set of single-nucleotide polymorphisms (SNPs) on a single chromosome of a chromosome pair that are associated statistically.

In the context of the embodiments of the invention, the term “genetic marker” or “molecular marker” or “marker” refers, without limitation, to a DNA sequence with a known physical location on a chromosome, and indicates the presence of at least one genotype, polymorphism or phenotype. According to the present invention, a non-liming list of genetic markers includes single nucleotide polymorphisms (SNPs), cleavable amplified polymorphic sequences (CAPS), amplified fragment length polymorphisms (AFLPs), restriction fragment length polymorphisms (RFLPs), simple sequence repeats (SSRs), insertion(s)/deletion(s) (“INDEL”(s)), inter-simple sequence repeats (ISSR), and random amplified polymorphic DNA (RAPD) sequences. Preferably, the genetic marker is a SNP. Genetic markers are used to track the inheritance of a nearby gene that has not yet been identified, but whose approximate location is known. The genetic marker itself may be a part of a gene or may have no known function. As used herein, the term “genetic marker” is interchangeable with “molecular marker” or “DNA marker” or “biomarker” and can also refer to a polynucleotide sequence complementary or corresponding to a genomic sequence, such as a sequence of a nucleic acid used as a probe or primer. A genetic marker can be physically located in a position on a chromosome that is within or outside of the genetic locus with which it is associated (i.e., is intragenic or extragenic, respectively). In some embodiments of the present invention, the one or more genetic markers comprise a combination of two or more genetic markers. It is also within the scope of the present invention that different combinations of genetic markers are used to identify different traits or phenotypic characteristics as disclosed inter alia.

It is further within the context of the present invention that a “marker” is an indicator for the presence of at least one phenotype, genotype, trait or polymorphism. Markers include, but are not limited to, single nucleotide polymorphisms (SNPs), cleavable amplified polymorphic sequences (CAPS), amplified fragment length polymorphisms (AFLPs), restriction fragment length polymorphisms (RFLPs), simple sequence repeats (SSRs), insertion(s)/deletion(s) (“INDEL”(s)), inter-simple sequence repeats (ISSR), and random amplified polymorphic DNA (RAPD) sequences. A marker is preferably inherited in codominant fashion (both alleles at a locus in a diploid heterozygote are readily detectable), with no environmental variance component. A “nucleic acid marker” as used herein means a nucleic acid molecule that is capable of being a marker for detecting a polymorphism, phenotype, or both associated with a trait of interest. A “marker assay” generally means a method for detecting a polymorphism at a particular locus using a particular method, e.g. measurement of at least one phenotype (such as a visually detectable trait, e.g. fruit yield and/or excellent parthenocarpy), restriction fragment length polymorphism (RFLP), single base extension, electrophoresis, sequence alignment, allelic specific oligonucleotide hybridization (ASO), random amplified polymorphic DNA (RAPD), microarray-based technologies, PCR-based technologies, and nucleic acid sequencing technologies, etc.

In the context of the embodiments of the invention, the term “polymorphism” refers, without limitation, to the presence in a population of two or more different forms of a gene, genetic marker, or inherited trait or a gene product obtainable, for example, through alternative splicing, DNA methylation, etc.

In the context of the embodiments of the invention, the term “gene encoding sequence” refers, without limitation, to the information encoded in a gene that is used to either make RNA molecules that code for proteins or to make non-coding RNA molecules that serve other functions.

In the context of the embodiments of the invention, the term “genetic determinant” refers, without limitation, to genetic patterns that can be associated to a given trait, or QTL such as the EP trait of this invention.

According to some embodiments of the above pepper or plant seed, the genome of said plant comprises a genetic marker and/or a gene encoding sequence associated with said EP trait, said genetic marker and/or gene sequence is selected from the group consisting of:
a. a genetic marker sequence selected from SEQ ID NO: 281-290 or any combination thereof, allele A at position 9570115 as indicated in pepper genome Capsicum annuum cv CM334 v1.55 or at position 12,239,886 as indicated in Capsicum annuum cv Maor genome ASM2707369v1 NCBI (Table 8), and/or a gene encoding a sequence selected from SEQ ID NO: 1-93 or any combination thereof and/or any combination thereof, associated with QTL1 on chromosome 1;
b. a genetic marker sequence selected from SEQ ID NO: 291-323 or any combination thereof, an allele selected from Table 9 or any combination thereof, and/or a gene encoding a sequence selected from SEQ ID NO: 94-280 or any combination thereof, associated with QTL2 on chromosome 10; and
c. any combination thereof.

According to some embodiments of the above pepper or plant seed, the high yield properties of parthenocarpic fruits comprise at least one of: early fruit setting, setting fruit at high temperature and/or setting parthenocarpic fruit at high temperature, elevated fruit number per plant, and/or elevated fruit weight per plant, as compared to a pepper plant having the same genetic background and lacking said EP trait associated with the genetic marker and/or gene sequence.

According to some embodiments of the above pepper or plant seed, the high yield properties comprise (i) increased parthenocarpic fruit number per plant of at least about 5%, particularly 10-25%, and/or (ii) increased fruit weight per plant of at least about 15%, particularly 20-35%, as compared to a pepper plant having the same genetic background and lacking said EP trait associated with the genetic marker and/or gene sequence. In one embodiment, the increased parthenocarpic fruit number per plant is of at least about 5%, of at least 10%, of at least 15%, of at least, of at least 20%, of at least 25%. In one embodiment, the increased fruit weight per plant of at least about 15%, of at least 20%, of at least 25%, of at least 30%, of at least 35%.

According to some embodiments of the above pepper or plant seed, the property of early fruit setting and/or setting fruit at high temperature and/or setting parthenocarpic fruit at high temperature, is associated with genes or DNA sequences located on Chromosome 10 and selected from at least one gene encoding a sequence selected from SEQ ID NO: 94-280 and/or at least one sequence selected from SEQ ID NO: 291-323 and/or an allele selected from Table 9 or any combination thereof. In the context of the embodiments of the invention, the term “chromosome” refers, without limitation, to structures made of protein and a single molecule of DNA that serve to carry the genomic information from cell to cell.

It is within the scope that the cultivated pepper plant or seed of the present invention comprising the at least one molecular marker and/or gene associated with QTL2, have an average EP value of above 3, particularly in the range of 3-8.

According to some aspects, the cultivated pepper plant or seed of the present invention comprising the at least one molecular marker and/or gene associated with QTL2 exhibits an increase in EP value of about 20-40% compared to a plant lacking said at least one QTL2 associated molecular marker and/or gene.

According to some embodiments of the above pepper or plant seed, the property of elevated fruit number per plant and/or elevated fruit weight per plant is associated with genes or DNA sequences located on Chromosome 1 and selected from at least one gene encoding a sequence selected from SEQ ID NO: 1-93 and/or at least one sequence selected from SEQ ID NO: 281-290 and/or allele A at position 9570115 as indicated in pepper genome Capsicum annuum cv CM334 v1.55 or at position 12,239,886 as indicated in Capsicum annuum cv Maor genome ASM2707369v1 NCBI (Table 8).

The cultivated pepper plant or seed comprising the at least one molecular marker and/or gene associated with QTL1 have 15-20% more fruits compared to a plant lacking said at least one QTL1 associated molecular marker and/or gene.

According to some embodiments of the above pepper or plant seed, the plant is capable of forming fruits from at least about 90% of the flowers on said plant.

According to some embodiments of the above pepper or plant seed, the plant produces at least 38 fruits per plant, such as at least 65 fruits per plant, particularly at least 90 fruits per plant.

According to some embodiments of the above pepper or plant seed, the plant has fruit yield of at least about 1.5 kg per plant, preferably at least 3.6 kg per plant, more preferably at least 5.0 kg per plant.

In the context of the embodiments of the invention, the term “high yield” refers, without limitation, to genetically enhanced cultivars of crops, such as parthenocarpic seedless fruits' pepper, that have an increased crop production or increased percentage of usable plant parts, preferably fruits. The fruit yield produced by a plant may be affected by parameters such as timing of fruit setting, temperature of setting fruit, number of fruits per plant and weight fruit per plant. According to some non-limiting embodiments, the “high yield” refers to an increase of parthenocarpic fruit number per plant of at least about 5%, particularly 10-25%, as compared to a pepper plant having the same genetic background and lacking said EP trait associated with the genetic marker and/or gene sequence. For example, the average individual fruit number of the high yield pepper hybrids of the present invention is in the range of between about at least 38 fruits per plant to about at least 90 fruits per plant. According to some non-limiting embodiments, the “high yield refers to an increase of fruit weight per plant of at least about 15%, particularly 20-35%, as compared to a pepper plant having the same genetic background and lacking said EP trait associated with the genetic marker and/or gene sequence. For example, the average individual fruit weight of the high yield pepper hybrids of the present invention is in the range of between about at least 1.5 kg per plant to about at least 5.0 kg per plant.

In the context of the embodiments of the invention, the term “earliness” refers, without limitation, to the rate of fruit development and more specifically to the time elapsing between planting of the seed and the subsequent harvesting. More preferably, it relates to the days from transplanting to first red fruit. Thus, in plants earliness is evaluated by measuring how rapid a state of ripeness is attained. Earliness has economic significance. The cultivation of early ripening plant species and varieties results in a more productive use of land, since the same field may yield more than one harvest per season. An enhanced or increased earliness implies a shorter duration of the growth phase of the plant, which leads to flowering and a ripening of the fruits to be harvested, which occur, further ahead in time than is normally the case. It is further disclosed mat in cultivated pepper, early flowering is generally associated with higher yield of ripe fruits.

According to some embodiments of the above pepper or plant seed, the plant produces a fruit type selected from the group consisting of: bell pepper, pointed pepper, half long pepper, Como di Toro pepper, sweet pepper including a dolce-type pepper, a big rectangular pepper, a conical pepper, a long conical pepper and a blocky-type pepper.

According to some embodiments of the above pepper or plant seed, the mature fruit of the plant is green, yellow, orange, red, ivory, brown, or purple.

According to some embodiments of the above pepper or plant seed, the pepper plant is an inbred, a dihaploid or a hybrid.

In the context of the embodiments of the invention, the term “inbreed” refers, without limitation, to the process of mating among closely related individuals or even self-fertilization in plants.

In the context of the embodiments of the invention, the term “dihaploid” refers, without limitation, to haploid plants that undergone a spontaneous or induced chromosome doubling in haploid cells during embryogenesis, thus resulting in a homozygous individual, with two identical homologs. As used herein, the term “dihaploid” is interchangeable with “doubled haploid (DH)”.

In the context of the embodiments of the invention, the term “diploid individual” (diploid organism) refers, without limitation, to an individual mat that has two sets of chromosomes, typically one from each of its two parents. However, it is understood that in some embodiments a diploid individual can receive its “maternal” and “paternal” chromosomes from the same single organism, such as when a plant is selfed to produce a subsequent generation of plants.

In the context of the embodiments of the invention, the term “hybrid” refers, without limitation, to a plant resulting directly or indirectly from crosses between different species, varieties or genotypes (e.g., a genetically heterozygous or mostly heterozygous individual). In the context of the embodiments of the invention, hybrid plant is a plant resulted from crosses between populations, breeds or cultivars within the genus Capsicum. According to some embodiments, the hybrid plant is preferably resulted from Capsicum annuum. The term “hybrid” is related to “hybrid plant” and “hybrid progeny”.

In the context of the embodiments of the invention, the term “population” refers, without limitation, to a genetically heterogeneous collection of plants sharing a common genetic derivation.

According to some embodiments of the above pepper or plant seed, from at least 60% of the fruits grown on said plant, are at least 95% are seedless.

According to some embodiments of the above pepper or plant seed, the EP trait is as found in seeds of Capsicum annuum CM-192-539, representative seeds of which were deposited with NCIMB Aberdeen AB21 9YA, Scotland, UK under accession number NCIMB 44203 on 04/08/2023.

According to some embodiments of the above pepper or plant seed, the plant further comprising within its genome at least one additional trait selected from the group consisting of, Taste, Nutritional value, insect resistance, resistance to bacterial, fungal or viral disease, and resistance to a non-biotic stress, wherein the additional trait is introduced by a method selected from the group consisting of breeding, genome editing, genetic determinant introgression and transformation.

In the context of the embodiments of the invention, the term “breeding” refers, without limitation, to any process that generates a progeny individual, such as selection, via combination, of genetic desirable traits in a single variety (hybrid), thus generating an improved new plant variety (progeny individual). In one embodiment, the non-limiting list of types of breeding include crossing, selfing, introgressing, backcrossing, doubled haploid derivative generation, and combinations thereof.

In the context of the embodiments of the invention, the term “variety” or “cultivar” used herein means a group of similar plants that by structural features and performance can be identified from other varieties within the same species.

In the context of the embodiments of the invention, the term “genome editing” or gene editing refers, without limitation, to the addition, removal, or alteration of a genetic material at a particular desired location in the genome. A non-limiting list of techniques for genome editing are restriction enzymes, zinc finger nucleases, prime editing, and Programmable Addition via Site-specific Targeting Elements (PASTE).

In the context of the embodiments of the invention, the term “genetic determinant introgression” refers, without limitation, to the incorporation of new genetic determinants or elements such as genes, alleles, QTLs (quantitative trait loci) or traits, into a line wherein essentially all of the desired morphological and physiological characteristics of the line are recovered, in addition to the genetically introgressed determinant. In genetic determinant introgression, one or a few genetic determinants are transferred to a desired genetic background, preferably by using backcrossing or hybridization.

In the context of the embodiments of the invention, the term “transformation” refers, without limitation, to a way to insert DNA from another organism (usually another plant), into the genome of a plant of interest. This includes both integration of the exogenous DNA into the host genome, and/or introduction of plasmid DNA containing the exogenous DNA into the plant cell. Such a transformation process results in the uptake, incorporation and expression of exogenous genetic material (exogenous DNA). Plant transformation may refer to the introduction of exogenous genes into plant cells, tissues or organs employing direct or indirect means developed by molecular and cellular biology. A non-limiting list of techniques for the transformation of plants that are well known to those of skill in the art and applicable to many crop species include, but are not limited to, electroporation, microprojectile bombardment, Agrobacterium-mediated transformation and direct DNA uptake by protoplasts.

According to some embodiments, the invention provides a plant part comprising at least one regenerable cell, pollen, ovule, fruit or seed.

In the context of the embodiments of the invention, the term “regenerable” refers, without limitation, to a plant part wherein 100% of the population produces a parthenocarpic pepper plant.

According to some embodiments of the above plant, the plant is further defined as a leaf, a bud, a meristem, an embryo, a root, a root tip, a stem, a flower, a fruit, or a cell.

According to some embodiments, the invention provides a pepper seed obtained from a crossing in which at least one of the parents is the pepper plant according to the invention, or which produces the pepper plant according to the invention.

According to some embodiments, the invention provides a tissue culture of regenerable cells, protoplasts or callus obtained from the pepper plant according to the invention.

According to some embodiments, the invention provides a pepper fruit or processed pepper fruit of a plant according to the invention.

According to some embodiments, the invention provides a pepper plant comprising an introgressed Excellent Parthenocarpy (EP) trait conferring production of elevated yield of commercially acceptable parthenocarpic seedless fruits, as compared to a pepper plant having the same genetic background and lacking said EP trait, said EP trait is associated with as genetic marker and/or gene sequence is selected from the group consisting of:
a. a genetic marker sequence selected from SEQ ID NO: 281-290 or any combination thereof, allele A at position 9570115 as indicated in pepper genome Capsicum annuum cv CM334 v1.55 or at position 12,239,886 as indicated in Capsicum annuum cv Maor genome ASM2707369v1 NCBI (Table 8), and/or a gene encoding a sequence selected from SEQ ID NO: 1-93 or any combination thereof, or any combination thereof, associated with QTL1 on chromosome 1;
b. a genetic marker sequence selected from SEQ ID NO: 291-323 or any combination thereof, an allele selected from Table 9 or any combination thereof, and/or a gene encoding a sequence selected from SEQ ID NO: 94-280 or any combination thereof, or any combination thereof, associated with QTL2 on chromosome 10; and
c. any combination thereof.

According to some embodiments of the above pepper plant, the plant produces elevated yield of commercially acceptable parthenocarpic seedless fruits, independent of exogenous parthenocarpy-inducing factors.

Exogenous parthenocarpy-inducing factors may induce hormones, auxins, gibberellins, and cytokinins, especially the first two, are well known to induce parthenocarpy and environmental conditions such as the atmospheric temperature and the daylight hours.

According to some embodiments, the invention provides a method for producing a pepper plant exhibiting high yield properties of parthenocarpic fruits, the method comprising steps of:
a. producing and selecting a first pepper plant as a donor male parent, comprising an Excellent Parthenocarpy (EP) trait conferring production of elevated yield of commercially acceptable parthenocarpic seedless fruits;
b. crossing said first pepper plant with a second pepper plant as a female parent comprising a cytoplasmic male sterile (CMS) trait, to produce progeny hybrid pepper plants;
c. selecting at least one progeny hybrid plant showing elevated yield of commercially acceptable parthenocarpic seedless fruits as compared to the yield of a pepper plant having the same genetic background and lacking said EP trait; and
d. optionally, backcrossing said at least one selected progeny plant with said male parent plant and/or repeating steps c-d.

In the context of the embodiments of the invention, the term “donor parent” refers, without limitation, to the line containing the gene or trait of interest and the recipient parent or recurrent parent refers to the pepper line that is used as the normal or regular branched parent line, which is preferably an elite or breeding plant line that is improved by adding the gene or trait of interest.

In the context of the embodiments of the invention, the terms “male parent” and “female parent” refer, without limitation, to a plant that pollinates (provides pollen) and to a plant that received the pollen, respectively. A female parent can be any pepper plant that is the recipient of pollen. Such female parents can be male sterile, for example, because of genic male sterility, cytoplasmic male sterility, or because they have been subject to manual emasculation of the stamens. According to some embodiments, the male parent is a parthenocarpic line and the female parent is a pepper plant comprising a cytoplasmic male sterile (CMS) trait. According to some non-limiting embodiments, the female parent is the line CM202-1257. According to some non-limiting embodiments, the male parent is the line CM192-359 or the line CM202-2258. Preferably, the male parent is CM192-359.

In the context of the embodiments of the invention, the term “progeny” refers, without limitation, to all descendants/offspring plants of the crossing between male and female parents. According to some non-limiting embodiments, the progeny is obtained from breeding of two plants or from self-fertilization (selfing). In the context of the embodiments of the invention, the term “selfing” refers, without limitation, to the production of seed by self-fertilization or self-pollination; i.e., pollen and ovule are from the same plant. The first progeny is the F1 generation; the second progeny is the F2 generation, and so on. In some embodiments, the progeny carries the EP (Excellent Parthenocarpy) trait developed in this invention. According to some non-limiting embodiments, the progeny is a hybrid pepper plant. According to some non-limiting embodiments, the progeny is TM198-57 or TM198-63. Preferably, the progeny is TM198-63.

According to some embodiments of the above method, the step of producing and selecting a pepper plant as a donor male parent comprising an Excellent Parthenocarpy (EP) trait comprises steps of screening F2 seeds of various pepper genetic sources for being the donor male parent comprising the EP trait by:
a. crossing F2 plants of the F2 seeds as a male parent, with a first preselected CMS line plant as a recurrent female parent, for producing a first parthenocarpic hybrid progeny set;
b. selecting from said first hybrid set a first parthenocarpic hybrid plant producing the highest parthenocarpic fruit yield relative to the other hybrid progeny plants derived from the same first recurrent female parental line;
c. selfing the F2 male parent plant of the first selected parthenocarpic hybrid to produce F3 seeds;
d. crossing plants of the F3 seeds as a male parent with the first CMS recurrent female parent to produce a second parthenocarpic hybrid progeny set and selecting from said second hybrid set a second parthenocarpic hybrid plant producing the highest parthenocarpic fruit yield relative to the other hybrid plants;
e. repeating steps a to d with the relevant/next generation seeds or plants to produce a fertile pepper plant as a donor male parent comprising the EP trait, conferring production of elevated yield of commercially acceptable parthenocarpic seedless fruits.

In the context of the embodiments of the invention, the term “recurrent” refers, without limitation, to any parent plant which is used recurrently in subsequent crossings, i.e., the same parent plant line is used in repeated crossings of resulting progenies. In some embodiments, the recurrent parent is a recurrent female parent. In the context of the embodiments of the invention, the term “genetic sources” refers, without limitation, to male parent lines used for generating new progenies (hybrid plants). According to some non-limiting embodiments of this invention, the male parent lines are CM202-2258 and CM-192-539, preferably CM-192-539.

According to some embodiments of the above method, the step of screening comprises steps of producing a doubled haploid (DH) genotype plants from haploid cells derived from various pepper genetic sources.

According to some embodiments of the above method, the method comprises steps of inbreeding a pepper plant which is characterized by said EP trait until the genetic composition of the progeny of such inbreeding becomes substantially stable.

According to some embodiments of the above method, the genome of the donor pepper plant comprising the EP trait, and the EP trait is associated with a genetic marker and/or a gene encoding sequence selected from the group consisting of:
a. a genetic marker sequence selected from SEQ ID NO: 281-290 or any combination thereof, allele A at position 9570115 as indicated in pepper genome Capsicum annuum cv CM334 v1.55 or at position 12,239,886 as indicated in Capsicum annuum cv Maor genome ASM2707369v1 NCBI (Table 8), and/or a gene encoding a sequence selected from SEQ ID NO: 1-93 or any combination thereof, or any combination thereof, associated with QTL1 on chromosome 1;
b. a genetic marker sequence selected from SEQ ID NO: 291-323 or any combination thereof, an allele selected from Table 9 or any combination thereof, and/or a gene encoding a sequence selected from SEQ ID NO: 94-280 or any combination thereof, or any combination thereof, associated with QTL2 on chromosome 10; and
c. any combination thereof.

According to some embodiments of the above method, the high yield properties of parthenocarpic fruits comprises at least one of: early fruit setting, setting fruit at high temperature and/or setting parthenocarpic fruit at high temperature, elevated fruit number per plant and elevated fruit weight per plant, as compared to a pepper plant having the same genetic background and lacking said EP trait associated with the genetic marker and/or gene sequence.

According to some embodiments of the above method, the high yield properties comprise (i) increased parthenocarpic fruit number per plant of at least about 5%, particularly 10-25%, and/or (ii) increased fruit weight per plant of at least about 15%, particularly 20-35%, as compared to a pepper plant having the same genetic background and lacking said introgressed EP trait associated with the genetic marker and/or gene sequence.

According to some embodiments of the above method, the property of early fruit setting and/or setting fruit at high temperature and/or setting parthenocarpic fruit at high temperature, is associated with genes or DNA sequences located on Chromosome 10 and selected from at least one gene encoding a sequence selected from SEQ ID NO: 94-280, an allele selected from Table 9 or any combination thereof, and/or at least one genetic marker sequence selected from SEQ ID NO: 291-323.

According to some embodiments of the above method, the property of elevated fruit number per plant and/or elevated fruit weight per plant is associated with genes or DNA sequences located on Chromosome 1 and selected from at least one gene encoding a sequence selected from SEQ ID NO: 1-93, allele A at position 9570115 as indicated in pepper genome Capsicum annuum cv CM334 v1.55 or at position 12,239,886 as indicated in Capsicum annuum cv Maor genome ASM2707369v1 NCBI (Table 8), and/or at least one genetic marker sequence selected from SEQ ID NO: 281-290.

According to some embodiments of the above method, the property of elevated fruit number per plant and/or elevated fruit weight per plant is associated with
genes or DNA sequences located on Chromosome 1 and selected from at least one gene encoding a sequence selected from SEQ ID NO: 1-93, allele A at position 9570115 as indicated in pepper genome Capsicum annuum cv CM334 v1.55 or at position 12,239,886 as indicated in Capsicum annuum cv Maor genome ASM2707369v1 NCBI (Table 8), and/or at least one genetic marker sequence selected from SEQ ID NO: 281-290;
genes or DNA sequences located on Chromosome 10 and selected from at least one gene encoding a sequence selected from SEQ ID NO: 94-280, an allele selected from Table 9 or any combination thereof, and/or at least one genetic marker sequence selected from SEQ ID NO: 291-323; and/or
interaction between one or more of the above genes and/or DNA sequences located on chromosomes 1 and 10. Such an interaction may lead to a synergistic effect with respect to the high fruit yield properties.

According to some embodiments, the invention provides a pepper seed or fruit produced by the method according to the invention.

According to some embodiments, the invention provides a pepper plant produced by the method according to the invention.

According to some embodiments, the invention provides an allele, haplotype, genetic marker or gene being inherited to progeny plant, and this allele, haplotype, genetic marker or gene is associated with an Excellent Parthenocarpy (EP) trait capable of conferring production of elevated yield of commercially acceptable parthenocarpic seedless fruits as compared to a pepper plant having the same genetic background and lacking said EP trait, said genetic marker and/or gene sequence is selected from the group consisting of:
a. a genetic marker sequence selected from SEQ ID NO: 281-290 or any combination thereof, allele A at position 9570115 as indicated in pepper genome Capsicum annuum cv CM334 v1.55 or at position 12,239,886 as indicated in Capsicum annuum cv Maor genome ASM2707369v1 NCBI (Table 8), and/or a gene encoding a sequence selected from SEQ ID NO: 1-93 or any combination thereof, located on Chromosome 1;
b. a genetic marker sequence selected from SEQ ID NO: 291-323 or any combination thereof, an allele selected from Table 9 or any combination thereof, and/or a gene encoding a sequence selected from SEQ ID NO: 94-280 or any combination thereof, located on chromosome 10; and
c. any combination thereof.

According to some embodiments of the above allele, haplotype, genetic marker or gene, the allele, haplotype, genetic marker or gene having at least 90% sequence identity, and the allele, haplotype, genetic marker or gene is associated with an Excellent Parthenocarpy (EP) trait capable of conferring production of elevated yield of commercially acceptable parthenocarpic seedless fruits as compared to a pepper plant having the same genetic background and lacking said EP trait.

In the context of the embodiments of the invention, the term “sequence identity” refers, without limitation, to the occurrence of exactly the same or having a specified percentage of nucleotide or amino acid in the same position in aligned sequences. According to the present invention, the percent of identity or homology between two sequences is a function of the number of identical positions shared by the sequences, taking into account the number of gaps, and the length of each gap, which needs to be introduced for optimal alignment of the two sequences. The comparison of sequences and determination of identity percent between two sequences can be accomplished using a mathematical algorithm as known in the relevant art. As used herein, the term “sequence identity” is interchangeable with “sequence homology”.

According to some embodiments, the invention provides isolated nucleotide sequences annealing with or comprising sequences selected from:
a. at least one of SEQ ID NO: 281-290, allele A at position 9570115 as indicated in pepper genome Capsicum annuum cv CM334 v1.55 or at position 12,239,886 as indicated in Capsicum annuum cv Maor genome ASM2707369v1 NCBI (Table 8), or any combination thereof;
b. at least one of SEQ ID NO: 291-323 or any combination thereof, an allele selected from Table 9 or any combination thereof; and
c. any combination thereof;
the nucleotide sequence is suitable for the detection and/or production of a pepper plant comprising an Excellent Parthenocarpy (EP) trait conferring production of elevated yield of commercially acceptable parthenocarpic seedless fruits, as compared to a pepper plant having the same genetic background and lacking said EP trait.

According to some embodiments, the invention provides isolated gene sequences encoding sequences selected from:
a. at least one of SEQ ID NO:1-93 or any combination thereof;
b. at least one of SEQ ID NO: 94-280 or any combination thereof; and
c. any combination thereof;
the gene sequence is suitable for the detection and/or production of a pepper plant comprising an Excellent Parthenocarpy (EP) trait conferring production of elevated yield of commercially acceptable parthenocarpic seedless fruits, as compared to a pepper plant having the same genetic background and lacking said EP trait.

According to the above sequences some embodiments, the invention provides the use isolated sequences, or sequences having at least 90% sequence identity with the sequences of the invention, for detection and/or production of a pepper plant comprising an Excellent Parthenocarpy (EP) trait conferring production of elevated yield of commercially acceptable parthenocarpic seedless fruits, as compared to a pepper plant having the same genetic background and lacking said EP trait.

According to some embodiments, the invention provides pepper genetic markers, sequences or elements, plants, seeds, fruits and plant products, as disclosed in the invention, for the use in multiple geographical- and/or weather-related environments and growth conditions.

In the context of the embodiments of the invention, the term “elements” refers, without limitation, to allele, haplotype, genetic marker or gene.

According to some embodiments, the invention provides the use of a seed deposited under NCIMB accession number 44203 on 04/08/2023 for the production of the pepper plant according to this invention.

According to some embodiments, the invention provides a method for increasing parthenocarpic seedless pepper fruit yield production to a commercially relevant extent in multiple geographical- and/or whether-related environments or areas or growth conditions comprising growing in said geographical area pepper plant according to this invention.

### Deposits:

The seed samples of Capsicum annuum CM-192-539 were deposited under NCIMB accession number 44203, with NCIMB, Ferguson Building, Craibstone Estate, Bucksburn, Aberdeen AB21 9YA, Scotland, UK on 04/08/2023 under the provisions of the Budapest Treaty in the name of Breedx Ltd.

The following examples are presented in order to more fully illustrate certain embodiments of the invention. They should in no way, however, be construed as limiting the broad scope of the invention. One skilled in the art can readily devise many variations and modifications of the principles disclosed herein without departing from the spirit and scope of the invention.

### EXAMPLES

In order to understand the invention and to see how it may be implemented in practice, a plurality of preferred embodiments will now be described, by way of non-limiting example only, with reference to the following examples.

### Example 1

### Yield Results of Pepper Lines TM198-57 and TM198-63

This example describes an experiment, where, pepper fruit yield of TM198-63 line, herein defined as having Excellent Parthenocarpy (EP) phenotype level of 8, was examined as compared to TM198-57 line, herein defined as having EP phenotype level of 5 (used as a control). Towards this end, both TM198-57 and TM198-63, F1 plants, were grown in Spain at a long harvesting cycle (harvesting period of about 8 months) and in Israel at a short harvesting cycle (harvesting period of about 4-5 months).

In the experiments, hybrid pepper lines were produced using genetic male sterility. The genetic male sterility was obtained via Cytoplasmic Male Sterility (CMS). It means that for each line, a CMS version (female version called A-line) and it's maintainer in fertile version (male version called B-line) were used. It is emphasized that the lines are isogenic except for the CMS trait.

The following F1 combinations (varieties) were produced for the analysis:
TM198-57 (sterile)
TM198-63 (sterile)

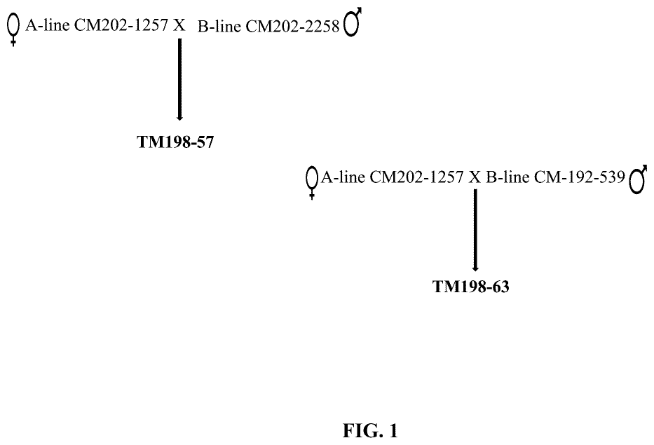

FIG. 1 presents a breeding scheme describing the resultant hybrid varieties and their parental lines. In this scheme, a hybrid seed was produced by crossing as a female parent of a first pepper plant comprising a cytoplasmic male sterile (CMS) trait (CM202-1257 line), with a second plant, which is parthenocarpic (CM202-2258 line or CM-192-539 line) to produce at least a first seed of a parthenocarpic seedless pepper plant (TM198-57 or TM198-63, respectively). A plant grown from such a seed, or a vegetative propagation thereof, is also provided by the invention, as well as a part of such a plant. In some embodiments, the plant part may further be defined as a leaf, a bud, a meristem, an embryo, a root, a root tip, a stem, a flower, a fruit, or a cell. In particular embodiments the plant part is further defined as a pepper fruit that lacks internal carpelloid structures.

### Experimental Plot in Israel

### Materials and Methods

Two separate experiments/growing cycles were done for the yield measuring, spring and fall experiments.

Experiment type (format); random blocks.

### Spring Experiment:

The experimental setup contained the two F1 combinations (varieties) in 4 random blocks for each treatment (Treatment=Variety).

The two varieties (two treatments) were sown on January 21^{st} using one tray for each variety. Germination was counted after 3 weeks.

### The Varieties/Treatments Used:

A=TM198-57
B=TM198-63

Each variety/treatment contained at least 120 plants, 30 plants per replica.

An illustrative experimental map is presented in Table 1 below.

TABLE 1
Plot experimental map
EXPERIMENT MAP

| | | | |
|---|---|---|---|
| B | A | B | A |
| A | B | A | B |
| Block 1 | Block 2 | Block 3 | Block 4 |

### Planting:

Location: Mivtahim south of Israel.
Field: Greenhouse

Experimental setup/plot: 60 plants per block, 30 plants for each treatment/variety (140 plants in a row, 20 plants in the borders).

The experiment contained 240 plants in total (0.125˜dunam)

Planting was performed in March.

### Harvesting:

Seven harvesting events were performed, the gap between each harvest was 2-3 weeks, depending on the ripening of the fruits.
Harvest Dates: 08/06, 22/06, 01/07, 13/07, 23/07, 10/08 and 01/09.

The harvest was done in the middle of the plot, 20 plants for each replica. Yield parameters were evaluated.

All fruits with 80% ripening were picked into boxes (per treatment) and measured for average weight and number of fruits per plant. Unmarketable or non-commercially acceptable fruits were not counted for the yield measuring.

### Yield Measuring was Performed for the Following Fruit Size Segments:

1. Fruits size of ˜6 cm
2. Fruit size between 6-8 cm
3. Fruit size between 8-15 cm
4. Fruit size above 15 cm

Each segment was measured for weight and number of fruits separately. Four measures were performed for each harvesting, per replica per treatment, 16 measurements for each treatment per harvest date. From this data, the total yield was calculated (total yield: weight/number of all fruits together).

The data was analyzed, and statistical analysis was calculated (weight and fruits number) for each treatment.

### Fall Experiment:

A similar experimental plan to the spring experiment was conducted in the fall with some minor changes.

The two varieties (two treatments) were sown on July 25th using one tray for each variety, and after three weeks germination was counted.

### Planting: (23.8)

Location: mivtahim south of Israel
Field: Greenhouse
Varieties/Treatments: as in the spring experiment
Experimental plot: 140 plants in a row, 40 plants in the border
Replicas: 25 plants per replica, 4 replicas per treatment/variety
Field map: as in the spring experiment

### Harvesting:

Harvesting and yield measuring: 15 plants in the middle of the plot were subjected to harvesting. The tested parameters of the harvesting (picking, segments etc.) were as in the spring experiment.
Harvest Dates: 09/11, 24/11, 08/12, 16/12 and 06/01.

### Results of Yield Experiments:

The data collected from the yield experiments was analyzed (data analysis with JMP). Because the experimental program was the same in spring and the fall, the experimental data of both experiments was analyzed together, to strengthen the confidence of the results by using more repeats for each treatment.

The results were analyzed by comparing the number of fruits per plant. Since the fruits are seedless, this type of analysis presents and evaluates the yield in the most accurate way.

Table 2 below summarizes the yield results with statistical analysis (weight and fruits number) for the sterile lines (treatments).

TABLE 2
Statistical analysis of yield results (weight and fruits number)

| | | No. | | Weight | |
|---|---|---|---|---|---|
| | | fruits/plant | | Kg fruits/ | sig. |
| Trial | Treatment | (n) | sig. n* | plant | wt* |
| Spring & | TM198-57 | 57.62 | b | 1.880 | b |
| fall | TM198-63 | 65.41 | a | 2.400 | a |
| SEM | | | 1.77 | | 0.064 |

*Different letters represent Tukey's HSD p = 0.05

As can be seen in Table 2, a significant increase was observed in the tested fruit yield parameters (fruit number and fruit weight per plant) of TM198-63 as compared to TM198-57. It is noted that both TM198-57 and TM198-63 have the same female parent (line CM202-1257), but a different male parental line (see FIG. 1). Thus, the results above clearly demonstrate that line CM 192-359, the male parental line of TM198-63 lead to about 13% increase in fruit number and about 27% increase in total yield per plant (fruit weight per plant), in comparison to TM198-57 (having line CM202-2258 as a male parent). Line CM 192-359 is herein shown to be a source for the EP trait associated with high yield fruit properties.

It can be summarized by the experimental results shown herein that there is a positive correlation between the parthenocarpy level and the fruit yield of the plant. The present invention demonstrates that the “Excellent Parthenocarpy” trait associated with the herein disclosed genetic markers or unique haplotype confers high fruit yield phenotype in pepper plants (in CMS background). In other words, the inventors showed that a unique haplotype (described below) is correlated with elevated seedless pepper fruit yield of at least 10% or more (e.g. up to about 30% increase) as compared to pepper plants lacking the unique haplotype.

### Experimental Plot in Spain

### First Experiment:

Transplanting: beginning of August
First harvest: beginning of November of the same year
Final harvest: mid. June of next year (about 7 months from first harvest)

TABLE 3
Yield results (fruit's weight and
number) of first experiment in Spain

| | | | Weight | Weight |
|---|---|---|---|---|
| | | | increase | increase |
| Variable | TM-198-57 | TM- 198-63 | (Kg) | (%) |
| No. of plants | 60.0 | 60.0 | | |
| M2 | 30.0 | 30.0 | | |
| Weight | 3.9 | 5.0 | 1.1 | 28.2 |
| Kg/plant | | | | |

The results in Table 3 show that the average fruit yield (weight per plant) was elevated by 1.1 Kg in TM-198-63 versus TM-198-57, an average increase of about 28% in the yield of parthenocarpic seedless fruits.

### Second Experiment:

Transplanting: beginning of August
First harvest: mid. November of the same year
Final harvest: end of July of the next year (about 8 months from first harvest)

TABLE 4
Yield results (fruit's weight and number)
of the second experiment in Spain

| | TM-198-57 | TM-198-63 |
|---|---|---|
| Weight | 2.34 | 2.91 |
| (Kg fruits/plant) | | |

The results in Table 4 show that the average fruit yield (weight per plant) was elevated by 0.57 Kg in TM-198-63 versus TM-198-57, an increase of about 24% in the yield of parthenocarpic seedless fruits.

### Example 2

### Identifying Unique Haplotypes for High Yield

A genetic analysis for identifying unique haplotypes associated with the disclosed EP trait and high yield characteristics was performed. The analysis was made by Genotype by Sequencing (GBS) technique on DNA samples derived from F2 population.

In this section, F2 population was produced to identify DNA region/s in the genome of line CM 192-539 linked to the unique EP (Excellent Parthenocarpy) trait developed by the inventors.

The male parental lines CM202-2258 and CM-192-539 were used as genetic resources of the hybrid plants TM198-57 and TM198-63, respectively, as presented in Table 5.

TABLE 5
Pepper lines and progeny populations

| Hybrid | EP | | |
|---|---|---|---|
| plant | level | Female line | Male line |
| TM198-57 | 5 | CM202-1257 | CM202-2258 |
| TM198-63 | 8 | CM202-1257 | CM-192-539 |

The inbred pepper line ‘Maor’ (bell-type, Capsicum annuum), as well as Capsicum annuum cv. CM334, were used as control lines (reference genomes), e.g. versus lines CM202-2258 and CM-192-539.

### Objective:

The genetic analysis was aimed to identify genomic regions where all three samples (CM202-2258, CM-192-539, ‘Maor’/CM334) have different haplotypes.

### General Workflow:

1. Produce sequencing data for each of the two CM202-2258 and CM-192-539 samples.
2. Mapping of the sequencing data to “Maor”/CM334 pepper variety genome assembly.
3. Variant calling for each sample.
4. Recalling variants for each sample.
5. Comparative analysis to identify unique regions across the genome.

### Workflow Description:

1. The genomic DNA (gDNA) samples of pepper lines CM202-2258 and CM-192-539 were sequenced.
2. The reads were aligned to the “Maor”/CM334 pepper variety, which is relatively similar to the two tested varieties. Alignment of the sequencing data of the two samples (CM202-2258, and CM-192-539) to “Maor”/CM334 reference genome was done.
3. Variants discovery and recalling was performed. Briefly, reads were aligned to the Maor/CM334 reference genome and variants were called for each variety. The recalling step is important when comparing between different samples since it provides another layer of validation from an additional dataset. In addition, it is used to homogenize the analysis between the different samples.

It is herein disclosed that the EP trait confers the following characteristics:
Early fruit setting/setting fruit at high temperature.
Yield increase, a result of increasing number of fruits as well as total fruit weight (kg) per plant.

In the experiment, the CMS background was used for phenotyping. To identify the phenotype, 195 individual plants of F2 population were crossed with a CMS female.

Towards this end, the following lines were used:
1. CM 192-539 line (male parent of TM198-63)—exhibiting high EP phenotype.
2. MAOR (a reference line/genome)—exhibiting very low EP

A cross between the above lines was performed in the spring season (March) to produce F1 generation plants.

In the fall, the F1 plants were grown to produce F2 generation progeny plants. The F2 plants were grown for the following purposes:
1. Sampling every plant in the population for DNA extraction, used for genotyping proposes.
2. Crossing each plant as a male parent, with the same female (CMS female) to produce a sterile hybrid, for phenotyping proposes.

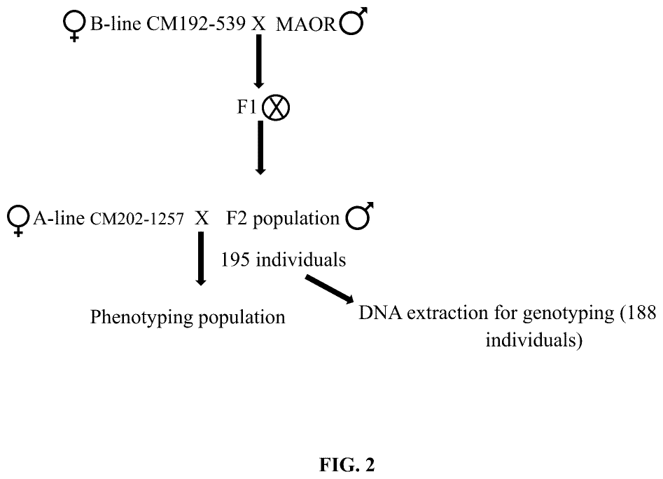

FIG. 2 presents a breeding scheme for generating F2 population for genotyping markers associated with the high yield trait of the present invention.

### DNA Treatments:

DNA extraction, measuring, and shipping:
DNA extractions were done with ISOLATE II Plant DNA Kit protocol.
DNA measuring was done with Qubit dsDNA BR Assay Kit protocol.
DNA was dried in 96 well plates and subjected to genotyping.
Total of 188 F2 individuals participated in the genotyping analysis process.

### Crosses:

In the spring, each individual plant of the F2 population was crossed with a corresponding female (CM202-1257 female line).

The crosses were done in a greenhouse located in “Netiv-Ha'asara” south of Israel.

From the crosses, 195 hybrids were collected for phenotype evaluation experiment that was done in the field (Net-house) in the fall (Arava south of Israel).

These hybrids included the 188 F2 individuals that were sampled for genotyping and used as a male parent in the cross, and additional 7 F2 individuals that their DNA was not collected.

### Genotyping:

Genotyping by sequencing was carried out by The Elshire Group (htt ps://www.elshiregroup.co.nz/). Libraries of the 188 individual plants were sequenced on a Novaseq platform in a paired-end with 150 bp read length. The raw-data was demultiplexed using the axe-demux tool (according to Murray and Borevitz, 2018) into the separated libraries. In the next steps, adapters were trimmed, and low-quality reads were removed with Trimommatic (according to Bolger et al., 2014). Finally, the GATK pipeline (according to McKenna et al., 2010) was used to detect polymorphic sites across the population. Briefly, BWA-MEM (Li, 2013) was used to align the reads to the Capsicum annuum cv. CM334 reference genome (https://www.nature.com/articles/ng.2877), and the HaplotypeCaller (Poplin et al., 2018) with default parameters was used to generate polymorphic positions summarized in a VCF file for association mapping.

### Phenotype Experiment:

Seeds of the 195 hybrids were sown on August 3^{rd} (40 seeds per hybrid). After 2-3 weeks, germination was counted.

In this experiment, 195 hybrids are referred to as 195 treatments.

Each hybrid was planted in 3 replicas, about 10 plants for each replica.

Phenotype was evaluated in 2 different ways:
1. Visual (qualitative) measurement: each replica received an internal EP grade (a value between 0-10), three independent grades were determined for each treatment.

EP grade or level or value herein means a grade given to each plot by visually evaluating the fruit yield in the particular plot. EP grade is a relative evaluation, where the relative point used is a commercial seedless pepper variety that has EP between 2.9-3.1. The EP grade represents the early fruit set; it means fruits that are observed on the plant up to 60-80 cm from the ground.
2. Harvesting (quantitative) measurement: fruits were picked from 5 plants of each replica, in the middle of the plot, and yield parameters were measured (weight and number of fruits). Three replicas for each treatment were measured.

### Measuring:

Visual measuring was done in 2 separate dates: 3/11 and 7/12.

Harvesting measurements were done on 12/12 and 1/2.

After phenotype collection, statistical analysis of the data was performed. Based on the combined data of the genotyping analysis and the phenotype analysis, unique genomic regions in line CM 192-539, related to the EP trait and yield increase phenotype, was identified in the pepper genome.

### Genotyping Results:

GBS for F2 population experiment:

In this trial, data collection and phenotypic analysis were performed 4 times through the season, 2 times for EP evaluation and 2 times for yield measurements (number of fruits and fruits weight).

Data analysis for phenotyping was done by JMP, followed by genotypic analysis.

Genotypic analysis done by the VCF file, was processed in Tassel 5 (Glaubitz et al., 2014). Sites with minor allele frequency <0.05 were filtered out from downstream analyses. Association mapping was performed with Tassel 5 using the generalized linear model (GLM) algorithm with 1000 permutations.

Also in this experiment, the number of fruits per plant was used for the analysis, due to the reason that the fruit weight parameter was not accurate (the fruits were on the plant for long period and lost their weight by shrinking due to water loss).

After analyzing the genotype and the phenotype, the results were combined to identify phenotype/genotype linkage in association with a QTL. It was chosen to use the results that have high LOD score (above 8).

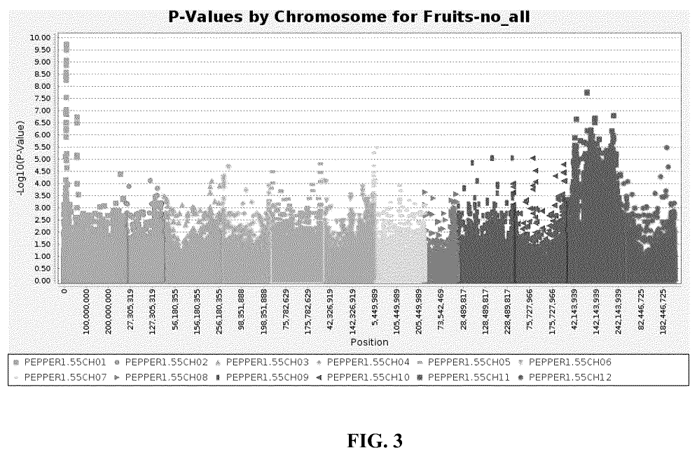

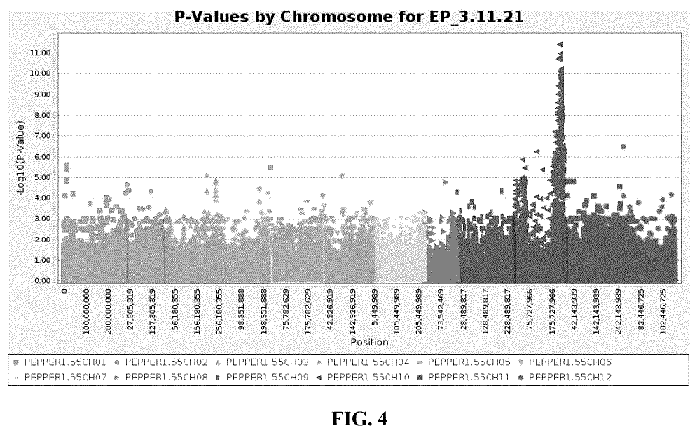

Reference is now made to FIG. 3 and FIG. 4, illustrating Manhattan PLOTS for the F2 population individuals according to fruits number parameter and EP qualitative evaluation, respectively (LOD score above 8 was referred to as a significant result).

As can be seen in the results, the inventors successfully found two clear genome areas (two QTLs) that match the defined criteria (EP trait).

The first genomic region, identified according to “number of fruits per plant” parameter, is located on QTL1 of chromosome 1 (Chr 1) (FIG. 3) and has the size of 4.5 Mbp (9-13.5 million bp on Chr 1). In this region, 93 sequences of candidate genes (see sequencing data SEQ ID NO: 1-93) and 11 SNPs (see SEQ ID NO: 281-290 and Table 8) were identified.

The second genomic area, identified according to EP evaluation/grade/level (e.g. the visual or qualitative measuring of EP in hot conditions/early fruit setting), is located on QTL2 of chromosome 10 (FIG. 4) and has the size of 19 Mbp (199-218 million bp on Chr 10). In this region, 187 sequences of candidate genes (see sequence data SEQ ID NO: 94-280 below) and 130 SNPs (see SEQ ID NO: 291-323 and Table 9) were identified.

From genetic data of CM192-539 sequence (a full genome sequencing was performed and compared to “MAOR”/CM334 genomic sequence), a total of 5000 SNP's unique to CM192-539 were found, 11 SNPs that are located on Chr 1, and 130 SNP's on Chr 10 of the tested F2 population (see Tables 6-9).

In Table 6, the sequence of each of the SEQ ID NO's includes the ‘Flanking Sequence Upstream’+‘CM-192-539’ unique sequence+‘Flanking Sequence Downstream’. The position indicated is relative to Maor ASM2707369v1 NCBI.

TABLE 6

| SEQ | | | | |
|---|---|---|---|---|
| ID | | | | |
| NO: | | | | |
| 281- | | | | |
| 290 | ID | Chr | Position | Seq |
| 281 | 1_12184754 | 1 | 12184754 | AATTTTGTTTTTTCCTATTC |
| | | | | CTAATCAAATTGGGATTAGA |
| | | | | GTTTTATTAGAACTATAGGC |
| | | | | CTTGTAAATATGGATGGCCT |
| | | | | GACATTTCGAGTTAATGGTA |
| | | | | A |
| 282 | 1_12245030 | 1 | 12245030 | GTCTTATTGTAGCACAGAGA |
| | | | | AAAGGGCATAAATAATCCCT |
| | | | | TATCTATAGGAGCAAGTATT |
| | | | | ATCTAAAAGTTTATTAATAT |
| | | | | TGATCTTTGGATATTCTTAT |
| | | | | ACAAAC |
| 283 | 1_12253581 | 1 | 12253581 | GTTTTTTTATAATTTGATAT |
| | | | | CTATTATGCATGGTTGTGAA |
| | | | | TGTATAACAAATTTATACAT |
| | | | | ATCATATATATACCTTCTAT |
| | | | | TCGAATTGAACCTCAGTTAT |
| | | | | TGTTGGACATTCAGTATGGA |
| | | | | TGT |
| 284 | 1_12257386 | | 12257386 | TTTCGGACTGTATAATTGGA |
| | | | | CTGGACACTAAATTTAGGAT |
| | | | | TGTTTGGTTTTGCTTGCTTG |
| | | | | CTTTGTTTTGTATGTTTTGT |
| | | | | GTGGTTTATACATTTGTAGT |
| | | | | G |
| 285 | 1_12258771 | 1 | 12258771 | CAGTTAATCATTAATTGTCC |
| | | | | AATTTGGACGAACTACGCAT |
| | | | | ACCTGATTCCCTTCCCTCGG |
| | | | | GGTGGGATATGTAGGCAACC |
| | | | | CTCGATGGGTTCGGTAATAT |
| | | | | T |
| 286 | 1_12339811 | 1 | 12339811 | ATCAGCAAAAGGTAGGTCTT |
| | | | | TGGGGTCGGCGGGACCGTTT |
| | | | | GGGTAGTCAACCGGGAGAAA |
| | | | | CGGCACGAACAAACCGTATT |
| | | | | TAGGCCAAATTGGTGTGCTA |
| | | | | T |
| 287 | 1_12372733 | 1 | 12372733 | TGTCTTCCCTCATATGTGAA |
| | | | | TCTGATTCTTTTTTTTTTAG |
| | | | | GCTGAACACGTAAAAAAAAA |
| | | | | AATTTAGATAATGAATGAGT |
| | | | | GACATTTAACACTTATATAG |
| | | | | GA |
| 288 | 1_12372743 | 1 | 12372743 | CATATGTGAATCTGATTCTT |
| | | | | TTTTTTTTAGGCTGAACACG |
| | | | | TAAAAAAAAAATTTTTTTTA |
| | | | | GATAATGAATGAGTGACATT |
| | | | | TAACACTTATATAGGATATG |
| | | | | CATCTT |
| 289 | 1_13098771 | 1 | 13098771 | CTGGTATTGCCAGTTCTAAC |
| | | | | AGAATCAAGTCTGTTCAACA |
| | | | | AATGAAAGTGGTATGCTTGA |
| | | | | ACGTACACAATGTGGCCTTT |
| | | | | TTTGCACTTCATCATAATAC |
| | | | | A |
| 290 | 1_13164788 | 1 | 13164788 | TAAAGAGGGTGAACTCTCAG |
| | | | | ATTAAATTACTCTTTCATGG |
| | | | | TTGCAAAAATTAAAAAAAAA |
| | | | | AGAATTGACATATTATTAAA |
| | | | | CAAATTAAATAACACTATAT |
| | | | | TAA |
| SEQ | | | | |
| ID | | | | |
| NO: | | | | |
| 291- | | | | |
| 323 | ID | Chr | Position | Seq |
| 291 | 10_199967383 | 10 | 199967383 | TGCAGGGATTGATTTCCTTA |
| | | | | TGTTTCCTAACATACCACCT |
| | | | | GGAGATCGTTGGGAGGCCAA |
| | | | | AGGTACTTATTAAAAAAATT |
| | | | | AGTTTGGCAAGTTTTTGGAG |
| | | | | AAAATAAGATACCATGATGA |
| | | | | AGTAACGATTATCATTATCT |
| | | | | CTTTCGGAGGAAAGA |
| 292 | 10_202615756 | 10 | 202615756 | ATGGCGATATTTGGTTGACT |
| | | | | CTTACATATTAGAAAACTAA |
| | | | | TTATATTTAATAAAAAAAAA |
| | | | | AAAAAGATAAAATAGACATT |
| | | | | TATGACATGTCTTCTACATC |
| | | | | TGC |
| 293 | 10_202629559 | 10 | 202629559 | CTTCACAAAGTGGGTAAAAG |
| | | | | TAGTAACTTTCAAGTCAGTG |
| | | | | ACAAAGAAGGTTGTGGTAGA |
| | | | | TTTTGTTCATGCCAATATCA |
| | | | | TTTGTAGATTTGGAATTCCT |
| | | | | A |
| 294 | 10_204401467 | 10 | 204401467 | CAAAATATCTCAAATATTAA |
| | | | | ACATCAACGTGATCGTAACA |
| | | | | CACACTTTCTAAAAAAAAAT |
| | | | | AGAATTTCCAATGGGAATTC |
| | | | | ACGACCCAAATATCAAAATC |
| 295 | 10_204479958 | 10 | 204479958 | ATCATAGGGGTGCATGGTTT |
| | | | | AAGATGAGTATAAAGAAGGG |
| | | | | AAAAATAAAGGAAAAATAAT |
| | | | | AAAAGTTCGGTGCTATTCAC |
| | | | | AAAAAAAATAAAATAATAAT |
| | | | | A |
| 296 | 10_209148899 | 10 | 209148899 | GGGTTTGAATATTGATTCAA |
| | | | | AAAACATTTCATGAAATCAT |
| | | | | TTTGATGAAAGTATATATAT |
| | | | | ATATATATATATATATATAT |
| | | | | ATATATATATATTGTATTGA |
| | | | | ACTCTTTTTCATGGAAATTG |
| | | | | AATTATAGCCCCT |
| 297 | 10_209509419 | 10 | 209509419 | TCCACTTTAATAATGAAAAC |
| | | | | CCTTTACAAACTAAAATATG |
| | | | | ATCTTTTTGGTAAAAAAAAA |
| | | | | AAAAGGGTGTTGATAGCTCT |
| | | | | AGCAACTCTGCTGGGGACTT |
| | | | | TT |
| 298 | 10_209510189 | 10 | 209510189 | AAACATCAAAAAGATTTTTC |
| | | | | TTTTGTTTCTTTAGTCAGCA |
| | | | | TTCATAATTTTAAAAAAAAA |
| | | | | AAAATTTATTAATAGAAACT |
| | | | | TTTTATAAAAGAAAAATTCA |
| | | | | A |
| 299 | 10_209589641 | 10 | 209589641 | ATCTCATTATATGTCATCTC |
| | | | | AAGAGCTTGACCACCTGCAT |
| | | | | CTAAATCTAGAAAAATCTTT |
| | | | | GTATTTTGATCAAGAGCCTC |
| | | | | TACAAAGTATGAGCAAGTAC |
| | | | | C |
| 300 | 10_210995208 | 10 | 210995208 | AAACAAAGATTATGATAAAG |
| | | | | CAAGATTTTATTATTCTGAA |
| | | | | AATAATGTCCAATTTTTCAT |
| | | | | GAGGGTCCTGAAAATAAAGT |
| | | | | AAACTCCCATTTTTTAGGAG |
| | | | | G |
| 301 | 10_211264971 | 10 | 211264971 | GAAATCCTTCCAAATGCAGC |
| | | | | ATCTCTTGTTTGACTCTTGA |
| | | | | CATTGGTGTTTGGCATTCTG |
| | | | | TAGATACGCAATGTTTGTAT |
| | | | | TAGGAAGTTGGCTAATTTTA |
| | | | | TAATCAGGTGGCATGGT |
| 302 | 10_211362818 | 10 | 211362818 | TTGGATATCGAAGTAAAACG |
| | | | | TTATGGTTGATCTACTTCAG |
| | | | | ACTATCAAAAAAGTCCACCA |
| | | | | AAGGATAGATTCGAGAATTA |
| | | | | TTGAATTTTTAGGGGCGTTT |
| | | | | T |
| 303 | 10_211380569 | 10 | 211380569 | TTATGTAGTATAATGCTAGT |
| | | | | ATTCGTTGTTTGGAAAGACG |
| | | | | TAACATTGATGTAGTGAGAT |
| | | | | TGAATAAATAAAAATACATC |
| | | | | TATTTTTCTATAATGTTAAT |
| | | | | A |
| | 10_211397098 | | 211397098 | ATATCTACATGTTCAATGTG |
| | | | | TTCTATCCATAATTGTCGTT |
| | | | | GATTTTCGTTGTAAGGTGGT |
| | | | | GTATAATCTCATTTAGTACT |
| | | | | GAATATATGTATAGAAGAAT |
| | | | | A |
| 305 | 10_211409339 | 10 | 211409339 | TATATTTTAATATTATAATA |
| | | | | GTTGAAAACATCTAGTACAT |
| | | | | TTTAAAAAGACAAAAAAAAA |
| | | | | AAAGAAAAAAGACTTATTAG |
| | | | | GCTAAAGCCCAGCAGAAGCA |
| | | | | ACC |
| 306 | 10_211561786 | 10 | 211561786 | TTATGTTTTTCAAAAAGTTT |
| | | | | TTTTACTATTCACCTCCGTT |
| | | | | AGTTAACCGTTAAAAAAAAA |
| | | | | AAAAGAGTAAGCCGTAAGAA |
| | | | | TTTATTACCTTTATCCTATT |
| | | | | TT |
| 307 | 10_212284490 | 10 | 212284490 | CCTAAAGACTAGGACAAAGC |
| | | | | TGGAAATAGATTGAAAATAG |
| | | | | GACTTTATTCTCTGACTTTA |
| | | | | TTTGCTAACAAGCATGATAT |
| | | | | AAGGACAGGTAAGCAAATAA |
| | | | | C |
| 308 | 10_212297792 | 10 | 212297792 | GATAAGCATCCCTTATAATA |
| | | | | GATGTTATTGAAGAGAATTT |
| | | | | TAAGAATAGATAAAAAAAAA |
| | | | | AACTCTAATATATTTGTCAT |
| | | | | TTCTGTTATAAAATGTATAG |
| | | | | TA |
| 309 | 10_212411948 | 10 | 212411948 | ATATGTTGCATCAGATAATT |
| | | | | TATTTATTGCAACATATAAT |
| | | | | CTACTTTTTTTAAAAAAAAA |
| | | | | AATAACTATCATCATATCAT |
| | | | | TAATCCAAATTTGCTTACTT |
| | | | | TTT |
| 310 | 10_212425128 | 10 | 212425128 | AATCATTAATTAAAATAAAA |
| | | | | ACTTGATTAGAATTGGACTT |
| | | | | ACTGCTTTCTTGGGGGGGGG |
| | | | | GGGGTTGAAATGATCAAGAA |
| | | | | ATTTTGTATTTTTATAGGTT |
| | | | | TTGGCCG |
| 311 | 10_212443574 | 10 | 212443574 | TACTTATCTATATATATGTG |
| | | | | GGGTGTAAAATAATAATTTA |
| | | | | GTATAATCTTATTGGATTAT |
| | | | | CAGTTTACCCAATAACCCAA |
| | | | | TAAAAAAATCGAAATTGAAT |
| | | | | CAATAACCCAATAA |
| 312 | 10_212498181 | 10 | 212498181 | TTAAAAGTTGAGGACATAAA |
| | | | | ATCATATTTAAAAAAGATTT |
| | | | | TTCAAAAATCTAAAAAAAAA |
| | | | | AAATTCAAAAAGACATGGTC |
| | | | | AAACACAACTCCAACTTTAT |
| | | | | CTT |
| 313 | 10_212498277 | 10 | 212498277 | ATCTTTAACTTCAACTCTAA |
| | | | | CTCCAAAGAAAGTAATTTTT |
| | | | | ATGACCAAATCGCTATTAAT |
| | | | | GTGTTGTTATATGTGACAAT |
| | | | | TTTAGAACCAATTGCTACCC |
| | | | | A |
| 314 | 10_212519400 | 10 | 212519400 | TGTTGACAAAATTTTTGTAT |
| | | | | GCCTTATTTCGGCTCATAAT |
| | | | | TATATCTTTTACATGTGCTG |
| | | | | GCCCTTTTGTCTTGCTTTGT |
| | | | | TCAATTGAAGAAGCTGGTAG |
| | | | | C |
| 315 | 10_212532367 | 10 | 212532367 | ATATAGAAGCTACTGCTGAA |
| | | | | CAATATTGATAATATGCTAC |
| | | | | TCTTTTTATGGAAATACGGA |
| | | | | GAACAGAAAATGCAGAATCG |
| | | | | TACGTAAGCGACAATAAATA |
| | | | | T |
| 316 | 10_212656886 | 10 | 212656886 | ATAAAGAGGTTTGGCAAGAA |
| | | | | GGGAAACTCAGTCCTCGATA |
| | | | | CATCGGTCCTTAAAGAATTC |
| | | | | TCAGCTATTTTAAAAAGGTA |
| | | | | GCTTATGAACTTGAGTTGTC |
| | | | | T |
| 317 | 10_212715663 | 10 | 212715663 | CAATTAGGAAAATGACTCAT |
| | | | | ATTGTCATTACCAGTAAAAA |
| | | | | TTTTATTTTTTAAAAAAAAA |
| | | | | AAAAAAAGGGTAAACAGGCT |
| | | | | CTTGCTCTCTGTTGAGTATA |
| | | | | G |
| 318 | 10_212727380 | 10 | 212727380 | CTAATACTTCCTTCGTTTCG |
| | | | | TTTTAGTTGATCCTTTTTCT |
| | | | | AAAAATATTTATTTTAATTT |
| | | | | AGTTGTCTCTTTGATGAAAT |
| | | | | CTAGAGAATTTTATTATATT |
| | | | | C |
| 319 | 10_212737143 | 10 | 212737143 | ATTTAGGACATCCAACGGTC |
| | | | | CAATTGAAATATGTGGTTTC |
| | | | | ATTTAGGATGATTTTTTTTT |
| | | | | TTTTACAGGAAGCTTGATTT |
| | | | | TTTCAAAATAACTTATTTAG |
| | | | | TGT |
| 320 | 10_212748182 | 10 | 212748182 | GAATCTACAACCCCATAAGC |
| | | | | TATTGACAATATGGAACCTA |
| | | | | CAATATAAAAGAATCATATT |
| | | | | GTATCAATTGTTATATAATT |
| | | | | ATACAAAATAAACTAAAATA |
| | | | | A |
| 321 | 10_213692474 | 10 | 213692474 | TGTACTGGTATGCATGTGAG |
| | | | | GTAGGCTAAATGCATAGGGT |
| | | | | TCATATGCATGAATAATAAT |
| | | | | AATAATAATAATAATAATAA |
| | | | | TAATAACTGACTAACTAACA |
| | | | | TGAGTGTGAGAAT |
| 322 | 10_213775078 | 10 | 213775078 | ATCCACCAAATCATTCAATC |
| | | | | TCAAATATAGCAAATTCAAC |
| | | | | AAATTATGATTAAAAAAAAA |
| | | | | AAGAGAACGGAGTAAAGAAA |
| | | | | TGAACCATTTGATAATCAAT |
| | | | | T |
| 323 | 10_213778888 | 10 | 213778888 | ATCACTTGTGACATTTTAAT |
| | | | | AACACAGCATATGTGTCATT |
| | | | | TCTTTCTTCTTCATTTGTCA |
| | | | | AACATATAACTCTACCTTTG |
| | | | | TTATACATCTCTCACATGGA |
| | | | | A |

The findings disclosed herein support the inventor's hypothesis that CM192-539 line, (the male parent of TM198-63 hybrid F2 population) has unique genes/DNA sequences that are in linkage to or associated with high yield characteristics (e.g. of parthenocarpic fruits) and/or with the EP trait.

Table 7 below summarizes unique genes/DNA marker sequences revealed by the present invention.

TABLE 7
Summary of line CM 192-539 unique
genes/DNA marker sequences

| | Markers | | Genes | |
|---|---|---|---|---|
| Chromosome | Total No. | SEQ ID NO | Total No. | SEQ ID NO |
| 1 | 10 | 281-290 | 93 | 1-93 |
| 10 | 33 | 291-323 | 186 | 94-280 |

It is emphasized that the above results show genomic regions with distinct haplotypes, namely, where samples of CM202-2258, CM-192-539 and ‘Maor’/CM334 have different haplotypes.

The genetic and phenotypic analysis results show that the CM-192-539 source line has a unique haplotype that is linked to the EP trait associated with the high yield phenotype and early fruit setting/setting fruit at high temperature and/or setting parthenocarpic fruit at high temperature, of the present invention. This haplotype is absent in the CM202-2258 control line which has significantly lower EP level and in the ‘Maor’/CM334 lines used as the reference lines.

### Example 3

### Genomic Analysis of QTL1 on Chromosome 1 (Chr 1) in F2 Population

A variance between the parental lines CM-192-539 and MAOR/CM334 was found within QTL1, located on gene Ca01g05320 (herein referred to as CaAGLL). The amino acid sequence encoded by this gene (Ca01g05320) is as set forth in SEQ ID NO: 27.

It is herein acknowledged that the CaAGLL gene belongs to a gene family called “Agamous like MADS box protein” (AGL62-like). This family belongs to a large gene family that is called transcription factor (TF) proteins. According to NCIMB, CaAGLL is a homolog of agamous-like MADS-box protein AGL29 [Capsicum annuum] (LOC124890154, LOC107863471), MADS-box transcription factor 27-like (Solanum lycopersicum) and to AT2G24840, AGAMOUS-LIKE 61, AGL61, DIA, DIANA in Arabidopsis. “Agamous like” proteins are reported to be involved in parthenocarpy.

The herein found variance in CaAGLL gene, was further analyzed on the F2 population. This analysis resulted in the identification of one SNP molecular marker located on the CaAGLL gene. The CaAGLL SNP (A instead of G) is located on Chr 1 positioned 9,570,115 bp on genome Ref #Capsicum annuum cv CM334 v1.55 and 12,239,886 bp on Maor Genome Ref #. The SNP causes a change of amino acid in the protein sequence from GLICYNE to SERINE. This change could influence the transcript level and the functionality of the protein and explain the involveness of this gene in parthenocarpy level in the tested F2 population.

This SNP was used for molecular marker-assisted analysis of the F2 population. The findings support that individuals that contain the allelic variation of CaAGLL A instead of G have 15-20% more fruits compared to individuals containing the WT allele (G) (see Table 8).

It is noted that this molecular variant or marker could explain 18-20% of the Excellent Parthenocarpy (EP) trait. In QTL terms it is considered as an important QTL for this trait.

TABLE 8
Unique SNP within CaAGLL gene on QTL1 of Chr 1

| | | No of | No of | CM- | | | | | |
|---|---|---|---|---|---|---|---|---|---|
| | | Fruits | Fruits | 192- | | | | | |
| Allele | Allele | (Average | (Average | 539 | Maor | Molecular | | R- | |
| a | b | allele a) | allele b) | P1 | P2 | Marker | Position | Square | p-Value |
| A | G | 137 | 120 | A | G | CaAGLL | 9570115 | 0.185 | p < 0.0001 |
| | | | | | | | (genome Ref# | | |
| | | | | | | | Capsicum | | |
| | | | | | | | annuum cv | | |
| | | | | | | | CM334 | | |
| | | | | | | | release1.55) | | |
| | | | | | | | 12,239,886 | | |
| | | | | | | | (genome Ref# | | |
| | | | | | | | Maor | | |
| | | | | | | | ASM2707369v1 | | |
| | | | | | | | NCBI) | | |

Tables 6 and 8 show unique SNPs for QTL1 located on chromosome 1 (i) between positions 09.0-13.5 Mbp relative to Capsicum annuum cv. CM334 reference genome, and (ii) at position 12,239,886 as indicated in Capsicum annuum cv Maor genome ASM2707369v1 NCBI, associated with increased fruit number and/or fruit weight property of the EP trait.

### Example 4

### Genomic Analysis of QTL2 on Chromosome 10 (Chr 10) in F2 Population

QTL2 on Chr 10 (EP in hot conditions/early fruit setting traits) was further analyzed in F2 population.

A total of 399 molecular markers were identified and from that list, molecular markers that segregate in mendelian proportion were filtered. From the filtered list, all the molecular markers with “R-Square” above 0.1 were selected. That yielded a list of 97 molecular markers with linkage to the EP trait. All these molecular markers are flanking and scattered on QTL2 (see Table 9).

The findings support that the allelic variations are associated with an increase of about 20-40% in the EP level (see Table 9, for example marker 146357 at position 210564909).

This QTL has R-square of 0.4 (the QTL Peak) meaning that QTL2 could explain 40% of the EP trait (e.g. marker 146357 at position 210564909 where a change from A to T was found).

TABLE 9
Unique SNPs on QTL2 of Chr 10 (Genome reference Capsicum annuum cv CM334 v1.55)

| | | | | | Position | | | | |
|---|---|---|---|---|---|---|---|---|---|
| | | | | | (genome Ref# | | | | |
| | | | | Molecular | Capsicum | | | | |
| | | EP | EP | Marker | annuum cv | | | CM-192- | |
| Allele | Allele | (Average | (Average | (Serial | CM334 | | | 539 | MAOR |
| a | b | allele a) | allele b) | Number) | (Release1.55) | R-Square | p-Value | P2 | P1 |
| C | T | 3.34 | 2.87 | 145808 | 204079966 | 0.365 | p < 0.0001 | C | T |
| T | C | 3.31 | 2.97 | 145933 | 206006853 | 0.214 | p < 0.0001 | T | C |
| T | A | 3.36 | 2.89 | 145935 | 206040933 | 0.371 | p < 0.0001 | T | A |
| C | T | 3.32 | 2.89 | 146059 | 207234334 | 0.276 | p < 0.0001 | C | T |
| T | C | 3.31 | 2.94 | 146067 | 207311998 | 0.237 | p < 0.0001 | T | C |
| A | G | 3.32 | 2.94 | 146094 | 208017242 | 0.272 | p < 0.0001 | A | G |
| C | T | 3.31 | 2.93 | 146281 | 209904182 | 0.114 | p < 0.0001 | C | T |
| T | C | 3.18 | 2.93 | 146282 | 209904264 | 0.103 | p < 0.0001 | T | C |
| G | A | 3.36 | 2.87 | 146356 | 210564835 | 0.391 | p < 0.0001 | G | A |
| T | A | 3.38 | 2.87 | 146357 | 210564909 | 0.417 | p < 0.0001 | T | A |
| C | T | 3.36 | 2.93 | 146396 | 210743072 | 0.295 | p < 0.0001 | C | T |
| G | A | 3.39 | 2.89 | 146398 | 210819645 | 0.393 | p < 0.0001 | G | A |
| A | G | 3.39 | 2.89 | 146399 | 210819791 | 0.393 | p < 0.0001 | A | G |
| T | G | 3.35 | 2.92 | 146400 | 210820018 | 0.3 | p < 0.0001 | T | G |
| G | A | 3.36 | 2.92 | 146401 | 210820058 | 0.319 | p < 0.0001 | G | A |
| C | T | 3.31 | 2.88 | 146407 | 210909279 | 0.311 | p < 0.0001 | C | T |
| A | C | 3.35 | 2.94 | 146534 | 213685989 | 0.289 | p < 0.0001 | A | C |
| A | C | 3.35 | 2.94 | 146535 | 213686017 | 0.296 | p < 0.0001 | A | C |
| G | A | 3.37 | 2.86 | 146573 | 214398109 | 0.403 | p < 0.0001 | G | A |
| C | T | 3.33 | 2.97 | 146601 | 214593001 | 0.23 | p < 0.0001 | C | T |
| T | C | 3.33 | 2.95 | 146636 | 214722404 | 0.255 | p < 0.0001 | T | C |
| C | G | 3.36 | 2.88 | 146649 | 214853116 | 0.37 | p < 0.0001 | C | G |
| A | G | 3.36 | 2.89 | 146701 | 215238239 | 0.351 | p < 0.0001 | A | G |
| A | G | 3.33 | 2.88 | 146703 | 215250773 | 0.359 | p < 0.0001 | A | G |
| T | C | 3.33 | 2.88 | 146704 | 215250835 | 0.359 | p < 0.0001 | T | C |
| A | G | 3.34 | 2.89 | 146810 | 215940270 | 0.356 | p < 0.0001 | A | G |
| G | A | 3.34 | 2.89 | 146834 | 216217080 | 0.342 | p < 0.0001 | G | A |
| T | A | 3.35 | 2.88 | 146924 | 216959296 | 0.378 | p < 0.0001 | T | A |
| A | G | 3.3 | 2.98 | 146959 | 217149552 | 0.173 | p < 0.0001 | A | G |
| C | T | 3.3 | 2.98 | 146960 | 217149565 | 0.173 | p < 0.0001 | C | T |
| T | C | 3.29 | 2.98 | 146961 | 217149575 | 0.168 | p < 0.0001 | T | C |
| T | G | 3.34 | 2.96 | 146965 | 217245544 | 0.254 | p < 0.0001 | T | G |
| G | T | 3.36 | 2.88 | 146999 | 217444730 | 0.38 | p < 0.0001 | T | G |
| A | G | 3.35 | 2.93 | 147009 | 217492476 | 0.288 | p < 0.0001 | A | G |
| A | C | 3.35 | 2.92 | 147010 | 217492488 | 0.295 | p < 0.0001 | A | C |
| A | G | 3.39 | 2.87 | 147013 | 217501066 | 0.437 | p < 0.0001 | A | G |
| C | T | 3.34 | 2.89 | 147014 | 217501349 | 0.359 | p < 0.0001 | C | T |
| C | A | 3.33 | 2.92 | 147019 | 217504354 | 0.276 | p < 0.0001 | C | A |
| C | G | 3.35 | 2.88 | 147021 | 217524569 | 0.365 | p < 0.0001 | C | G |
| G | C | 3.35 | 2.88 | 147030 | 217538084 | 0.371 | p < 0.0001 | G | C |
| G | A | 3.35 | 2.88 | 147031 | 217538473 | 0.371 | p < 0.0001 | G | A |
| A | G | 3.35 | 2.92 | 147035 | 217613274 | 0.318 | p < 0.0001 | A | G |
| C | G | 3.35 | 2.92 | 147036 | 217613308 | 0.318 | p < 0.0001 | C | G |
| T | C | 3.35 | 2.92 | 147037 | 217613345 | 0.318 | p < 0.0001 | T | C |
| G | A | 3.35 | 2.92 | 147038 | 217613384 | 0.311 | p < 0.0001 | G | A |
| G | A | 3.43 | 3.02 | 147039 | 217613401 | 0.218 | p < 0.0001 | G | A |
| A | G | 3.4 | 2.91 | 147040 | 217613486 | 0.375 | p < 0.0001 | A | G |
| T | G | 3.35 | 2.92 | 147041 | 217613553 | 0.318 | p < 0.0001 | T | G |
| A | G | 3.35 | 2.92 | 147042 | 217613566 | 0.318 | p < 0.0001 | A | G |
| C | T | 3.35 | 2.92 | 147043 | 217613583 | 0.318 | p < 0.0001 | C | T |
| C | A | 3.35 | 2.92 | 147044 | 217613587 | 0.318 | p < 0.0001 | C | A |
| C | T | 3.35 | 2.88 | 147045 | 217613736 | 0.371 | p < 0.0001 | C | T |
| G | A | 3.35 | 2.89 | 147046 | 217613791 | 0.375 | p < 0.0001 | G | A |
| A | G | 3.35 | 2.92 | 147047 | 217613899 | 0.303 | p < 0.0001 | A | G |
| A | T | 3.35 | 2.91 | 147048 | 217613910 | 0.313 | p < 0.0001 | A | T |
| A | G | 3.36 | 2.88 | 147062 | 217629488 | 0.384 | p < 0.0001 | A | G |
| G | A | 3.35 | 2.88 | 147063 | 217629537 | 0.38 | p < 0.0001 | G | A |
| A | C | 3.35 | 2.88 | 147064 | 217629540 | 0.38 | p < 0.0001 | A | C |
| T | G | 3.28 | 2.96 | 147065 | 217631692 | 0.19 | p < 0.0001 | T | G |
| C | A | 3.28 | 2.96 | 147066 | 217631722 | 0.19 | p < 0.0001 | C | A |
| A | T | 3.28 | 2.96 | 147067 | 217631735 | 0.19 | p < 0.0001 | A | T |
| T | G | 3.28 | 2.96 | 147068 | 217631744 | 0.19 | p < 0.0001 | T | G |
| G | C | 3.28 | 2.96 | 147069 | 217631746 | 0.19 | p < 0.0001 | G | C |
| C | A | 3.35 | 2.88 | 147167 | 217802876 | 0.379 | p < 0.0001 | C | A |
| T | C | 3.36 | 2.88 | 147168 | 217802908 | 0.376 | p < 0.0001 | T | C |
| G | A | 3.35 | 2.88 | 147174 | 217874088 | 0.372 | p < 0.0001 | G | A |
| T | C | 3.35 | 2.88 | 147175 | 217874098 | 0.373 | p < 0.0001 | T | C |
| G | C | 3.35 | 2.88 | 147176 | 217874137 | 0.372 | p < 0.0001 | G | C |
| C | T | 3.35 | 2.89 | 147180 | 217874624 | 0.363 | p < 0.0001 | C | T |
| T | A | 3.35 | 2.89 | 147181 | 217874686 | 0.362 | p < 0.0001 | T | A |
| C | A | 3.35 | 2.89 | 147182 | 217874705 | 0.362 | p < 0.0001 | C | A |
| A | G | 3.32 | 2.92 | 147183 | 217875456 | 0.292 | p < 0.0001 | A | G |
| G | A | 3.32 | 2.93 | 147184 | 217875502 | 0.285 | p < 0.0001 | G | A |
| T | A | 3.32 | 2.92 | 147185 | 217875804 | 0.289 | p < 0.0001 | T | A |
| C | T | 3.35 | 2.88 | 147186 | 217876561 | 0.379 | p < 0.0001 | C | T |
| T | C | 3.35 | 2.89 | 147187 | 217876777 | 0.344 | p < 0.0001 | T | C |
| A | G | 3.35 | 2.88 | 147188 | 217876872 | 0.374 | p < 0.0001 | A | G |
| G | T | 3.35 | 2.88 | 147189 | 217876940 | 0.374 | p < 0.0001 | G | T |
| A | G | 3.35 | 2.89 | 147190 | 217876961 | 0.342 | p < 0.0001 | A | G |
| G | C | 3.35 | 2.88 | 147191 | 217876970 | 0.374 | p < 0.0001 | G | C |
| C | A | 2.28 | 2.94 | 147192 | 217877084 | 0.213 | p < 0.0001 | C | A |
| T | C | 3.29 | 2.94 | 147193 | 217877116 | 0.219 | p < 0.0001 | T | C |
| T | C | 3.29 | 2.95 | 147194 | 217877164 | 0.217 | p < 0.0001 | T | C |
| G | T | 3.3 | 2.95 | 147195 | 217877182 | 0.232 | p < 0.0001 | G | T |
| T | C | 3.36 | 2.88 | 147197 | 217933099 | 0.376 | p < 0.0001 | T | C |
| G | A | 3.39 | 2.99 | 147198 | 217933230 | 0.329 | p < 0.0001 | G | A |
| G | A | 3.4 | 2.99 | 147199 | 217933311 | 0.353 | p < 0.0001 | G | A |
| G | A | 3.41 | 2.99 | 147200 | 217933467 | 0.349 | p < 0.0001 | G | A |
| A | G | 3.39 | 2.99 | 147201 | 217933523 | 0.329 | p < 0.0001 | A | G |
| C | T | 3.39 | 2.99 | 147202 | 217933527 | 0.329 | p < 0.0001 | C | T |
| G | A | 3.34 | 2.9 | 147204 | 217933778 | 0.355 | p < 0.0001 | G | A |
| A | G | 3.35 | 2.9 | 147206 | 217933882 | 0.362 | p < 0.0001 | A | G |
| A | G | 3.34 | 2.9 | 147207 | 217933937 | 0.353 | p < 0.0001 | A | G |
| G | A | 3.36 | 2.88 | 147208 | 217934044 | 0.386 | p < 0.0001 | G | A |
| A | C | 3.34 | 2.89 | 147215 | 217949286 | 0.354 | p < 0.0001 | C | A |
| T | A | 3.35 | 2.88 | 147224 | 217950632 | 0.379 | p < 0.0001 | T | A |
| A | G | 3.31 | 2.97 | 147227 | 217954424 | 0.224 | p < 0.0001 | A | G |

Tables 6 and 9 show unique SNPs for QTL2 located on chromosome 10 (i) between positions 204-218 Mbp relative to Capsicum annuum cv. CM334 reference genome, and (ii) between positions 199-214 Mbp relative to Maor genome reference #ASM 2707369v1 NCBI, associated with the EP trait expressed by or having the property of early fruit setting/setting fruit at high temperature and/or setting parthenocarpic fruit at high temperature.

By combining the yield results and the genetic data derived from the experiments above, it is strongly evidenced that CM 192-539 line is a source of the unique EP (excellent parthenocarpy) trait which led to:
a. Yield increase (fruit number and/or weight), for which unique genes/DNA sequences associated with the trait were identified on QTL1 of chromosome 1 (SEQ ID NO: 1-93, SEQ ID NO: 281-290 and Table 8); and
b. Early fruit setting/setting fruit at high temperature and/or setting parthenocarpic fruit at high temperature, for which unique genes/DNA sequences associated with the trait were identified on QTL2 of Chromosome 10 (SEQ ID NO: 94-280, SEQ ID NO: 291-323 and Table 9).

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof. As used herein the terms “comprises”, “comprising”, “includes”, “including”, “having” and their conjugates mean “including but not limited to”. The term “consisting of” means “including and limited to”.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “average” refers to the mean value as obtained by measuring a predetermined parameter in each plant of a certain plant population and calculating the mean value according to the number of plants in said population.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Whenever the term “about” is used, it is meant to refer to a measurable value such as an amount, a temporal duration, and the like, and is meant to encompass variations of ±20%, ±10%, ±5%, ±1%, or ±0.1% from the specified value, as such variations are appropriate to perform the disclosed methods.

All publications, patent applications, patents, and other references mentioned in the disclosures of these publications in their entireties are hereby incorporated by reference into this application in order to more fully describe the state of the art to which this invention pertains. In case of conflict, the patent specification, including definitions, will prevail. In addition, the materials, methods, and examples are illustrative only and not intended to be limiting. Throughout this application various publications, published patent applications and published patents are referenced.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description. While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention. Various embodiments have been presented. Each of these embodiments may of course include features from other embodiments presented, and embodiments not specifically described may include various features described herein.

### REFERENCES

K. D. Murray, J. O. Borevitz, Axe: rapid, competitive sequence read demultiplexing using a trie, Bioinformatics 34 (2018) 3924-3925.
A. McKenna, M. Hanna, E. Banks, A. Sivachenko, K. Cibulskis, A. Kernytsky, K. Garimella, D. Altshuler, S. Gabriel, M. Daly, M. A. DePristo, The genome analysis toolkit: a mapreduce framework for analyzing.
A. McKenna, M. Hanna, E. Banks, A. Sivachenko, K. Cibulskis, A. Kernytsky, K. Garimella, D. Altshuler, S. Gabriel, M. Daly, M. A. DePristo, The genome analysis toolkit: a mapreduce framework for analyzing.
Poplin, V. Ruano-Rubio, M. A. DePristo, T. J. Fennell, M. O. Carneiro, G. A. Van der Auwera, D. E. Kling, L. D. Gauthier, A. Levy-Moonshine, D. Roazen, K. Shakir, J. Thibault, S. Chandran, C. Whelan, M. Lek, S. Gabriel, M. J. Daly, B. Neale, D. G. MacArthur, E. Banks, Scaling accurate genetic variant discovery to tens of thousands of samples, bioRxiv (2018), 201178.
R. J. Elshire, J. C. Glaubitz, Q. Sun, J. A. Poland, K. Kawamoto, E. S. Buckler, S. E. Mitchell, A. Robust, Simple genotyping-by-sequencing (GBS) approach for high diversity species, PLoS One 6 (2011), e19379.
A Tiwari, A. Vivian-Smith, R. E. Voorrips, M. E. Habets, L. B. Xue, R. Offringa, E. P. Heuvelink. Parthenocarpic potential in Capsicum annuumL. is enhanced by carpelloid structures and controlled by a single recessive gene. BMC Plant Biology 11.1 (2011): 1-15.
I. Honda, H. Matsunaga, H., Kikuchi, K., Matsuo, S., & Fukuda, M. Identification of pepper (Capsicum annuum L.) accessions with large or small fruit that have a high degree of parthenocarpy. Scientia horticulturae 135 (2012): 68-70.

What is claimed is:

1. A cultivated pepper plant or seed capable of producing high yield properties of parthenocarpic fruits, wherein said plant or seed comprises at least one Quantitative Trait Locus (QTL) for an Excellent Parthenocarpy (EP) trait, said EP trait conferring production of elevated yield properties of parthenocarpic seedless fruits, as compared to a pepper plant having the same genetic background and lacking said EP trait, said QTL is selected from QTL1 located 09.0-13.5 Mbp on chromosome 1, and QTL2 located 199-218 Mbp on chromosome 10.

2. The cultivated pepper plant or seed according to claim 1, wherein said plant is male sterile, the male sterility is selected from a cytoplasmic male-sterile (CMS) trait, a genomic male sterile trait (GMS) or a combination thereof, and/or wherein
said pepper plant or seed is an inbred, a dihaploid or a hybrid and/or a genome edited plant, such as a plant or seed produced using the CRISPR/Cas system.

3. The cultivated pepper plant or seed according to claim 1, wherein
a. QTL1 located on chromosome 1 is associated with the property of elevated fruit number per plant and/or elevated fruit weight per plant, and
b. QTL2 located on chromosome 10 is associated with the property of early fruit setting and/or setting parthenocarpic fruits at high temperature and/or setting fruit at high temperature.

4. The cultivated pepper plant or seed according to claim 1, wherein the genome of said plant comprises at least one molecular marker and/or gene associated with the at least one QTL, the at least one molecular marker and/or gene is selected from the group consisting of:
a. a molecular marker selected from (i) SEQ ID NO: 281-290 or any combination thereof, (ii) allele A at position 9570115 as indicated in pepper genome Capsicum annuum cv CM334 v1.55 or at position 12,239,886 as indicated in Capsicum annuum cv Maor genome ASM2707369v1 NCBI, and/or (iii) a gene encoding a sequence selected from SEQ ID NO: 1-93 or any combination thereof, (iv) any combination thereof, associated with QTL1 on chromosome 1;
b. a molecular marker selected from (i) SEQ ID NO: 291-323 or any combination thereof, (ii) an allele selected from the group consisting of:

| | Position (Capsicum annuum cv |
|---|---|
| Allele | CM334 v1.55 reference genome) |
| C | 204079966 |
| T | 206006853 |
| T | 206040933 |
| C | 207234334 |
| T | 207311998 |
| A | 208017242 |
| C | 209904182 |
| T | 209904264 |
| G | 210564835 |
| T | 210564909 |
| C | 210743072 |
| G | 210819645 |
| A | 210819791 |
| T | 210820018 |
| G | 210820058 |
| C | 210909279 |
| A | 213685989 |
| A | 213686017 |
| G | 214398109 |
| C | 214593001 |
| T | 214722404 |
| C | 214853116 |
| A | 215238239 |
| A | 215250773 |
| T | 215250835 |
| A | 215940270 |
| G | 216217080 |
| T | 216959296 |
| A | 217149552 |
| C | 217149565 |
| T | 217149575 |
| T | 217245544 |
| G | 217444730 |
| A | 217492476 |
| A | 217492488 |
| A | 217501066 |
| C | 217501349 |
| C | 217504354 |
| C | 217524569 |
| G | 217538084 |
| G | 217538473 |
| A | 217613274 |
| C | 217613308 |
| T | 217613345 |
| G | 217613384 |
| G | 217613401 |
| A | 217613486 |
| T | 217613553 |
| A | 217613566 |
| C | 217613583 |
| C | 217613587 |
| C | 217613736 |
| G | 217613791 |
| A | 217613899 |
| A | 217613910 |
| A | 217629488 |
| G | 217629537 |
| A | 217629540 |
| T | 217631692 |
| C | 217631722 |
| A | 217631735 |
| T | 217631744 |
| G | 217631746 |
| C | 217802876 |
| T | 217802908 |
| G | 217874088 |
| T | 217874098 |
| G | 217874137 |
| C | 217874624 |
| T | 217874686 |
| C | 217874705 |
| A | 217875456 |
| G | 217875502 |
| T | 217875804 |
| C | 217876561 |
| T | 217876777 |
| A | 217876872 |
| G | 217876940 |
| A | 217876961 |
| G | 217876970 |
| C | 217877084 |
| T | 217877116 |
| T | 217877164 |
| G | 217877182 |
| T | 217933099 |
| G | 217933230 |
| G | 217933311 |
| G | 217933467 |
| A | 217933523 |
| C | 217933527 |
| G | 217933778 |
| A | 217933882 |
| A | 217933937 |
| G | 217934044 |
| A | 217949286 |
| T | 217950632 |
| A | 217954424 |

or any combination thereof, and/or (iii) a gene encoding a sequence selected from SEQ ID NO: 94-280 or any combination thereof, (iv) any combination thereof, associated with QTL2 on chromosome 10; and
c. any combination thereof.

5. The cultivated pepper plant or seed according to claim 1, wherein the high yield properties of parthenocarpic fruits comprise at least one of: early fruit setting, setting fruit at high temperature, setting parthenocarpic fruits at high temperature, elevated fruit number per plant and/or elevated fruit weight per plant.

6. The cultivated pepper plant or seed according to claim 5, wherein
a. the property of elevated fruit number per plant and/or elevated fruit weight per plant is associated with molecular markers and/or genes selected from (i) SEQ ID NO: 281-290 or any combination thereof, (ii) allele A at position 9570115 as indicated in pepper genome Capsicum annuum cv CM334 v1.55 or at position 12,239,886 as indicated in Capsicum annuum cv Maor genome ASM2707369v1 NCBI, and/or (iii) a gene encoding a sequence selected from SEQ ID NO: 1-93 or any combination thereof, (iv) any combination thereof, associated with QTL1 on chromosome, and
b. the property of early fruit setting and/or setting fruit at high temperature and/or setting parthenocarpic fruits at high temperature, is associated with molecular markers and/or genes selected from (i) SEQ ID NO: 291-323 or any combination thereof, (ii) an allele selected from the group consisting of:

| | Position (Capsicum annuum cv |
|---|---|
| Allele | CM334 v1.55 reference genome) |
| C | 204079966 |
| T | 206006853 |
| T | 206040933 |
| C | 207234334 |
| T | 207311998 |
| A | 208017242 |
| C | 209904182 |
| T | 209904264 |
| G | 210564835 |
| T | 210564909 |
| C | 210743072 |
| G | 210819645 |
| A | 210819791 |
| T | 210820018 |
| G | 210820058 |
| C | 210909279 |
| A | 213685989 |
| A | 213686017 |
| G | 214398109 |
| C | 214593001 |
| T | 214722404 |
| C | 214853116 |
| A | 215238239 |
| A | 215250773 |
| T | 215250835 |
| A | 215940270 |
| G | 216217080 |
| T | 216959296 |
| A | 217149552 |
| C | 217149565 |
| T | 217149575 |
| T | 217245544 |
| G | 217444730 |
| A | 217492476 |
| A | 217492488 |
| A | 217501066 |
| C | 217501349 |
| C | 217504354 |
| C | 217524569 |
| G | 217538084 |
| G | 217538473 |
| A | 217613274 |
| C | 217613308 |
| T | 217613345 |
| G | 217613384 |
| G | 217613401 |
| A | 217613486 |
| T | 217613553 |
| A | 217613566 |
| C | 217613583 |
| C | 217613587 |
| C | 217613736 |
| G | 217613791 |
| A | 217613899 |
| A | 217613910 |
| A | 217629488 |
| G | 217629537 |
| A | 217629540 |
| T | 217631692 |
| C | 217631722 |
| A | 217631735 |
| T | 217631744 |
| G | 217631746 |
| C | 217802876 |
| T | 217802908 |
| G | 217874088 |
| T | 217874098 |
| G | 217874137 |
| C | 217874624 |
| T | 217874686 |
| C | 217874705 |
| A | 217875456 |
| G | 217875502 |
| T | 217875804 |
| C | 217876561 |
| T | 217876777 |
| A | 217876872 |
| G | 217876940 |
| A | 217876961 |
| G | 217876970 |
| C | 217877084 |
| T | 217877116 |
| T | 217877164 |
| G | 217877182 |
| T | 217933099 |
| G | 217933230 |
| G | 217933311 |
| G | 217933467 |
| A | 217933523 |
| C | 217933527 |
| G | 217933778 |
| A | 217933882 |
| A | 217933937 |
| G | 217934044 |
| A | 217949286 |
| T | 217950632 |
| A | 217954424 |

c. or any combination thereof, and/or (iii) a gene encoding a sequence selected from SEQ ID NO: 94-280 or any combination thereof, (iv) any combination thereof, associated with QTL2 on chromosome 10.

7. The cultivated pepper plant or seed according to claim 4, wherein
a. the plant comprising the at least one molecular marker and/or gene associated with QTL1 have 15-20% more fruits compared to a plant lacking said at least one QTL1 associated molecular marker and/or gene, and
b. the plant comprising the at least one molecular marker and/or gene associated with QTL2 have an average EP value of above 3, particularly in the range of 3-8, or wherein the plant comprising the at least one molecular marker and/or gene associated with QTL2 exhibits an increase in EP value of about 20-40% compared to a plant lacking said at least one QTL2 associated molecular marker and/or gene.

8. The cultivated pepper plant or seed according to claim 1, wherein
a. said plant is capable of forming fruits from at least about 90% of the flowers on said plant,
b. the high yield properties comprise (i) increased parthenocarpic fruit number per plant of at least about 5%, particularly 10-25%, and/or (ii) increased fruit weight per plant of at least about 15%, particularly 20-35%,
c. said plant produces at least 38 fruits per plant, such as at least 65 fruits per plant, particularly at least 90 fruits per plant,
d. said plant has fruit yield of at least about 1.5 kg per plant, preferably at least 3.6 kg per plant, more preferably at least 5.0 kg per plant,
e. said plant produces a fruit type selected from the group consisting of: bell pepper, pointed pepper, half long pepper, Como di Toro pepper, sweet pepper including a dolce-type pepper, a big rectangular pepper, a conical pepper, a long conical pepper and a blocky-type pepper,
f. the mature fruit of the plant is green, yellow, orange, red, ivory, brown, or purple, and/or
g. from at least 60% of the fruits grown on said plant, at least 95% are seedless.

9. The cultivated pepper plant or seed according to claim 1 or a progeny thereof, wherein said at least one QTL for an Excellent Parthenocarpy (EP) trait is as found in seeds of Capsicum annuum CM-192-539, representative seeds of which was deposited with NCIMB Aberdeen AB21 9YA, Scotland, UK under accession number NCIMB 44203 on 04/08/2023.

10. A plant part of a cultivated pepper plant or seed according to claim 1, wherein said plant part is defined as a leaf, a bud, a meristem, an embryo, a root, a root tip, a stem, a flower, a fruit, seed, pollen, ovule a cell, regenerable cell, tissue culture of regenerable cells, protoplasts or callus, or processed pepper fruit derived from a cultivated pepper plant or seed according to claim 1.

11. A method for producing a pepper plant or seed according to claim 1, wherein said method comprises steps of:
a. producing and selecting a first pepper plant as a donor male parent, comprising at least one Quantitative Trait Locus (QTL) for an Excellent Parthenocarpy (EP) trait, said EP trait conferring production of elevated yield properties of parthenocarpic seedless fruits, as compared to a pepper plant having the same genetic background and lacking said EP trait, said QTL is selected from QTL1 located 09.0-13.5 Mbp on chromosome 1, and QTL2 located 199-218 Mbp on chromosome 10;
b. crossing said first pepper plant with a second pepper plant as a female parent comprising a cytoplasmic male sterile (CMS) trait, to produce progeny hybrid pepper plants;
c. selecting at least one progeny hybrid plant showing elevated yield properties of parthenocarpic seedless fruits as compared to the yield of a pepper plant having the same genetic background and lacking said EP trait; and
d. optionally, backcrossing said at least one selected progeny plant with said male parent plant and/or repeating steps c-d.

12. The method according to claim 11, wherein said step of producing and selecting a pepper plant as a donor male parent comprises steps of screening F2 seeds of various pepper genetic sources for being the donor male parent comprising the EP trait by:
a. crossing F2 plants of the F2 seeds as a male parent, with a first preselected CMS line plant as a recurrent female parent, for producing a first parthenocarpic hybrid progeny set;
b. selecting from said first hybrid set a first parthenocarpic hybrid plant producing the highest parthenocarpic fruit yield relative to the other hybrid progeny plants derived from the same first recurrent female parental line;
c. selfing the F2 male parent plant of the first selected parthenocarpic hybrid to produce F3 seeds;
d. crossing plants of the F3 seeds as a male parent with the first CMS recurrent female parent to produce a second parthenocarpic hybrid progeny set and selecting from said second hybrid set a second parthenocarpic hybrid plant producing the highest parthenocarpic fruit yield relative to the other hybrid plants;
e. repeating steps a to d with the relevant/next generation seeds or plants to produce a fertile pepper plant as a donor male parent comprising the EP trait, conferring production of elevated yield of commercially acceptable parthenocarpic seedless fruits.

13. The method according to claim 12, wherein said step of screening comprises steps of producing a doubled haploid (DH) genotype plants from haploid cells derived from various pepper genetic sources.

14. A method for detecting or selecting cultivated pepper plant or seed according to claim 1, capable of producing high yield properties of parthenocarpic fruits, said method comprising the steps of:
assaying pepper plants for at least one molecular marker genetically linked to at least one QTL selected from QTL1 located 09.0-13.5 Mbp on chromosome 1 and QTL2 located 199-218 Mbp on chromosome 10, said molecular marker is selected from:
a. a molecular marker selected from (i) SEQ ID NO: 281-290 or any combination thereof, (ii) allele A at position 9570115 as indicated in pepper genome Capsicum annuum cv CM334 v1.55 or at position 12,239,886 as indicated in Capsicum annuum cv Maor genome ASM2707369v1 NCBI, and/or (iii) a gene encoding a sequence selected from SEQ ID NO: 1-93 or any combination thereof, (iv) or any combination thereof, associated with QTL1 on chromosome 1;
b. a molecular marker selected from (i) SEQ ID NO: 291-323 or any combination thereof, (ii) an allele selected from the group consisting of:

| | Position (Capsicum annuum cv |
|---|---|
| Allele | CM334 v1.55 reference genome) |
| C | 204079966 |
| T | 206006853 |
| T | 206040933 |
| C | 207234334 |
| T | 207311998 |
| A | 208017242 |
| C | 209904182 |
| T | 209904264 |
| G | 210564835 |
| T | 210564909 |
| C | 210743072 |
| G | 210819645 |
| A | 210819791 |
| T | 210820018 |
| G | 210820058 |
| C | 210909279 |
| A | 213685989 |
| A | 213686017 |
| G | 214398109 |
| C | 214593001 |
| T | 214722404 |
| C | 214853116 |
| A | 215238239 |
| A | 215250773 |
| T | 215250835 |
| A | 215940270 |
| G | 216217080 |
| T | 216959296 |
| A | 217149552 |
| C | 217149565 |
| T | 217149575 |
| T | 217245544 |
| G | 217444730 |
| A | 217492476 |
| A | 217492488 |
| A | 217501066 |
| C | 217501349 |
| C | 217504354 |
| C | 217524569 |
| G | 217538084 |
| G | 217538473 |
| A | 217613274 |
| C | 217613308 |
| T | 217613345 |
| G | 217613384 |
| G | 217613401 |
| A | 217613486 |
| T | 217613553 |
| A | 217613566 |
| C | 217613583 |
| C | 217613587 |
| C | 217613736 |
| G | 217613791 |
| A | 217613899 |
| A | 217613910 |
| A | 217629488 |
| G | 217629537 |
| A | 217629540 |
| T | 217631692 |
| C | 217631722 |
| A | 217631735 |
| T | 217631744 |
| G | 217631746 |
| C | 217802876 |
| T | 217802908 |
| G | 217874088 |
| T | 217874098 |
| G | 217874137 |
| C | 217874624 |
| T | 217874686 |
| C | 217874705 |
| A | 217875456 |
| G | 217875502 |
| T | 217875804 |
| C | 217876561 |
| T | 217876777 |
| A | 217876872 |
| G | 217876940 |
| A | 217876961 |
| G | 217876970 |
| C | 217877084 |
| T | 217877116 |
| T | 217877164 |
| G | 217877182 |
| T | 217933099 |
| G | 217933230 |
| G | 217933311 |
| G | 217933467 |
| A | 217933523 |
| C | 217933527 |
| G | 217933778 |
| A | 217933882 |
| A | 217933937 |
| G | 217934044 |
| A | 217949286 |
| T | 217950632 |
| A | 217954424 |

or any combination thereof, and/or (iii) a gene encoding a sequence selected from SEQ ID NO: 94-280 or any combination thereof, (iv) any combination thereof, associated with QTL2 on chromosome 10; and
c. any combination thereof; and
selecting a plant comprising at least one of the molecular markers associated with at least one of the QTLs for EP trait conferring production of elevated yield of parthenocarpic seedless fruits, as compared to a pepper plant having the same genetic background and lacking said EP trait.

15. A Method for the production of a pepper (Capsicum annuum) plant exhibiting high yield properties of parthenocarpic fruits, by using a doubled haploid generation technique on plant material comprising the QTL as defined in claim 1 to generate a doubled haploid line comprising said trait and the QTL as defined in claim 1.

16. An allele, haplotype, QTL, molecular marker or gene being inherited to progeny plant, wherein said allele, haplotype, QTL, molecular marker or gene is associated with an Excellent Parthenocarpy (EP) trait capable of conferring production of elevated yield of parthenocarpic seedless fruits as compared to a pepper plant having the same genetic background and lacking said allele, haplotype, QTL, molecular marker or gene, said molecular marker and/or gene sequence is selected from the group consisting of:
a. a molecular marker selected from (i) SEQ ID NO: 281-290 or any combination thereof, (ii) allele A at position 9570115 as indicated in pepper genome Capsicum annuum cv CM334 v1.55 or at position 12,239,886 as indicated in Capsicum annuum cv Maor genome ASM2707369v1 NCBI, and/or (iii) a gene encoding a sequence selected from SEQ ID NO: 1-93 or any combination thereof, (iv) or any combination thereof, associated with QTL1 located 09.0-13.5 Mbp on chromosome 1;
b. a molecular marker selected from (i) SEQ ID NO: 291-323 or any combination thereof, (ii) an allele selected from the group consisting of:

| | Position (Capsicum annuum cv |
|---|---|
| Allele | CM334 v1.55 reference genome) |
| C | 204079966 |
| T | 206006853 |
| T | 206040933 |
| C | 207234334 |
| T | 207311998 |
| A | 208017242 |
| C | 209904182 |
| T | 209904264 |
| G | 210564835 |
| T | 210564909 |
| C | 210743072 |
| G | 210819645 |
| A | 210819791 |
| T | 210820018 |
| G | 210820058 |
| C | 210909279 |
| A | 213685989 |
| A | 213686017 |
| G | 214398109 |
| C | 214593001 |
| T | 214722404 |
| C | 214853116 |
| A | 215238239 |
| A | 215250773 |
| T | 215250835 |
| A | 215940270 |
| G | 216217080 |
| T | 216959296 |
| A | 217149552 |
| C | 217149565 |
| T | 217149575 |
| T | 217245544 |
| G | 217444730 |
| A | 217492476 |
| A | 217492488 |
| A | 217501066 |
| C | 217501349 |
| C | 217504354 |
| C | 217524569 |
| G | 217538084 |
| G | 217538473 |
| A | 217613274 |
| C | 217613308 |
| T | 217613345 |
| G | 217613384 |
| G | 217613401 |
| A | 217613486 |
| T | 217613553 |
| A | 217613566 |
| C | 217613583 |
| C | 217613587 |
| C | 217613736 |
| G | 217613791 |
| A | 217613899 |
| A | 217613910 |
| A | 217629488 |
| G | 217629537 |
| A | 217629540 |
| T | 217631692 |
| C | 217631722 |
| A | 217631735 |
| T | 217631744 |
| G | 217631746 |
| C | 217802876 |
| T | 217802908 |
| G | 217874088 |
| T | 217874098 |
| G | 217874137 |
| C | 217874624 |
| T | 217874686 |
| C | 217874705 |
| A | 217875456 |
| G | 217875502 |
| T | 217875804 |
| C | 217876561 |
| T | 217876777 |
| A | 217876872 |
| G | 217876940 |
| A | 217876961 |
| G | 217876970 |
| C | 217877084 |
| T | 217877116 |
| T | 217877164 |
| G | 217877182 |
| T | 217933099 |
| G | 217933230 |
| G | 217933311 |
| G | 217933467 |
| A | 217933523 |
| C | 217933527 |
| G | 217933778 |
| A | 217933882 |
| A | 217933937 |
| G | 217934044 |
| A | 217949286 |
| T | 217950632 |
| A | 217954424 |

or any combination thereof, and/or (iii) a gene encoding a sequence selected from SEQ ID NO: 94-280 or any combination thereof, (iv) any combination thereof, associated with QTL2 located 199-218 Mbp on chromosome 10; and
c. any combination thereof.

17. Use of a molecular marker selected from the group consisting of
a. a molecular marker selected from (i) SEQ ID NO: 281-290 or any combination thereof, (ii) allele A at position 9570115 as indicated in pepper genome Capsicum annuum cv CM334 v1.55 or at position 12,239,886 as indicated in Capsicum annuum cv Maor genome ASM2707369v1 NCBI, and/or (iii) a gene encoding a sequence selected from SEQ ID NO: 1-93 or any combination thereof, (iv) any combination thereof associated with QTL1 located 09-13.5 Mbp on chromosome 1;
b. a molecular marker selected from (i) SEQ ID NO: 291-323 or any combination thereof, (ii) an allele selected from the group consisting of:

| | Position (Capsicum annuum cv |
|---|---|
| Allele | CM334 v1.55 reference genome) |
| C | 204079966 |
| T | 206006853 |
| T | 206040933 |
| C | 207234334 |
| T | 207311998 |
| A | 208017242 |
| C | 209904182 |
| T | 209904264 |
| G | 210564835 |
| T | 210564909 |
| C | 210743072 |
| G | 210819645 |
| A | 210819791 |
| T | 210820018 |
| G | 210820058 |
| C | 210909279 |
| A | 213685989 |
| A | 213686017 |
| G | 214398109 |
| C | 214593001 |
| T | 214722404 |
| C | 214853116 |
| A | 215238239 |
| A | 215250773 |
| T | 215250835 |
| A | 215940270 |
| G | 216217080 |
| T | 216959296 |
| A | 217149552 |
| C | 217149565 |
| T | 217149575 |
| T | 217245544 |
| G | 217444730 |
| A | 217492476 |
| A | 217492488 |
| A | 217501066 |
| C | 217501349 |
| C | 217504354 |
| C | 217524569 |
| G | 217538084 |
| G | 217538473 |
| A | 217613274 |
| C | 217613308 |
| T | 217613345 |
| G | 217613384 |
| G | 217613401 |
| A | 217613486 |
| T | 217613553 |
| A | 217613566 |
| C | 217613583 |
| C | 217613587 |
| C | 217613736 |
| G | 217613791 |
| A | 217613899 |
| A | 217613910 |
| A | 217629488 |
| G | 217629537 |
| A | 217629540 |
| T | 217631692 |
| C | 217631722 |
| A | 217631735 |
| T | 217631744 |
| G | 217631746 |
| C | 217802876 |
| T | 217802908 |
| G | 217874088 |
| T | 217874098 |
| G | 217874137 |
| C | 217874624 |
| T | 217874686 |
| C | 217874705 |
| A | 217875456 |
| G | 217875502 |
| T | 217875804 |
| C | 217876561 |
| T | 217876777 |
| A | 217876872 |
| G | 217876940 |
| A | 217876961 |
| G | 217876970 |
| C | 217877084 |
| T | 217877116 |
| T | 217877164 |
| G | 217877182 |
| T | 217933099 |
| G | 217933230 |
| G | 217933311 |
| G | 217933467 |
| A | 217933523 |
| C | 217933527 |
| G | 217933778 |
| A | 217933882 |
| A | 217933937 |
| G | 217934044 |
| A | 217949286 |
| T | 217950632 |
| A | 217954424 |

or any combination thereof, and/or (iii) a gene encoding a sequence selected from SEQ ID NO: 94-280 or any combination thereof, (iv) any combination thereof, associated with QTL2 located 199-218 Mbp on chromosome 10; and
c. any combination thereof;
to identify or develop pepper plants comprising an Excellent Parthenocarpy (EP) trait conferring production of elevated yield of commercially acceptable parthenocarpic seedless fruits, as defined in claim 1, and/or identify the at least one QTL, and/or to develop other markers linked to the at least one QTL.

18. A method for increasing parthenocarpic seedless pepper fruit yield production to a commercially relevant extent in multiple geographical- and/or whether-related environments or areas or growth conditions comprising growing in said geographical area pepper plant or seed according to claim 1.

19. A genome modified pepper plant or seed capable of producing high yield properties of parthenocarpic fruits, wherein said plant or seed comprises EP trait conferring production of elevated yield properties of parthenocarpic seedless fruits, as compared to a pepper plant having the same genetic background and lacking said EP trait, said plant comprises genome modifications associated with said EP trait selected from:
a. at least one genome modification on chromosome 1 selected from (i) SEQ ID NO: 281-290 or any combination thereof, (ii) allele A at position 9570115 as indicated in pepper genome Capsicum annuum cv CM334 v1.55 or at position 12,239,886 as indicated in Capsicum annuum cv Maor genome ASM2707369v1 NCBI, (iii) any combination thereof;
b. at least one genome modification on chromosome 10 selected from (i) SEQ ID NO: 291-323 or any combination thereof, (ii) an allele selected from the group consisting of:

| | Position (Capsicum annuum cv |
|---|---|
| Allele | CM334 v1.55 reference genome) |
| C | 204079966 |
| T | 206006853 |
| T | 206040933 |
| C | 207234334 |
| T | 207311998 |
| A | 208017242 |
| C | 209904182 |
| T | 209904264 |
| G | 210564835 |
| T | 210564909 |
| C | 210743072 |
| G | 210819645 |
| A | 210819791 |
| T | 210820018 |
| G | 210820058 |
| C | 210909279 |
| A | 213685989 |
| A | 213686017 |
| G | 214398109 |
| C | 214593001 |
| T | 214722404 |
| C | 214853116 |
| A | 215238239 |
| A | 215250773 |
| T | 215250835 |
| A | 215940270 |
| G | 216217080 |
| T | 216959296 |
| A | 217149552 |
| C | 217149565 |
| T | 217149575 |
| T | 217245544 |
| G | 217444730 |
| A | 217492476 |
| A | 217492488 |
| A | 217501066 |
| C | 217501349 |
| C | 217504354 |
| C | 217524569 |
| G | 217538084 |
| G | 217538473 |
| A | 217613274 |
| C | 217613308 |
| T | 217613345 |
| G | 217613384 |
| G | 217613401 |
| A | 217613486 |
| T | 217613553 |
| A | 217613566 |
| C | 217613583 |
| C | 217613587 |
| C | 217613736 |
| G | 217613791 |
| A | 217613899 |
| A | 217613910 |
| A | 217629488 |
| G | 217629537 |
| A | 217629540 |
| T | 217631692 |
| C | 217631722 |
| A | 217631735 |
| T | 217631744 |
| G | 217631746 |
| C | 217802876 |
| T | 217802908 |
| G | 217874088 |
| T | 217874098 |
| G | 217874137 |
| C | 217874624 |
| T | 217874686 |
| C | 217874705 |
| A | 217875456 |
| G | 217875502 |
| T | 217875804 |
| C | 217876561 |
| T | 217876777 |
| A | 217876872 |
| G | 217876940 |
| A | 217876961 |
| G | 217876970 |
| C | 217877084 |
| T | 217877116 |
| T | 217877164 |
| G | 217877182 |
| T | 217933099 |
| G | 217933230 |
| G | 217933311 |
| G | 217933467 |
| A | 217933523 |
| C | 217933527 |
| G | 217933778 |
| A | 217933882 |
| A | 217933937 |
| G | 217934044 |
| A | 217949286 |
| T | 217950632 |
| A | 217954424 |

or any combination thereof, (iii) any combination thereof; and
c. any combination thereof.

20. The genome modified pepper plant or seed according to claim 19, wherein said genome modifications are produced using targeted genome editing technique, such as using the CRISPR/Cas9 system.
